(19) United States
(12) Patent Application Publication
(10) Pub. No.: US 2025/0223259 A1
(43) Pub. Date: Jul. 10, 2025

(54) PROTEASE INHIBITORS FOR TREATING OR PREVENTING CORONAVIRUS INFECTION

(71) Applicant: Merck Sharp & Dohme LLC, Rahway, NJ (US)

(72) Inventors: Brian T. Campbell, Doylestown, PA (US); Wonsuk Chang, Princeton, NJ (US); Timothy J. Hartingh, Richlandtown, PA (US); Danielle M. Hurzy, Garnet Valley, PA (US); Michael J. Kelly, III, Paoli, PA (US); Franca-Maria Klingler, Letchworth Garden City (GB); Mark E. Layton, Harleysville, PA (US); John A. McCauley, Maple Glen, PA (US); Christopher Charles Nawrat, Ardmore, PA (US); Craig A. Parish, Tenafly, NJ (US); James J. Perkins, Churchville, PA (US); Anthony J. Roecker, Santee, CA (US); Manuel de Lera Ruiz, Perkasie, PA (US); John D. Schreier, Harleysville, PA (US); Valerie W. Shurtleff, Philadelphia, PA (US); Jing Su, Scotch Plains, NJ (US); Quang T. Truong, Morganville, NJ (US)

(73) Assignee: Merck Sharp & Dohme LLC, Rahway, NJ (US)

(21) Appl. No.: 18/724,365

(22) PCT Filed: Jan. 5, 2023
(86) PCT No.: PCT/US2023/010161

§ 371 Date: Jun. 26, 2024

Related U.S. Application Data
(60) Provisional application No. 63/430,444, filed on Dec. 6, 2022.
(60) Provisional application No. 63/297,391, filed on Jan. 7, 2022.

(51) Int. Cl. C07D 207/16; A61K 31/40; A61K 31/438; A61P 31/14; C07D 211/60; C07D 491/20

(52) U.S. Cl. CPC C07D 207/16; A61K 31/40; A61K 31/438; A61P 31/14; C07D 211/60; C07D 491/20

(57) ABSTRACT

The present invention provides a compound of Formula I wherein A, M, R¹, R², R^{3a}, R^{3b}, and subscripts m and n are as described herein and pharmaceutical compositions comprising one or more said compounds, and methods for using said compounds for the treatment, inhibition, or amelioration of one or more disease states that could benefit from inhibition of a coronavirus, including SARS-CoV, MERS-CoV and SARS-CoV-2. The compounds of this invention could further be used in combination with other therapeutically effective agents, including but not limited to, other drugs useful for the treatment of coronavirus infection. The invention furthermore relates to processes for preparing compounds of Formula I, and pharmaceutical compositions which comprise compounds of Formula I and pharmaceutically acceptable salts thereof.

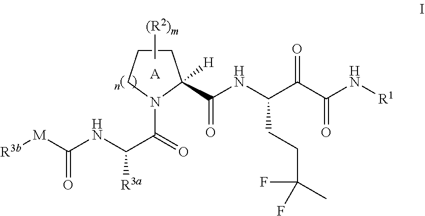

### FIELD OF THE INVENTION

The present invention relates to certain protease inhibitors, pharmaceutical compositions comprising such inhibitors, and methods for using said compounds for the treatment, inhibition or amelioration of one or more disease states that could benefit from inhibition of a coronavirus, including SARS-CoV, MERS-CoV and SARS-CoV-2.

### BACKGROUND OF THE INVENTION

Coronaviruses (CoVs) are large, enveloped, positive-stranded, RNA viruses that comprise the Coronavirinae subfamily in the Nirovirales order. CoVs are further classified into four genera: alpha coronavirus, beta coronavirus, gamma coronavirus and delta coronavirus. Alpha and beta CoVs infect humans and other mammals, whereas the gamma and delta CoVs infect only animals (e.g., birds, sea mammals, pigs). CoV infection can result in a wide range of acute to chronic diseases of the respiratory, enteric and central nervous systems (Fields Virology Emerging Viruses Vol. 1. 2021. pp. 410-412).

To date, seven different coronaviruses that cause disease in humans have been identified: HCoV-229E, HCoV-NL63, HCoV-OC43, HCoV—HKU1, severe acute respiratory syndrome coronavirus (SARS-CoV), Middle East respiratory syndrome coronavirus (MERS-CoV) and, most recently SARS-CoV-2. HCoV-229E, HCoV-NL63, HCoV-OC43 and HCoV—HKU1 circulate on a yearly basis and cause mild symptoms similar to a common cold (Fori D, Cagliani R, Clerici M, and Sironi M. 2017. Trends in Microbiology, January 2017, Vol. 25, No. 1. 35-48). SARS-CoV, MERS-CoV and SARS-CoV-2 however, which have emerged in three zoonotic CoV transmission events over the last 21 years, are associated with mild to severe symptoms of respiratory infection such as fever, cough, dyspnea, pneumonia and acute respiratory distress syndrome that can ultimately lead to death.

The SARS-CoV epidemic in 2002 to 2003 was contained, but it resulted in 8,000 SARS-CoV infections and more than 800 deaths (Fields Virology Emerging Viruses Vol. 1. 2021. pp. 438). Camel-human zoonotic transmission of MERS-CoV occurred in Saudi Arabia in 2012. Although human to human transmission has been documented, most de novo infections occur as a result of camel-human interactions, and outbreaks are generally localized to the Arabian Peninsula (Zaki A M, van Zaki A M, van Boheemen S, Bestebroer T M, Osterhaus A, Fouchier RAM. 2012 N Engl J Med 367:1814-1820). The fatality rate of MERS infection is about 36% (http://www.who.int/csr/don/16-october-2014-mers/en/). SARS-CoV-2, the pandemic strain causal of COVID-19, is of bat origin, and transmission from bat to humans may have occurred directly or via an unknown intermediate host animal (Lu R, Zhao X, Li J, et al. 2020. Lancet; 395(10224):565-574). SARS-CoV-2 is now a pandemic CoV and has resulted, as of December 2021, in a worldwide health and economic crisis with global deaths exceeding 5 million (JHU CSSE COVID-19 Data https://github.com/CSSEGISandData/COVID-19). These three well-characterized zoonotic events, and the likelihood of future spillover events with novel CoVs, underscores the need for broad-spectrum CoV antiviral therapies that will be active against both existing CoVs, such as MERS-CoV and SARS-CoV-2, and also CoVs that may emerge in the future.

CoV particles consist of a cell-derived lipid membrane containing structural proteins spike (S), membrane (M), envelope (E), and nucleocapsid (N) (Fields Virology Emerging Viruses Vol. 1 2021 pp. 416-417). The virion also contains a large (25-32 kb) non-segmented positive-sense single-strand viral RNA genome that, similar to cellular mRNAs, is 5′-capped, contains 5′ and 3′ untranslated regions (UTRs) and a 3′ polyadenylated tail. All CoV viral genomes contain six basic common genes: two long open reading frames (la and Ib) that encode two polypeptides that constitute the non-structural proteins (nsps) that form the multiprotein replicase-transcription complex (RTC) and four open reading frames for the structural proteins S, M, E and N that make up the virion. Depending on the CoV, one to eight additional genes, called accessory genes, can be encoded in the genome. The genomic organization amongst all CoVs is conserved and invariant across different genera such that the gene sequence is always 1a, 1b, S, M, E and N.

CoV replication is initiated through binding of the S protein to a specific cell surface receptor. SARS-CoV and SARS-CoV-2, for example, engage the angiotensin converting enzyme 2 (ACE-2) on cells of the upper respiratory tract (Lu R, Zhao X, Li J, et al. 2020. Lancet; 395(10224):565-574). Viral attachment leads to either viral endocytosis followed by fusion of the viral and endosome membranes, or direct fusion of the viral and cellular plasma members at the cell surface, to release virions into the cytoplasm. After entry, the viral genomic RNA is uncoated and serves as a template for cap-dependent translation of Orf 1a and Orf 1b to produce the viral polypeptides pp1a and pp1ab (Fung S, Liu D, 2019. Annu. Rev. Microbiol. 73: 529-57). Cleavage of the viral polypeptides to yield the individual replisome proteins is carried out by the viral papain-like protease (PLPro or nsp3) and 3CL main protease (Mpro or nsp5). The nsps form double-membraned vesicles and assemble to form RTCs responsible for genome replication, sub-genomic RNA (sgRNA) synthesis and transcription of the sgRNAs. The sgRNA serve as templates from which the mRNAs encoding for the structural and accessory proteins are translated. Assembly of new viral particles occurs in the endoplasmic reticulum—golgi intermediate complex and mature particles are released through secretory vesicles.

Vaccines for prevention of COVID-19 have been developed using the S protein of SARS-CoV-2 as an antigen to elicit a protective immune response (Kryikidis et. al. npj Vaccines 28 (2021) 6:28). Vaccines based on mRNA/lipid nanoparticle and replication-defective adenoviruses vectored platforms have both been demonstrated to be highly effective for prevention of serious illness. However, there is limited data on the effectiveness of these vaccines for transmission of SARS-CoV-2. A liability of using the S protein for vaccine development is that the amino acid sequence is highly variable, enabling the SARS-CoV-2 to adapt to immune pressure (Chen R E et al. Nature Medicine. Mar. 4, 2021). Multiple independent spike mutations have been detected, even in the absence of vaccine selective pressure, and some variants will likely lead to reduced efficacy in vaccine clinical trials conducted where those variants are circulating.

Given the limitations of the current vaccines and the potential for zoonotic emergence of new pandemic strains, there is an urgent need for broad-spectrum anti-coronaviral treatment and prophylactic regimens. An anti-coronavirus intervention with efficacy against SARS-CoV, SARS-CoV-2, and the more distantly related MERS-CoV would be expected to have broad-spectrum activity against both SARS-CoV-2 and future CoVs that may emerge through zoonotic events.

### SUMMARY OF THE INVENTION

The present invention provides compounds of Formula I:

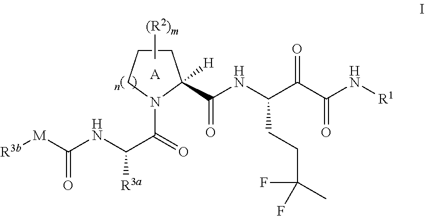

and pharmaceutically acceptable salts thereof. The compounds of Formula I are protease inhibitors, and as such may be useful in the treatment, inhibition, or amelioration of one or more disease states that could benefit from inhibition of a coronavirus, including SARS-CoV, MERS-CoV and SARS-CoV-2. Thus, the present invention also provides a method for prophylaxis or treatment of a coronavirus infection (e.g., a SARS-CoV, a SARS-CoV-2 or a MERS-CoV infection), comprising administering an effective amount of the compound of any of the compounds of Formula I disclosed herein or a pharmaceutically acceptable salt thereof to a patient in need thereof.

The compounds of this invention could further be used in combination with other therapeutically effective agents (second therapeutic agents), including but not limited to, other drugs useful for the treatment of coronavirus infection. The invention furthermore relates to processes for preparing compounds of Formula I, and pharmaceutical compositions which comprise compounds of Formula I and pharmaceutically acceptable salts thereof.

### DETAILED DESCRIPTION OF THE INVENTION

In one aspect, the present invention is a compound of Formula I:

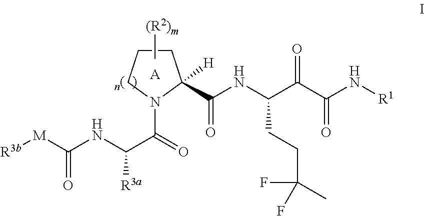

or a pharmaceutically acceptable salt thereof, wherein:
R¹ is
   (a) C₁-C₆ alkyl,
   (b) —(CH₂)ₚ—R^{1c}, wherein R^{1c} is:
      (i) C₃-C₆ cycloalkyl;
      (ii) a 5- to 6-membered saturated heterocycloalkyl containing 1 to 2 heteroatoms independently selected from N, O, or S;
      (iii) a 5- to 6-membered heteroaryl containing 1 to 3 heteroatoms independently selected from N, O, or S; or
      (iv) phenyl;
      wherein R^{1c} is unsubstituted or substituted by halo, C₁-C₃ alkyl, C₁-C₃ fluoroalkyl, —O—C₁-C₃ alkyl, —O—C₁-C₃ fluoroalkyl, or —C₃-C₄ cycloalkyl; or
   (c) H;
each R² is independently fluoro, hydroxy, C₁-C₃ alkyl, C₁-C₃ fluoroalkyl, —O—C₁-C₃ alkyl, —O—C₁-C₃ fluoroalkyl, or ring R^{2cy},
   wherein ring R^{2cy} is cyclopropyl, cyclobutyl, cyclopentyl, oxetanyl, tetrahydrofuryl, or bicyclo[1.1.1]pentyl;
      wherein ring R^{2cy} is unsubstituted or substituted by 1 to 2 R^{2ca} substituents independently selected from fluoro, hydroxy, C₁-C₃ alkyl, C₁-C₃ fluoroalkyl, —O—C₁-C₃ alkyl, or —O—C₁-C₃ fluoroalkyl;
or alternatively, any two R², together with the carbon atom(s) to which they are attached form ring A′ to form a bicyclic ring system with illustrated ring A;
   wherein ring A′ is a 3- to 6-membered cycloalkyl, tetrahydrofuran or tetrahydropyranyl ring;
      wherein ring A′ is unsubstituted or substituted by 1 to 4 R^{a1} substituents independently selected from the group consisting of fluoro, hydroxy, C₁-C₃ alkyl, C₁-C₃ fluoroalkyl, —O—C₁-C₃ alkyl, or —O—C₁-C₃ fluoroalkyl;
         or alternatively, two R^{a1} substituents, when substituted on a common carbon atom, together with the carbon atom to which they are attached, form ring A″;
         wherein ring A″ is a 3- to 6-membered cycloalkyl, tetrahydrofuran or tetrahydropyranyl ring;
R^{3a} is
   (a) C₁-C₆ alkyl,
   (b) C₁-C₃ fluoroalkyl,
   (c) —CH₂O—(C₁-C₆ alkyl),
   (d) a group of the formula

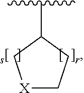

wherein X is —CH₂—, —CF₂— or —O—;
(e) a group of the formula —(CH₂)ₜ—Y^{3c}
   wherein Y^{3c} is phenyl or a 5- to 6-membered heteroaryl containing 1 to 3 heteroatoms independently selected from N, O, or S,
      wherein Y^{3c} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl;
M is —O— or —N(H)—;
R^{3b} is
   (a) C₁-C₆ alkyl, or
   (b) a group of the formula —(CH₂)ᵤ—Y^{3b}
      wherein Y^{3b} is:
      (i) phenyl;
      (ii) C₃-C₆ cycloalkyl;
      (iii) a 5- to 6-membered saturated heterocycloalkyl containing 1 to 2 heteroatoms independently selected from N, O, or S; or
      (iv) a 5- to 6-membered heteroaryl containing 1 to 3 heteroatoms independently selected from N, O, or S;
      wherein Y^{3b} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl;
subscript m is 0, 1, 2 or 3;
subscript n is 1 or 2;
subscript p is 0, 1, or 2;
subscript r is 0, 1, or 2;
subscript s is 0, 1, or 2;
subscript t is 0 or 1; and
subscript u is 0 or 1.

In another aspect of the invention, the compound of Formula I is

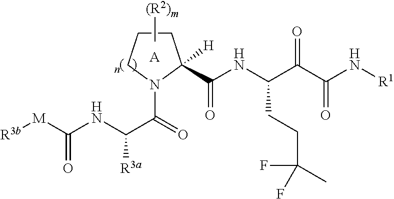

or a pharmaceutically acceptable salt thereof, wherein:
R¹ is
   (a) C₁-C₆ alkyl,
   (b) C₁-C₆ alkoxy,
   (c) C₁-C₆ fluoroalkyl,
   (d) —(CH₂)ₚ—R^{1c}, wherein R^{1c} is:
      (i) C₃-C₆ cycloalkyl;
      (ii) a 5- to 6-membered saturated heterocycloalkyl containing 1 to 2 heteroatoms independently selected from N, O, or S;
      (iii) a 5- to 6-membered heteroaryl containing 1 to 3 heteroatoms independently selected from N, O, or S; or
      (iv) phenyl;
      wherein R^{1c} is unsubstituted or substituted by halo, C₁-C₃ alkyl, C₁-C₃ fluoroalkyl, —O—C₁-C₃ alkyl, —O—C₁-C₃ fluoroalkyl, or —C₃-C₄ cycloalkyl; or
   (e) H;
each R² is independently fluoro, hydroxy, C₁-C₃ alkyl, C₁-C₃ fluoroalkyl, —O—C₁-C₃ alkyl, —O—C₁-C₃ fluoroalkyl, or ring R^{2cy},
   wherein ring R^{2cy} is cyclopropyl, cyclobutyl, cyclopentyl, oxetanyl, tetrahydrofuryl, or bicyclo[1.1.1]pentyl;
      wherein ring R^{2cy} is unsubstituted or substituted by 1 to 2 R^{2ca} substituents independently selected from fluoro, chloro, hydroxy, C₁-C₃ alkyl, C₁-C₃ fluoroalkyl, —O—C₁-C₃ alkyl, or —O—C₁-C₃ fluoroalkyl;
or alternatively, any two R², together with the carbon atom(s) to which they are attached form ring A′ to form a bicyclic ring system with illustrated ring A;
   wherein ring A′ is a 3- to 6-membered cycloalkyl, tetrahydrofuran or tetrahydropyranyl ring;
      wherein ring A′ is unsubstituted or substituted by 1 to 4 R^{a1} substituents independently selected from the group consisting of fluoro, hydroxy, C₁-C₃ alkyl, C₁-C₃ fluoroalkyl, —O—C₁-C₃ alkyl, or —O—C₁-C₃ fluoroalkyl;
         or alternatively, two R^{a1} substituents, when substituted on a common carbon atom, together with the carbon atom to which they are attached, form ring A″;
         wherein ring A″ is a 3- to 6-membered cycloalkyl, tetrahydrofuran or tetrahydropyranyl ring;
R^{3a} is
   (a) C₁-C₆ alkyl,
   (b) C₁-C₆ alkoxy,
   (c) C₁-C₃ fluoroalkyl,
   (d) —CH₂O—(C₁-C₆ alkyl),
   (e) a group of the formula

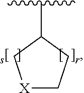

wherein X is —CH₂—, —CF₂— or —O—;
(f) a group of the formula —(CH₂)ₜ—Y^{3c}
   wherein Y^{3c} is phenyl or a 5- to 6-membered heteroaryl containing 1 to 3 heteroatoms independently selected from N, O, or S,
      wherein Y^{3c} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl;
M is —O— or —N(H)—;
R^{3b} is
   (a) C₁-C₆ alkyl, or
   (b) a group of the formula —(CH₂)ᵤ—Y^{3b}
      wherein Y^{3b} is:
         (i) phenyl;
         (ii) C₃-C₆ cycloalkyl;
         (iii) a 5- to 6-membered saturated heterocycloalkyl containing 1 to 2 heteroatoms independently selected from N, O, or S; or
         (iv) a 5- to 6-membered heteroaryl containing 1 to 3 heteroatoms independently selected from N, O, or S;
         wherein Y^{3b} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl;
subscript m is 0, 1, 2 or 3;
subscript n is 1 or 2;
subscript p is 0, 1, or 2;
subscript r is 0, 1, or 2;
subscript s is 0, 1, or 2;
subscript t is 0 or 1; and
subscript u is 0 or 1.

In some embodiments of the present invention, the group

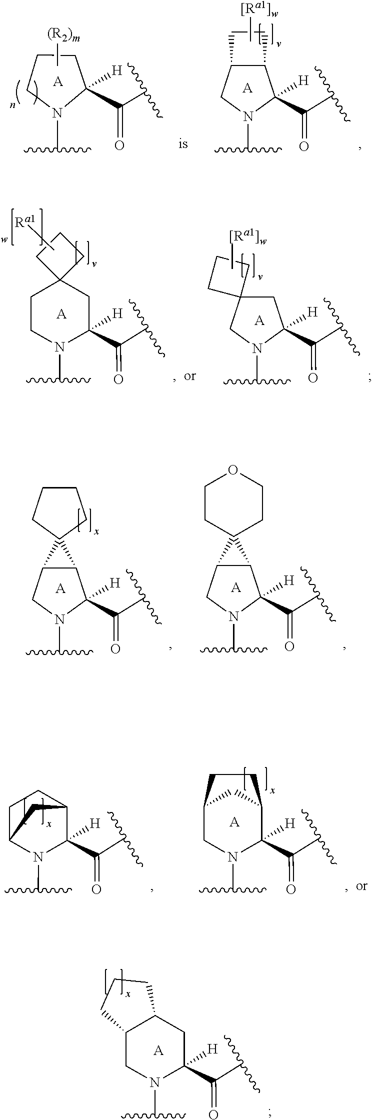

subscript v is 0, 1, 2, or 3;
subscript w is 0, 1, 2, 3, or 4; and
subscript x is 1 or 2.

In specific embodiments of the present invention, the group

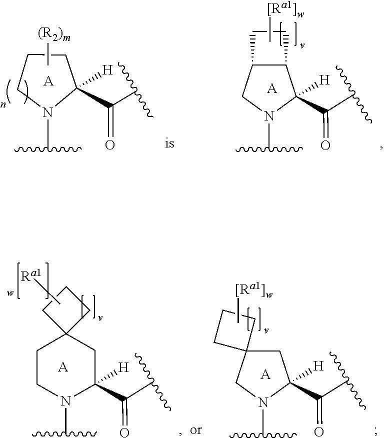

subscript v is 0, 1, 2, or 3;
subscript w is 0, 1, 2, 3, or 4; and
each R^{a1} is independently fluoro or methyl.

In certain embodiments of the present invention, the group

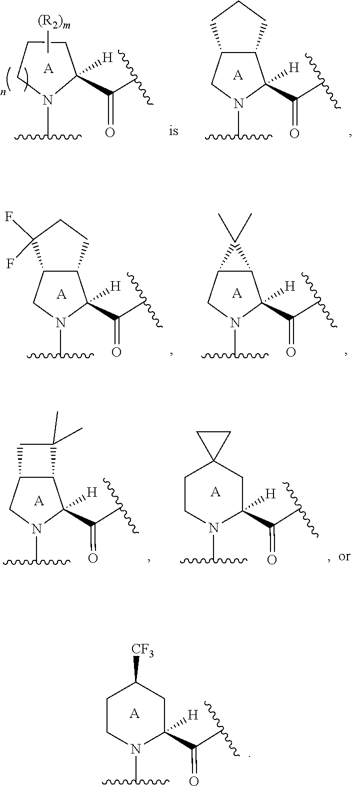

In some embodiments of the present invention, R¹ is C₁-C₄ alkyl or C₃-C₆ cycloalkyl. In specific embodiments, R¹ is methyl or cyclopropyl.

In certain embodiments of the present invention, R^{3a} is t-butyl.

In some embodiments of the present invention, M is —O—.

In some embodiments of the present invention, R^{3b} is
(a) C₁-C₆ alkyl, or
(b) a group of the formula —(CH₂)ᵤ—Y^{3b}
   wherein Y^{3b} is phenyl or C₃-C₆ cycloalkyl,
   wherein Y^{3b} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl.

In certain embodiments of the present invention, R^{3b} is C₁-C₆ alkyl. For instance, in specific embodiments, R^{3b} is methyl.

In certain embodiments of the present invention, the group

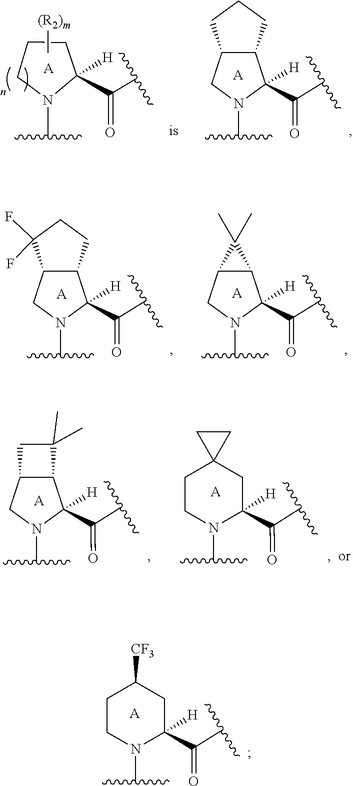

R¹ is methyl or cyclopropyl;
R^{3a} is t-butyl;
M is —O—; and
R^{3b} is methyl.

In some embodiments of the invention one or more of the hydrogen atoms in the compound of formula I are deuterated.

Reference to the specific classes and subclasses set forth above is meant to include all combinations of particular and preferred groups unless stated otherwise.

Specific embodiments of the present invention include, but are not limited to the compounds disclosed in Examples 1 to 79, or pharmaceutically acceptable salts thereof.

Other specific embodiments include compounds enumerated below or pharmaceutically acceptable salts thereof:
Methyl ((2S)-1-((2S)-4-cyclobutyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((2S)-1-((2S)-4-(bicyclo[1.1.1]pentan-1-yl)-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((2S)-1-((2S)-4-cyclopentyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((2S)-1-(3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((2S)-1-(3-(((S)-1-(cyclopropylamino)-6,6-difluoro-1,2-dioxoheptan-3-yl)carbamoyl)-8-oxa-2-azaspiro[4.5]decan-2-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-5-methylpiperidin-1-yl)-3,3-dimethyl-1-oxobutan-2-yl)carbamate;
Methyl ((2S)-1-((6S)-6-(((S)-1-(cyclopropylamino)-6,6-difluoro-1,2-dioxoheptan-3-yl)carbamoyl)-1,1-difluoro-5-azaspiro[2.4]heptan-5-yl)-3,3-dimethyl-1-oxobutan-2-yl)carbamate;
Methyl ((2S)-1-(7-(((S)-1-(cyclopropylamino)-6,6-difluoro-1,2-dioxoheptan-3-yl)carbamoyl)-8-azaspiro[4.5]decan-8-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-4,4-dimethylpiperidin-1-yl)-3,3-dimethyl-1-oxobutan-2-yl)carbamate;
Methyl ((2S)-1-(6-(((S)-1-(cyclopropylamino)-6,6-difluoro-1,2-dioxoheptan-3-yl)carbamoyl)-7-azaspiro[3.5]nonan-7-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-3-azabicyclo[3.2.0]heptan-3-yl)-3,3-dimethyl-1-oxobutan-2-yl)carbamate;
Methyl ((2S)-1-(7-(((S)-6,6-difluoro-1-(methylamino)-1,2-dioxoheptan-3-yl)carbamoyl)-6-azaspiro[3.4]octan-6-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-4,4-difluoropiperidin-1-yl)-3,3-dimethyl-1-oxobutan-2-yl)carbamate;
Methyl ((2S)-1-(7-(((S)-1-(cyclopropylamino)-6,6-difluoro-1,2-dioxoheptan-3-yl)carbamoyl)-6-azaspiro[3.4]octan-6-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-3-methylpiperidin-1-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-3-methylpyrrolidin-1-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-5,5-dimethylpiperidin-1-yl)-3,3-dimethyl-1-oxobutan-2-yl)carbamate;
Methyl ((2S)-1-((2S)-2-(((S)-1-(cyclopropylamino)-6,6-difluoro-1,2-dioxoheptan-3-yl)carbamoyl)-4-(tetrahydrofuran-3-yl)pyrrolidin-1-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)piperidin-1-yl)-3,3-dimethyl-1-oxobutan-2-yl)carbamate;
Methyl ((2S)-1-(3-(((S)-1-(cyclopropylamino)-6,6-difluoro-1,2-dioxoheptan-3-yl)carbamoyl)-8,8-difluoro-2-azaspiro[4.5]decan-2-yl)-3,3-dimethyl-1-oxobutan-2-yl)carbamate;
Methyl ((2S)-1-((2S)-2-(((S)-1-(cyclopropylamino)-6,6-difluoro-1,2-dioxoheptan-3-yl)carbamoyl)-4-(oxetan-3-yl)pyrrolidin-1-yl)-3,3-dimethyl-1-oxobutan-2-yl)carbamate;
Methyl ((2S)-1-((2S)-2-(((S)-1-(cyclopropylamino)-6,6-difluoro-1,2-dioxoheptan-3-yl)carbamoyl)-4-hydroxy-4-(trifluoromethyl)piperidin-1-yl)-3,3-dimethyl-1-oxobutan-2-yl)carbamate;
Methyl (1-cyclobutyl-2-((S)-3-(((S)-1-(cyclopropylamino)-6,6-difluoro-1,2-dioxoheptan-3-yl)carbamoyl)-2-azaspiro[4.5]decan-2-yl)-2-oxoethyl)carbamate;
Methyl ((2S)-1-(2-(((S)-1-(cyclopropylamino)-6,6-difluoro-1,2-dioxoheptan-3-yl)carbamoyl)-3-ethylpyrrolidin-1-yl)-3,3-dimethyl-1-oxobutan-2-yl)carbamate;
Methyl ((2S)-1-(2-(((S)-1-(cyclopropylamino)-6,6-difluoro-1,2-dioxoheptan-3-yl)carbamoyl)-3,3-dimethylpyrrolidin-1-yl)-3,3-dimethyl-1-oxobutan-2-yl)carbamate; and
Methyl ((2S)-1-((1R,4S)-3-(((S)-1-(cyclopropylamino)-6,6-difluoro-1,2-dioxoheptan-3-yl)carbamoyl)-2-azabicyclo[2.2.1]heptan-2-yl)-3,3-dimethyl-1-oxobutan-2-yl)carbamate.

Other specific embodiments include compounds enumerated below or pharmaceutically acceptable salts thereof:
Methyl ((S)-1-((2S,4(S))-4-cyclobutyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2S,4(R))-4-cyclobutyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2S,4(R))-4-(bicyclo[1.1.1]pentan-1-yl)-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2S,4(S))-4-(bicyclo[1.1.1]pentan-1-yl)-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2S,4(R))-4-cyclopentyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2S,4(S))-4-cyclopentyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Isopropyl ((S)-1-((S)-3-(((S)-1-(cyclopropylamino)-6,6-difluoro-1,2-dioxoheptan-3-yl)carbamoyl)-2-azaspiro[4.5]decan-2-yl)-3,3-dimethyl-1-oxobutan-2-yl)carbamate;
Cyclopentyl ((S)-1-((S)-3-(((S)-1-(cyclopropylamino)-6,6-difluoro-1,2-dioxoheptan-3-yl)carbamoyl)-2-azaspiro[4.5]decan-2-yl)-3,3-dimethyl-1-oxobutan-2-yl)carbamate;
Methyl ((S)-1-((1R,2S,5S)-2-(((S)-1-(cyclopropylamino)-6,6-difluoro-1,2-dioxoheptan-3-yl)carbamoyl)-6,6-dimethyl-3-azabicyclo[3.1.0]hexan-3-yl)-3,3-dimethyl-1-oxobutan-2-yl)carbamate;
Methyl ((S)-1-((S)-3-(((S)-1-(benzylamino)-6,6-difluoro-1,2-dioxoheptan-3-yl)carbamoyl)-2-azaspiro[4.5]decan-2-yl)-3,3-dimethyl-1-oxobutan-2-yl)carbamate;
Methyl ((S)-1-((S)-3-(((S)-1-(ethylamino)-6,6-difluoro-1,2-dioxoheptan-3-yl)carbamoyl)-2-azaspiro[4.5]decan-2-yl)-3,3-dimethyl-1-oxobutan-2-yl)carbamate;
Methyl ((S)-1-((S)-3-(((S)-6,6-difluoro-1-(isobutylamino)-1,2-dioxoheptan-3-yl)carbamoyl)-2-azaspiro[4.5]decan-2-yl)-3,3-dimethyl-1-oxobutan-2-yl)carbamate;
Methyl ((S)-1-((S)-3-(((S)-1-(cyclopropylamino)-6,6-difluoro-1,2-dioxoheptan-3-yl)carbamoyl)-2-azaspiro[4.4]nonan-2-yl)-3,3-dimethyl-1-oxobutan-2-yl)carbamate;
Methyl ((R)-1-((S)-3-(((S)-1-(cyclopropylamino)-6,6-difluoro-1,2-dioxoheptan-3-yl)carbamoyl)-2-azaspiro[4.5]decan-2-yl)-3-methoxy-1-oxopropan-2-yl)carbamate;
Methyl ((S)-1-((S)-3-(((S)-1-(cyclopropylamino)-6,6-difluoro-1,2-dioxoheptan-3-yl)carbamoyl)-2-azaspiro[4.5]decan-2-yl)-3-methoxy-1-oxopropan-2-yl)carbamate;
Methyl ((S)-1-((S)-3-(((S)-6,6-difluoro-1,2-dioxo-1-(o-tolylamino)heptan-3-yl)carbamoyl)-2-azaspiro[4.5]decan-2-yl)-3,3-dimethyl-1-oxobutan-2-yl)carbamate;
Methyl ((S)-1-((S)-3-(((S)-6,6-difluoro-1-(isopropylamino)-1,2-dioxoheptan-3-yl)carbamoyl)-2-azaspiro[4.5]decan-2-yl)-3,3-dimethyl-1-oxobutan-2-yl)carbamate;
Methyl ((S)-1-((S)-3-(((S)-1-(cyclopropylamino)-6,6-difluoro-1,2-dioxoheptan-3-yl)carbamoyl)-2-azaspiro[4.5]decan-2-yl)-1-oxopropan-2-yl)carbamate;
Tert-butyl ((S)-1-((S)-3-(((S)-1-(cyclopropylamino)-6,6-difluoro-1,2-dioxoheptan-3-yl)carbamoyl)-2-azaspiro[4.5]decan-2-yl)-3-methyl-1-oxobutan-2-yl)carbamate;
Methyl ((S)-1-((S)-3-(((S)-1-(cyclohexylamino)-6,6-difluoro-1,2-dioxoheptan-3-yl)carbamoyl)-2-azaspiro[4.5]decan-2-yl)-3,3-dimethyl-1-oxobutan-2-yl)carbamate;
Methyl ((S)-1-((S)-3-(((S)-6,6-difluoro-1-((2-morpholinoethyl)amino)-1,2-dioxoheptan-3-yl)carbamoyl)-2-azaspiro[4.5]decan-2-yl)-3,3-dimethyl-1-oxobutan-2-yl)carbamate;
Methyl ((S)-1-((S)-3-(((S)-1-(tert-butylamino)-6,6-difluoro-1,2-dioxoheptan-3-yl)carbamoyl)-2-azaspiro[4.5]decan-2-yl)-3,3-dimethyl-1-oxobutan-2-yl)carbamate;
Methyl (1-((S)-3-(((S)-1-(cyclopropylamino)-6,6-difluoro-1,2-dioxoheptan-3-yl)carbamoyl)-2-azaspiro[4.5]decane-2-carbonyl)cyclopropyl)carbamate;
Methyl ((S)-1-((S)-5-(((S)-1-amino-6,6-difluoro-1,2-dioxoheptan-3-yl)carbamoyl)-6-azaspiro[2.5]octan-6-yl)-3,3-dimethyl-1-oxobutan-2-yl)carbamate;
Methyl ((S)-1-((S)-5-(((S)-6,6-difluoro-1-(methylamino)-1,2-dioxoheptan-3-yl)carbamoyl)-6-azaspiro[2.5]octan-6-yl)-3,3-dimethyl-1-oxobutan-2-yl)carbamate;
Methyl ((S)-1-((2S,4R)-2-(((S)-6,6-difluoro-1-(methylamino)-1,2-dioxoheptan-3-yl)carbamoyl)-4-(trifluoromethyl)piperidin-1-yl)-3,3-dimethyl-1-oxobutan-2-yl)carbamate;
Methyl ((S)-1-((2(S),3(R))-3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2(S),3(S))-3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2(R),3(R))-3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((2(R),3(S))-3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate;
Methyl ((S)-1-((1S,3aR,6aS)-1-(((S)-6,6-difluoro-1-(methylamino)-1,2-dioxoheptan-3-yl)carbamoyl)hexahydrocyclopenta[c]pyrrol-2(1H)-yl)-3,3-dimethyl-1-oxobutan-2-yl)carbamate;
Methyl ((S)-1-((1R,2S,5R)-2-(((S)-1-(cyclopropylamino)-6,6-difluoro-1,2-dioxoheptan-3-yl)carbamoyl)-7,7-dimethyl-3-azabicyclo[3.2.0]heptan-3-yl)-3,3-dimethyl-1-oxobutan-2-yl)carbamate,
Methyl ((S)-1-((3S,4aR,8aR)-3-(((S)-1-(cyclopropylamino)-6,6-difluoro-1,2-dioxoheptan-3-yl)carbamoyl)octahydroisoquinolin-2(1H)-yl)-3,3-dimethyl-1-oxobutan-2-yl)carbamate;
Methyl ((S)-1-((2S,4R)-2-(((S)-6,6-difluoro-1-(methylamino)-1,2-dioxoheptan-3-yl)carbamoyl)-4-(trifluoromethyl)pyrrolidin-1-yl)-3,3-dimethyl-1-oxobutan-2-yl)carbamate;
Methyl ((S)-1-((R)-(3-(((S)-1-(cyclopropylamino)-6,6-difluoro-1,2-dioxoheptan-3-yl)carbamoyl)-8-oxa-2-azaspiro[4.5]decan-2-yl)-3,3-dimethyl-1-oxobutan-2-yl)carbamate;
Methyl ((S)-1-((S)-(3-(((S)-1-(cyclopropylamino)-6,6-difluoro-1,2-dioxoheptan-3-yl)carbamoyl)-8-oxa-2-azaspiro[4.5]decan-2-yl)-3,3-dimethyl-1-oxobutan-2-yl)carbamate;
Methyl ((S)-1-((S)-5-(((S)-1-(cyclopropylamino)-6,6-difluoro-1,2-dioxoheptan-3-yl)carbamoyl)-6-azaspiro[2.5]octan-6-yl)-3,3-dimethyl-1-oxobutan-2-yl)carbamate;
Methyl ((S)-1-(2(R),5(R)-(((S)-1-(cyclopropylamino)-6,6-difluoro-1,2-dioxoheptan-3-yl)carbamoyl)-5-methylpiperidin-1-yl)-3,3-dimethyl-1-oxobutan-2-yl)carbamate;
Methyl ((S)-1-(2(R),5(S)-(((S)-1-(cyclopropylamino)-6,6-difluoro-1,2-dioxoheptan-3-yl)carbamoyl)-5-methylpiperidin-1-yl)-3,3-dimethyl-1-oxobutan-2-yl)carbamate;
Methyl ((S)-1-(2(S),5(R)-(((S)-1-(cyclopropylamino)-6,6-difluoro-1,2-dioxoheptan-3-yl)carbamoyl)-5-methylpiperidin-1-yl)-3,3-dimethyl-1-oxobutan-2-yl)carbamate;
Methyl ((S)-1-(2(S),5(S)-(((S)-1-(cyclopropylamino)-6,6-difluoro-1,2-dioxoheptan-3-yl)carbamoyl)-5-methylpiperidin-1-yl)-3,3-dimethyl-1-oxobutan-2-yl)carbamate;
Methyl ((S)-1-((1S,3aR,6aS)-1-(((S)-1-(cyclopropylamino)-6,6-difluoro-1,2-dioxoheptan-3-yl)carbamoyl)hexahydrocyclopenta[c]pyrrol-2(1H)-yl)-3,3-dimethyl-1-oxobutan-2-yl)carbamate;
Methyl ((S)-1-((3(R),6S)-6-(((S)-1-(cyclopropylamino)-6,6-difluoro-1,2-dioxoheptan-3-yl)carbamoyl)-1,1-difluoro-5-azaspiro[2.4]heptan-5-yl)-3,3-dimethyl-1-oxobutan-2-yl)carbamate;
Methyl ((S)-1-((3(S),6S)-6-(((S)-1-(cyclopropylamino)-6,6-difluoro-1,2-dioxoheptan-3-yl)carbamoyl)-1,1-difluoro-5-azaspiro[2.4]heptan-5-yl)-3,3-dimethyl-1-oxobutan-2-yl)carbamate;
Methyl ((S)-1-((1S,3aR,6aS)-1-(((S)-1-(cyclopropylamino)-6,6-difluoro-1,2-dioxoheptan-3-yl)carbamoyl)-4,4-difluorohexahydrocyclopenta[c]pyrrol-2(1H)-yl)-3,3-dimethyl-1-oxobutan-2-yl)carbamate;
Methyl ((S)-1-(R)-(7-(((S)-1-(cyclopropylamino)-6,6-difluoro-1,2-dioxoheptan-3-yl)carbamoyl)-8-azaspiro[4.5]decan-8-yl)-3,3-dimethyl-1-oxobutan-2-yl)carbamateazaspiro[4.5]decan-8-yl)-3,3-dimethyl-1-oxobutan-2-yl)carbamate;
Methyl ((S)-1-(S)-(7-(((S)-1-(cyclopropylamino)-6,6-difluoro-1,2-dioxoheptan-3-yl)carbamoyl)-8-azaspiro[4.5]decan-8-yl)-3,3-dimethyl-1-oxobutan-2-yl)carbamateazaspiro[4.5]decan-8-yl)-3,3-dimethyl-1-oxobutan-2-yl)carbamate;
Methyl ((S)-1-((1S,3aR,6aS)-1-(((S)-6,6-difluoro-1-(methylamino)-1,2-dioxoheptan-3-yl)carbamoyl)-4,4-difluorohexahydrocyclopenta[c]pyrrol-2(1H)-yl)-3,3-dimethyl-1-oxobutan-2-yl)carbamate;
Methyl ((S)-1-((1R,2S,5S)-2-(((S)-1-(cyclopropylamino)-6,6-difluoro-1,2-dioxoheptan-3-yl)carbamoyl)-6,6,7,7-tetramethyl-3-azabicyclo[3.2.0]heptan-3-yl)-3,3-dimethyl-1-oxobutan-2-yl)carbamate;
Methyl ((S)-1-((R)-(2-(((S)-1-(cyclopropylamino)-6,6-difluoro-1,2-dioxoheptan-3-yl)carbamoyl)-4,4-dimethylpiperidin-1-yl)-3,3-dimethyl-1-oxobutan-2-yl)carbamate;
Methyl ((S)-1-((S)-(2-(((S)-1-(cyclopropylamino)-6,6-difluoro-1,2-dioxoheptan-3-yl)carbamoyl)-4,4-dimethylpiperidin-1-yl)-3,3-dimethyl-1-oxobutan-2-yl)carbamate;
Methyl ((S)-1-((1R,2S,5S)-2-(((S)-1-(cyclopropylamino)-6,6-difluoro-1,2-dioxoheptan-3-yl)carbamoyl)-3-azaspiro[bicyclo[3.1.0]hexane-6,1′-cyclopentan]-3-yl)-3,3-dimethyl-1-oxobutan-2-yl)carbamate;
Methyl ((S)-1-((1R,2S,5S)-2-(((S)-6,6-difluoro-1-(methylamino)-1,2-dioxoheptan-3-yl)carbamoyl)-6,6-dimethyl-3-azabicyclo[3.1.0]hexan-3-yl)-3,3-dimethyl-1-oxobutan-2-yl)carbamate;
Methyl ((S)-1-((1R,2S,5S)-2-(((S)-6,6-difluoro-1-(methylamino)-1,2-dioxoheptan-3-yl)carbamoyl)tetrahydro-3-azaspiro[bicyclo[3.1.0]hexane-6,4′-pyran]-3-yl)-3,3-dimethyl-1-oxobutan-2-yl)carbamate;
Methyl ((S)-1-((R)-(6-(((S)-1-(cyclopropylamino)-6,6-difluoro-1,2-dioxoheptan-3-yl)carbamoyl)-7-azaspiro[3.5]nonan-7-yl)-3,3-dimethyl-1-oxobutan-2-yl)carbamate;
Methyl ((S)-1-((S)-(6-(((S)-1-(cyclopropylamino)-6,6-difluoro-1,2-dioxoheptan-3-yl)carbamoyl)-7-azaspiro[3.5]nonan-7-yl)-3,3-dimethyl-1-oxobutan-2-yl)carbamate;
Methyl ((S)-1-((1R,2S,5S)-2-(((S)-1-amino-6,6-difluoro-1,2-dioxoheptan-3-yl)carbamoyl)-6,6,7,7-tetramethyl-3-azabicyclo[3.2.0]heptan-3-yl)-3,3-dimethyl-1-oxobutan-2-yl)carbamate;
Methyl ((S)-1-((1S,2S,5R)-2-(((S)-1-(cyclopropylamino)-6,6-difluoro-1,2-dioxoheptan-3-yl)carbamoyl)-3-azabicyclo[3.2.0]heptan-3-yl)-3,3-dimethyl-1-oxobutan-2-yl)carbamate;
Methyl ((S)-1-((1R,2R,5S)-2-(((S)-1-(cyclopropylamino)-6,6-difluoro-1,2-dioxoheptan-3-yl)carbamoyl)-3-azabicyclo[3.2.0]heptan-3-yl)-3,3-dimethyl-1-oxobutan-2-yl)carbamate;
Methyl ((S)-1-((R)-7-(((S)-6,6-difluoro-1-(methylamino)-1,2-dioxoheptan-3-yl)carbamoyl)-6-azaspiro[3.4]octan-6-yl)-3,3-dimethyl-1-oxobutan-2-yl)carbamate;
Methyl ((S)-1-((S)-7-(((S)-6,6-difluoro-1-(methylamino)-1,2-dioxoheptan-3-yl)carbamoyl)-6-azaspiro[3.4]octan-6-yl)-3,3-dimethyl-1-oxobutan-2-yl)carbamate;
Methyl ((S)-1-(R)-(2-(((S)-1-(cyclopropylamino)-6,6-difluoro-1,2-dioxoheptan-3-yl)carbamoyl)-4,4-difluoropiperidin-1-yl)-3,3-dimethyl-1-oxobutan-2-yl)carbamate;
Methyl ((S)-1-(S)-(2-(((S)-1-(cyclopropylamino)-6,6-difluoro-1,2-dioxoheptan-3-yl)carbamoyl)-4,4-difluoropiperidin-1-yl)-3,3-dimethyl-1-oxobutan-2-yl)carbamate;
Methyl ((R)-3-((S)-3-(((S)-1-(cyclopropylamino)-6,6-difluoro-1,2-dioxoheptan-3-yl)carbamoyl)-2-azaspiro[4.5]decan-2-yl)-1,1,1-trifluoro-3-oxopropan-2-yl)carbamate;
Methyl ((S)-1-(S)-7-(((S)-1-(cyclopropylamino)-6,6-difluoro-1,2-dioxoheptan-3-yl)carbamoyl)-6-azaspiro[3.4]octan-6-yl)-3,3-dimethyl-1-oxobutan-2-yl)carbamate;
Methyl ((S)-1-(S)-7-(((S)-1-(cyclopropylamino)-6,6-difluoro-1,2-dioxoheptan-3-yl)carbamoyl)-6-azaspiro[3.4]octan-6-yl)-3,3-dimethyl-1-oxobutan-2-yl)carbamate;
Methyl ((S)-1-((2S,4R)-2-(((S)-1-(cyclopropylamino)-6,6-difluoro-1,2-dioxoheptan-3-yl)carbamoyl)-3,3-dimethyl-4-(trifluoromethoxy)pyrrolidin-1-yl)-3,3-dimethyl-1-oxobutan-2-yl)carbamate;
Methyl ((S)-1-((2(R),3(R))-2-(((S)-1-(cyclopropylamino)-6,6-difluoro-1,2-dioxoheptan-3-yl)carbamoyl)-3-methylpiperidin-1-yl)-3,3-dimethyl-1-oxobutan-2-yl)carbamate;
Methyl ((S)-1-((2(R),3(S))-2-(((S)-1-(cyclopropylamino)-6,6-difluoro-1,2-dioxoheptan-3-yl)carbamoyl)-3-methylpiperidin-1-yl)-3,3-dimethyl-1-oxobutan-2-yl)carbamate;
Methyl ((S)-1-((2(S),3(R))-2-(((S)-1-(cyclopropylamino)-6,6-difluoro-1,2-dioxoheptan-3-yl)carbamoyl)-3-methylpiperidin-1-yl)-3,3-dimethyl-1-oxobutan-2-yl)carbamate;
Methyl ((S)-1-((2(S),3(S))-2-(((S)-1-(cyclopropylamino)-6,6-difluoro-1,2-dioxoheptan-3-yl)carbamoyl)-3-methylpiperidin-1-yl)-3,3-dimethyl-1-oxobutan-2-yl)carbamate;
Isopropyl ((S)-1-cyclohexyl-2-((S)-3-(((S)-1-(cyclopropylamino)-6,6-difluoro-1,2-dioxoheptan-3-yl)carbamoyl)-2-azaspiro[4.5]decan-2-yl)-2-oxoethyl)carbamate;
Methyl ((S)-1-((2(R),3(R))-2-(((S)-1-(cyclopropylamino)-6,6-difluoro-1,2-dioxoheptan-3-yl)carbamoyl)-3-methylpyrrolidin-1-yl)-3,3-dimethyl-1-oxobutan-2-yl)carbamate;
Methyl ((S)-1-((2(R),3(S))-2-(((S)-1-(cyclopropylamino)-6,6-difluoro-1,2-dioxoheptan-3-yl)carbamoyl)-3-methylpyrrolidin-1-yl)-3,3-dimethyl-1-oxobutan-2-yl)carbamate;
Methyl ((S)-1-((2(S),3(R))-2-(((S)-1-(cyclopropylamino)-6,6-difluoro-1,2-dioxoheptan-3-yl)carbamoyl)-3-methylpyrrolidin-1-yl)-3,3-dimethyl-1-oxobutan-2-yl)carbamate;
Methyl ((S)-1-((2(S),3(S))-2-(((S)-1-(cyclopropylamino)-6,6-difluoro-1,2-dioxoheptan-3-yl)carbamoyl)-3-methylpyrrolidin-1-yl)-3,3-dimethyl-1-oxobutan-2-yl)carbamate;
Methyl ((S)-1-(R)-2-(((S)-1-(cyclopropylamino)-6,6-difluoro-1,2-dioxoheptan-3-yl)carbamoyl)-5,5-dimethylpiperidin-1-yl)-3,3-dimethyl-1-oxobutan-2-yl)carbamate;
Methyl ((S)-1-(S)-2-(((S)-1-(cyclopropylamino)-6,6-difluoro-1,2-dioxoheptan-3-yl)carbamoyl)-5,5-dimethylpiperidin-1-yl)-3,3-dimethyl-1-oxobutan-2-yl)carbamate;
Methyl ((S)-1-((S)-7-(((S)-6,6-difluoro-1-(methylamino)-1,2-dioxoheptan-3-yl)carbamoyl)-2,2-difluoro-6-azaspiro[3.4]octan-6-yl)-3,3-dimethyl-1-oxobutan-2-yl)carbamate;
Methyl ((S)-1-((2S,4(R))-2-(((S)-1-(cyclopropylamino)-6,6-difluoro-1,2-dioxoheptan-3-yl)carbamoyl)-4-((R)-tetrahydrofuran-3-yl)pyrrolidin-1-yl)-3,3-dimethyl-1-oxobutan-2-yl)carbamate;
Methyl ((S)-1-((2S,4(R))-2-(((S)-1-(cyclopropylamino)-6,6-difluoro-1,2-dioxoheptan-3-yl)carbamoyl)-4-((S)-tetrahydrofuran-3-yl)pyrrolidin-1-yl)-3,3-dimethyl-1-oxobutan-2-yl)carbamate;
Methyl ((S)-1-((2S,4(S))-2-(((S)-1-(cyclopropylamino)-6,6-difluoro-1,2-dioxoheptan-3-yl)carbamoyl)-4-((R)-tetrahydrofuran-3-yl)pyrrolidin-1-yl)-3,3-dimethyl-1-oxobutan-2-yl)carbamate;
Methyl ((S)-1-((2S,4(S))-2-(((S)-1-(cyclopropylamino)-6,6-difluoro-1,2-dioxoheptan-3-yl)carbamoyl)-4-((S)-tetrahydrofuran-3-yl)pyrrolidin-1-yl)-3,3-dimethyl-1-oxobutan-2-yl)carbamate;
Methyl ((S)-1-((S)-2-(((S)-1-(cyclopropylamino)-6,6-difluoro-1,2-dioxoheptan-3-yl)carbamoyl)piperidin-1-yl)-3,3-dimethyl-1-oxobutan-2-yl)carbamate;
Methyl ((S)-1-((R)-2-(((S)-1-(cyclopropylamino)-6,6-difluoro-1,2-dioxoheptan-3-yl)carbamoyl)piperidin-1-yl)-3,3-dimethyl-1-oxobutan-2-yl)carbamate;
Methyl ((S)-1-((R)-(3-(((S)-1-(cyclopropylamino)-6,6-difluoro-1,2-dioxoheptan-3-yl)carbamoyl)-8,8-difluoro-2-azaspiro[4.5]decan-2-yl)-3,3-dimethyl-1-oxobutan-2-yl)carbamate;
Methyl ((S)-1-((S)-(3-(((S)-1-(cyclopropylamino)-6,6-difluoro-1,2-dioxoheptan-3-yl)carbamoyl)-8,8-difluoro-2-azaspiro[4.5]decan-2-yl)-3,3-dimethyl-1-oxobutan-2-yl)carbamate;
Methyl ((S)-1-((2S, 4(R))-2-(((S)-1-(cyclopropylamino)-6,6-difluoro-1,2-dioxoheptan-3-yl)carbamoyl)-4-(oxetan-3-yl)pyrrolidin-1-yl)-3,3-dimethyl-1-oxobutan-2-yl)carbamate;
Methyl ((S)-1-((2S, 4(S))-2-(((S)-1-(cyclopropylamino)-6,6-difluoro-1,2-dioxoheptan-3-yl)carbamoyl)-4-(oxetan-3-yl)pyrrolidin-1-yl)-3,3-dimethyl-1-oxobutan-2-yl)carbamate;
Methyl ((S)-1-((2S, 4(R))-2-(((S)-1-(cyclopropylamino)-6,6-difluoro-1,2-dioxoheptan-3-yl)carbamoyl)-4-hydroxy-4-(trifluoromethyl)piperidin-1-yl)-3,3-dimethyl-1-oxobutan-2-yl)carbamate;
Methyl ((S)-1-((2S, 4(S))-2-(((S)-1-(cyclopropylamino)-6,6-difluoro-1,2-dioxoheptan-3-yl)carbamoyl)-4-hydroxy-4-(trifluoromethyl)piperidin-1-yl)-3,3-dimethyl-1-oxobutan-2-yl)carbamate;
Methyl ((S)-1-((S)-3-(((S)-1-(cyclopropylamino)-6,6-difluoro-1,2-dioxoheptan-3-yl)carbamoyl)-2-azabicyclo[2.2.2]octan-2-yl)-3,3-dimethyl-1-oxobutan-2-yl)carbamate;
Methyl ((S)-1-((1R,2S,5S)-2-(((S)-1-(cyclopropylamino)-6,6-difluoro-1,2-dioxoheptan-3-yl)carbamoyl)tetrahydro-3-azaspiro[bicyclo[3.1.0]hexane-6,4′-pyran]-3-yl)-3,3-dimethyl-1-oxobutan-2-yl)carbamate;
Methyl ((S)-1-((2S,4R)-2-(((S)-1-(cyclopropylamino)-6,6-difluoro-1,2-dioxoheptan-3-yl)carbamoyl)-4-(trifluoromethyl)piperidin-1-yl)-3,3-dimethyl-1-oxobutan-2-yl)carbamate,
Methyl ((S)-1-((S)-3-(((S)-6,6-difluoro-1-(methylamino)-1,2-dioxoheptan-3-yl)carbamoyl)-2-azaspiro[4.5]decan-2-yl)-3,3-dimethyl-1-oxobutan-2-yl)carbamate;
Methyl ((R)-1-cyclobutyl-2-((S)-3-(((S)-1-(cyclopropylamino)-6,6-difluoro-1,2-dioxoheptan-3-yl)carbamoyl)-2-azaspiro[4.5]decan-2-yl)-2-oxoethyl)carbamate;
Methyl ((S)-1-cyclobutyl-2-((S)-3-(((S)-1-(cyclopropylamino)-6,6-difluoro-1,2-dioxoheptan-3-yl)carbamoyl)-2-azaspiro[4.5]decan-2-yl)-2-oxoethyl)carbamate;
Methyl ((S)-2-((2S,4R)-2-(((S)-1-(cyclopropylamino)-6,6-difluoro-1,2-dioxoheptan-3-yl)carbamoyl)-4-(trifluoromethyl)pyrrolidin-1-yl)-1-(4,4-difluorocyclohexyl)-2-oxoethyl)carbamate;
Methyl ((S)-2-((2S,4R)-2-(((S)-1-(cyclopropylamino)-6,6-difluoro-1,2-dioxoheptan-3-yl)carbamoyl)-4-(trifluoromethyl)piperidin-1-yl)-2-oxo-1-phenylethyl)carbamate;
Benzyl ((S)-1-cyclohexyl-2-((1R,2S,5S)-2-(((S)-1-(cyclopropylamino)-6,6-difluoro-1,2-dioxoheptan-3-yl)carbamoyl)-3-azaspiro[bicyclo[3.1.0]hexane-6,1′-cyclohexan]-3-yl)-2-oxoethyl)carbamate;
Methyl ((S)-1-((2S,4R)-2-(((S)-1-(cyclopropylamino)-6,6-difluoro-1,2-dioxoheptan-3-yl)carbamoyl)-4-(trifluoromethyl)pyrrolidin-1-yl)-3,3-dimethyl-1-oxobutan-2-yl)carbamate;
Methyl ((S)-1-((2S,4R)-2-(((S)-1-(cyclopropylamino)-6,6-difluoro-1,2-dioxoheptan-3-yl)carbamoyl)-4-(trifluoromethyl)piperidin-1-yl)-1-oxo-3-phenylpropan-2-yl)carbamate;
Methyl ((S)-1-((1R,2S,5S)-2-(((S)-1-(cyclopropylamino)-6,6-difluoro-1,2-dioxoheptan-3-yl)carbamoyl)-3-azaspiro[bicyclo[3.1.0]hexane-6,1′-cyclohexan]-3-yl)-3,3-dimethyl-1-oxobutan-2-yl)carbamate;
Methyl ((S)-1-((2S,4R)-2-(((S)-1-(cyclopropylamino)-6,6-difluoro-1,2-dioxoheptan-3-yl)carbamoyl)-4-(trifluoromethoxy)pyrrolidin-1-yl)-3,3-dimethyl-1-oxobutan-2-yl)carbamate;
Methyl ((R)-1-((2S,4R)-2-(((S)-1-(cyclopropylamino)-6,6-difluoro-1,2-dioxoheptan-3-yl)carbamoyl)-4-(trifluoromethyl)pyrrolidin-1-yl)-3-fluoro-3-methyl-1-oxobutan-2-yl)carbamate;
Methyl ((S)-1-((2(R),3(R))-2-(((S)-1-(cyclopropylamino)-6,6-difluoro-1,2-dioxoheptan-3-yl)carbamoyl)-3-ethylpyrrolidin-1-yl)-3,3-dimethyl-1-oxobutan-2-yl)carbamate;
Methyl ((S)-1-((2(R),3(S))-2-(((S)-1-(cyclopropylamino)-6,6-difluoro-1,2-dioxoheptan-3-yl)carbamoyl)-3-ethylpyrrolidin-1-yl)-3,3-dimethyl-1-oxobutan-2-yl)carbamate;
Methyl ((S)-1-((2(S),3(R))-2-(((S)-1-(cyclopropylamino)-6,6-difluoro-1,2-dioxoheptan-3-yl)carbamoyl)-3-ethylpyrrolidin-1-yl)-3,3-dimethyl-1-oxobutan-2-yl)carbamate;
Methyl ((S)-1-((2(S),3(S))-2-(((S)-1-(cyclopropylamino)-6,6-difluoro-1,2-dioxoheptan-3-yl)carbamoyl)-3-ethylpyrrolidin-1-yl)-3,3-dimethyl-1-oxobutan-2-yl)carbamate;
Methyl ((S)-1-((R)-2-(((S)-1-(cyclopropylamino)-6,6-difluoro-1,2-dioxoheptan-3-yl)carbamoyl)-3,3-dimethylpyrrolidin-1-yl)-3,3-dimethyl-1-oxobutan-2-yl)carbamate;
Methyl ((S)-1-((S)-2-(((S)-1-(cyclopropylamino)-6,6-difluoro-1,2-dioxoheptan-3-yl)carbamoyl)-3,3-dimethylpyrrolidin-1-yl)-3,3-dimethyl-1-oxobutan-2-yl)carbamate;
Methyl ((S)-2-((S)-3-(((S)-1-(cyclopropylamino)-6,6-difluoro-1,2-dioxoheptan-3-yl)carbamoyl)-2-azaspiro[4.5]decan-2-yl)-2-oxo-1-(tetrahydro-2H-pyran-4-yl)ethyl)carbamate;
(S)—N—((S)-1-(cyclopropylamino)-6,6-difluoro-1,2-dioxoheptan-3-yl)-2-((S)-3,3-dimethyl-2-(3-methylureido)butanoyl)-2-azaspiro[4.5]decane-3-carboxamide;
Methyl ((S)-1-((S)-2-(((S)-1-(cyclopropylamino)-6,6-difluoro-1,2-dioxoheptan-3-yl)carbamoyl)-4,4-dimethylpyrrolidin-1-yl)-3,3-dimethyl-1-oxobutan-2-yl)carbamate;
Methyl ((S)-1-((1R, 3(R),4S)-3-(((S)-1-(cyclopropylamino)-6,6-difluoro-1,2-dioxoheptan-3-yl)carbamoyl)-2-azabicyclo[2.2.1]heptan-2-yl)-3,3-dimethyl-1-oxobutan-2-yl)carbamate;
Methyl ((S)-1-((1R, 3(S),4S)-3-(((S)-1-(cyclopropylamino)-6,6-difluoro-1,2-dioxoheptan-3-yl)carbamoyl)-2-azabicyclo[2.2.1]heptan-2-yl)-3,3-dimethyl-1-oxobutan-2-yl)carbamate;
Methyl ((S)-1-((S)-3-(((S)-1-amino-6,6-difluoro-1,2-dioxoheptan-3-yl)carbamoyl)-2-azaspiro[4.5]decan-2-yl)-3,3-dimethyl-1-oxobutan-2-yl)carbamate; and
Methyl ((S)-1-((S)-3-(((S)-6,6-difluoro-1,2-dioxo-1-((pyridin-4-ylmethyl)amino)heptan-3-yl)carbamoyl)-2-azaspiro[4.5]decan-2-yl)-3,3-dimethyl-1-oxobutan-2-yl)carbamate.

Other specific embodiments include compounds enumerated below or pharmaceutically acceptable salts thereof:
Methyl ((S)-1-((1R,2S,5S)-2-(((S)-1-(cyclopropylamino)-6,6-difluoro-1,2-dioxoheptan-3-yl)carbamoyl)-6,6-dimethyl-3-azabicyclo[3.1.0]hexan-3-yl)-3,3-dimethyl-1-oxobutan-2-yl)carbamate;
Methyl ((S)-1-((S)-5-(((S)-6,6-difluoro-1-(methylamino)-1,2-dioxoheptan-3-yl)carbamoyl)-6-azaspiro[2.5]octan-6-yl)-3,3-dimethyl-1-oxobutan-2-yl)carbamate;
Methyl ((S)-1-((2S,4R)-2-(((S)-6,6-difluoro-1-(methylamino)-1,2-dioxoheptan-3-yl)carbamoyl)-4-(trifluoromethyl)piperidin-1-yl)-3,3-dimethyl-1-oxobutan-2-yl)carbamate;
Methyl ((S)-1-((1S,3aR,6aS)-1-(((S)-6,6-difluoro-1-(methylamino)-1,2-dioxoheptan-3-yl)carbamoyl)hexahydrocyclopenta[c]pyrrol-2(1H)-yl)-3,3-dimethyl-1-oxobutan-2-yl)carbamate;
Methyl ((S)-1-((1R,2S,5R)-2-(((S)-1-(cyclopropylamino)-6,6-difluoro-1,2-dioxoheptan-3-yl)carbamoyl)-7,7-dimethyl-3-azabicyclo[3.2.0]heptan-3-yl)-3,3-dimethyl-1-oxobutan-2-yl)carbamate;
Methyl ((S)-1-((1S,3aR,6aS)-1-(((S)-1-(cyclopropylamino)-6,6-difluoro-1,2-dioxoheptan-3-yl)carbamoyl)hexahydrocyclopenta[c]pyrrol-2(1H)-yl)-3,3-dimethyl-1-oxobutan-2-yl)carbamate;
Methyl ((S)-1-((1S,3aR,6aS)-1-(((S)-1-(cyclopropylamino)-6,6-difluoro-1,2-dioxoheptan-3-yl)carbamoyl)-4,4-difluorohexahydrocyclopenta[c]pyrrol-2(1H)-yl)-3,3-dimethyl-1-oxobutan-2-yl)carbamate; and
Methyl ((S)-1-((1S,3aR,6aS)-1-(((S)-6,6-difluoro-1-(methylamino)-1,2-dioxoheptan-3-yl)carbamoyl)-4,4-difluorohexahydrocyclopenta[c]pyrrol-2(1H)-yl)-3,3-dimethyl-1-oxobutan-2-yl)carbamate.

Also included within the scope of the present invention is a pharmaceutical composition which is comprised of a compound of Formula I as described above or a pharmaceutically acceptable salt thereof, and a pharmaceutically acceptable carrier. The pharmaceutical composition can be, for example, in the form of an orally administered tablet or capsule. The invention is also contemplated to encompass a pharmaceutical composition which is comprised of a pharmaceutically acceptable carrier and any of the compounds specifically disclosed in the present application, including pharmaceutically acceptable salts thereof. These and other aspects of the invention will be apparent from the teachings contained herein.

The invention also includes compositions for inhibiting protease in a coronavirus, treating a disease caused by a coronavirus, treating coronavirus infection and preventing coronavirus infection, in a mammal, comprising a compound of the invention in a pharmaceutically acceptable carrier. These compositions may optionally include other antiviral agents. The compositions can be added to blood, blood products, or mammalian organs in order to effect the desired inhibitions.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Also included are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. Also, included are the basic nitrogen-containing groups that may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

If the compounds of Formula I simultaneously contain acidic and basic groups in the molecule the invention also includes, in addition to the salt forms mentioned, inner salts or betaines (zwitterions).

The present invention encompasses all stereoisomeric forms of the compounds of Formula I. Unless a specific stereochemistry is indicated, the present invention is meant to comprehend all such isomeric forms of these compounds. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both each individual enantiomer and mixtures thereof, are embraced within the Formula. When a particular configuration is depicted, that enantiomer (either (R) or (S), at that center) is intended. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

Unless a specific enantiomer or diastereomer is indicated, the invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. The present invention includes all such isomers, as well as salts, solvates (including hydrates) and solvated salts of such racemates, enantiomers, diastereomers and tautomers and mixtures thereof.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (H) and deuterium (H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

When any variable occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant invention in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” (with one or more substituents) should be understood as meaning that the group in question is either unsubstituted or may be substituted with one or more substituents.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁-C₆ alkyl esters and —C₁-C₆ substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “preventing,” or “prophylaxis,” as used herein, refers to reducing the likelihood of contracting disease or disorder described herein, or reducing the severity of a disease or disorder described herein.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group is unsubstituted.

The term “fluoroalkyl,” as used herein refers to an alkyl group as defined above, wherein one or more of the alkyl group's hydrogen atoms has been replaced with a fluorine. In one embodiment, a fluoroalkyl group has from 1 to 6 carbon atoms. In another embodiment, a haloalkyl group is substituted with from 1 to 3 F atoms. Non-limiting examples of fluoroalkyl groups include —CH₂F, —CHF₂, —CF₃, and —CH₂CF₃. The term “C₁-C₆ fluoroalkyl” refers to a fluoroalkyl group having from 1 to 6 carbon atoms.

The term “halo,” as used herein, means —F, —Cl, —Br or —I.

The term “cycloalkyl” means a monocyclic or bicyclic saturated aliphatic hydrocarbon group having the specified number of carbon atoms. For example, “cycloalkyl” includes cyclopropyl, cyclobutyl, cyclopentyl, cyclohexyl, and so on. Bicyclic cycloalkyl ring systems include fused ring systems, where two rings share two atoms, spiro ring systems, where two rings share one atom, and bridged systems.

The term “aryl”, as used herein, represents a stable bicyclic or tricyclic ring system of up to 10 atoms in each ring, wherein at least one ring is aromatic, and all of the ring atoms are carbon. Bicyclic and tricyclic ring systems include fused ring systems, where two rings share two atoms, and spiro ring systems, where two rings share one atom.

The term “heteroaryl”, as used herein, represents a stable monocyclic or bicyclic ring system of up to 10 atoms in each ring, wherein at least one ring is aromatic, and at least one ring contains from 1 to 4 heteroatoms selected from the group consisting of O, N and S. Bicyclic heteroaryl ring systems include fused ring systems, where two rings share two atoms, and spiro ring systems, where two rings share one atom. Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiophenyl, benzoxazolyl, carbazolyl, carbolinyl, cinnolinyl, dihydroindenyl, furanyl, indolinyl, indolyl, indolazinyl, indazolyl, isobenzofuranyl, isoindolyl, isoquinolyl, isothiazolyl, isoxazolyl, naphthalenyl, naphthpyridinyl, oxadiazolyl, oxazolyl, oxazoline, isoxazoline, pyranyl, pyrazinyl, pyrazolyl, pyrazolopyrimidinyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, dihydrobenzodioxinyl, dihydropyrazoloxazinyl, dihydropyrazolyothiazinedioxidyl, methylenedioxybenzene, benzothiazolyl, benzothienyl, quinolinyl, isoquinolinyl, oxazolyl, tetra-hydroquinoline and 3-oxo-3,4dihydro-2N-benzo[b][1,4]thiazine. If the heteroaryl contains nitrogen atoms, it is understood that the corresponding N-oxides thereof are also encompassed by this definition.

The term “heterocycloalkyl”, “heterocycle,” or “heterocyclyl” as used herein is intended to mean a stable nonaromatic monocyclic or bicyclic ring system of up to 10 atoms in each ring, unless otherwise specified, containing from 1 to 4 heteroatoms selected from the group consisting of O, N, S, SO, or SO₂. In some embodiments, heterocycloalkyl are saturated. Bicyclic heterocyclic ring systems include fused ring systems, where two rings share two atoms, and spiro ring systems, where two rings share one atom. “Heterocycloalkyl” therefore includes, but is not limited to the following: azaspirononanyl, azaspirooctanyl, azetidinyl, dioxanyl, oxadiazaspirodecenyl, oxaspirooctanyl, oxazolidinonyl, piperazinyl, piperidinyl, pyrrolidinyl, morpholinyl, thiomorpholinyl, tetrahydrofuranyl, tetrahydropyranyl, dihydropiperidinyl, tetrahydrothiophenyl and the like. If the heterocycle contains a nitrogen, it is understood that the corresponding N-oxides thereof are also encompassed by this definition.

“Celite®” (Fluka) diatomite is diatomaceous earth and can be referred to as “celite”.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., R²) occurs more than one time in any constituent or in Formula I, its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

The invention also relates to medicaments containing at least one compound of the Formula I and/or of a pharmaceutically acceptable salt of the compound of the Formula I and/or an optionally stereoisomeric form of the compound of the Formula I or a pharmaceutically acceptable salt of the stereoisomeric form of the compound of Formula I, together with a pharmaceutically suitable and pharmaceutically acceptable vehicle, additive and/or other active substances and auxiliaries.

The term “patient” used herein is taken to mean mammals such as primates, humans, sheep, horses, cattle, pigs, dogs, cats, rats, and mice.

The term “coronavirus” includes HCoV-229E, HCoV-NL63, HCoV—OC43, HCoV—HKU1, severe acute respiratory syndrome coronavirus (SARS-CoV), Middle East respiratory syndrome coronavirus (MERS-CoV) and SARS-CoV-2.

The medicaments according to the invention can be administered by oral, inhalative, rectal or transdermal administration or by subcutaneous, intraarticular, intraperitoneal or intravenous injection. Oral administration is preferred. Coating of stents with compounds of the Formula (I) and other surfaces which come into contact with blood in the body is possible.

The invention also relates to a process for the production of a medicament, which comprises bringing at least one compound of the Formula (I) into a suitable administration form using a pharmaceutically suitable and pharmaceutically acceptable carrier and optionally further suitable active substances, additives or auxiliaries.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the protease inhibitors of the instant invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the protease inhibitors, when used for the indicated effects, will range between about 0.01 mg per kg of body weight per day (mg/kg/day) to about 30 mg/kg/day, for instance, 0.01-20 mg/kg/day, 0.01-15 mg/kg/day, 0.01-10 mg/kg/day or 0.01-5 mg/kg/day (unless specified otherwise, amounts of active ingredients are on free base basis). For example, an 80 kg patient would receive between about 0.8 mg/day and 2.4 g/day, e.g., 0.8-1600 mg/day, 0.8-1200 mg/day, 0.8-800 mg/kg/day, or 0.8-400 mg/day. A suitably prepared medicament for once a day administration would thus contain between 0.8 mg and 2.4 g, between 0.8 mg and 1600 mg, between 0.8 mg and 1200 mg, between 0.8 mg and 800 mg, or between 0.8 and 400 mg, e.g., 1 mg, 4 mg, 8 mg, 10 mg, 20 mg, 40 mg, 80 mg, 160 mg, 200 mg, 300 mg, or 400 mg. Advantageously, the protease inhibitors may be administered in divided doses of two, three, or four times daily. For administration twice a day, a suitably prepared medicament would contain between 0.4 mg and 1.2 g, between 0.4 mg and 800 mg, between 0.4 mg and 600 mg, between 0.4 mg and 400 mg, or between 0.4 and 200 mg, e.g., 0.5 mg, 2 mg, 4 mg, 5 mg, 10 mg, 20 mg, 40 mg, 80 mg, 100 mg, 150 mg, or 200 mg.

Intravenously, the patient would receive the active ingredient in quantities sufficient to deliver between 0.01-15 mg/kg/day, e.g., 0.01-7.5 mg/kg/day or 0.1-5 mg/kg/day. Such quantities may be administered in a number of suitable ways, e.g., large volumes of low concentrations of active ingredient during one extended period of time or several times a day, low volumes of high concentrations of active ingredient during a short period of time, e.g., once a day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily determined by a person having ordinary skill in the art.

Compounds of Formula I can be administered both as a monotherapy and in combination with additional therapeutic agents (also referred to herein as “second therapeutic agents”), including other antivirals or treatments of coronavirus infection.

The protease inhibitors of the instant invention can also be co-administered with suitable antivirals, including, but not limited to, agents that inhibit the replication of viruses such as nucleoside polymerase inhibitors, agents that induce viral error catastrophe protease inhibitors, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors. In a class of the invention, the protease inhibitors of the instant invention can be co-administered with a nucleoside polymerase inhibitor, a protease inhibitor, or a combination thereof. Skilled practitioners will acknowledge that such antivirals in some cases may be co-administered as prodrugs.

Polymerase inhibitors that can be co-administered with the protease inhibitors of the instant invention include, but are not limited to, clevudine, remdesivir (VEKLURY), favipiravir (AVIGAN) and AT-527.

Protease inhibitors that can be co-administered with the protease inhibitors of the instant invention include, but are not limited to, camostat mesylate, upamostat, SLV213, PF-0083523, CDI-45205, ALG-097111, GC-376 and TJC-0642.

Agents that induce viral error catastrophe that can be co-administered with the protease inhibitors of the invention include molnupiravir.

eEF1A inhibitors that can be co-administered with the protease inhibitors of the instant invention include, but are not limited to, plitidepsin.

Androgen receptor antagonists that can be co-administered with the protease inhibitors of the instant invention include, but are not limited to, proxalutamide.

Dihydroorotate dehydrogenase (DHODH) inhibitors that can be co-administered with the protease inhibitors of the instant invention include, but are not limited to, PTC299 and brequinlar.

Sphingosine kinase inhibitors that can be co-administered with the protease inhibitors of the instant invention include, but are not limited to, opaganib.

MEK inhibitors that can be co-administered with the protease inhibitors of the instant invention include, but are not limited to, ATR-002.

Antimalarials that can be co-administered with the protease inhibitors of the instant invention include, but are not limited to, tafenoquine (ARAKODA).

CCR5 inhibitors that can be co-administered with the protease inhibitors of the instant invention include, but are not limited to, maraviroc and vicriviroc.

PIKfyve kinase inhibitors that can be co-administered with the protease inhibitors of the instant invention include, but are not limited to, Apilimod.

Serine protease inhibitors that can be co-administered with the protease inhibitors of the instant invention include, but are not limited to, nafamostat mesylate.

Glycosylation inhibitors that can be co-administered with the protease inhibitors of the instant invention include, but are not limited to, WP1122.

Alternatively or additionally, one or more additional pharmacologically active agents may be administered in combination with a compound of the invention. The additional active agent (or agents) is intended to mean a pharmaceutically active agent (or agents) that is active in the body, including pro-drugs that convert to pharmaceutically active form after administration, which is different from the compound of the invention, and also includes free-acid, free-base and pharmaceutically acceptable salts of said additional active agents when such forms are sold commercially or are otherwise chemically possible. Generally, any suitable additional active agent or agents, including but not limited to polymerase nucleoside inhibitors, protease inhibitors, agents that induce viral error catastrophe, eEFTA inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors can be used in any combination with the compound of the invention in a single dosage formulation (a fixed dose drug combination), or may be administered to the patient in one or more separate dosage formulations which allows for concurrent or sequential administration of the active agents (co-administration of the separate active agents).

Typical doses of the protease inhibitors of the invention in combination with other suitable polymerase nucleoside inhibitors, protease inhibitors, agents that induce viral error catastrophe, eEFTA inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors may be the same as those doses of the protease inhibitors administered without coadministration of additional polymerase nucleoside inhibitors, protease inhibitors, agents that induce viral error catastrophe, eEF1A inhibitors, androgen receptor antagonists, Dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors, or may be substantially less that those doses of protease inhibitors administered without coadministration of polymerase nucleoside inhibitors, protease inhibitors, agents that induce viral catastrophe, eEF1A inhibitors, androgen receptor antagonists, dihydroorotate dehydrogenase (DHODH) inhibitors, sphingosine kinase inhibitors, MEK inhibitors, antimalarials, CCR5 inhibitors, PIKfyve kinase inhibitors, serine protease inhibitors and glycosylation inhibitors depending on a patient's therapeutic needs.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e., prevent, inhibit or ameliorate) the viral condition or treat the progression of the disease in a host.

The compounds of the invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of each of the compounds when administered individually as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

The present invention is not limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the relevant art and are intended to fall within the scope of the appended claims.

### GENERAL PROCEDURES

Starting materials and intermediates were purchased or were prepared using known procedures described in the chemical synthetic literature or as otherwise described. The preparation of the various starting materials used herein is well within the skill of a person versed in the art. Routes applied to the synthesis of compounds of Formula I are described in the following schemes. In some cases, the sequence of reaction steps may be varied to facilitate reactions or to avoid unwanted reaction products. In some cases, the final product may be further modified, for example, by manipulation of substituents. These manipulations may include, but are not limited to, reduction, oxidation, alkylation, acylation, and hydrolysis reactions which are commonly known to those skilled in the art. Because the schemes are an illustration, the invention should not be construed as being limited by the chemical reactions and conditions expressed. The examples described below are provided so that the invention might be more fully understood. These examples are illustrative only and should not be construed as limiting the invention in any way

It is understood that a chiral center in a compound may exist in the S or R absolute configuration, or as a mixture of both. Within a molecule, each bond drawn as a straight line from a chiral center includes both the R and S stereoisomers as well as mixtures thereof Δn asterisk denotes a stereocenter in a single configuration, either R or S. Absolute stereochemistry of separate stereoisomers in the examples and intermediates are not determined unless stated otherwise in an example or explicitly in the nomenclature.

Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are carried out by employing reactions as shown in the schemes and examples herein, in addition to other standard manipulations such as ester hydrolysis, cleavage of protecting groups, etc., as may be known in the literature or exemplified in the experimental procedures.

Reactions sensitive to moisture or air were performed under nitrogen or argon using anhydrous solvents and reagents. The progress of reactions was determined by either liquid chromatography-mass spectrometry (LCMS) or analytical thin layer chromatography (TLC) usually performed with Merck KGaA glass-backed TLC plates, silica gel 60 F₂₅₄.

Analytical LCMS was commonly performed on a Waters SQD single quadrupole mass spectrometer with electrospray ionization in positive ion detection mode (mass range set at 150-900 daltons, data collected in centroid mode and scan time set to 0.2 seconds) and a Waters Acquity UPLC system (binary solvent manager, sample manager, and TUV). The column used was a Waters Acquity BEH C18 1×50 mm, 1.7 μm, heated to 50° C. The mobile phases used were modified with either acidic or basic additives. The acidic mobile phase consisted of 0.1% trifluoroacetic acid in water for Solvent A and 100% acetonitrile for Solvent B. A two-minute run was established at a flow rate of 0.3 ml/min with Initial conditions of 95% Solvent A and ramping up to 99% Solvent B at 1.60 minutes and holding at 99% Solvent B for 0.40 minutes. The injection volume was 0.5 μL using partial loop needle overfill injection mode. The TUV monitored wavelength 215 or 254 nm with a sampling rate of 20 points/second, normal filter constant and absorbance data mode. The basic mobile phase consisted of 0.1% ammonium hydroxide in water for solvent A and 100% Acetonitrile for solvent B. A two-minute run was established at a flow rate of 0.3 ml/min with initial conditions of 99% Solvent A and ramping up to 99% Solvent B at 1.90 minutes and holding at 99% Solvent B for 0.10 minutes. A five-minute run was established at a flow rate of 0.3 ml/min with initial conditions of 95% Solvent A and ramping up to 99% Solvent B at 4.90 minutes and holding at 99% Solvent B for 0.10 minutes. For both methods, the injection volume was 5.0 μL using Partial Loop Needle Overfill Injection mode. The TUV monitored wavelength 215 nm with a sampling rate of 20 points/second, normal filter constant and absorbance data mode. Alternatively, a commonly used system consisted of a Waters ZQ™ platform with electrospray ionization in positive ion detection mode with an Agilent 1100 series HPLC with autosampler. The column was commonly a Waters Xterra MS C18, 3.0×50 mm, 5 μm or a Waters Acquity UPLC® BEH C18 1.0×50 mm, 1.7 μm. The flow rate was 1 mL/min, and the injection volume was 10 μL. UV detection was in the range 210-400 nm. The mobile phase consisted of solvent A (water plus 0.05% TFA) and solvent B (MeCN plus 0.05% TFA) with a gradient of 100% solvent A for 0.7 min changing to 100% solvent B over 3.75 min, maintained for 1.1 min, then reverting to 100% solvent A over 0.2 min.

Preparative reverse-phase chromatography was generally carried out on a Teledyne ISCO ACCQPrep HP125 or HP150 apparatus equipped with UV and ELSD detectors. The UV detector typically monitored wavelengths of 215 and 254 nm. The column was commonly one of the following: Waters XBridge Prep C18 OBD 5 μm 30×150 mm, Waters XBridge Prep C18 OBD 5 μm 30×250 mm, Waters XBridge Prep C18 OBD 5 μm 50×250 mm, Waters SunFire Prep C18 OBD 5 μm 30×150 mm, Waters SunFire Prep C18 OBD 10 μm 30×150 mm, Waters SunFire Prep C18 OBD 5 μm 50×250 mm, Waters SunFire Prep C18 OBD 10 μm 50×250 mm, or Phenomenex Luna Prep C18 5 μm 50×250 mm. The mobile phases consisted of mixtures of 0.1% TFA in acetonitrile with 0.1% TFA in water or mixtures of 100% acetonitrile with 5 mM (NH₄)HCO₃. Alternatively, a commonly used system was a Waters Chromatography Workstation configured with an LCMS system consisting of: Waters ZQ™ single quad MS system with Electrospray Ionization, Waters 2525 Gradient Pump, Waters 2767 Injector/Collector, Waters 996 PDA Detector. MS conditions were: 150-750 amu, positive electrospray, collection triggered by MS. Columns used were commonly a Waters SunFire C18 5 μm 30×150 mm, a Boston Green ODS 5 μm 150×30 mm, or a YMC-Actus Triart C18 5 μm 150×30 mm column. The mobile phases consisted of mixtures of acetonitrile (10-100%) in water containing 0.1% TFA. Flow rates were maintained at 50 mL/min, and the UV detection range was 210-400 nm. An additional preparative HPLC system used was a Gilson Workstation consisting of: Gilson GX-281 Injector/Collector, Gilson UV/VIS-155 Detector, Gilson 333 and 334 Pumps, and either a Phenomenex Gemini-NX C18 5 μm 50×250 mm column, a Waters XBridge Prep C18 OBD 5 μm 30×250 mm, or a Welch Xtimate C18 5 μm 150×25 mm. The mobile phases consisted of mixtures of acetonitrile (0-75%) in water containing 5 mM (NH₄)HCO₃. Flow rates were maintained at 50 mL/min for the Waters XBridge column, 90 mL/min for the Phenomenex Gemini column, and 25 mL/min for the Welch Xtimate column. The UV detection range was 210-400 nm. Mobile phase gradients were optimized for the individual compounds.

Flash chromatography was usually performed using an ISCO CombiFlash Rf apparatus, a Biotage® Flash Chromatography apparatus (Dyax Corp.), or an ISCO CombiFlash® Companion XL apparatus on silica gel (60 Å pore size) in pre-packed RediSep Rf, RediSep Rf Gold, or SepaFlash columns. Mobile phases generally consisted of mixtures of hexanes or dichloromethane with EtOAc, 3:1 EtOAc:EtOH, or MeOH. Mobile phase gradients were optimized for the individual compounds.

Chiral chromatography was commonly performed by supercritical fluid chromatography with a column chosen from one of the following: Daicel CHIRALPAK AD-H 2×25 cm, Daicel CHIRALPAK AD-H 3×25 cm, YMC Chiral ART Cellulose-SC, Lux Cellulose-25 μm 30×250 mm, or Exsil Chiral-NR 8 μm 30×250 mm. Mobile phases consisted of mixtures of CO₂ with methanol, ethanol, isopropanol+0.10% diethylamine, isopropanol+0.10% NH₄OH, or 1:1 isopropanol:hexanes+0.1% 2 M NH₃/MeOH. Mobile phase gradients were optimized for the individual compounds. Pressure was typically maintained at 100 bar, and flow rates ranged from 50-200 mL/min. UV monitoring was generally carried out at 220 or 205 nM.

¹H NMR data were typically acquired using using a Bruker NEO 500 MHz NMR spectrometer equipped with a room temperature 5 mm BBF iProbe, a Bruker Avance NEO 400 MHz NMR spectrometer equipped with a Bruker PI HR-BBO400S1-BBF/H/D-5.0—Z SP probe, or a Bruker Avance III 500 MHz NMR spectrometer equipped with a Bruker 5 mm PABBO probe. Chemical shift values are reported in delta (δ) units, parts per million (ppm). Chemical shifts for ¹H NMR spectra are given relative to signals for residual non-deuterated solvent (CDCl₃ referenced at δ 7.26 ppm; DMSO-d₆ referenced at δ 2.50 ppm and CD₃OD referenced at δ 3.31 ppm). Multiplets are reported by the following abbreviations: s=singlet, d=doublet, t=triplet, q=quartet, dd=doublet of doublets, m=multiplet or overlap of nonequivalent resonances. Coupling constants (J) are reported in Hertz (Hz). When compounds appear as mixtures of rotamers by NMR, spectral data corresponding to the major species observed in solution are reported.

### Abbreviations:

AOP is tris(dimethylamino)(3H-1,2,3-triazolo[4,5-b]pyridin-3-yloxy)phosphorus hexafluorophosphate; aq. is aqueous; BAST is N,N-bis(2-methoxyethyl)aminosulfur trifluoride; Bn is benzyl; Boc is tert-butoxycarbonyl; Cbz is benzyloxycarbonyl; DAST is diethylaminosulfur trifluoride; DCM is dichloromethane; DIBAL is diisobutylaluminium hydride; DIEA or DIPEA is N,N-diisopropylethylamine; DMF is N,N-dimethylformamide; DMP is Dess-Martin periodinane; DMS is dimethylsulfide; DMSO is dimethyl sulfoxide; EDC or EDCI is 1-ethyl-3-(3-dimethylaminopropyl)carbodiimide; ELSD is evaporative light scattering detector; Et is ethyl; HATU is (1-[Bis(dimethylamino)methylene]-1H-1,2,3-triazolo[4,5-b]pyridinium 3-oxide hexafluorophosphate; HMDS is hexamethyldisilazide or bis(trimethylsilyl)amide; HOBt is hydroxybenzotriazole; HPLC is high-pressure liquid chromatography; LAH is lithium aluminum hydride; LCMS is liquid chromatography-mass spectrometry; mCPBA is meta-chloroperoxybenzoic acid; Me is methyl; MTBE is methyl tert-butyl ether; OAc is acetate; OMe is methoxy; NMR is nuclear magnetic resonance; Ph is phenyl; RT or rt is room temperature; sat. is saturated; TBAF is tetrabutylammonium fluoride; TEMPO is (2,2,6,6-tetramethylpiperidin-1-yl)oxy; ^{t}Bu is tert-butyl; TBS is tert-butyldimethylsilyl; TEA is triethylamine; TFA is trifluoroacetic acid; THF is tetrahydrofuran; TMS is trimethylsilyl; UV is ultraviolet.

As illustrated in Scheme A, in general, compounds of the invention can be prepared by amide coupling of an appropriately functionalized amine A-1 and an acid of type Int-1 to provide compounds of formula A-2. Esters A-2 can be hydrolyzed to yield acids of formula A-3, which can be coupled with amines of formula Int-2 to afford products of formula A-4. Alternatively, compounds of type A-4 may be prepared from an amine A-1 and an acid of type Int-1 bearing a protecting group in place of the acyl group containing R^{b}. The protecting group may be removed and replaced with an appropriate acyl group to afford the corresponding hydroxyamide of formula A-4. Hydroxyamides A-4 can be oxidized to afford ketoamides of formula A-5. In some embodiments, stereoisomers may be separated during the course of the synthesis. Amines of type A-1, acids of type Int-1, and amines of type Int-2 are commercially available or may be synthesized from appropriate intermediates.

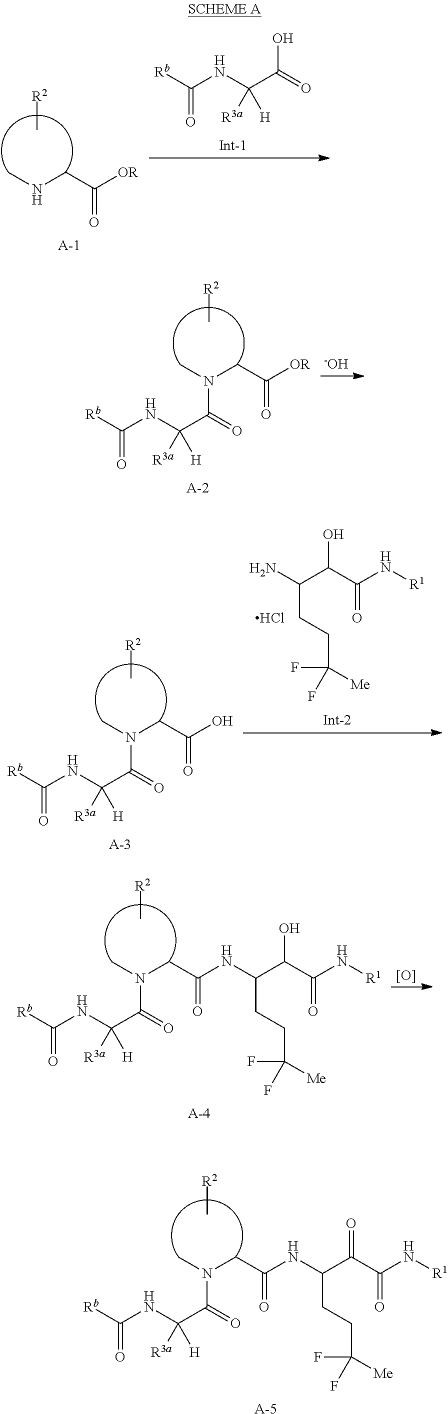

As illustrated in Scheme B, in general, compounds of the invention can be prepared by amide coupling of an appropriately functionalized acid B-1 and an amine of type Int-2 to provide compounds of formula B-2, which can be deprotected to afford amines of formula B-3. Amines B-3 can be coupled with acids of type Int-1 to yield hydroxyamides of formula B-4, which can undergo oxidation to afford ketoamides of formula B-5. In some embodiments, stereoisomers may be separated during the course of the synthesis. Acids of type B-1, amines of type Int-2, and acids of type Int-1 are commercially available or may be synthesized from appropriate intermediates.

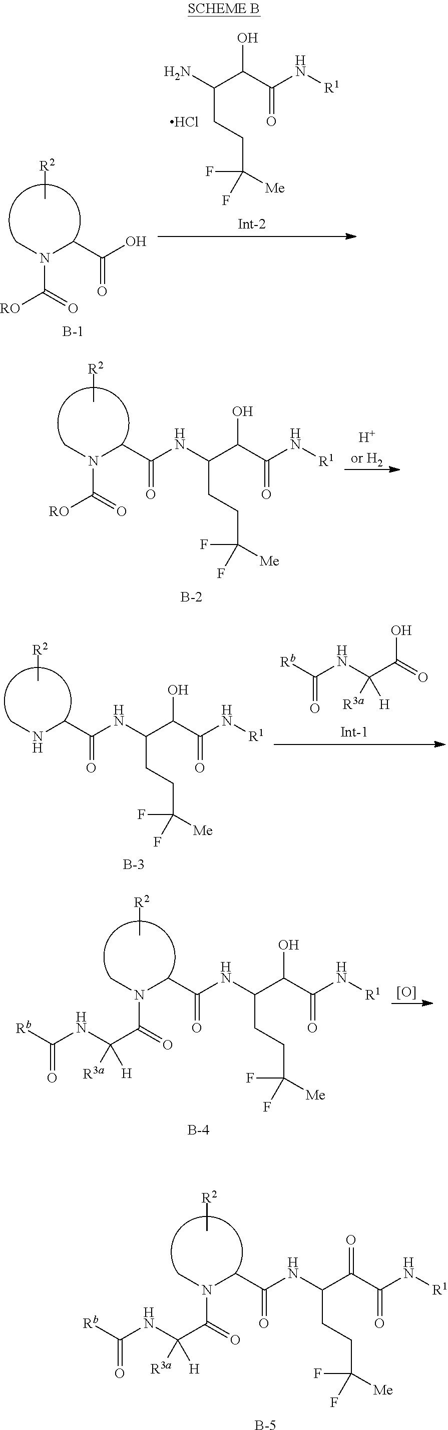

As illustrated in Scheme C, in general, compounds of the invention can be prepared by amide coupling of an appropriately functionalized amine C-1 and an acid of type Int-1 to provide compounds of formula C-2. Esters C-2 can be hydrolyzed to yield acids of formula C-3, which can be coupled with amines of formula Int-3 to afford products of formula C-4. Acetals C-4 can be hydrolyzed under acidic conditions to provide aldehydes of type C-5, which can undergo a Passerini reaction to afford compounds of formula C-6. In some embodiments, trifluoroacetic acid may be used in place of acetic acid, affording the trifluoroacetate instead of the acetate product. Compounds C-6 can be hydrolyzed to hydroxyamides of type C-7, which can be oxidized to deliver ketoamides of formula C-8. In some embodiments, stereoisomers may be separated during the course of the synthesis. Amines of type C-1, acids of type Int-1, and amines of type Int-3 are commercially available or may be synthesized from appropriate intermediates.

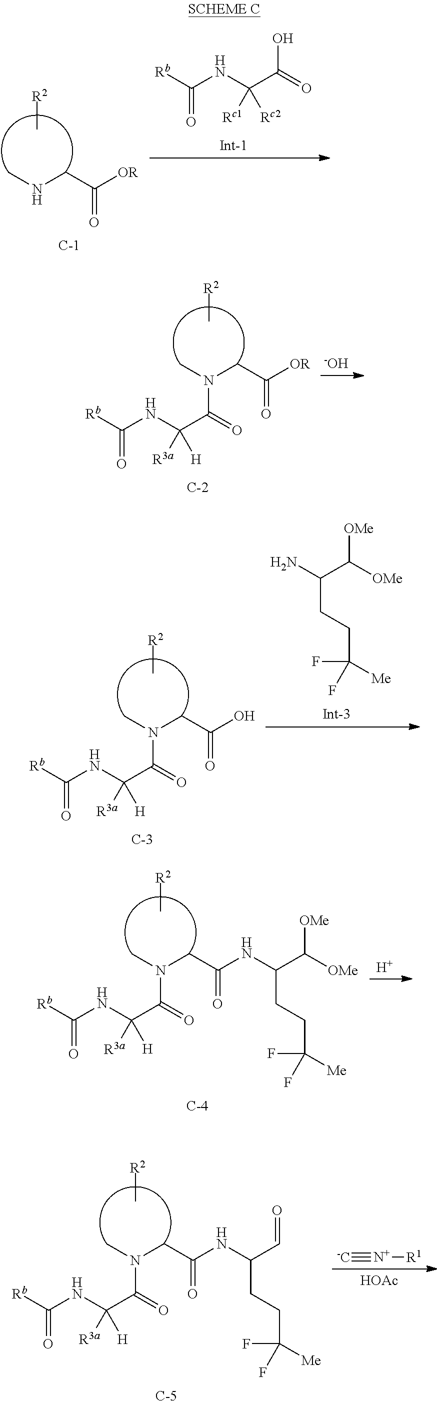

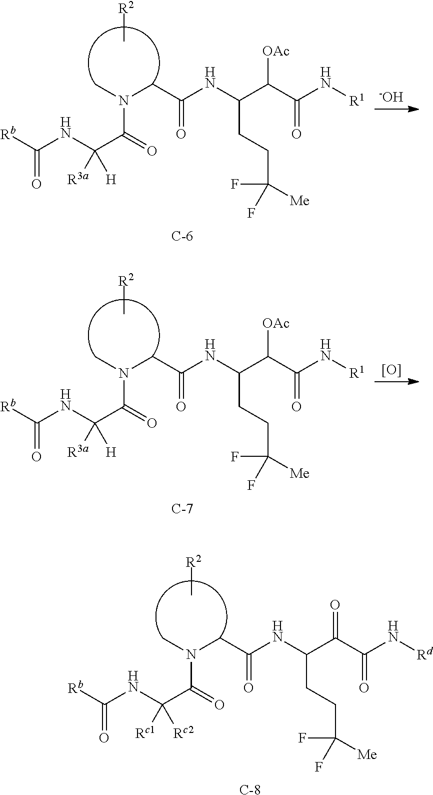

As illustrated in Scheme D, in general, compounds of the invention can be prepared by amide coupling of an appropriately functionalized acid D-1 and an amine of type Int-3 to provide compounds of formula D-2, which can be deprotected to afford amines of formula D-3. Amines D-3 can be coupled with acids of type Int-1 to yield compounds of formula D-4. Acetals D-4 can be hydrolyzed under acidic conditions to provide aldehydes of type D-5, which can undergo a Passerini reaction to afford compounds of formula D-6. In some embodiments, trifluoroacetic acid may be used in place of acetic acid, affording the trifluoroacetate instead of the acetate product. Compounds D-6 can be hydrolyzed to hydroxyamides of type D-7, which can be oxidized to deliver ketoamides of formula D-8. In some embodiments, R² is a group that can be transformed into a different substituent during the course of the synthesis. In some embodiments, stereoisomers may be separated during the course of the synthesis. Acids of type D-1, amines of type Int-3, and acids of type Int-1 are commercially available or may be synthesized from appropriate intermediates.

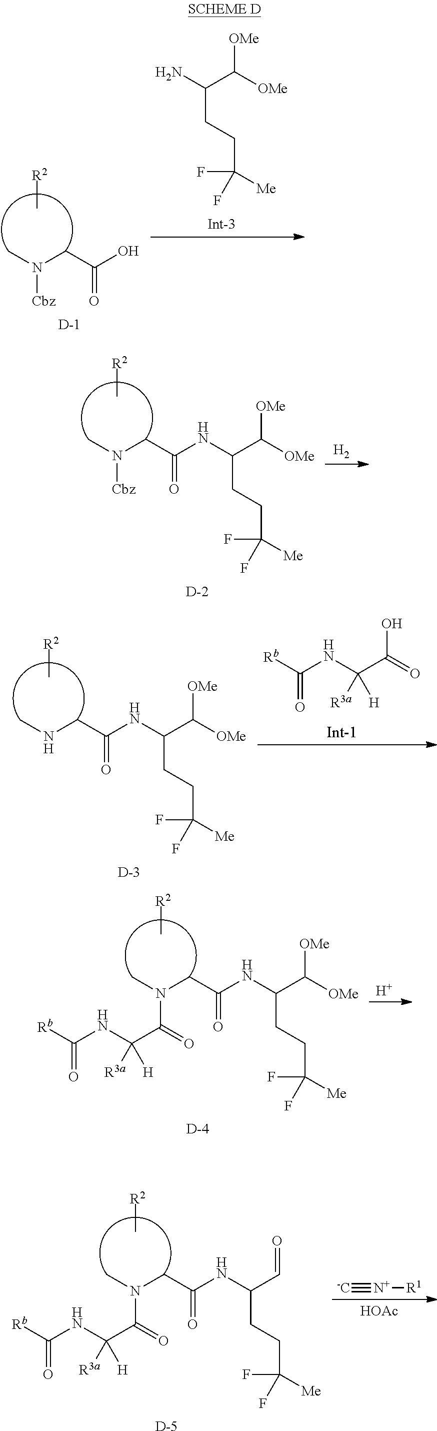

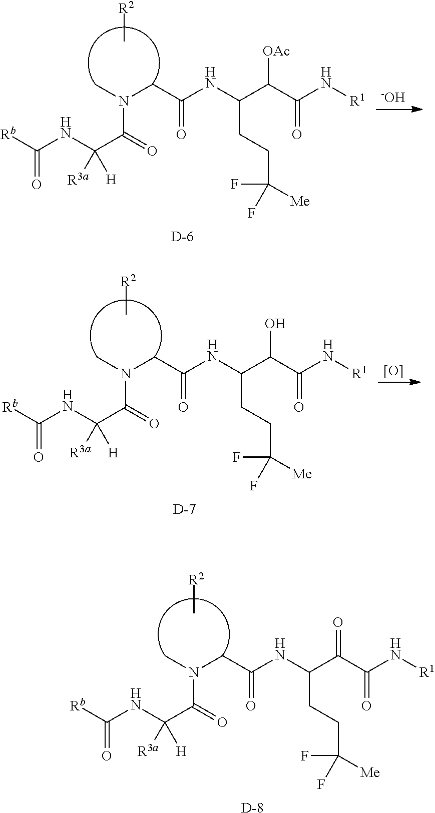

As illustrated in Scheme E, in general, compounds of the invention can be prepared from intermediate D-5 by treatment with trimethylsilyl cyanide to give cyanohydrins of formula E-1. These products can be converted to methyl esters of formula E-2 by treatment with acid and methanol. Compounds of type E-2 can be hydrolyzed to provide the corresponding acids E-3, which can then be joined with amines under amide coupling conditions to provide compounds of formula E-4. Hydroxyamides E-4 can be oxidized to deliver the corresponding ketoamides of formula E-5. In some embodiments, stereoisomers may be separated during the course of the synthesis.

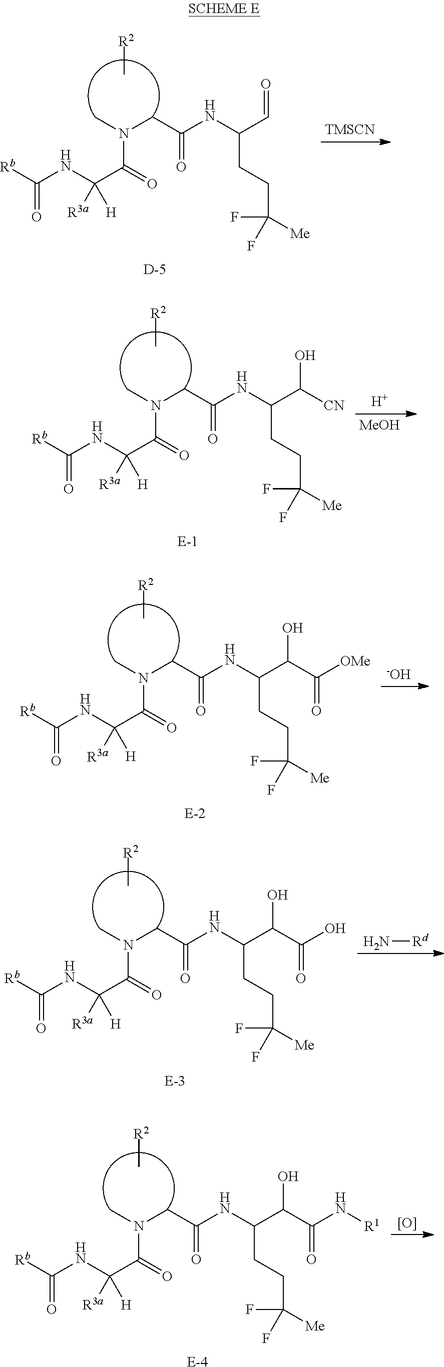

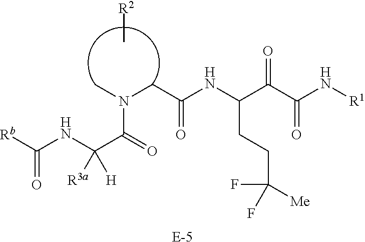

As illustrated in Scheme F, in general, compounds of the invention can be prepared from intermediate E-1 by treatment with hydrogen peroxide to yield hydroxyamides of formula F-1. Oxidation can then provide ketoamides of formula F-2.

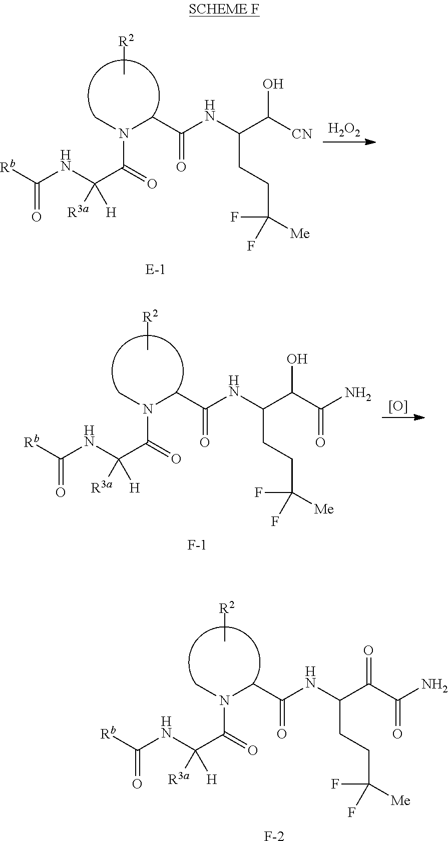

### SYNTHESIS OF INTERMEDIATES

In the section below, the preparation of certain intermediates useful in preparing the compounds of the invention are described.

### Intermediate 1

### Methyl (S)-6-azaspiro[2.5]octane-5-carboxylate hydrochloride

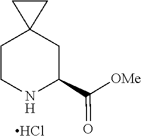 [SMILES: COC(=O)[C@@H]1CC2(CCN1)CC2 ; Cl]

### Step 1: Methyl (S)-6-azaspiro[2.5]octane-5-carboxylate hydrochloride

To a solution of (S)-6-(tert-butoxycarbonyl)-6-azaspiro[2.5]octane-5-carboxylic acid (1.00 g, 3.92 mmol) in MeOH (6.53 mL) was added thionyl chloride (858 μL, 11.8 mmol) at 0° C. slowly over 2 minutes. The reaction mixture was warmed to ambient temperature and stirred overnight. The solvent was removed under reduced pressure to give the title compound. LRMS m/z: (M+H)⁺ calculated 170.1; found 170.2.

The following compounds were prepared in essentially the same manner:

| Intermediate | | | LRMS m/z |
|---|---|---|---|
| No. | Structure | Name | (M + H)⁺ |
| 2 | 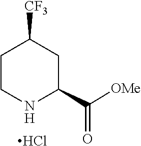 [SMILES: COC(=O)[C@@H]1C[C@H](C(F)(F)F)CCN1 ; Cl] | Methyl (2S,4R)-4- (trifluoromethyl)piperidine-2- carboxylate hydrochloride | Calculated: 212.1 Found: 212.1 |
| 3 | 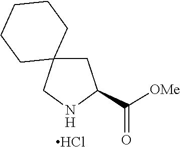 [SMILES: COC(=O)[C@@H]1CC2(CCCCC2)CN1 ; Cl] | Methyl (S)-2- azaspiro[4.5]decane-3- carboxylate hydrochloride | Calculated: 198.1 Found: 198.2 |
| 4 | 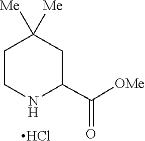 [SMILES: COC(=O)C1CC(C)(C)CCN1 ; Cl] | Methyl 4,4- dimethylpiperidine-2- carboxylate hydrochloride | Calculated: 172.1 Found: 172.2 |
| 5 | 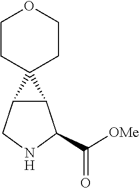 [SMILES: COC(=O)[C@H]1NC[C@H]2[C@@H]1C21CCOCC1] | Methyl (1R,2S,5S)-tetrahydro- 3- azaspiro[bicyclo[3.1.0]hexane- 6,4′-pyran]-2-carboxylate hydrochloride | Calculated: 212.1 Found: 212.2 |
| 6 | 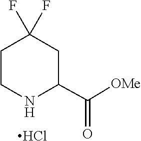 [SMILES: COC(=O)C1CC(F)(F)CCN1 ; Cl] | Methyl 4,4-difluoropiperidine- 2-carboxylate hydrochloride | Calculated: 180.1 Found: 180.1 |
| 7 | 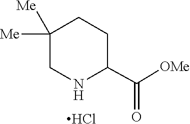 [SMILES: COC(=O)C1CCC(C)(C)CN1 ; Cl] | Methyl 5,5- dimethylpiperidine-2- carboxylate hydrochloride | Calculated: 172.1 Found: 172.2 |
| 8 | 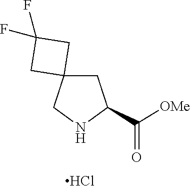 [SMILES: COC(=O)[C@@H]1CC2(CN1)CC(F)(F)C2 ; Cl] | Methyl (S)-2,2-difluoro-6- azaspiro[3.4]octane-7- carboxylate hydrochloride | Calculated: 206.1 Found: 206.1 |
| 9 | 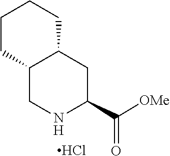 [SMILES: COC(=O)[C@@H]1C[C@H]2CCCC[C@H]2CN1 ; Cl] | Methyl (3S,4aR,8aR)- decahydroisoquinoline-3- carboxylate | Calculated: 198.1 Found: 198.2 |

### Intermediate 10

### Methyl (S)-6-azaspiro[2.5]octane-5-carboxylate hydrochloride

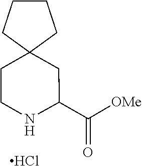 [SMILES: COC(=O)C1CC2(CCCC2)CCN1 ; Cl]

### Step 1: Methyl 8-azaspiro[4.5]decane-7-carboxylate hydrochloride

To a solution of 8-azaspiro[4.5]decane-7-carboxylic acid, HCl (950 mg, 4.32 mmol) in MeOH (7.21 mL) was added thionyl chloride (947 μL, 13.0 mmol) at 0° C. slowly over 2 minutes. The mixture was warmed to ambient temperature and stirred for 18 hours. The solvent was removed under reduced pressure to give the title compound. LRMS m/z: (M+H)⁺ calculated 198.1; found 198.0.

The following compounds were prepared in essentially the same manner:

| Intermediate | | | LRMS m/z |
|---|---|---|---|
| No. | Structure | Name | (M + H)⁺ |
| 11 | 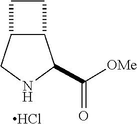 [SMILES: COC(=O)[C@H]1NC[C@@H]2CC[C@@H]21 ; Cl] | Rac-methyl (1R,2S,5S)- 6,6,7,7-tetramethyl-3- azabicyclo[3.2.0] heptane-2-carboxylate hydrochloride | Calculated: 156.1 Found: 156.1 |
| 12 | 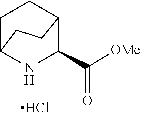 [SMILES: COC(=O)[C@H]1NC2CCC1CC2 ; Cl] | Methyl (S)-2- azabicyclo[2.2.2]octane-3- carboxylate hydrochloride | Calculated: 170.1 Found: 170.2 |
| 13 | 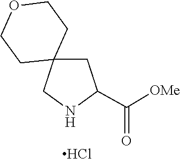 [SMILES: COC(=O)C1CC2(CCOCC2)CN1 ; Cl] | Methyl 8-oxa-2- azaspiro[4.5]decane-3- carboxylate hydrochloride | Calculated: 200.1 Found: 200.1 |

### Intermediate 14

### Rac-ethyl (1S,3aS,6aS)-4,4-difluorooctahydrocyclopenta[c]pyrrole-1-carboxylate hydrochloride

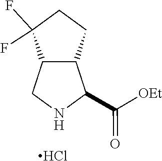

### Step 1: Rac-2-(tert-butyl) 1-ethyl (1S,3aS,6aS)-4,4-difluorohexahydrocyclopenta[c]pyrrole-1,2(1H)-dicarboxylate

To a stirred solution of 2-(tert-butyl) 1-ethyl (1S,3aR,6aS)-4-oxohexahydrocyclopenta[c]pyrrole-1,2(1H)-dicarboxylate (450 mg, 1.51 mmol), and CH₂Cl₂ (15.1 mL) was added BAST (7.57 mL, 7.57 mmol) and the reaction was stirred at room temperature overnight. The reaction was diluted with CH₂Cl₂ and then washed with saturated aqueous NaHCO₃ and brine, dried (MgSO₄), filtered, and concentrated under reduced pressure. DMSO (5 mL) was added to the residue, which was filtered and the filtrate purified by reverse phase HPLC (Waters SunFire Prep C18 OBD 10 μm 50×250 mm) eluting with a gradient of 5-95% acetonitrile/water+0.1% TFA over 20 minutes. Fractions containing product were combined, basified with saturated aq. NaHCO₃, and then extracted with CH₂Cl₂. The combined organic portions were washed with brine, dried (MgSO₄), filtered, and concentrated under reduced pressure to give the title compound. LRMS m/z: (M+H)⁺ calculated 320.2; found 320.3.

### Step 2: Rac-ethyl (1S,3aS,6aS)-4,4-difluorooctahydrocyclopenta[c]pyrrole-1-carboxylate hydrochloride

To a stirred solution of 2-(tert-butyl) 1-ethyl (1S,3aS,6aS)-4,4-difluorohexahydrocyclopenta[c]pyrrole-1,2(1H)-dicarboxylate (330 mg, 1.03 mmol) and CH₂Cl₂ (15.1 mL) at room temperature was added TFA (0.796 mL, 10.3 mmol). The mixture was stirred for 2 hours at room temperature. The solvent was removed under reduced pressure. The residue was dissolved in 10 mL of 4 M HCl in EtOAc and stirred for 5 minutes before the solvent was removed under reduced pressure. The residue was azeotroped with EtOAc (3×10 ml) to give the title compound. LRMS m/z: (M+H)⁺ calculated 220.1; found 220.2.

### Intermediate 15

### Benzyl (2S,4(R or S))-4-hydroxy-4-(trifluoromethyl)piperidine-2-carboxylate hydrochloride

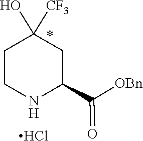 [SMILES: O=C(OCc1ccccc1)[C@@H]1CC(O)(C(F)(F)F)CCN1 ; Cl]

### Step 1: 2-benzyl 1-(tert-butyl) (S)-4-oxopiperidine-1,2-dicarboxylate

To a stirred solution of (S)-1-(tert-butoxycarbonyl)-4-oxopiperidine-2-carboxylic acid (2 g, 8.22 mmol) and DMF (15.1 mL) at ambient temperature was added cesium carbonate (2.68 g, 8.22 mmol) followed by benzyl bromide (1.22 mL, 10.3 mmol). The mixture was stirred at room temperature for 18 hours. The mixture was diluted with H₂O and then extracted with EtOAc (2×50 mL). The combined organic portions were washed with saturated aq. NaHCO₃ and brine, dried (MgSO₄), filtered, and concentrated under reduced pressure. The residue was purified by column chromatography on silica gel eluting with 0-100% EtOAc in hexanes to give the title compound. LRMS m/z: (M+H)⁺ calculated 334.2; found 334.3.

### Step 2: 2-Benzyl 1-(tert-butyl) (2S,4(R or S))-4-hydroxy-4-(trifluoromethyl)piperidine-1,2-dicarboxylate

To a stirred solution of 2-benzyl 1-(tert-butyl) (S)-4-oxopiperidine-1,2-dicarboxylate (500 mg, 1.50 mmol), and THF (7.5 mL) at 0° C. was added trifluoromethyltrimethylsilane (665 μL, 4.50 mmol) followed by TBAF (150 μL, 0.150 mmol) and the reaction was stirred for 30 minutes at 0° C. followed by the removal of the ice bath. After 30 minutes, the reaction was diluted with EtOAc and then washed with 0.1 N HCl and brine, dried (MgSO₄), filtered and concentrated under reduced pressure. The residue was dissolved in THF (7.50 mL) and then treated with TBAF (1.5 mL, 1 eq.) and stirred for 30 minutes. The reaction was diluted with EtOAc and then washed with 0.1 N HCl and brine, dried (MgSO₄), filtered, and concentrated under reduced pressure. DMSO (5 mL) was added to the residue, which was filtered and the filtrate purified by reverse phase HPLC (Waters SunFire Prep C18 OBD 10 μm 50×250 mm) eluting with a gradient of 5-95% Acetonitrile/Water+0.1% TFA over 20 minutes. The fractions that contained product were combined, basified with saturated aqueous NaHCO₃, and then extracted with CH₂Cl₂. The combined organic portions were washed with brine, dried (MgSO₄), filtered, and concentrated under reduced pressure to give the title compound. LRMS m/z: (M+H-Boc)⁺ calculated 304.1; found 304.2.

### Step 3: Benzyl (2S,4(R or S))-4-hydroxy-4-(trifluoromethyl)piperidine-2-carboxylate hydrochloride

To a stirred solution of benzyl 1-(tert-butyl) (2SA(R or S))-4-hydroxy-4-(trifluoromethyl)piperidine-1,2-dicarboxylate(230 mg, 0.570 mmol), and CH₂Cl₂ (2.85 mL) at room temperature was added TFA (0.439 mL, 5.7 mmol). The mixture was stirred for 0.5 hours at room temperature. The solvent was removed under reduced pressure. The residue was dissolved in 5 mL of 4M HCl in EtOAc and stirred for 5 minutes before the solvent was removed under reduced pressure to give the title compound. LRMS m/z: (M+H)⁺ calculated 304.3; found 304.2.

### Intermediate 16

### (S)-2-(tert-butoxcarbonyl)-2-azaspiro[4.4]nonane-3-carboxylic acid

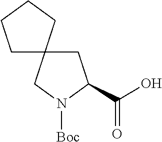 [SMILES: CC(C)(C)OC(=O)N1CC2(CCCC2)C[C@H]1C(=O)O]

### Step 1: (3R,7aS)-3-phenyltetrahydro-3H,5H-pyrrolo[1,2-c]oxazol-5-one

To a mixture of L-pyroglutaminol (0.30 kg, 2.6 mol) and benzaldehyde (0.28 kg, 2.6 mol, 0.26 L) in toluene (2.0 L) was added p-toluenesulfonic acid (4.5 g, 26 mmol) at 20° C. The reaction mixture was heated to 135° C. and stirred for 12 hours. The reaction mixture was concentrated under reduced pressure and the residue was purified by silica gel chromatography eluting with ethyl acetate:petroleum ether (1:10-1:0) to give the title compound. ¹H NMR (400 MHz, DMSO-d₆) δ 7.32-7.40 (m, 5H), 6.09 (s, 1H), 4.15 (m, 2H), 3.42-3.46 (m, 1H), 2.70-2.73 (m, 1H), 2.43-2.49 (m, 1H), 2.19-2.28 (m, 1H), 1.90-1.98 (m, 1H).

### Step 2: (3′R,7a'S)-3′-Phenyldihydro-1′H,3′H,5′H-spiro[cyclopentane-1,6′-pyrrolo[1,2-c]oxazol]-5′-one

To a solution of (3R,7aS)-3-phenyltetrahydro-3H,5H-pyrrolo[1,2-c]oxazol-5-one (0.19 kg, 0.95 mol) in THF (400 mL) under an inert atmosphere of nitrogen at −70° C. was added a solution of LiHMDS (1 M in THF, 2.4 L) slowly to keep the internal temperature below −70° C. The reaction mixture was stirred at −70° C. for 1 hour then 1,4-diiodobutane (0.32 kg, 1.0 mol) was added. The reaction mixture was warmed to room temperature and stirred for 1 hour at 20° C. The reaction mixture was treated with saturated aqueous ammonium chloride solution (1.2 L) and the resulting mixture was extracted with MTBE (3×500 mL). The combined organic phases were washed with brine, dried over Na₂SO₄, filtered, and concentrated under reduced pressure to give the title compound. ¹H NMR (400 MHz, CDCl₃) δ 7.32-7.47 (m, 5H), 6.32 (s, 1H), 4.22-4.25 (m, 1H), 4.07-4.12 (m, 1H), 3.49-3.53 (m, 1H), 2.28-2.32 (m, 1H), 1.9-2.01 (m, 1H), 1.5-1.75 (m, 4H), 1.50-1.66 (m, 1H), 1.47-1.49 (m, 2H), 1.13-1.30 (m, 2H).

### Step 3: (S)-(2-Benzyl-2-azaspiro[4.4]nonan-3-yl)methanol

To a solution of lithium aluminum hydride (72 g, 1.9 mol) and THF (1.7 L) under an atmosphere of nitrogen at 20° C. was added a solution of (3′R,7a'S)-3′-phenyldihydro-1′H,3′H,5′H-spiro[cyclopentane-1,6′-pyrrolo[1,2-c]oxazol]-5′-one (0.24 kg, 0.95 mol) in THF (0.74 L) at 20° C. The reaction mixture was warmed to 65° C. and stirred for 2 hours. The reaction mixture was cooled to 0° C. in an ice bath and carefully quenched with water (72 mL). The mixture was treated with 15% aqueous sodium hydroxide solution (72 mL) and additional water (220 mL). Solid sodium sulfate (100 g) was added and the slurry was stirred for 30 minutes, filtered, and concentrated under reduced pressure. The residue was purified by silica gel chromatography eluting with ethyl acetate:petroleum ether (1:100-1:0) to give the title compound. ¹H NMR (400 MHz, DMSO-d₆) δ 7.29 (m, 4H), 7.19-7.22 (m, 1H), 4.40-4.43 (m, 1H), 4.06 (d, J=13.2 Hz, 1H), 3.49-3.51 (m, 1H), 3.25-3.30 (m, 1H), 2.67-2.75 (m, 1H), 2.57-2.61 (m, 1H), 2.04-2.11 (m, 1H), 1.77-1.85 (m, 1H), 1.43-1.53 (m, 10H).

### Step 4: tert-Butyl (S)-3-(hydroxymethyl)-2-azaspiro[4.4]nonane-2-carboxylate

To a mixture of (S)-(2-benzyl-2-azaspiro[4.4]nonan-3-yl)methanol (70 g, 0.28 mol) and di-tert-butyl dicarbonate (68 g, 0.31 mol) in MeOH (700 mL) under an inert atmosphere of nitrogen was charged solid palladium hydroxide on carbon (10 wt %, 7.0 g, 50 mmol). The atmosphere was evacuated and backfilled with hydrogen three times. The mixture was stirred under a hydrogen atmosphere (15 psi) at room temperature for 12 hours. The catalyst was carefully removed by filtration under nitrogen atmosphere and the filtrate was concentrated under reduced pressure to give the title compound. ¹H NMR (400 MHz, DMSO-d₆) δ 4.66 (br s, 1H), 3.66 (br s, 1H), 3.56-3.64 (m, 1H), 3.34-3.44 (m, 1H), 3.21-3.33 (m, 1H), 2.85-3.02 (m, 1H), 1.67-1.86 (m, 2H), 1.44-1.67 (m, 8H), 1.31-1.44 (m, 11H).

### Step 5: (S)-2-(tert-Butoxycarbonyl)-2-azaspiro[4.4]nonane-3-carboxylic acid

To a solution of tert-butyl (S)-3-(hydroxymethyl)-2-azaspiro[4.4]nonane-2-carboxylate (0.30 kg, 1.2 mol) in MeCN (1.2 L) and H₂O (0.60 L) was added TEMPO (55 g, 0.35 mol), diacetoxyiodobenzene (0.95 kg, 2.9 mol) and NaHCO₃ (99 g, 1.2 mol). The reaction mixture was stirred at room temperature for 2 hours. The reaction mixture was diluted with water and extracted with ethyl acetate. The combined organic phases were washed with brine, dried over Na₂SO₄, filtered, and concentrated under reduced pressure. The residue was purified by silica gel chromatography eluting with ethyl acetate:petroleum ether (1:30-1:0) to give the title compound. ¹H NMR (400 MHz, MeOD) δ 4.17-4.25 (m, 1H), 3.31-3.36 (m, 1H), 3.23-3.27 (m, 1H), 2.20-2.24 (m, 1H), 1.87-1.92 (m, 1H), 1.61-1.76 (m, 5H), 1.47-1.61 (m, 3H), 1.43-1.54 (m, 9H).

### Intermediate 17

### (S)-2-(tert-Butoxycarbonyl)-2-azaspiro[4.5]decane-3-carboxylic acid

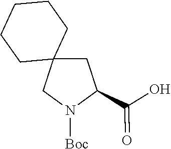 [SMILES: CC(C)(C)OC(=O)N1CC2(CCCCC2)C[C@H]1C(=O)O]

### Step 1: (3′R,7a'S)-3′-Phenyldihydro-1′H,3′H,5′H-spiro[cyclohexane-1,6′-pyrrolo[1,2-c]oxazol]-5′-one

To a solution of (3R,7aS)-3-phenyltetrahydro-3H,5H-pyrrolo[1,2-c]oxazol-5-one (0.20 kg, 0.98 mol) in THF (800 mL) under an inert atmosphere of nitrogen at −65° C. was added slowly a solution of LiHMDS (1.00 M in THF, 2.5 L, 2.5 mol). The reaction mixture was stirred at −65° C. for 1 hour then a solution of diiodopentane (0.35 kg, 1.1 mol) in THF (400 mL) was added. The reaction mixture was warmed to room temperature and stirred for 2 hours. The reaction mixture was quenched by addition of saturated aqueous ammonium chloride solution (2.5 L) and the resulting aqueous mixture was extracted with MTBE. The combined organic layers were washed with brine, dried over Na₂SO₄, filtered, and concentrated under reduced pressure to give the title compound. ¹H NMR (400 MHz, DMSO-d₆) δ 7.40-7.33 (m, 5H), 6.06 (s, 1H), 4.22 (dd, J=6.2, 7.80 Hz, 1H), 4.18-4.08 (m, 1H), 3.43 (t, J=8.1 Hz, 1H), 2.42-2.30 (m, 1H), 1.75-1.52 (m, 6H), 1.51-1.32 (m, 3H), 1.30-1.13 (m, 2H).

### Step 2: (S)-(2-Benzyl-2-azaspiro[4.5]decan-3-yl)methanol

To a suspension of lithium aluminum hydride (70 g, 1.8 mol) in THF (750 mL) under an inert atmosphere of nitrogen at room temperature was added a solution of (3′R,7a'S)-3′-phenyldihydro-1′H,3′H,5′H-spiro[cyclohexane-1,6′-pyrrolo[1,2-c]oxazol]-5′-one (0.25 kg, 0.92 mol) in THF (1 L). The reaction mixture was stirred at room temperature for 2 hours. The reaction mixture was cooled to 0° C. in an ice bath, then carefully quenched with water (70 mL). The mixture was treated with 15% aqueous sodium hydroxide solution (70 mL) and additional water (210 mL). Solid sodium sulfate (100 g) was added, and the slurry was stirred for 30 minutes, filtered, and concentrated under reduced pressure. The residue was purified by silica gel chromatography eluting with ethyl acetate:petroleum ether (1:100-1:0) to give the title compound. ¹H NMR (400 MHz, DMSO-d₆) δ 7.29 (d, J=4.4 Hz, 4H), 7.21 (qd, J=4.20, 8.5 Hz, 1H), 4.42 (t, J=5.2 Hz, 1H), 4.08 (d, J=13.4 Hz, 1H), 3.51 (td, J=5.2, 10.5 Hz, 1H), 3.21 (d, J=13.6 Hz, 1H), 2.66 (d, J=9.0 Hz, 1H), 2.64-2.56 (m, 1H), 1.90 (d, J=9.0 Hz, 1H), 1.66 (dd, J=8.3, 12.8 Hz, 1H), 1.46-1.16 (m, 12H).

### Step 3: (S)-(2-Azaspiro[4.5]decan-3-yl)methanol

To a solution of (S)-(2-benzyl-2-azaspiro[4.5]decan-3-yl)methanol (0.11 kg, 0.43 mol) in MeOH (70 mL) under an atmosphere of nitrogen was added palladium on carbon (10 wt %, 11 g, 43 mmol). The atmosphere was evacuated and backfilled with hydrogen three times then the mixture was stirred under a hydrogen atmosphere (50 psi) at room temperature for 16 hours. The mixture was carefully filtered under nitrogen atmosphere and the filtrate was concentrated under reduced pressure to give the title compound. ¹H NMR (400 MHz, DMSO-d₆) δ 5.02 (br s, 2H), 3.54-3.23 (m, 3H), 2.78-2.55 (m, 2H), 1.71-1.65 (m, 1H), 1.37 (br s, 9H), 1.22 (dd, J=8.70, 12.6 Hz, 1H).

### Step 4: tert-Butyl (S)-3-(hydroxymethyl)-2-azaspiro[4.5]decane-2-carboxylate

To a solution of (S)-(2-azaspiro[4.5]decan-3-yl)methanol (0.11 kg, 0.65 mol) in THF (550 mL) and H₂O (550 mL) at 0° C. was added di-tert-butyl dicarbonate (0.14 kg, 0.65 mol, 150 mL) and sodium carbonate (0.21 kg, 1.9 mol). The reaction mixture was warmed to room temperature and stirred for 3 hours. The reaction mixture was diluted with water and extracted with EtOAc. The combined organic layers were washed with brine, dried over Na₂SO₄, filtered, and concentrated under reduced pressure to give the title compound. ¹H NMR (400 MHz, DMSO-d₆) δ 4.64 (br s, 1H), 3.69 (br s, 1H), 3.55-3.35 (m, 3H), 2.85-2.69 (m, 1H), 1.84 (br dd, J=8.0, 12.3 Hz, 1H), 1.64-1.28 (m, 18H), 1.26-1.20 (m, 2H).

### Step 5: (S)-2-(tert-Butoxycarbonyl)-2-azaspiro[4.5]decane-3-carboxylic acid

To a solution of tert-butyl (S)-3-(hydroxymethyl)-2-azaspiro[4.5]decane-2-carboxylate (0.15 kg, 0.56 mol) in MeCN (600 mL) and H₂O (300 mL) was added TEMPO (18 g, 0.11 mmol), diacetoxyiodobenzene (0.45 kg, 1.4 mol) and NaHCO₃ (47 g, 0.56 mol) at room temperature. The reaction mixture was stirred at room temperature for 12 hours. The reaction mixture was diluted with water and extracted with ethyl acetate. The combined organic phases were washed with brine, dried over Na₂SO₄, filtered, and concentrated under reduced pressure. The resulting residue was purified by silica gel chromatography eluting with ethyl acetate:petroleum ether (1:30-1:0) to give the title compound. ¹H NMR (400 MHz, DMSO-d₆) δ 12.4 (br s, 1H), 4.19-3.98 (m, 1H), 3.28 (br s, 1H), 3.08-2.93 (m, 1H), 2.22-2.06 (m, 1H), 1.66-1.53 (m, 1H), 1.48-1.25 (m, 19H).

### Intermediate 18

### 2-(tert-Butoxycarbonyl)-8,8-difluoro-2-azaspiro[4.5]decane-3-carboxylic acid

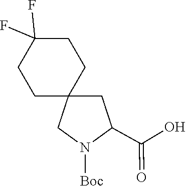 [SMILES: CC(C)(C)OC(=O)N1CC2(CCC(F)(F)CC2)CC1C(=O)O]

### Step 1: (4,4-Difluorocyclohexyl)methanol

To a solution of ethyl 4-oxocyclohexane-1-carboxylate (0.16 kg, 0.97 mol) in DCM (4 L) at 0° C. was added DAST (0.31 kg, 1.9 mol). The reaction mixture was stirred at room temperature for 16 hours. The resulting mixture was cooled to 0° C. and the pH was adjusted to 6 with saturated aqueous potassium carbonate solution. The resulting solution was extracted with dichloromethane. The combined organic layers were washed with brine, dried over Na₂SO₄, filtered and concentrated to give a mixture of ethyl 4,4-difluorocyclohexane-1-carboxylate and ethyl 4-fluorocyclohex-3-ene-1-carboxylate. To a solution of the crude residue (0.19 kg, 0.97 mol) in DCM (1.7 L) was added mCPBA (0.17 kg, 0.84 mmol) at room temperature. The reaction mixture was stirred at room temperature for 20 hours, then filtered. The solid filter cake was washed with dichloromethane. The filtrate was washed with saturated aqueous sodium thiosulfate and brine, then dried over Na₂SO₄, filtered, and concentrated to give a mixture of ethyl 4,4-difluorocyclohexane-1-carboxylate and ethyl 6-fluoro-7-oxabicyclo[4.1.0]heptane-3-carboxylate. A solution of the crude residue in THF (300 mL) was added dropwise to a solution of lithium aluminum hydride (79 g, 2.1 mol) in THF (2.0 L) under an atmosphere of nitrogen at 0° C. The reaction mixture was stirred for 3 hours at 0° C. The reaction mixture was carefully treated with water (79 mL) followed by 15% aqueous sodium hydroxide solution (79 mL) and additional water (237 mL). The resulting suspension was stirred for 15 minutes, then solid sodium sulfate was added, and the solids were removed by filtration. The filter cake was washed with dichloromethane and the filtrate was concentrated under reduced pressure. The residue was purified by silica gel chromatography eluting with ethyl acetate:petroleum ether (0:1-1:0) to give the title compound.

### Step 2: 4,4-Difluorocyclohexane-1-carbaldehyde

To a solution of oxalyl chloride (0.15 kg, 1.2 mol) in dichloromethane (2.5 L) under an atmosphere of nitrogen at −78° C. was added DMSO (0.18 kg, 2.3 mol). The mixture was stirred at −78° C. for 1 hour, and then a solution of (4,4-difluorocyclohexyl)methanol (0.11 kg, 0.77 mol) in dichloromethane (500 mL) was added. The reaction mixture was stirred at −78° C. for 2 hours, and then triethylamine (0.39 kg, 3.8 mol) was added. The reaction mixture was warmed to 15° C. and stirred for 12 hours. The reaction mixture was quenched with water and the aqueous layer was extracted with dichloromethane. The combined organic layers were washed with brine, dried over Na₂SO₄, filtered, and concentrated to give the title compound, which was used without further purification.

### Step 3: 4-((4,4-Difluorocyclohexylidene)methyl)morpholine

To a solution of 4,4-difluorocyclohexane-1-carbaldehyde (0.11 kg, 0.77 mmol) in toluene (0.77 L) under an atmosphere of nitrogen was added 4 Å MS (0.11 kg) and morpholine (80 g, 0.92 mol). The reaction mixture was warmed to 50° C. and stirred for 12 hours. The reaction mixture was concentrated under reduced pressure to give the title compound, which was used in the next step without further purification.

### Step 4: Ethyl 3-bromo-2-(hydroxyimino)propanoate

To a mixture of ethyl bromopyruvate (0.50 kg, 2.6 mol) in dichloromethane (2.5 L) and water (1.0 L) was added NH₂OH·HCl (0.18 kg, 2.6 mol). The reaction mixture was stirred at room temperature for 12 hours. The reaction mixture was concentrated under reduced pressure. The residue was dissolved in dichloromethane and washed with 6% hydrochloric acid and brine, dried over Na₂SO₄, filtered, and concentrated under reduced pressure to give the title compound. ¹H NMR (400 MHz, CDCl₃) δ 4.36-4.41 (m, 2H), 4.27 (s, 2H), 1.37-1.43 (m, 3H).

### Step 5: Ethyl 9,9-difluoro-1-morpholino-2-oxa-3-azaspiro[5.5]undec-3-ene-4-carboxylate

To a solution of 4-((4,4-difluorocyclohexylidene)methyl)morpholine (0.18 kg, 0.81 mol) in dichloromethane (2.0 L) was added ethyl 3-bromo-2-(hydroxyimino)propanoate (0.20 kg, 0.97 mol) and K₂CO₃ (0.22 kg, 1.6 mol). The reaction mixture was heated to 60° C. for 12 hours. The reaction mixture was cooled, filtered and the solids were washed dichloromethane. The filtrate was concentrated under reduced pressure to give the title compound, which was used without further purification.

### Step 6: Ethyl 8,8-difluoro-2-azaspiro[4.5]decane-3-carboxylate

To a solution of ethyl 9,9-difluoro-1-morpholino-2-oxa-3-azaspiro[5.5]undec-3-ene-4-carboxylate (36 g, 52 mmol, 50% purity) in EtOH (0.72 L) under an atmosphere of nitrogen was added Raney-Ni (36 g, 83 mmol). The mixture was placed under a hydrogen atmosphere (1 MPa) and heated to 50° C. for 8 hours. The reaction mixture was cooled to room temperature, filtered, and the solids washed with EtOH. The combined filtrates gave a solution of the title compound in EtOH, which was used without further purification.

### Step 7: 2-(tert-Butyl) 3-ethyl 8,8-difluoro-2-azaspiro[4.5]decane-2,3-dicarboxylate

To a solution of ethyl 8,8-difluoro-2-azaspiro[4.5]decane-3-carboxylate (13 g, 52 mmol) in EtOH (1.7 L) was added di-tert-butyl dicarbonate (17 g, 78 mmol) and triethylamine (16 g, 0.16 mol). The resulting mixture was stirred at room temperature for 12 hours then concentrated under reduced pressure. The residue was purified by silica gel chromatography eluting with petroleum ether:ethyl acetate (0:1-1:1). The resulting material was then further purified by preparative HPLC (Agela DuraShell C18 10 μM 250×70 mm) using a mobile phase gradient of 45-70% MeCN/water (10 mM NH₄HCO₃) over 20 min to give the title compound.

### Step 8: 2-(tert-Butoxycarbonyl)-8,8-difluoro-2-azaspiro[4.5]decane-3-carboxylic acid

To a solution of 2-(tert-butyl) 3-ethyl 8,8-difluoro-2-azaspiro[4.5]decane-2,3-dicarboxylate (11 g, 32 mmol) in THF (35 mL), EtOH (35 mL), and H₂O (35 mL) at 0° C. was added LiOH·H₂O (2.7 g, 63 mmol). The reaction mixture was warmed to room temperature and stirred for 12 hours. The mixture was cooled to 0° C. and the aqueous phase was washed with ethyl acetate. The aqueous phase was then acidified to pH 3 with 1 N hydrochloric acid and extracted with ethyl acetate. The combined organic layers were washed with brine, dried over Na₂SO₄, filtered, and concentrated to give the title compound, which was used without further purification. ¹H NMR (400 MHz, CDCl₃) δ 4.30-4.42 (m, 1H), 3.21-3.57 (m, 2H), 1.90-2.45 (m, 6H), 1.58-1.81 (m, 4H), 1.44-1.51 (m, 9H).

### Intermediate 19

### Benzyl (2S)-4-hydroxy-4-(trifluoromethyl)piperidine-2-carboxylate hydrochloride

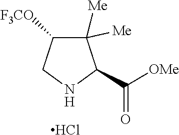 [SMILES: COC(=O)[C@H]1NC[C@H](OC(F)(F)F)C1(C)C ; Cl]

### Step 1: 1-(tert-Butyl) 2-methyl (2S,4R)-4-hydroxy-3,3-dimethylpyrrolidine-1,2-dicarboxylate

To a stirred solution of (2S,4R)-1-(tert-butoxycarbonyl)-4-hydroxy-3,3-dimethylpyrrolidine-2-carboxylic acid (780 mg, 3.01 mmol) in DCM (10 mL) and MeOH (1 mL) was added (diazomethyl)trimethylsilane (1.81 mL, 3.61 mmol, 2 M in hexane) at 25° C. and stirred at 25° C. for 1 hour. The reaction mixture was quenched with AcOH (0.2 mL) and then the solvent was removed under reduced pressure. The residue was purified by flash silica gel chromatography eluting with 0-22% EtOAc in petroleum ether to give the title compound. LRMS m/z: (M+H)⁺ calculated 174.2; found 174.1.

### Step 2: 1-(tert-Butyl) 2-methyl (2S,4R)-3,3-dimethyl-4-(((methylthio)carbonothioyl) oxy)pyrrolidine-1,2-dicarboxylate

To a solution of NaH (143 mg, 3.57 mmol, 60% in oil) in THF (1 mL) was added dropwise 1-(tert-butyl) 2-methyl (2S,4R)-4-hydroxy-3,3-dimethylpyrrolidine-1,2-dicarboxylate (650 mg, 2.378 mmol) in THF (6 mL) at 0° C. The mixture was stirred at 0° C. for 0.5 h. Then carbon disulfide (0.216 mL, 3.57 mmol) was added to the mixture. The mixture was stirred at 0° C. for 10 min. Iodomethane (1.48 mL, 23.8 mmol) was added to the mixture. The mixture was stirred at 25° C. for 16 h. The reaction mixture was quenched with H₂O (5 mL) and extracted with EtOAc (3×10 mL). The combined organic phases were washed with brine (5 mL), dried over anhydrous Na₂SO₄, and filtered. The solvent was removed under reduced pressure. The residue was purified by RP-HPLC (Boston Green ODS 5 μm 150×30 mm) eluting with a gradient of 60-90% Acetonitrile/Water+0.1% TFA over 10 minutes to give the title compound. LRMS m/z: (M+H-Boc)⁺ calculated 264.1; found 264.1.

### Step 3: Methyl (2S,4R)-3,3-dimethyl-4-(trifluoromethoxy)pyrrolidine-2-carboxylate

To a solution of 1,3-dibromo-5,5-dimethylhydantoin (0.724 g, 2.53 mmol) in DCM (9 mL) at −78° C. was added dropwise pyridine hydrofluoride (1.792 mL, 13.92 mmol). The mixture was stirred at −78° C. for 10 min. 1-(tert-butyl) 2-methyl (2S,4R)-3,3-dimethyl-4-(((methylthio)carbonothioyl)oxy)pyrrolidine-1,2-dicarboxylate (0.23 g, 0.63 mmol) in DCM (3 mL) was added. The mixture was stirred at −78° C. for 1 hour followed by the removal of the cooling bath. The mixture was stirred at 25° C. for 16 hours. The pH of the reaction mixture was adjusted to 7 with saturated aq. NaHCO₃ (10 mL). The mixture was filtered and the solvent was removed under reduced pressure. The residue was purified directly by RP-HPLC (YMC-Actus Triart C18 5 μm 150×30 mm) eluting with a gradient of 30-50% acetonitrile/water+0.1% TFA over 12 minutes to give the title compound. LRMS m/z: (M+H-Boc)⁺ calculated 242.1; found 242.1.

### Intermediate 20

### (1R,2S,5S)-3-((Benzyloxy)carbonyl)-6,6-dimethyl-3-azabicyclo[3.1.0]hexane-2-carboxylic acid

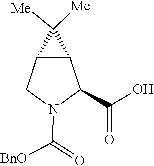 [SMILES: CC1(C)[C@@H]2[C@@H](C(=O)O)N(C(=O)OCc3ccccc3)C[C@@H]21]

### Step 1: 3-Benzyl 2-methyl (1R,2S,5S)-6,6-dimethyl-3-azabicyclo[3.1.0]hexane-2,3-dicarboxylate

A solution of methyl (1R,2S,5S)-6,6-dimethyl-3-azabicyclo[3.1.0]hexane-2-carboxylate hydrochloride (14 g, 68.1 mmol) in DCM (272 mL) was cooled to 0° C. and treated with N,N-diisopropylethylamine (23.8 mL, 136 mmol). Benzyl chloroformate (9.68 mL, 68.1 mmol) was added slowly and the mixture was stirred at 0° C. for 1 h. The mixture was warmed to RT and diluted with aqueous NH₄Cl and dichloromethane. The aqueous layer was extracted with three portions of DCM. The combined organic layers were dried over Na₂SO₄, filtered, and concentrated in vacuo to give the title compound. LRMS m/z: (M+H)⁺ calculated 304.1; found 304.2.

### Step 2:(1R,2S,5S)-3-((benzloxy)carbonyl)-6,6-dimethyl-3-azabicyclo[3.1.0]hexane-2-carboxylic acid

A solution of 3-benzyl 2-methyl (1R,2S,5S)-6,6-dimethyl-3-azabicyclo[3.1.0]hexane-2,3-dicarboxylate (20.7 g, 68.1 mmol) in ethanol (272 mL) was treated with 1 N aqueous sodium hydroxide (340 mL, 340 mmol). The mixture was stirred at RT for 15 h, then concentrated to remove EtOH, and treated carefully with conc. HCl (39.7 mL, 476 mmol). The mixture was diluted with DCM and water. The aqueous layer was extracted with three portions of DCM. The combined organic layers were dried over Na₂SO₄, filtered, and concentrated to afford the title compound. LRMS m/z: (M+H)⁺ calculated 290.1; found 290.1.

### Intermediate 21

### Trans-methyl-5-methylpiperidine-2-carboxylate hydrochloride

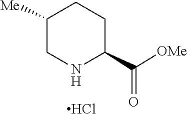 [SMILES: COC(=O)[C@@H]1CC[C@@H](C)CN1 ; Cl]

### Step 1: trans-Methyl-5-methylpiperidine-2-carboxylate hydrochloride

A solution of methyl 5-methylpiperidine-2-carboxylate (800 mg, 5.09 mmol) in 2 mL water was purified by reverse-phase HPLC (Phenomenex Luna Prep C18 5 μm 50×250 mm) 0-30% CH₃CN/water+0.1% TFA modifier over 30 min. Fractions that contained desired product (first-eluting stereoisomer) were combined and the solvent was removed under reduced pressure to give the TFA salt. The residue was dissolved in 4 N HCl in dioxane (2 mL)/MeOH (2 mL) and the solvent was removed under reduced pressure to give the title compound. LRMS m/z: (M+H)⁺ calculated 158.1; found 158.2.

### Intermediate 22

### Methyl (1R,2S,5S)-6,6,7,7-tetramethyl-3-azabicyclo[3.2.0]heptane-2-carboxylate hydrochloride

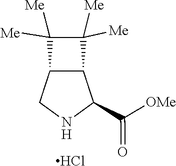 [SMILES: COC(=O)[C@H]1NC[C@H]2[C@@H]1C(C)(C)C2(C)C ; Cl]

### Step 1: (S)-3,3-Dimethyltetrahydro-3H,5H-pyrrolo[1,2-c]oxazol-5-one

To a solution of (S)-5-(hydroxymethyl)pyrrolidin-2-one (300 g, 2.60 mol) and 4-methylbenzenesulfonic acid (2.24 g, 13.0 mmol) in toluene (7.5 L) under nitrogen atmosphere was added 2-dimethoxypropane (1.09 kg, 10.4 mol). The flask was equipped with a Dean-Stark apparatus and then the mixture was heated to reflux and stirred for 4 hours. The solvent was removed under reduced pressure to give the title compound, which was used without further purification.

### Step 2: (7aS)-3,3-Dimethyl-6-(phenylsulfinyl)tetrahydro-3H,5H-pyrrolo[1,2-c]oxazol-5-one

Potassium tert-butoxide (516 g, 4601 mmol) was added to THF (3.4 L) under nitrogen atmosphere and the mixture was stirred for 1 hour at ambient temperature. (S)-3,3-dimethyltetrahydro-3H,5H-pyrrolo[1,2-c]oxazol-5-one (340 g, 2191 mmol) in THF (1.7 L) was added dropwise. After the addition was complete, the mixture was stirred for 1 h. Methyl benzenesulfinate (855 g, 5.48 mol) in THF (1.7 L) was add dropwise over 1 hour. The mixture was cooled to 0° C. before H₂O (5 L) was added. The pH of the mixture was adjusted to 4-6 by acetic acid, and then extracted with EtOAc (3×2 L). The combined organic fractions were washed with brine (5 L), dried over anhydrous Na₂SO₄, filtered, and the concentrated under reduced pressure to give the title compound, which was used without further purification.

### Step 3: (S)-3,3-Dimethyl-1,7a-dihydro-3H,5H-pyrrolo[1,2-c]oxazol-5-one

To a stirred solution of a solution of (7aS)-3,3-dimethyl-6-(phenylsulfinyl)tetrahydro-3H,5H-pyrrolo[1,2-c]oxazol-5-one (514 g, 1.84 mol) in toluene (5.14 L, 10 V) under nitrogen was added sodium phosphate, dibasic (784 g, 5.52 mol). The solution was stirred and heated at reflux overnight. The mixture was cooled to room temperature before being filtered, washing with toluene (500 mL). The filtrate was concentrated under reduced pressure. The residue was purified by column chromatography on silica gel, eluting with 5% EtOAc in DCM to give the title compound.

### Step 4: (5aS,7aR,7bS)-3,3,6,6,7,7-Hexamethylhexahydro-3H,5H-cyclobuta[3,4]pyrrolo[1,2-c]oxazol-5-one

To a stirred solution of (S)-3,3-dimethyl-1,7a-dihydro-3H,5H-pyrrolo[1,2-c]oxazol-5-one (8 g, 52.29 mmol) in MeCN (1.6 L) in a quartz flask was added 2,3-dimethylbut-2-ene (44 g, 522.9 mmol). The mixture was photolyzed (wavelength: 365 nm) for 4 days. The reaction solvent was removed under reduced pressure. The residue was purified on C18 silica gel eluting with a gradient of 35-65% acetonitrile/water+0.1% NH₄HCO₃ over 10 minutes to give the title compound.

### Step 5: (1S,4S,5R)-4-(Hydroxymethyl)-6,6,7,7-tetramethyl-3-azabicyclo[3.2.0]heptan-2-one

To a stirred solution of a solution (5aS,7aR,7bS)-3,3,6,6,7,7-hexamethylhexahydro-3H,5H-cyclobuta[3,4]pyrrolo[1,2-c]oxazol-5-one (4.6 g, 19 mmol) in MeOH (92 mL) under nitrogen was added 4-methylbenzenesulfonic acid (0.334 g, 1.94 mmol). The mixture was heated to reflux and stirred for 2 hours. The mixture was cooled to room temperature and then the reaction solvent was removed under reduced pressure. The residue was purified by column chromatography on silica gel, eluting with 5% MeOH in DCM to give the title compound.

### Step 6: tert-Butyl (1R,2S,5S)-2-(hydroxymethyl)-6,6,7,7-tetramethyl-3-azabicyclo[3.2.0]heptane-3-carboxylate

To a stirred solution of (1S,4S,5R)-4-(hydroxymethyl)-6,6,7,7-tetramethyl-3-azabicyclo[3.2.0]heptan-2-one (4.8 g, 24 mmol) in toluene (48 mL) at ambient temperature under nitrogen was added BH₃·DMS solution (9.2 mL, 97 mmol). The resulting solution was stirred at ambient temperature for 10 minutes and then heated to reflux for 2 hours. The reaction was cooled to room temperature and the pH of the mixture was adjusted to 14 with 6 M NaOH. THF (40 mL) was added to the mixture followed by Boc₂O (23 mL, 97 mmol). The mixture was stirred for 16 hours at room temperature. The mixture was extracted with EtOAc (2×50 mL). The combined organic fractions were dried (MgSO₄), filtered, and concentrated under reduced pressure. The residue was purified by column chromatography on silica gel eluting with 0-30% EtOAc in petroleum ether over 60 minutes to give the title compound.

### Step 7: 3-(tert-Butyl) 2-methyl (1R,2S,5S)-6,6,7,7-tetramethyl-3-azabicyclo[3.2.0]heptane-2,3-dicarboxylate

To a stirred solution of tert-butyl (1R,2S,5S)-2-(hydroxymethyl)-6,6,7,7-tetramethyl-3-azabicyclo[3.2.0]heptane-3-carboxylate (4.5 g, 16 mmol, 1.0 equiv) in acetonitrile (45 mL) and water (45 mL) under nitrogen was added TEMPO (0.496 g, 3.18 mmol) followed by phenyl-λ³-iodanediyl diacetate (11.3 g, 34.9 mmol). The mixture was stirred at ambient temperature for 2 hours. K₂CO₃ (21.9 g, 159 mmol) and iodomethane (22.5 g, 159 mmol) were added and then the reaction was stirred overnight at room temperature. EtOAc (40 mL) was added and the mixture was extracted three times. The combined organic fractions were washed with brine (100 mL) and the solvent was removed under reduced pressure. The residue was purified by column chromatography on silica gel eluting with 5% EtOAc in petroleum ether to give the title compound. LRMS m/z: (M+H)⁺ calculated 312.2; found 312.0.

### Step 8: Methyl (1R,2S,5S)-6,6,7,7-tetramethyl-3-azabicyclo[3.2.0]heptane-2-carboxylate hydrochloride

To a stirred solution of 3-(tert-butyl) 2-methyl (1R,2S,5S)-6,6,7,7-tetramethyl-3-azabicyclo[3.2.0]heptane-2,3-dicarboxylate (125 mg, 0.401 mmol) in MeOH (2 mL) and DCM (4 mL) was added saturated HCl/EtOAc solution (12 mL) at 10° C. The resulting mixture was stirred for 1 h. The reaction mixture was concentrated to give the title compound. LRMS m/z: (M+H)⁺ calculated 212.2; found 212.1.

### Intermediate 23

### Benzyl (1R,2S,5R)-7,7-dimethyl-3-azabicyclo[3.2.0]heptane-2-carboxylate hydrochloride

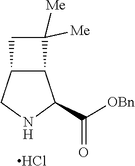 [SMILES: CC1(C)C[C@H]2CN[C@H](C(=O)OCc3ccccc3)[C@H]21 ; Cl]

### Step 1: (5aR,7aR,7bS)-3,3,7,7-tetramethyl-hexahydro-3H,5H-cyclobuta[3,4]pyrrolo[1,2-c]oxazol-5-one

A solution of (S)-3,3-dimethyl-1,7a-dihydro-3H,5H-pyrrolo[1,2-c]oxazol-5-one (2 g, 13 mmol, 1.0 equiv.) in acetonitrile (400 mL, 200 V) under an atmosphere of nitrogen was purged with 2-methylprop-1-ene. The reaction mixture was photolyzed (wavelength: 365 nm) for 4 days. The reaction mixture was concentrated and the residue was purified by column chromatography eluting with 25-55% MeCN/0.1% NH₄CO₃ in water over 30 min to give the title compound as a mixture with other isomers.

### Step 2: (1R,4S,5R)-4-(Hydroxymethyl)-6,6-dimethyl-3-azabicyclo[3.2.0]heptan-2-one

A solution of 1-methyl-4-(methylsulfonyl)benzene (0.281 g, 1.648 mmol, 0.1 equiv) and (1R,4S,5R)-4-(hydroxymethyl)-6,6-dimethyl-3-azabicyclo[3.2.0]heptan-2-one (3.45 g, 16.5 mmol, 1.0 equiv) in MeOH (35 mL, 10 V) under an atmosphere of nitrogen was heated to reflux and stirred for 2 h. The mixture was cooled to RT and concentrated. The residue was purified by column chromatography on silica gel eluting with 9:1 DCM:MeOH to give the title compound as a mixture with other isomers.

### Step 3: tert-Butyl (1R,2S,5R)-2-(hydroxymethyl)-7,7-dimethyl-3-azabicyclo[3.2.0]heptane-3-carboxylate

To a solution of LAH (3.1 g, 82 mmol, 6.0 equiv) in dry THF (25 mL, 11 V) under an atmosphere of nitrogen was added dropwise a solution of 4-(hydroxymethyl)-6,6-dimethyl-3-azabicyclo[3.2.0]heptan-2-one (2.3 g, 14 mmol, 1.0 equiv) in dry THF (25 mL, 11 V). The solution was stirred overnight at reflux. The mixture was cooled to 0° C. before water (6 mL) was added dropwise with stirring. 10 M aqueous NaOH (2 mL) was then added together with anhydrous Na₂SO₄ (10 g). The mixture was allowed to warm to room temperature and the solution was stirred for 20 min, then filtered. The filter cake was washed with THF (100 mL) and the filtrate was concentrated under reduced pressure. The residue was dissolved in in THF (25 mL), and 10 M aqueous NaOH (25 mL) was added. Di-tert-butyl dicarbonate (14.8 g, 68.0 mmol, 5.0 equiv) was added, and the mixture was stirred overnight. EtOAc (50 mL) and water (50 mL) were added and the mixture was extracted three times. The combined organic fractions were concentrated under reduced pressure. The residue was dissolved in MeOH (50 mL). The mixture was treated with NaOH (3.0 equiv.) and stirred for 1 h at room temperature. H₂O (100 mL) was added and the mixture was extracted with EtOAc (2×50 mL). The combined organics were concentrated to give the title compound as a mixture with other isomers.

### Step 4: 2-Benzyl 3-(tert-butyl) (1R,2S,5R)-7,7-dimethyl-3-azabicyclo[3.2.0]heptane-2,3-dicarboxylate

A solution of tert-butyl 2-(hydroxymethyl)-7,7-dimethyl-3-azabicyclo[3.2.0]heptane-3-carboxylate (2.3 g, 9.0 mmol, 1.0 equiv.) in acetonitrile (25 mL, 11 V) and water (25 mL, 11 V) under an atmosphere of nitrogen was treated with phenyl-λ³-iodanediyl diacetate (11.6 g, 36.0 mmol, 4.0 equiv.) and TEMPO (0.56 g, 3.6 mmol, 0.4 equiv.). The mixture was stirred for 2 h before K₂CO₃ (7.47 g, 54.0 mmol, 6.0 equiv.) and (bromomethyl)benzene (1.54 g, 9.01 mmol, 1.0 equiv) were added. The reaction was stirred overnight. The reaction mixture was concentrated and the residue was purified by column chromatography on C₁₈ silica gel eluting with 40-80% (0.1% NH₄CO₃ in H₂O)/MeCN. The mixture was further purified by SFC, (Exsil Chiral-NR 8 μm 30×250 mm) eluting with 15% (1:1 IPA:hexanes+0.1% 2 M NH₃/MeOH) in CO₂ at 100 bar and a flow rate of 70 mL/min, monitoring at 205 nM, to give the title compound (first-eluting isomer). LRMS m/z: (M+H)⁺ calculated 360.2; found 360.3. ¹H NMR (400 MHz, DMSO-d₆) δ 7.33-7.40 (m, 5H), 5.06-5.21 (m, 2H), 4.31-4.40 (m, 1H), 3.28-3.38 (m, 2H), 2.85-2.88 (m, 9H), 1.93-1.95 (m, 1H), 1.25-1.55 (m, 10H), 1.15-1.25 (m, 3H), 0.85-0.95 (m, 3H).

### Step 5: Benzyl (1R,2S,5R)-7,7-dimethyl-3-azabicyclo[3.2.0]heptane-2-carboxylate hydrochloride

A solution of 2-benzyl 3-(tert-butyl) (1R,2S,5R)-7,7-dimethyl-3-azabicyclo[3.2.0]heptane-2,3-dicarboxylate (150 mg, 0.417 mmol) in 4 M HCl in dioxane (3 mL, 12 mmol) was stirred at RT for 1 h. The reaction mixture was concentrated to give the title compound. LRMS m/z: (M+H)⁺ calculated 260.2; found 260.1.

### Intermediate 24

### Methyl (1R,2S,5S)-3-azaspiro[bicyclo[3.1.0]hexane-6,1′-cyclopentane]-2-carboxylate hydrochloride

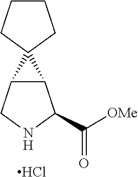 [SMILES: COC(=O)[C@H]1NC[C@H]2[C@@H]1C21CCCC1 ; Cl]

### Step 1: tert-Butyl (S)-4-(cyclopentylidenemethyl)-2,2-dimethyloxazolidine-3-carboxylate

To a stirred solution of bromo(cyclopentyl)triphenyl-λ⁵-phosphane (224 g, 545 mmol) in THF (2.2 L) at 0° C. was added n-BuLi (392 mL, 980 mmol) and the solution was stirred for 30 minutes. Tert-butyl (R)-4-formyl-2,2-dimethyloxazolidine-3-carboxylate (100 g, 436 mmol) in THF (896 ml). The mixture was allowed to warm to ambient temperature and then stirred for 3 hours. The reaction was quenched by addition of water (2 L) and extracted with EtOAc (2×2 L). The combined organic layers were washed with brine and dried over anhydrous sodium sulfate. The solvent was removed under reduced pressure and the crude residue was purified by column chromatography on C18 silica gel eluting with a gradient of 65-95% acetonitrile/water+0.1% NH₄HCO₃ over 30 minutes to give the title compound.

### Step 2: tert-Butyl (S)-(2-((1-cyclopentylidene-3-hydroxypropan-2-yl)amino)-2-oxoethyl)carbamate

To a stirred solution of tert-butyl (S)-4-(cyclopentylidenemethyl)-2,2-dimethyloxazolidine-3-carboxylate (72.3 g, 257 mmol) in DCM (723 mL) under nitrogen atmosphere was added 2,6-dimethylpyridine (83 g, 770 mmol) and trimethylsilyl trifluoromethanesulfonate (143 g, 642 mmol) at ambient temperature. The mixture was stirred for 3 hours. The mixture was cooled to 0° C., triethylamine (78 g, 770 mmol) was added, and the solution was stirred for 5 minutes. 2,5-dioxopyrrolidin-1-yl (tert-butoxycarbonyl)glycinate (77 g, 280 mmol) was added and the solution was stirred overnight at ambient temperature. The reaction mixture was concentrated under reduced pressure and the residue was purified by column chromatography on C18 silica gel eluting with a gradient of 15-45% acetonitrile/water+0.05% TFA over 30 minutes to give the title compound.

### Step 3: tert-Butyl (S)-(2-(4-(cyclopentylidenemethyl)-2,2-dimethyloxazolidin-3-yl)-2-oxoethyl)carbamate

To a stirred solution of tert-butyl (S)-(2-((1-cyclopentylidene-3-hydroxypropan-2-yl)amino)-2-oxoethyl)carbamate (49.5 g, 166 mmol) in toluene (495 mL) at ambient temperature was added 2,2-dimethoxypropane (173 g, 1660 mmol) followed by 4-methylbenzenesulfonic acid (0.571 g, 3.32 mmol). The mixture was stirred and heated at 80° C. overnight. The reaction mixture was concentrated under reduced pressure and the residue was purified by column chromatography on C18 silica gel eluting with a gradient of 15-45% acetonitrile/water+0.1% NH₄HCO₃ over 30 minutes to give the title compound.

### Step 4: (S)-2-(4-(Cyclopentylidenemethyl)-2,2-dimethyloxazolidin-3-yl)-2-oxoethane-1-diazonium

To a stirred solution of a tert-butyl (S)-(2-(4-(cyclopentylidenemethyl)-2,2-dimethyloxazolidin-3-yl)-2-oxoethyl)carbamate (42.5 g, 126 mmol) and pyridine (29.8 g, 377 mmol) in anhydrous acetonitrile (213 mL) under nitrogen atmosphere at 0° C. was added tetrafluoro(nitroso)-λ⁵-borane (29.3 g, 251 mmol) in a single portion. After 10 minutes, pyrrolidine (72.3 g, 1.02 mol) was added followed by the removal of the cooling bath. After 1 hour, the reaction mixture was concentrated under reduced pressure and the residue was purified by column chromatography eluting with a gradient of 0-30% EtOAc in petroleum ether over 30 minutes to give the title compound.

### Step 5: (5a′S,6a′R,6b′S)-3′,3′-Dimethyltetrahydro-3′H,5′H-spiro[cyclopentane-1,6′-cyclopropa[3,4]pyrrolo[1,2-c]oxazol]-5′-one

To a stirred solution of a (S)-2-(4-(cyclopentylidenemethyl)-2,2-dimethyloxazolidin-3-yl)-2-oxoethane-1-diazonium (26.0 g, 104 mmol) and toluene (1.06 L) at ambient temperature was added diacetoxyrhodium (1.11 g, 2.51 mmol). The mixture was stirred and heated at 70° C. for 1 hour. The reaction mixture was concentrated under reduced pressure and the residue was purified by column chromatography eluting with a gradient of 0-30% EtOAc in petroleum ether over 30 minutes to give title compound.

### Step 6: (1R,2S,5S)-2-(Hydroxymethyl)-3-azaspiro[bicyclo[3.1.0]hexane-6,1′-cyclopentan]-4-one

To a stirred solution of a (5a′S,6a′R,6b′S)-3′,3′-dimethyltetrahydro-3′H,5′H-spiro[cyclopentane-1,6′-cyclopropa[3,4]pyrrolo[1,2-c]oxazol]-5′-one (19 g, 86 mmol) and MeOH (380 mL) under nitrogen atmosphere at ambient temperature was added 4-methylbenzenesulfonic acid (1.48 g, 8.59 mmol). The mixture was stirred and heated at reflux for 2 hours. The reaction mixture was concentrated under reduced pressure and the residue was purified by column chromatography eluting with a gradient of 0-30% MeOH in DCM over 30 minutes to give the title compound.

### Step 7: tert-Butyl (1R,2S,5S)-2-(hydroxymethyl)-3-azaspiro[bicyclo[3.1.0]hexane-6,1′-cyclopentane]-3-carboxylate

To a stirred solution of a (1R,2S,5S)-2-(hydroxymethyl)-3-azaspiro[bicyclo[3.1.0]hexane-6,1′-cyclopentan]-4-one (11 g, 61 mmol) in THF (110 mL) under nitrogen atmosphere at ambient temperature was added a solution of LiAlH₄ (13.8 g, 364 mmol) in THF (110 mL). The mixture was stirred and heated at reflux overnight. The reaction was cooled to 0° C. and then was quenched by slow addition of 1.6 mL H₂O followed by 1.6 mL 10% aqueous NaOH solution. Na₂SO₄ (3.2 g) was added the mixture was stirred for 30 minutes at ambient temperature. The solids were filtered and the filtrate was concentrated under reduced pressure. The residue was dissolved in THF (20 mL) and then treated with NaOH (180 mmol) and Boc₂O (70.5 mL, 303 mmol). The mixture was stirred overnight at ambient temperature. The mixture was extracted with EtOAc (40 mL) and the organic phase was washed with brine (40 mL). The combined organic layers were dried over anhydrous Na₂SO₄ and the solvent was removed under reduced pressure. The residue was dissolved in MeOH (470 mL) and then treated with NaOH (3.0 equiv.). The mixture was stirred at ambient temperature for 5 hours and then concentrated under reduced pressure. H₂O (500 mL) was added and the mixture was extracted with EtOAc (2×500 mL). The combined organic layers were dried over anhydrous sodium sulfate, filtered, concentrated under reduced pressure to give the title compound, which was used in the next step without further purification.

### Step 8: 3-(tert-Butyl) 2-methyl (1R,2S,5S)-3-azaspiro[bicyclo[3.1.0]hexane-6,1′-cyclopentane]-2,3-dicarboxylate

To a stirred solution of tert-butyl (1R,2S,5S)-2-(hydroxymethyl)-3-azaspiro[bicyclo[3.1.0]hexane-6,1′-cyclopentane]-3-carboxylate (1.6 g, 6.0 mmol) in acetonitrile (16.0 mL) and water (16.0 mL) under nitrogen atmosphere at ambient temperature was added phenyl-λ³-iodanediyl diacetate (4.24 g, 13.2 mmol) and TEMPO (0.187 g, 1.20 mmol). The mixture was stirred at room temperature for 2 hours. To the mixture was added potassium carbonate (4.96 g, 35.9 mmol) and iodomethane (8.49 g, 59.8 mmol) and the mixture was stirred overnight at room temperature. The reaction mixture was filtered and concentrated under reduced pressure and the residue was purified by chromatography eluting with 7% EtOAc in petroleum ether to provide crude material. The material was further purified by SFC purification (ART Cellulose-SC 5 μm 3×25 cm) eluting with 2% ethanol/CO₂ at 50 mL/min), to give the title compound. LRMS m/z: (M−^{t}Bu+2H)⁺ calculated 240.1; found 240.0.

### Step 9: Methyl (1R,2S,5S)-3-azaspiro[bicyclo[3.1.0]hexane-6,1′-cyclopentane]-2-carboxylate hydrochloride

A solution of 3-(tert-butyl) 2-methyl (1R,2S,5S)-3-azaspiro[bicyclo[3.1.0]hexane-6,1′-cyclopentane]-2,3-dicarboxylate (200 mg, 0.677 mmol) in saturated HCl/EtOAc solution (2 mL) was stirred at RT for 1 h. The reaction mixture was concentrated to give the title compound. LRMS m/z: (M+H)⁺ calculated 196.1; found 196.1.

### Intermediate 25

### (1R,2S,5S)-3-((benzyloxy)carbonyl)-3-azaspiro[bicyclo[3.1.0]hexane-6,1′-cyclohexane]-2-carboxylic acid

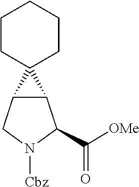 [SMILES: COC(=O)[C@@H]1[C@@H]2[C@H](CN1C(=O)OCc1ccccc1)C21CCCCC1]

### Step 1: (3R,7aS)-3-Phenyl-6-(phenylthio)tetrahydro-3H,5H-pyrrolo[1,2-c]oxazol-5-one

To a solution of (3R,7aS)-3-phenyltetrahydro-3H,5H-pyrrolo[1,2-c]oxazol-5-one (0.23 kg, 1.1 mol) in THF (1.3 L) at −70° C. was added dropwise LiHMDS (1 M, 1.1 L, 1.1 mol) followed by (phenyldisulfanyl)benzene (0.25 kg, 1.1 mol). The reaction mixture was stirred at −70° C. for 5 hours. The mixture was quenched with water and extracted with ethyl acetate. The combined organic layers were washed with brine, dried over Na₂SO₄, filtered, and concentrated to give the title compound. LRMS m/z [M+H]⁺ calculated 312.1; found 312.2.

### Step 2: (3R,7aS)-3-Phenyl-6-(phenylsulfinyl)tetrahydro-3H,5H-pyrrolo[1,2-c]oxazol-5-one

To a solution of (3R,7aS)-3-phenyl-6-(phenylthio)tetrahy dro-3H,5H-pyrrolo[1,2-c]oxazol-5-one (0.50 kg, 1.6 mol) at 0° C. in DCM (3.0 L) was added mCPBA (0.32 kg, 1.6 mol, 85% purity) in several batches. The reaction mixture was stirred at 0° C. for 5 hours. The mixture was quenched by the addition of aqueous Na₂SO₃ (500 mL) and extracted with DCM. The combined organic layers were dried over Na₂SO₄, filtered, and concentrated to give the title compound. LRMS m z [M+H]⁺ calculated 328.1; found 328.1.

### Step 3: (3R,7aS)-3-Phenyl-1,7a-dihydro-3H,5H-pyrrolo[1,2-c]oxazol-5-one

To a mixture of (3R,7aS)-3-phenyl-6-(phenylsulfinyl)tetrahydro-3H,5H-pyrrolo[1,2-c]oxazol-5-one (0.40 kg, 1.2 mol) in toluene (2.4 L) was added pyridine (0.39 kg, 4.9 mol, 0.39 L). The reaction mixture was stirred at 110° C. for 6 hours, then concentrated under reduced pressure. The residue was purified by silica gel chromatography eluting with ethyl acetate:petroleum ether (1:20-1:1) to give the title compound. ¹H NMR (400 MHz, DMSO-d₆): δ 7.58 (dd, J=1.8, 5.8 Hz, 1H), 7.48-7.53 (m, 2H), 7.38-7.46 (m, 3H), 6.20 (dd, J=1.4, 5.8 Hz, 1H), 5.98 (s, 1H), 4.65-4.85 (m, 1H), 4.28 (t, J=7.6 Hz, 1H), 3.27-3.43 (m, 2H).

### Step 4: N′-Cyclohexnlidene-4-methylbenzenesulfonohydrazide

To a mixture of 4-methylbenzenesulfonohydrazide (0.47 kg, 2.6 mol) in MeOH (600 mL) at 25° C. was added cyclohexanone (0.25 kg, 2.6 mol). The reaction mixture was stirred at 25° C. for 10 hours. The mixture was diluted in MeOH and filtered to give the title compound. ¹H NMR (400 MHz, DMSO): δ 10.13 (s, 1H), 7.74 (d, J=8.4 Hz, 2H), 7.40 (d, J=8.2 Hz, 2H), 2.39 (s, 3H), 2.27 (br s, 2H), 2.13-2.04 (m, 2H), 1.52 (br s, 6H).

### Step 5: Sodium 2-cyclohexylidene-1-tosylhydrazin-1-ide

To a solution of N′-cyclohexylidene-4-methylbenzenesulfonohydrazide (0.50 kg, 1.9 mol) in MeOH (3.0 L) was added NaOMe (0.10 kg, 1.9 mol) in MeOH (500 mL). The reaction mixture was stirred at 20° C. for 1 hour then concentrated under reduced pressure. The residue was slurried in MTBE and filtered to give the title compound.

### Step 6: (3a′S,6′R,8a′S,8b′S)-6′-Phenyl-3a′,8′,8a′,8b′-tetrahydro-4′H,6′H-spiro[cyclohexane-1,3′-pyrazolo[3′,4′:3,4]pyrrolo[1,2-c]oxazol]-4′-one

To a solution of (3R,7aS)-3-phenyl-1,7a-dihydro-3H,5H-pyrrolo[1,2-c]oxazol-5-one (0.18 kg, 0.87 mmol) in chlorobenzene (1.0 L) under an atmosphere of nitrogen was added sodium 2-cyclohexylidene-1-tosylhydrazin-1-ide (0.38 kg, 1.3 mol). The reaction mixture was stirred at 135° C. for 4 hours then filtered and concentrated under reduced pressure to give the title compound, which was used without further purification. ¹H NMR (400 MHz, DMSO-d₆): δ 7.21-7.31 (m, 5H), 6.27 (s, 1H), 5.22 (dd, J=1.4, 8.6 Hz, 1H), 4.38 (dd, J=6.6, 7.8 Hz, 1H), 4.23 (ddd, J=1.4, 6.6, 9.6 Hz, 1H), 3.44 (dd, J=8.2, 9.6 Hz, 1H), 2.69 (d, J=8.4 Hz, 1H), 1.78-2.07 (m, 6H), 1.50-1.77 (m, 5H), 1.33-1.48 (m, 1H).

### Step 7: (3′R,5a′S,6a′R,6b′S)-3′-phenyltetrahydro-3′H,5′H-spiro[cyclohexane-1,6′-cyclopropa[3,4]pyrrolo[1,2-c]oxazol]-5′-one

A solution of (3a′S,6′R,8a′S,8b′S)-6′-phenyl-3a′,8′,8a′,8b′-tetrahydro-4′H,6′H-spiro[cyclohexane-1,3′-pyrazolo[3′,4′:3,4]pyrrolo[1,2-c]oxazol]-4′-one (28 g, 90 mmol) in toluene (1.0 L) was placed in a photoreactor and irradiated at 365 nm at 50° C. for 5 hours. The reaction mixture was concentrated under reduced pressure to give the title compound, which was used without further purification. LRMS m/z [M+H]⁺ calculated 284.2; found 284.1.

### Step 8: ((1R,2S,5S)-3-Benzyl-3-azaspiro[bicyclo[3.1.0]hexane-6,1′-cyclohexan]-2-yl)methanol

To a suspension of lithium aluminum hydride (12 g, 0.32 mol) in THF (150 mL) under an atmosphere of nitrogen at room temperature was added a solution of (3′R,5a′S,6a′R,6b′S)-3′-phenyltetrahydro-3′H,5′H-spiro[cyclohexane-1,6′-cyclopropa[3,4]pyrrolo[1,2-c]oxazol]-5′-one (60 g, 0.21 mol) in THF (150 mL). The reaction mixture was stirred at 66° C. for 2 hours. The reaction mixture was cooled to 0° C. in an ice bath then carefully quenched with aqueous sodium sulfate (20 mL) until a white precipitate formed. The mixture was diluted with ethyl acetate (500 mL), filtered through a pad of celite and concentrated under reduced pressure. The residue was purified by silica gel chromatography eluting with ethyl acetate:petroleum ether (1:10-1:2) to give the title compound. ¹H NMR (400 MHz, DMSO-d₆) δ 7.11-7.27 (m, 5H), 3.72 (d, J=13.2 Hz, 1H), 3.39-3.54 (m, 3H), 3.16 (dd, J=5.6, 10.0 Hz, 1H), 2.70 (br d, J=1.4 Hz, 1H), 2.19 (dd, J=1.8, 9.8 Hz, 1H), 1.36-1.48 (m, 6H), 1.10-1.28 (m, 6H).

### Step 9: Benzyl (1R,2S,5S)-2-(hydroxymethyl)-3-azaspiro[bicyclo[3.1.0]hexane-6,1′-cyclohexane]-3-carboxylate

To a solution of ((1R,2S,5S)-3-benzyl-3-azaspiro[bicyclo[3.1.0]hexane-6,1′-cyclohexan]-2-yl)methanol (35 g, 0.13 mol) in EtOAc (160 mL) and acetic acid (80 mL) under an atmosphere of nitrogen was added palladium on carbon (10 wt %, 5.0 g, 20 mmol). The atmosphere was evacuated and backfilled with hydrogen three times, and then the mixture was stirred under a hydrogen atmosphere (30 psi) at 30° C. for 12 hours. The mixture was carefully filtered under nitrogen atmosphere through a pad of celite and the filtrate was concentrated under reduced pressure. The residue was dissolved in 2-methyltetrahydrofuran (200 mL), then treated with a saturated aqueous solution of NaHCO₃ (35 g, 0.41 mol, 16 mL) diluted in H₂O (100 mL), and benzyl chloroformate (39 g, 0.23 mol). The reaction mixture was stirred at 25° C. for 4 hours. The reaction mixture was diluted with water and extracted with ethyl acetate. The combined organic layers were washed with 1 M HCl and water, then dried over MgSO₄, filtered and concentrated under reduced pressure. The residue was purified by silica gel chromatography eluting with ethyl acetate:petroleum ether (1:10-1:2) to give the title compound. LRMS m z [M+H]⁺ calculated: 316.2; found 316.1. ¹H NMR (400 MHz, CDCl₃) δ 7.31-7.44 (m, 5H), 5.08-5.21 (m, 2H), 3.97 (dd, J=3.2, 7.2 Hz, 1H), 3.71-3.90 (m, 2H), 3.57-3.69 (m, 2H), 3.49-3.55 (m, 1H), 1.52 (br s, 6H), 1.13-1.42 (m, 6H).

### Step 10: (1R,2S,5S)-3-((Benzyloxy)carbonyl)-3-azaspiro[bicyclo[3.1.0]hexane-6,1′-cyclohexane]-2-carboxylic acid

To a solution of benzyl (1R,2S,5S)-2-(hydroxymethyl)-3-azaspiro[bicyclo[3.1.0]hexane-6,1′-cyclohexane]-3-carboxylate (40 g, 0.13 mol) in acetonitrile (120 mL) and water (120 mL) was added (diacetoxyiodo)benzene (0.12 kg, 0.38 mol) and TEMPO (4.0 g, 25 mmol). The reaction mixture was stirred at 20° C. for 12 hours. The reaction mixture was diluted with water and extracted with ethyl acetate. The combined organic layers were concentrated under reduced pressure. The residue was purified by silica gel chromatography eluting with ethyl acetate/petroleum ether (0-100%) to give the title compound. LRMS m/z [M+H]⁺ calculated 330.2; found 330.1. ¹H NMR (400 MHz, CDCl₃) δ 10.63 (br s, 1H), 7.22-7.47 (m, 5H), 5.33 (s, 1H), 5.07-5.22 (m, 2H), 4.30 (d, J=17.2 Hz, 1H), 3.67-3.85 (m, 1H), 3.56 (dd, J=11.0, 15.0 Hz, 1H), 1.63 (d, J=7.2 Hz, 1H).

### Intermediate 26

### (1R,2S,5S)-3-(tert-Butoxycarbonyl)tetrahydro-3-azaspiro[bicyclo[3.1.0]hexane-6,4′-pyran]-2-carboxylic acid

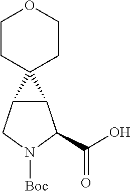 [SMILES: CC(C)(C)OC(=O)N1C[C@H]2[C@@H]([C@H]1C(=O)O)C21CCOCC1]

### Step 1: Sodium 2-(tetrahydro-4H-pyran-4-ylidene)-1-tosylhydrazin-1-ide

To a solution of 4-methyl-N′-(tetrahydro-4H-pyran-4-ylidene)benzenesulfonohydrazide (500 g, 1.86 mol, 1 equiv.) in MeOH (2.50 L) was added a solution of NaOMe (101 g, 1.86 mol, 1 equiv.) in MeOH (500 mL). The mixture was stirred for 60 min at 20° C. The mixture was concentrated under reduced pressure. The residue was slurried in MTBE (1 L) and filtered to give the title compound.

### Step 2: (3a′S,6′R,8a′S,8b′S)-6′-Phenyl-2,3,3a′,5,6,8′,8a′,8b′-octahydro-4′H,6′H-spiro[pyran-4,3′-pyrazolo[3′,4′:3,4]pyrrolo[1,2-c]oxazol]-4′-one

To a solution of (3R,7aS)-3-phenyl-1,7a-dihydro-3H,5H-pyrrolo[1,2-c]oxazol-5-one (100 g, 497 mmol, 1 equiv.) in PhCl (1.50 L) was added sodium 2-(tetrahydro-4H-pyran-4-ylidene)-1-tosylhydrazin-1-ide (144 g, 497 mmol, 1 equiv.). The mixture was heated to 135° C. under an atmosphere of nitrogen and stirred for 4 h. The mixture was filtered and concentrated. The residue was purified by flash column chromatography on silica gel eluting with EtOAc/hexanes (0-50%) to give the title compound.

### Step 3: (3R,5aS,6aR,6bS)-3-Phenyloctahydro-3H,5H-spiro[cyclopropa[3,4]pyrrolo[1,2-c]oxazole-6,4′-pyran]-5-one

A solution of (3a′S,6′R,8a′S,8b′S)-6′-phenyl-2,3,3a′,5,6,8′,8a′,8b′-octahydro-4′H,6′H-spiro[pyran-4,3′-pyrazolo[3′,4′:3,4]pyrrolo[1,2-c]oxazol]-4′-one (90 g, 290 mmol, 1 equiv.) in toluene (2.10 L) was placed in a photoreactor and irradiated at 365 nm at 50° C. for 5 hours. The mixture was concentrated under reduced pressure to give the title compound, which was used without further purification.

### Step 4: ((1R,2S,5S)-3-Benzyltetrahydro-3-azaspiro[bicyclo[3.1.0]hexane-6,4′-pyran]-2-yl)methanol

To a suspension of LAH (15.0 g, 395 mmol, 1.5 equiv.) in THF (150 mL) at 25° C. under an atmosphere of nitrogen was added a solution of (3R,5aS,6aR,6bS)-3-phenyloctahydro-3H,5H-spiro[cyclopropa[3,4]pyrrolo[1,2-c]oxazole-6,4′-pyran]-5-one (75 g, 260 mmol, 1 equiv.) in THF (300 mL). The mixture was stirred at 66° C. for 2 h, then cooled to 0° C. before aqueous Na₂SO₄ (20 mL) was added. The mixture was diluted with EtOAc (500 mL), filtered through Celite, dried over Na₂SO₄, filtered, and concentrated under reduced pressure to give the title compound, which was used without further purification.

### Step 5: ((1R,2S,5S)-Tetrahydro-3-azaspiro[bicyclo[3.1.0]hexane-6,4′-pyran]-2-yl)methanol acetate

A solution of ((1R,2S,5S)-3-benzyltetrahydro-3-azaspiro[bicyclo[3.1.0]hexane-6,4′-pyran]-2-yl)methanol (60 g, 220 mmol, 1 equiv.) 10 wt % Pd/C (9 g) in EtOAc (80 mL) and HOAc (40 mL) was stirred for 12 h at 30° C. under an atmosphere of H₂ (30 psi). The mixture was filtered through Celite and the filtrate was concentrated to give the title compound, which was used without further purification.

### Step 6: tert-Butyl (1R,2S,5S)-2-(hydroxymethyl)tetrahydro-3-azaspiro[bicyclo[3.1.0]hexane-6,4′-pyran]-3-carboxylate

A solution of ((1R,2S,5S)-tetrahydro-3-azaspiro[bicyclo[3.1.0]hexane-6,4′-pyran]-2-yl)methanol acetate (24 g, 130 mmol, 1 equiv.) in THF (72 mL) and NaOH (10.5 g, 262 mmol, 2 equiv.) in H₂O (72 mL) was treated with Boc₂O (42.9 g, 196 mmol, 1.5 equiv.) and stirred vigorously at 25° C. for 4 h. The mixture was diluted with water (30 mL) and extracted with EtOAc (3×50 mL). The organic phase was dried (MgSO₄) and concentrated. The residue was purified by column chromatography on silica gel, eluting with 10:1-2:1 petroleum ether:ethyl acetate to give the title compound. LRMS m/z: (M−^{t}Bu+2H)⁺ calculated 228.1; found 228.1. ¹H NMR (400 MHz, CDCl₃) δ 1.44 (s, 14H), 3.35-3.45 (m, 1H), 3.51-3.58 (m, 1H), 3.63-3.99 (m, 8H).

### Step 7: (1R,2S,5S)-3-(tert-Butoxycarbonyl)tetrahydro-3-azaspiro[bicyclo[3.1.0]hexane-6,4′-pyran]-2-carboxylic acid

To a solution of tert-butyl (1R,2S,5S)-2-(hydroxymethyl)tetrahydro-3 azaspiro[bicyclo[3.1.0]hexane-6,4′-pyran]-3-carboxylate (30 g, 110 mmol, 1 equiv.) in MeCN (90 mL) and H₂O (90 mL) was added TEMPO (3.33 g, 21.2 mmol, 0.2 equiv.) and PhI(OAc)₂ (102 g, 318 mmol, 3 equiv.). The mixture was stirred vigorously at 20° C. for 12 h, then diluted with water (30 mL) and extracted with EtOAc (3×50 mL). The combined extracts were concentrated under reduced pressure. The residue was purified by flash column chromatography on silica gel eluting with EtOAc/petroleum ether (0-100%). LRMS m/z: (M−^{t}Bu+2H)⁺ calculated 242.1; found 242.0. ¹H NMR (400 MHz, CDCl₃) δ 1.20-1.33 (m, 1H) 1.40-1.48 (m, 10H) 1.50-1.63 (m, 4H) 1.78 (d, J=7.4 Hz, 1H) 3.38-3.52 (m, 1H) 3.60-3.83 (m, 5H) 4.12-4.26 (m, 1H)

### Intermediate 27

### (2S,4R)-1-(((4-bromobenzyl)oxy)carbonyl)-4-(trifluoromethoxy)pyrrolidine-2-carboxylic acid

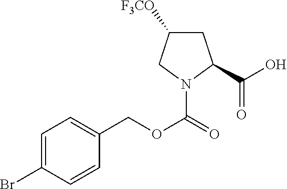 [SMILES: O=C(O)[C@@H]1C[C@@H](OC(F)(F)F)CN1C(=O)OCc1ccc(Br)cc1]

### Step 1: Methyl (2S,4R)-4-hydroxypyrrolidine-2-carboxylate

To a solution of (2S,4R)-4-hydroxypyrrolidine-2-carboxylic acid (0.40 kg, 3.0 mol) in methanol (2.4 L) at 0° C. was added SOCl₂ (0.54 kg, 4.6 mol, 0.33 L). The reaction mixture was stirred at 20° C. for 12 hours. The mixture was filtered and concentrated under reduced pressure to give the title compound, which was used without further purification.

### Step 2: 1-Benzyl 2-methyl (2S,4R)-4-hydroxypyrrolidine-1,2-dicarboxylate

To a mixture of methyl (2S,4R)-4-hydroxypyrrolidine-2-carboxylate (0.26 kg, 1.8 mol) in H₂O (3.6 L) at 20° C. was added Na₂CO₃ (0.48 kg, 4.5 mol), benzyl chloroformate (0.34 kg, 2.0 mol, 0.28 L) and dioxane (360 mL). The reaction mixture was stirred at 20° C. for 16 hours, then cooled to 0° C. and the pH was adjusted to 2 by adding 1 M HCl. The mixture was extracted with ethyl acetate. The combined organic layers were washed with brine, dried over Na₂SO₄, filtered, and concentrated under reduced pressure to give the title compound. LRMS m/z [M+H]⁺ calculated 280.1; found 280.1.

### Step 3: 1-Benzyl 2-methyl (2S,4R)-4-(((methylthio)carbonothioyl)oxy)pyrrolidine-1,2-dicarboxylate

To a solution of 1-benzyl 2-methyl (2S,4R)-4-hydroxypyrrolidine-1,2-dicarboxylate (0.25 kg, 0.90 mol) in THF (2.5 L) at 0° C. was added slowly NaH (43 g, 1.1 mol, 60% purity). The reaction mixture was stirred at 25° C. for 30 minutes, then cooled to 0° C. before CS₂ (0.10 kg, 1.3 mol, 81 mL) was added dropwise. The reaction mixture was stirred at 25° C. for 30 minutes, then cooled to 0° C. before methyl iodide (0.64 kg, 4.5 mol, 0.28 L) was added dropwise. The reaction mixture was stirred at 25° C. for 16 hours. The reaction mixture was quenched with water and extracted with ethyl acetate. The combined organics were washed with brine, dried over Na₂SO₄, filtered, and concentrated under reduced pressure. The residue was purified by preparative HPLC (Boston Uni C18) eluting with 52-82% MeCN/water with 0.1% TFA to give the title compound. LRMS m/z [M+H]⁺ calculated: 370.1; found 370.0. ¹H NMR (400 MHz, DMSO-d₆) δ 7.30-7.40 (m, 5H), 5.91 (s, 1H), 5.00-5.15 (m, 2H), 4.42-4.52 (m, 1H), 3.74-3.82 (m, 1H), 3.62 (d, J=41.2 Hz, 3H), 2.59-2.65 (m, 1H), 2.55 (d, J=19.2, 3H), 2.35-2.41 (m, 1H).

### Step 4: 1-(4-Bromobenzyl) 2-methyl (2S,4R)-4-(trifluoromethoxy)pyrrolidine-1,2-dicarboxylate

To a mixture of 1,3-dibromo-5,5-dimethylhydantoin (0.44 kg, 1.5 mol) in DCM (1.0 L) at −78° C. was added hydrogen fluoride pyridine (0.88 kg, 8.9 mol, 0.80 L). The reaction mixture was stirred at −78° C. for 10 minutes. A solution of 1-benzyl 2-methyl (2S,4R)-4-(((methylthio)carbonothioyl)oxy)pyrrolidine-1,2-dicarboxylate (0.15 kg, 0.41 mol) in DCM (1.0 L) was then added at −78° C. The reaction mixture was stirred at −78° C. for 1 hour, then at 20° C. for 16 hours. The mixture was then cooled to 0° C., the pH was adjusted to 7 by adding solid NaHCO₃ in small portions, and the mixture was extracted with DCM. The combined organics were washed with brine, dried over Na₂SO₄, filtered, and concentrated under reduced pressure. The residue was purified by silica gel chromatography eluting with ethyl acetate:petroleum ether (1:100-1:10) to give the title compound. ¹H NMR (400 MHz, DMSO-d₆) δ 7.22-7.78 (m, 4H), 4.95-5.15 (m, 3H), 4.39-4.52 (m, 1H), 3.59-3.74 (m, 5H), 2.55-2.61 (m, 1H), 2.28-2.35 (m, 1H).

### Step 5: (2S,4R)-1-(((4-Bromobenzyl)oxy)carbonyl)-4-(trifluoromethoxy)pyrrolidine-2-carboxylic acid

To a solution of 1-(4-bromobenzyl) 2-methyl (2S,4R)-4-(trifluoromethoxy)pyrrolidine-1,2-dicarboxylate (300 mg, 0.704 mmol) in THF (3 mL) and H₂O (1 mL) was added lithium hydroxide (84 mg, 3.5 mmol) at RT. The mixture was stirred at 25° C. for 16 h. The mixture was concentrated under reduced pressure. HCl (0.5 M) was added to the mixture to adjust the pH to 5. Water (20 mL) was added and the mixture was extracted with DCM (3×20 mL). The combined organic fractions were washed with brine, dried (Na₂SO₄), and filtered. The solvent was evaporated under reduced pressure the title compound. LRMS m/z: (M+H)⁺ calculated 412.0; found 412.2, 414.2.

### Intermediate 28

### Methyl (2S,4R)-4-(trifluoromethyl)pyrrolidine-2-carboxylate

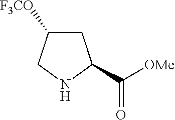 [SMILES: COC(=O)[C@@H]1C[C@@H](OC(F)(F)F)CN1]

### Step 1: 1-tert-Butyl 2-methyl (2S,4R)-4-hydroxypyrrolidine-1,2-dicarboxylate

To a mixture of methyl (2S,4R)-4-hydroxypyrrolidine-2-carboxylate (0.30 kg, 2.1 mol) in DCM (3.0 L) at room temperature was added NEt₃ (0.42 kg, 4.1 mol) and di-tert-butyl dicarbonate (0.50 kg, 2.3 mol). The reaction mixture was stirred at room temperature for 16 hours. The reaction was quenched with brine (1.0 L) and the organic layer was dried over Na₂SO₄, filtered, and concentrated under reduced pressure. The residue was purified by silica gel chromatography eluting with petroleum ether:ethyl acetate (5:1) to give the title compound.

### Step 2: 1-tert-Butyl 2-methyl (2S)-4-oxopyrrolidine-1,2-dicarboxylate

To a mixture of 1-tert-butyl 2-methyl (2S,4R)-4-hydroxypyrrolidine-1,2-dicarboxylate (0.50 kg, 2.0 mol) in DCM (5.0 L) was added trichloroisocyanuric acid (0.47 kg, 2.0 mol). The mixture was cooled to 0° C. and TEMPO (16 g, 0.10 mol) was added. The mixture was stirred at 0° C. for 30 minutes and then stirred at room temperature for 2 hours. The reaction mixture was poured into ice water and extracted with DCM. The combined organic layers were washed with brine, dried over Na₂SO₄, filtered, and concentrated under reduced pressure. The residue was purified by silica gel chromatography eluting with petroleum ether:ethyl acetate (8:1) to give the title compound.

### Step 3: 1-tert-butyl 2-methyl (2S)-4-hydroxy-4-(trifluoromethyl)pyrrolidine-1,2-dicarboxylate

To a mixture of 1-tert-butyl 2-methyl (2S)-4-oxopyrrolidine-1,2-dicarboxylate (0.43 kg, 1.8 mol) in THF (8.6 L) was added trifluoromethyltrimethylsilane (0.30 kg, 2.1 mol). The reaction mixture was cooled to 5° C. and TBAF (56 g, 1.8 mol) was added. The reaction mixture was stirred at room temperature for 16 hours, then another batch of TBAF (1.1 kg, 3.5 mol) was added and the mixture was stirred at room temperature for 2 hours. The reaction mixture was quenched with saturated aqueous NaCl and extracted into ethyl acetate. The combined organic layers were washed with brine, dried over Na₂SO₄, filtered, and concentrated under reduced pressure. The residue was purified by silica gel chromatography eluting with petroleum ether:ethyl acetate (8:1) to give the title compound.

### Step 4: tert-Butyl (2S)-4-hydroxy-2-(hydroxymethyl)-4-(trifluoromethyl)pyrrolidine-1-carboxylate

To a mixture of 1-tert-butyl 2-methyl (2S)-4-hydroxy-4-(trifluoromethyl)pyrrolidine-1,2-dicarboxylate (0.38 kg, 1.2 mol) in THF (3.8 L) and EtOH (1.9 L) at room temperature was added LiCl (51 g, 1.2 mol) and NaBH₄ (92 g, 2.4 mol). The reaction mixture was stirred at room temperature for 16 hours. The reaction mixture was quenched with saturated aqueous NH₄Cl and extracted with ethyl acetate. The combined organic layers were washed with brine, dried over Na₂SO₄, filtered and concentrated under reduced pressure to give the title compound, which was used directly in the next step.

### Step 5: tert-Butyl (2S)-2-{[(tert-butyldimethylsilyl)oxy]methyl}-4-hydroxy-4-(trifluoromethyl)pyrrolidine-1-carboxylate

To a mixture of tert-butyl (2S)-4-hydroxy-2-(hydroxymethyl)-4-(trifluoromethyl)pyrrolidine-1-carboxylate (0.32 kg, 1.1 mol) in DCM (4.8 L) was added triethylamine (0.23 kg, 2.2 mol), DMAP (27 g, 0.22 mol) and TBSCl (0.19 kg, 1.2 mol). The reaction mixture was stirred at room temperature for 16 hours. The reaction mixture was diluted with water and extracted with DCM. The combined organic layers were washed with brine, dried over Na₂SO₄, filtered and concentrated under reduced pressure. The residue was purified by silica gel chromatography eluting with petroleum ether:ethyl acetate (50:1) to give the title compound.

### Step 6: tert-butyl (2S)-2-(((tert-butyldimethylsilyl)oxy)methyl)-4-(trifluoromethyl)-2,3-dihydropyrrole-1-carboxylate

To a mixture of NaH (0.11 kg, 2.8 mol) in THF (3.7 L) was added a solution of tert-butyl (2S)-2-(((tert-butyldimethylsilyl)oxy)methyl)-4-hydroxy-4-(trifluoromethyl)pyrrolidine-1-carboxylate (0.37 kg, 0.93 mol) in THF (1.85 L) at room temperature. The reaction mixture was stirred at room temperature for 16 hours, then a solution of CF₃SO₂Cl (0.19 kg, 1.1 mol) in THF (1.85 L) was added dropwise. The reaction mixture was stirred at room temperature for 3 hours. The reaction mixture was slowly poured into ice water and extracted with ethyl acetate. The combined organic layers were washed with brine, dried over Na₂SO₄, filtered and concentrated under reduced pressure. The residue was purified by silica gel chromatography eluting with petroleum ether:ethyl acetate (100:1) to give the title compound.

### Step 7: tert-Butyl (2S)-2-(hydroxymethyl)-4-(trifluoromethyl)-2,3-dihydropyrrole-1-carboxylate

To a solution of tert-butyl (2S)-2-(((tert-butyldimethylsilyl)oxy)methyl)-4-(trifluoromethyl)-2,3-dihydropyrrole-1-carboxylate (0.28 kg, 0.73 mol) in MeOH (2.8 L) at room temperature was added NH₄F (54 g, 1.5 mol). The reaction mixture was stirred at 60° C. for 16 hours. The reaction mixture was cooled to room temperature, then quenched with brine and extracted with ethyl acetate. The combined organic layers were dried over Na₂SO₄, filtered, and concentrated under reduced pressure. The residue was purified by silica gel chromatography eluting with petroleum ether:ethyl acetate (7:1) to give the title compound.

### Step 8: tert-Butyl (2S,4R)-2-(hydroxymethyl)-4-(trifluoromethyl)pyrrolidine-1-carboxylate

To a mixture of tert-butyl (2S)-2-(hydroxymethyl)-4-(trifluoromethyl)-2,3-dihydropyrrole-1-carboxylate (0.17 kg, 0.64 mol) in DCM (1.7 L) was added [Ir(cod)(py)PCy₃]·PF₆ (10 g, 13 mmol) under an atmosphere of nitrogen at room temperature. The atmosphere was evacuated and backfilled with hydrogen three times. The mixture was stirred under a hydrogen atmosphere using a balloon at room temperature for 16 hours. The reaction mixture was filtered through a pad of celite and the filtrate was concentrated under reduced pressure to give the title compound, which was used without further purification.

### Step 9: (2S,4R)-1-(tert-Butoxycarbonyl)-4-(trifluoromethyl)pyrrolidine-2-carboxylic acid

To a solution of tert-butyl (2S,4R)-2-(hydroxymethyl)-4-(trifluoromethyl)pyrrolidine-1-carboxylate (0.17 kg, 0.63 mol) in MeCN (2.0 L) and NaH₂PO₄ aqueous buffer (1.4 L) at 45° C. was added TEMPO (9.9 g, 63 mmol) followed by the dropwise, simultaneous addition of two oxidant solutions. The first oxidant solution contained NaClO₂ (0.14 kg, 1.3 mol) dissolved in water (0.68 L) and the second oxidant solution contained NaClO (37 mL, 0.55 mol) dissolved in water (0.68 L). The reaction mixture was stirred at 45° C. for 16 hours. The reaction mixture was cooled to room temperature and a saturated aqueous solution of Na₂SO₃ (1.7 L) was added dropwise until the reaction mixture became colorless. The pH of the mixture was adjusted to 3 with the addition of 1 M HCl. The mixture was extracted with ethyl acetate. The combined organic layers were washed with brine, dried over Na₂SO₄, filtered, and concentrated under reduced pressure. The residue was recrystallized from DCM:hexane (1:20) to give the title compound.

### Step 10: (2S,4R)-4-(trifluoromethyl)pyrrolidine-2-carboxylic acid hydrochloride

To a mixture of (2S,4R)-1-(tert-butoxycarbonyl)-4-(trifluoromethyl)pyrrolidine-2-carboxylic acid (0.13 kg, 0.46 mol) in ethyl acetate (0.65 L) was added 4 M HCl in ethyl acetate (0.65 L, 71 mol). The reaction mixture was stirred at room temperature for 2 hours. The mixture was concentrated under reduced pressure and the residue was recrystallized from ethyl acetate to give the title compound.

### Step 11: (2S,4R)-1-((benzyloxy)carbonyl)-4-(trifluoromethyl)pyrrolidine-2-carboxylic acid

To a mixture of (2S,4R)-4-(trifluoromethyl)pyrrolidine-2-carboxylic acid hydrochloride (95 g, 0.43 mol) in THF (2.4 L) was added triethylamine (0.11 kg, 1.1 mol) and N-(benzyloxycarbonyloxy)succinimide (0.11 kg, 0.43 mol). The reaction mixture was stirred at room temperature for 2 hours. The mixture was treated with brine and ethyl acetate. The organic layer was washed with brine, dried over Na₂SO₄, filtered, and concentrated under reduced pressure. The residue was purified by silica gel chromatography eluting with petroleum ether:(DCM:MeOH:formic acid (10:1:0.05)) (4:1) to give the title compound. ¹H NMR (300 MHz, CDCl₃): δ 7.29-7.45 (m, 5H), 5.10-5.33 (m, 2H), 4.52-4.66 (m, 1H), 3.77-3.95 (m, 1H), 3.57-3.75 (m, 1H), 3.00-3.23 (m, 1H), 2.28-2.56 (m, 2H).

### Step 12: 1-Benzyl 2-methyl (2S,4R)-4-(trifluoromethyl)pyrrolidine-1,2-dicarboxylate

To a solution of (2S,4R)-1-((benzyloxy)carbonyl)-4-(trifluoromethyl)pyrrolidine-2-carboxylic acid (250 mg, 0.79 mmol) in MeOH (1.58 mL) at 0° C. was slowly added thionyl chloride (173 μL, 2.36 mmol). The mixture was warmed to RT and stirred for 30 minutes. The mixture was concentrated under reduced pressure to give the title compound. LRMS m/z: (M+H)⁺ calculated 332.1; found 332.2.

### Step 13: Methyl (2S,4R)-4-(trifluoromethyl)pyrrolidine-2-carboxylate

To a solution of 1-benzyl 2-methyl (2S,4R)-4-(trifluoromethyl)pyrrolidine-1,2-dicarboxylate (260 mg, 0.79 mmol) in ethanol (2.95 mL) was added 20 wt % palladium hydroxide on carbon (55 mg, 0.078 mmol). The reaction was stirred under a balloon of H₂ for 2 hours. The reaction was filtered, washing with EtOH and MeOH, and concentrated. The crude residue was dissolved in DCM and concentrated to give the title compound. LRMS m/z: (M+H)⁺ calculated 198.1; found 198.1.

### Intermediate 29

### Methyl (2(S or R),3(R or S))-3-cyclopropylpyrrolidine-2-carboxylate hydrochloride

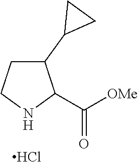 [SMILES: COC(=O)C1NCCC1C1CC1 ; Cl]

### Step 1: 1-Benzyl 2-methyl (2(S or R),3(R or S))-3-cyclopropylpyrrolidine-1,2-dicarboxylate

To a solution of 1-benzyl 2-methyl 4,5-dihydro-1H-pyrrole-1,2-dicarboxylate (10 g, 38 mmol) in EtOH (40 mL) was added saturated aq. NH₄Cl (3 mL), zinc (7.51 g, 115 mmol), copper(II) trifluoromethanesulfonate (2.77 g, 7.65 mmol) and cyclopropyl bromide (9.20 mL, 115 mmol) at 25° C. The mixture was heated to 50° C. and stirred for 40 hours. The reaction was cooled to ambient temperature and then the mixture was filtered and the solvent was removed under reduced pressure. The residue was purified by RP-HPLC (Boston Uni C18 5 μm 40×150 mm) eluting with 40-70% acetonitrile/water+0.1% TFA over 10 minutes at 60 mL/min to give a mixture of compounds. The stereoisomers were separated by SFC purification, (AD-H 20×250 mm) eluting with 20% IPA/CO₂/0.1% NH₄OH at 200 m/min to give the title compound (second-eluting stereoisomer). LRMS m/z: (M+H)⁺ calculated 304.2; found 304.1.

### Step 2: Methyl (2(S or R),3(R or S))-3-cyclopropylpyrrolidine-2-carboxylate hydrochloride

To a solution of 1-benzyl 2-methyl (2(S or R),3(R or S))-3-cyclopropylpyrrolidine-1,2-dicarboxylate (450 mg, 1.48 mmol) in DCM (1 mL) was added TEA (0.620 mL, 4.45 mmol), triethylsilane (0.711 mL, 4.45 mmol) and palladium(II) chloride (132 mg, 0.742 mmol) at 25° C. under nitrogen atmosphere. The mixture was stirred at 25° C. for 16 h. The solvent was removed under reduced pressure to give the title compound. LRMS m/z: (M+H)⁺ calculated 170.1; found 170.1.

### Intermediate 30

### (2S,3S)-3-(Benzyl((S)-1-phenylethyl)amino)-6,6-difluoro-2-hydroxyheptanoic acid

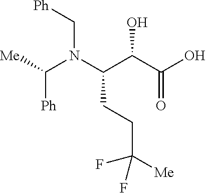 [SMILES: C[C@@H](c1ccccc1)N(Cc1ccccc1)[C@@H](CCC(C)(F)F)[C@H](O)C(=O)O]

### Step 1: Methyl 4,4-difluoropentanoate

To a stirred solution of methyl 4-oxopentanoate (710 g, 5.46 mol, 1.00 equiv.) in DCM (7.1 L) was added DAST (2.64 kg, 16.4 mol, 3.00 equiv.) dropwise at 10° C. under nitrogen atmosphere. The mixture was stirred at RT for 72 h. The reaction was quenched by the addition of NaHCO₃ (7.0 L) at 0° C. The resulting mixture was extracted with CH₂Cl₂ (2×1.5 L). The combined organic layers were washed with NaHCO₃ (2×5.0 L), brine (1×5.0 L), dried over anhydrous Na₂SO₄, and filtered to give a solution of the title compound, which was used without further purification.

### Step 2: 4,4-Difluoropentanal

To a solution of ethyl 4,4-difluoropentanoate in DCM from the previous step was added DIBAL (1 M in DCM, 6.55 L, 6.55 mol, 1.20 equiv.) dropwise at −78° C. under nitrogen atmosphere. The resulting mixture was stirred for 2 h at −78° C. The mixture was acidified to pH 2-3 with HCl (2 M). The resulting mixture was extracted with CH₂Cl₂ (2×1 L), dried over anhydrous Na₂SO₄, and filtered to give a solution of the title compound, which was used without further purification.

### Step 3: tert-Butyl (E)-6,6-difluorohept-2-enoate

To the solution of 4,4-difluoropentanal in DCM from the previous step was added tert-butyl 2-(triphenyl-λ⁵-phosphanylidene)acetate (2.05 kg, 5.45 mol, 1.00 equiv.), then stirred overnight at room temperature under nitrogen atmosphere. The resulting mixture was concentrated under vacuum. The residue was purified by silica gel column chromatography, eluting with 0-5% ethyl acetate in petroleum ether to afford the title compound.

### Step 4: tert-Butyl (2S,3S)-3-(benzyl((S)-1-phenylethyl)amino)-6,6-difluoro-2-hydroxyheptanoate

To a stirred solution of benzyl[(1S)-1-phenylethyl]amine (730 g, 3.45 mol, 1.20 equiv.) in THF (6.34 L) was added n-hexyllithium (1.70 L, 3.74 mol, 1.30 equiv.) dropwise at −60° C. under nitrogen atmosphere. The resulting mixture was stirred for 30 min at −60° C. To the stirred solution was added tert-butyl (2E)-6,6-difluorohept-2-enoate (634 g, 2.88 mol, 1.00 equiv.) in THF (634 mL) dropwise at −60° C. The resulting mixture was stirred for 30 min at −60° C. Then to the solution was added (1S)-7,7-dimethyl-1-[(oxaziridine-2-sulfonyl)methyl]bicyclo[2.2.1]heptan-2-one (1.05 kg, 4.03 mol, 1.40 equiv.) in portions at −60° C. The resulting mixture was stirred for 2 h at −60° C. The reaction was quenched with AcOH (311 g, 5180 mmol, 1.80 equiv.) at −60° C. The mixture was basified to pH 8 with aqueous NaHCO₃. The resulting mixture was extracted with EtOAc (2×3 L). The combined organic layers were washed with brine (1×5 L) and dried over anhydrous Na₂SO₄. After filtration, the filtrate was concentrated under reduced pressure. The residue was purified by trituration with MTBE (5 L). The resulting mixture was filtered and the filtrate was concentrated under reduced pressure. The residue was purified by silica gel column chromatography eluting with 0-10% ethyl acetate in petroleum ether to give the title compound. LRMS m/z: (M+H)⁺ calculated 448.3; found 448.3.

### Step 5: (2S,3S)-3-(Benzyl((S)-1-phenylethyl)amino)-6,6-difluoro-2-hydroxyheptanoic acid

A solution of tert-butyl (2S,3S)-3-(benzyl((1S)-1-phenylethyl)amino)-6,6-difluoro-2-hydroxyheptanoate (980 g, 2.19 mol, 1.00 equiv.) in toluene (4.9 L) was treated with TFA (1.50 kg, 13.1 mol, 6.00 equiv.) for 5 h at 50° C. under nitrogen atmosphere. The resulting mixture was concentrated under vacuum. The residue was purified by silica gel column chromatography eluting with 0-45% ethyl acetate in petroleum ether to give the title compound. LRMS m/z: (M+H)⁺ calculated 392.2; found 392.2.

### Intermediate 31

### (2S,3S)-3-amino-6,6-difluoro-2-hydroxy-N-methylheptanamide, HCl

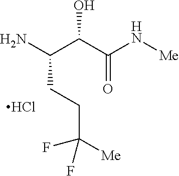 [SMILES: Cl ; CNC(=O)[C@@H](O)[C@@H](N)CCC(C)(F)F]

### Step 1: (2S,3S)-3-(benzyl((S)-1-phenylethyl)amino)-6,6-difluoro-2-hydroxy-N-methylheptanamide

A stirred solution of (2S,3S)-3-(benzyl((S)-1-phenylethyl)amino)-6,6-difluoro-2-hydroxyheptanoic acid (45 g, 120 mmol), methylamine HCl (16 g, 230 mmol) and CH₂Cl₂ (460 mL) was treated with NMM (50.6 ml, 460 mmol) followed by HATU (54.6 g, 144 mmol). The mixture was stirred at ambient temperature overnight. The mixture was diluted with saturated aqueous NaHCO₃ and then extracted with CH₂Cl₂. The organic portion was washed with brine and dried (MgSO₄), and then the solvent was removed under reduced pressure. The residue was dissolved in 300 mL 1 N HCl and then washed with 500 mL of Et₂O. The aqueous portion was separated, basified with 1 N NaOH and then extracted with EtOAc. The combined organic portions were washed with brine, dried (MgSO₄), and filtered. The solvent was removed under reduced pressure to give the title compound. LRMS m/z: (M+H)⁺ calculated 406.2; found 406.4.

### Step 2: (2S,3S)-3-amino-6,6-difluoro-2-hydroxy-N-methylheptanamide hydrochloride

A solution of (2S,3S)-3-(benzyl((S)-1-phenylethyl)amino)-6,6-difluoro-2-hydroxy-N-methylheptanamide (7.5 g, 19 mmol) in EtOH (185 mL) was treated with acetic acid (3.18 mL, 55.6 mmol) and 10 wt % Pd/C (1.12 g, 0.927 mmol) and then stirred under 1 atm H₂ for 18 hours. The mixture was filtered through celite and the celite pad was washed with EtOH. The solvent was removed under reduced pressure and then the residue was treated with 50 mL MeOH followed by 20 mL 4 N HCl in dioxane. The mixture was stirred for 5 minutes and then the solvent was removed under reduced pressure to give title compound. LRMS m/z: (M+H)⁺ calculated 211.2; found 211.2.

The following compound was prepared in essentially the same manner:

| Intermediate | | | LRMS m/z |
|---|---|---|---|
| No. | Structure | Name | (M + H)⁺ |
| 32 | 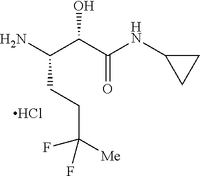 [SMILES: Cl ; CC(F)(F)CC[C@H](N)[C@H](O)C(=O)NC1CC1] | (2S,3S)-3-amino-N- cyclopropyl-6,6-difluoro- 2-hydroxyheptanamide hydrochloride | Calculated: 237.1 Found: 237.2 |

### Intermediate 33

### (S)-5,5-difluoro-1,1-dimethoxyhexan-2-amine

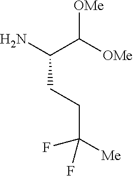 [SMILES: COC(OC)[C@@H](N)CCC(C)(F)F]

### Step 1: 3-(tert-Butyl) 4-methyl (R)-2,2-dimethyloxazolidine-3,4-dicarboxylate

To a stirred solution of methyl (2R)-2-((tert-butoxycarbonyl)amino)-3-hydroxypropanoate (800 g, 3.65 mol, 1.00 equiv.) and 2,2-dimethoxypropane (3.80 kg, 36.5 mol, 10.0 equiv.) in acetone (8 L) was added BF₃·Et₂O (26 g, 180 mmol, 0.05 equiv) dropwise at room temperature. The resulting mixture was stirred for 3 h at room temperature. The resulting mixture was concentrated under reduced pressure. The residue was purified by silica gel column chromatography eluting with petroleum ether:ethyl acetate (10:1) to afford the title compound. LRMS m/z: (M+H)⁺ calculated 260.2; found 260.2.

### Step 2: tert-Butyl (S)-4-(hydroxymethyl)-2,2-dimethyloxazolidine-3-carboxylate

To a stirred solution of 3-tert-butyl 4-methyl (4R)-2,2-dimethyl-1,3-oxazolidine-3,4-dicarboxylate (800 g, 3.09 mol, 1.00 equiv.) in THF (6.4 L) was added LiAlH₄ (234 g, 6.17 mol, 2.00 equiv) dropwise at 0° C. under nitrogen atmosphere. The resulting mixture was stirred for overnight at room temperature under nitrogen atmosphere. The reaction was quenched with water/ice at 0° C. The resulting mixture was concentrated under reduced pressure and diluted with 2:1 petroleum ether:ethyl acetate (1 L). The resulting mixture was filtered, the filter cake was washed with 2:1 petroleum ether:ethyl acetate (1 L), and the filtrate was concentrated under reduced pressure. The residue was purified by silica gel column chromatography eluting with petroleum ether:ethyl acetate (3:1) to afford the title compound. LRMS m/z: (M+H)⁺ calculated 232.2; found 232.2.

### Step 3: tert-Butyl (R)-4-formyl-2,2-dimethyloxazolidine-3-carboxylate

To a stirred solution of oxalyl chloride (469 g, 3.70 mol, 1.50 equiv.) in DCM (3 L) was added a solution of DMSO (578 g, 7.39 mol, 3.00 equiv.) in DCM (1 L) at −78° C. under nitrogen atmosphere. To the above, a solution of tert-butyl (4S)-4-(hydroxymethyl)-2,2-dimethyl-1,3-oxazolidine-3-carboxylate (570 g, 2.46 mol, 1.00 equiv.) in DCM (5 L) was added dropwise at −60° C. The mixture was stirred for 30 min at −60° C. under nitrogen atmosphere. A solution of TEA (1.50 kg, 14.8 mol, 6.00 equiv.) in DCM (3 L) was added to the above solution at −50° C. and the resulting mixture was stirred for 30 min at −45° C. under nitrogen atmosphere. The resulting mixture was stirred at 0° C. for 1 h. The reaction was quenched by the addition of HCl (6 L, 0.5 M) at room temperature. The resulting mixture was extracted with CH₂Cl₂ (2×1 L). The combined organic layers were washed with water (1×1 L) and dried over anhydrous Na₂SO₄. The mixture was filtered and the filtrate was concentrated under reduced pressure. The residue was purified by silica gel column chromatography eluting with petroleum ether:ethyl acetate (10:1) to afford the title compound.

### Step 4: tert-Butyl (S,E)-2,2-dimethyl-4-(3-oxobut-1-en-1-yl)oxazolidine-3-carboxylate

A mixture of tert-butyl 4-formyl-2,2-dimethyl-1,3-oxazolidine-3-carboxylate (550 g, 2.40 mol, 1.00 equiv.) and 1-(triphenyl-λ⁵-phosphanylidene)propan-2-one (802 g, 2.52 mol, 1.05 equiv.) in THF (7.5 L) was stirred overnight at 50° C. under nitrogen atmosphere. The resulting mixture was concentrated under reduced pressure and diluted with 4:1 petroleum ether:ethyl acetate (2 L). The mixture was filtered, the filter cake was washed with 4:1 petroleum ether:ethyl acetate (1 L), and the filtrate was concentrated under reduced pressure. The residue was purified by silica gel column chromatography eluting with petroleum ether:ethyl acetate (6:1) to afford the title compound. LRMS m/z: (M+H)⁺ calculated 270.2; found 270.2.

### Step 5: tert-Butyl (S)-2,2-dimethyl-4-(3-oxobutyl)oxazolidine-3-carboxylate

To a stirred solution of tert-butyl (4S)-2,2-dimethyl-4-[(1E)-3-oxobut-1-en-1-yl]-1,3-oxazolidine-3-carboxylate (580 g, 2.15 mol, 1.00 equiv) in MeOH (5 L) was added Pd/C (57.3 g, 538 mmol, 0.25 equiv) at room temperature. The mixture was placed under an atmosphere of H₂ and stirred overnight at room temperature. The resulting mixture was filtered, the filter cake was washed with MeOH (200 mL), and the filtrate was concentrated under reduced pressure to afford the title compound. LRMS m/z: (M+H)⁺ calculated 272.2; found 272.2.

### Step 6: tert-Butyl (S)-4-(3,3-difluorobutyl)-2,2-dimethyloxazolidine-3-carboxylate

To a stirred mixture of tert-butyl (4S)-2,2-dimethyl-4-(3-oxobutyl)-1,3-oxazolidine-3-carboxylate (570 g, 2.10 mol, 1.00 equiv) in diethyl(trifluoro-λ⁴-sulfanyl)amine (4 L) at room temperature under air atmosphere for 48 h. The resulting mixture was diluted with Et₂O (6 L). The reaction was quenched with saturated NaHCO₃ (3 L) at 0° C. The resulting mixture was extracted with EtOAc (2×2 L). The combined organic layers were washed with sat. aq. NaHCO₃ (1 L) and dried over anhydrous Na₂SO₄. After filtration, the filtrate was concentrated under reduced pressure. The residue was purified by silica gel column chromatography eluting with petroleum ether:ethyl acetate (7:1) to afford the title compound. LRMS m/z: (M+H)⁺ calculated 294.2; found 294.2.

### Step 7: Benzyl (S)-(5,5-difluoro-1-hydroxyhexan-2-yl)carbamate

To a stirred solution of tert-butyl (4S)-4-(3,3-difluorobutyl)-2,2-dimethyl-1,3-oxazolidine-3-carboxylate (355 g, 1.21 mol, 1.00 equiv.) in MeOH (2 L) was added HCl (441 g, 12.1 mol, 10.0 equiv.) dropwise at room temperature. The reaction mixture was stirred for 30 min at room temperature. The mixture was concentrated under reduced pressure, then taken up in THF (4 L) and H₂O (2 L) at room temperature. K₂CO₃ (335 g, 2.42 mol, 2.00 equiv.) was added. To the above mixture was added benzyl chloroformate (248 g, 1.45 mol, 1.20 equiv.) dropwise at 0° C. The resulting mixture was stirred for additional 2 h at room temperature. The resulting mixture was extracted with EtOAc (3×1 L). The combined organic layers were washed with water (1 L) and dried over anhydrous Na₂SO₄. The mixture was filtered and the filtrate was concentrated under reduced pressure. The residue was purified by silica gel column chromatography eluting with petroleum ether:ethyl acetate (1:1) to afford title compound. LRMS m/z: (M+H)⁺ calculated 288.1; found 288.1.

### Step 8: Benzyl (S)-(5,5-difluoro-1-oxohexan-2-yl)carbamate

To a stirred solution of benzyl N-((2S)-5,5-difluoro-1-hydroxyhexan-2-yl)carbamate (290 g, 1.01 mol, 1.00 equiv.) in CH₂Cl₂ (3 L) was added Dess-Martin periodinane (514 g, 1.21 mol, 1.20 equiv.) in portions at room temperature under air atmosphere. The resulting mixture was stirred overnight at room temperature. The reaction was quenched with saturated Na₂S₂O₃ (aq.) and saturated NaHCO₃(aq.) at 0° C. The resulting mixture was extracted with CH₂Cl₂ (2×2 L). The combined organic layers were washed with water (1 L) and dried over anhydrous Na₂SO₄. The mixture was filtered and the filtrate was concentrated under reduced pressure. The residue was purified by silica gel column chromatography eluting with petroleum ether:ethyl acetate (3:1) to afford title compound. LRMS m/z: (M+H)⁺ calculated 286.1; found 286.1.

### Step 9: benzyl (S)-(5,5-difluoro-1,1-dimethoxyhexan-2-yl)carbamate

To a stirred solution of benzyl N-(5,5-difluoro-1-oxohexan-2-yl)carbamate (176 g, 617 mmol, 1.00 equiv.) and trimethyl orthoformate (78.6 g, 740 mmol, 1.20 equiv) in MeOH (1.5 L) was added para-toluene sulfonate (10.6 g, 61.7 mmol, 0.10 equiv) in portions at room temperature. The resulting mixture was stirred overnight at room temperature. The resulting mixture was concentrated under reduced pressure. The residue was dissolved in EtOAc (2 L) and saturated aqueous NaHCO₃ (1 L). The resulting mixture was extracted with EtOAc (2×1.5 L). The combined organic layers were dried over anhydrous Na₂SO₄. The mixture as filtered and the filtrate was concentrated under reduced pressure. The residue was purified by trituration with 3:1 petroleum ether:ethyl acetate (1 L) and the solids were collected by filtration. The crude product was purified by SFC (Lux Cellulose-25 μm 30×250 mm) eluting with 15% (1:1 isopropanol:hexanes)/CO₂ at 100 bar to afford the title compound. LRMS m/z: (M+H)⁺ calculated 332.2; found 332.2.

### Step 10: (S)-5,5-difluoro-1,1-dimethoxyhexan-2-amine

To a stirred solution of benzyl (S)-(5,5-difluoro-1,1-dimethoxyhexan-2-yl)carbamate (1.5 g, 4.53 mmol) in EtOH (34.8 ml) was added 20 wt % Pd(OH)₂ on carbon (0.318 g, 0.453 mmol). The flask was purged, placed under H_{2 (g)} using a balloon and stirred under atmospheric H_{2 (g)} at RT for 1 hr. The mixture was filtered carefully through a prepacked celite filter and the catalyst was washed with EtOAc. The filtrate was concentrated to give the title compound. LRMS m/z: (M+H)⁺ calculated 198.1; found 198.2.

### Intermediate 34

### (3S)-3-amino-6,6-difluoro-2-hydroxyheptanamide

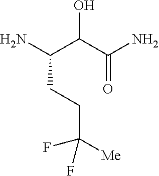 [SMILES: CC(F)(F)CC[C@H](N)C(O)C(N)=O]

### Step 1: Benzyl (S)-(5,5-difluoro-1-oxohexan-2-yl)carbamate

A solution of benzyl (S)-(5,5-difluoro-1,1-dimethoxyhexan-2-yl)carbamate (700 mg, 2.11 mmol) in acetone (7.04 mL)/water (7.04 mL) was treated with Dowex® 50WX8 Hydrogen Form 200-400 mesh (2.1 g) and then shaken and heated to 70° C. for 18 hours. The mixture was filtered and then the solvent was removed under reduced pressure. The residue was dissolved in EtOAc and then washed with saturated aqueous NaHCO₃ and brine, dried (MgSO₄), and filtered. The solvent was removed under reduced pressure to give the title compound. LRMS m/z: (M+H)⁺ calculated 286.1; found 286.2.

### Step 2: Benzyl ((2S)-1-cyano-5,5-difluoro-1-hydroxyhexan-2-yl)carbamate

To a stirred solution of benzyl (S)-(5,5-difluoro-1-oxohexan-2-yl)carbamate (125 mg, 0.438 mmol) and MeOH (2.19 mL) at 0° C. was added cesium fluoride (66.6 mg, 0.438 mmol) followed by dropwise addition of trimethylsilyl cyanide (147 μL, 1.10 mmol). After 30 minutes the cooling bath was removed and the solution was stirred overnight. The reaction was quenched with saturated aqueous NaHCO₃ and then extracted with EtOAc. The organic portion was washed with brine, dried (MgSO₄), and filtered. The solvent was removed under reduced pressure to give the title compound. LRMS m/z: (M+Na)⁺ calculated 335.1; found 335.1.

### Step 3: Benzyl ((3S)-1-amino-6,6-difluoro-2-hydroxy-1-oxoheptan-3-yl)carbamate

To a stirred solution of benzyl ((2S)-1-cyano-5,5-difluoro-1-hydroxyhexan-2-yl)carbamate (130 mg, 0.416 mmol) and MeOH (2.08 mL) at ambient temperature was added lithium hydroxide, H₂O (21.8 mg, 0.520 mmol) followed by hydrogen peroxide (425 μL, 4.16 mmol) and the mixture was stirred overnight. The reaction was quenched with saturated aqueous sodium thiosulfate and then extracted with EtOAc. The organic portion was washed with brine, dried (MgSO₄), and filtered, and the solvent was removed under reduced pressure. DMSO (5 mL) was added to the residue, which was filtered and the filtrate purified by reverse-phase HPLC (Waters SunFire® OBD 10 μm 30×150 mm) eluting with a gradient of 5-95% acetonitrile/water+0.1% TFA over 15 minutes. The fractions that contained product were combined, basified with saturated aqueous NaHCO₃, and then extracted with CH₂Cl₂. The organic portion was dried (MgSO₄) and filtered. The solvent was removed under reduced pressure to give the title compound. LRMS m/z: (M+H)⁺ calculated 331.1; found 331.3.

### Step 4: (3S)-3-Amino-6,6-difluoro-2-hydroxyheptanamide

A solution of benzyl ((3S)-1-amino-6,6-difluoro-2-hydroxy-1-oxoheptan-3-yl)carbamate (100 mg, 0.30 mmol) in EtOH (0.250 mL)/EtOAc (1.26 mL) was treated with 20% palladium hydroxide (21 mg, 0.030 mmol) and then stirred under 1 atm H₂ for 1 hour. The mixture was filtered through celite and the celite pad was washed with EtOH. The filtrate was concentrated under reduced pressure to give the title compound. LRMS m/z: (M+H)⁺ calculated 197.2; found 197.1.

### Intermediate 35

### (2S)-4(R or S)-Cyclobutyl-1-((S)-2-((methoxycarbonyl)amino)-3,3-dimethylbutanoyl)pyrrolidine-2-carboxylic acid

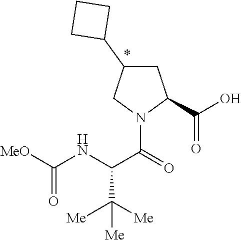 [SMILES: COC(=O)N[C@H](C(=O)N1CC(C2CCC2)C[C@H]1C(=O)O)C(C)(C)C]

### Step 1: 2-Benzyl 1-(tert-butyl) (2S)-4-bromopyrrolidine-1,2-dicarboxylate

To a stirred solution of (2S)-4-bromo-1-(tert-butoxycarbonyl)pyrrolidine-2-carboxylic acid (2 g, 7 mmol) in DMF (34.0 mL) at ambient temperature was added cesium carbonate (1.11 g, 3.40 mmol) followed by benzyl bromide (0.809 mL, 6.80 mmol). The mixture was stirred at room temperature for 18 hours. The mixture was diluted with H₂O and then extracted with EtOAc (2×50 mL). The combined organic portions were washed with saturated aqueous NaHCO₃ and brine, dried (MgSO₄), and filtered. The solvent was removed under reduced pressure to give the title compound. LRMS m/z: (M+H)⁺ calculated 384.1; found 384.1, 386.1.

### Step 2: Benzyl (2S)-4-bromopyrrolidine-2-carboxylate hydrochloride

To a stirred solution of 2-benzyl 1-(tert-butyl) (2S)-4-bromopyrrolidine-1,2-dicarboxylate (2.6 g, 6.8 mmol), and CH₂Cl₂ (33.8 mL) at ambient temperature was added TFA (5.21 mL, 67.7 mmol). The mixture was stirred for 3 hours at ambient temperature. The solvent was removed under reduced pressure. The residue was dissolved in 20 mL of 4M HCl in EtOAc and stirred for 5 minutes. The solvent was removed under reduced pressure and then the residue was azeotroped with EtOAc (3×10 ml) to give the title compound. LRMS m/z: (M+H)⁺ calculated 285.0; found 284.0, 286.0.

### Step 3: Benzyl (2S)-4-bromo-1-((S)-2-((methoxycarbonyl)amino)-3,3-dimethylbutanoyl)pyrrolidine-2-carboxylate

To a stirred solution of (S)-2-((methoxycarbonyl)amino)-3,3-dimethylbutanoic acid (1.18 g, 6.24 mmol), benzyl (2S)-4-bromopyrrolidine-2-carboxylate hydrochloride (2 g, 6 mmol), and N-methylmorpholine (2.74 mL, 25.0 mmol) in DMF (25.0 mL) at ambient temperature was added HATU (2.85 g, 7.49 mmol). The mixture was stirred at ambient temperature for 18 hours. The mixture was diluted with H₂O and then extracted with EtOAc (2×50 mL). The combined organic portions were washed with saturated aqueous NaHCO₃ and brine, dried (MgSO₄), and filtered. The solvent was removed under reduced pressure and the residue was purified by column chromatography on silica gel eluting with 0-100% EtOAc in hexanes to give the title compound. LRMS m/z: (M+H)⁺ calculated 456.1; found 455.1, 457.1.

### Step 4: Benzyl (2S)-4(S or R)-cyclobutyl-1-((S)-2-((methoxycarbonyl)amino)-3,3-dimethylbutanoyl)pyrrolidine-2-carboxylate

Nickel(II) chloride hexahydrate (19.57 mg, 0.082 mmol) and 2,6-bis((4R)-4-phenyl-2-oxazolinyl)pyridine (30.4 mg, 0.082 mmol) were combined in 3 mL acetonitrile and stirred for 1 hour to provide light blue suspension. To a suspension of benzyl (2S)-4-bromo-1-((S)-2-((methoxycarbonyl)amino)-3,3-dimethylbutanoyl)pyrrolidine-2-carboxylate (750 mg, 1.6 mmol), sodium carbonate (524 mg, 4.94 mmol) and acetonitrile (16.5 mL) at ambient temperature was added (Ir[dF(CF₃)ppy]₂(dtbbpy))PF₆ followed by the pre-made nickel complex and (hydroxy-bis(trimethylsilyl)silyl)-trimethylsilane (1.52 mL, 4.94 mmol). The mixture was purged with nitrogen for 30 seconds and then the vial was sealed with parafilm. The mixture was then irradiated in a Penn photoreactor (100% intensity, 1000 rpm stir rate, 5400 rpm fan) for 1 hour. The mixture was diluted with EtOAc and then washed with H₂O, saturated aqueous NaHCO₃, and brine, dried (MgSO₄), and filtered. The solvent was removed under reduced pressure. The residue was purified by column chromatography on silica gel eluting with 0-100% EtOAc in hexanes to provide racemic material. The stereoisomers were separated by SFC purification using a Chiralpak AD-H column (2×25 cm), eluting with 35% methanol/CO₂, at 100 bar and 90 mL/min to give the title compound (second-eluting stereoisomer). LRMS m/z: (M+H)⁺ calculated 431.3; found 431.3.

### Step 5: (2S)-4(S or R)-cyclobutyl-1-((S)-2-((methoxycarbonyl)amino)-3,3-dimethylbutanoyl) pyrrolidine-2-carboxylic acid

To a stirred solution of benzyl (2S)-4(R or S)-cyclobutyl-1-((S)-2-((methoxycarbonyl)amino)-3,3-dimethylbutanoyl)pyrrolidine-2-carboxylate (150 mg, 0.35 mmol) in ethanol (2 mL) at ambient temperature was added 1 N NaOH (0.383 mL, 0.383 mmol). The mixture was stirred for 30 minutes at ambient temperature and then the solvent was removed under reduced pressure. 0.5 mL 1 N HCl was added to the residue. The mixture was stirred for 5 minutes and then the solvent was removed under reduced pressure and azeotroped with THF to give the title compound. LRMS m/z: (M+H)⁺ calculated 341.2; found 341.2.

The following compounds were prepared in essentially the same manner:

| Intermediate | | | LRMS m/z |
|---|---|---|---|
| No. | Structure | Name | (M + H)⁺ |
| 36 | 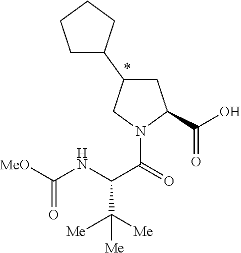 [SMILES: COC(=O)N[C@H](C(=O)N1CC(C2CCCC2)C[C@H]1C(=O)O)C(C)(C)C] | (2S,4(S or R))-4-Cyclopentyl-1- ((S)-2- ((methoxycarbonyl)amino)-3,3- dimethylbutanoyl)pyrrolidine-2- carboxylic acid | Calculated: 355.2 Found: 355.3 |
| 37 | 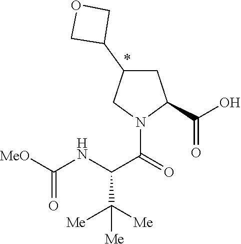 [SMILES: COC(=O)N[C@H](C(=O)N1CC(C2COC2)C[C@H]1C(=O)O)C(C)(C)C] | (2S,4(S or R))-1-((S)-2- ((Methoxycarbonyl)amino)-3,3- dimethylbutanoyl)-4-(oxetan-3- yl)pyrrolidine-2-carboxylic acid | Calculated: 343.2 Found: 343.2 |
| 38 | 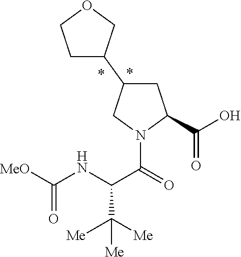 [SMILES: COC(=O)N[C@H](C(=O)N1CC(C2CCOC2)C[C@H]1C(=O)O)C(C)(C)C] | (2S,4(S or R))-1-((S)-2- ((Methoxycarbonyl)amino)-3,3- dimethylbutanoyl)-4-((R or S)- tetrahydrofuran-3- yl)pyrrolidine-2-carboxylic acid | Calculated: 357.2 Found: 357.3 |

### Intermediate 39

### (S)-2-((tert-butoxycarbonyl)amino)-3-(5-methyl-1,2,4-oxadiazol-3-yl)propanoic acid

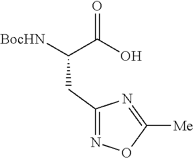 [SMILES: Cc1nc(C[C@H](NC(=O)OC(C)(C)C)C(=O)O)no1]

### Step 1: 2-chloro-N-hydroxyethanimidamide

To a stirred solution of hydroxylamine hydrochloride (736 g, 10.6 mol, 1 equiv.) in ethanol (8 L) was added sodium ethoxide (721 g, 10.6 mol, 1 equiv.) in portions at 0° C. under nitrogen atmosphere. Chloroacetonitrile (800 g, 10.6 mol, 1 equiv.) was added to the solution. The resulting mixture was stirred for 1 h at room temperature under nitrogen atmosphere. The resulting mixture was filtered, and the filter cake was washed with ethanol (2×1 L). The filtrate was concentrated under reduced pressure to provide the title compound, which was used without further purification.

### Step 2: 3-(chloromethyl)-5-methyl-1,2,4-oxadiazole

To a stirred solution of 2-chloro-N-hydroxyethanimidamide (800 g, 7.37 mol, 1 eq.) and Na₂CO₃ (937 g, 8.85 mol, 1.2 equiv.) in DMF (2.80 L) was added acetic anhydride (2.26 kg, 22.1 mol, 3 equiv.) dropwise at room temperature under hydrogen atmosphere. The resulting mixture was stirred overnight at 110° C. under nitrogen atmosphere. The precipitated solids were collected by filtration and washed with ethyl acetate (2×500 mL). The organic phase was extracted with ethyl acetate (2×500 mL). The combined organic layers were washed with brine (3×1 L) and dried over anhydrous Na₂SO₄. After filtration, the filtrate was concentrated under reduced pressure to afford the title compound, which was used without further purification.

### Step 3: 2-amino-3-(5-methyl-1,2,4-oxadiazol-3-yl) propanoic acid

To a stirred solution of methyl 2-[(diphenylmethylidene)amino]acetate (1.26 kg, 4.98 mol, 1.25 equiv.) in THF (5.50 L) was added LiHMDS (1.04 kg, 6.22 mol, 1.5 equiv.) dropwise at −78° C. under nitrogen atmosphere over 0.5 h. To the above mixture was added 3-(chloromethyl)-5-methyl-1,2,4-oxadiazole (550 g, 4.15 mol, 1 equiv.) in THF (1.00 L) dropwise over 1 h at −78° C. The resulting mixture was stirred for additional 12 h at room temperature. The resulting mixture was diluted with water (1 L). The mixture was acidified to pH 5 with HCl (3 M) and stirred for 0.5 h to afford the title compound. This mixture was used without further purification.

### Step 4: 2-[(tert-butoxycarbonvl)amino]-3-(5-methyl-1,2,4-oxadiazol-3-yl) propanoic acid

To a stirred solution of 2-amino-3-(5-methyl-1,2,4-oxadiazol-3-yl) propanoic acid (91.6 g, 0.54 mol, 1 equiv.) and K₂CO₃ (908 g, 1.34 mol, 2.5 equiv.) in THF (910 mL) and H₂O (455 mL) were added di-tert-butyl dicarbonate (140 g, 0.64 mol, 1.2 equiv.) dropwise at room temperature under nitrogen atmosphere for 2 h. The resulting mixture was diluted with water (500 mL). The resulting mixture was extracted with DCM (2×500 mL). The combined organic layers were washed with brine (1 L) and dried over anhydrous Na₂SO₄. After filtration, the filtrate was concentrated under reduced pressure. The residue was purified by silica gel column chromatography eluting with 2:1 petroleum ether:ethyl acetate to afford the title compound.

### Step 5: Methyl (2S)-2-[(tert-butoxycarbonyl)amino]-3-(5-methyl-1,2,4-oxadiazol-3-yl)propanoate

To a stirred solution of 2-[(tert-butoxycarbonyl)amino]-3-(5-methyl-1,2,4-oxadiazol-3-yl) propanoic acid (112 g, 413 mmol, 1 equiv.) and K₂CO₃ (114 g, 826 mmol, 2 equiv.) in DMF (1.12 L) were added methyl iodide (117 g, 826 mmol, 2 equiv.) dropwise at 40° C. under nitrogen atmosphere for 16 h. The resulting mixture was diluted with water (1.00 L). The resulting mixture was extracted with DCM (2×500 mL). The combined organic layers were washed with brine (2×1 L) and dried over anhydrous Na₂SO₄. After filtration, the filtrate was concentrated under reduced pressure. The crude product by chiral preparative HPLC (CHIRALPAK IG, 7×25 cm, 10 μm) eluting with hexanes and 45:10 EtOH:DCM to afford the title compound. LRMS m/z: (M+H)⁺ calculated 285.1; found 286.1.

### Step 6: (S)-2-((tert-butoxycarbonyl)amino)-3-(5-methyl-1,2,4-oxadiazol-3-yl)propanoic acid

To a stirred solution of methyl (S)-2-((tert-butoxycarbonyl)amino)-3-(5-methyl-1,2,4-oxadiazol-3-yl)propanoate (3 g, 11 mmol) in MeOH (20 mL) and water (2.2 mL) was added lithium hydroxide (0.302 g, 12.6 mmol). The reaction mixture was stirred at ambient temperature overnight. The reaction was concentrated under reduced pressure. DCM (100 mL) was added to the residue and the mixture was acidified with formic acid (1.2 mL, 32 mmol), which was washed with water (30 mL). The aqueous portion was separated and extracted with DCM. The combined organic portions were washed with brine, dried over Na₂SO₄, filtered, and concentrated under reduced pressure to give the title compound. LRMS m/z: (M+H)⁺ calculated 271.1; found 272.2.

### EXAMPLES

### From Scheme C

### Example 1

### Methyl ((S)-1-((2S,4(S or R))-4-cyclobutyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate

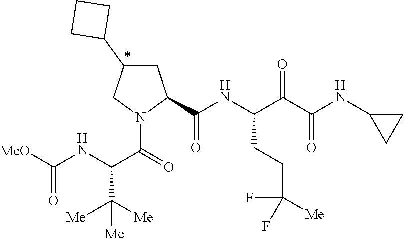 [SMILES: COC(=O)N[C@H](C(=O)N1CC(C2CCC2)C[C@H]1C(=O)N[C@@H](CCC(C)(F)F)C(=O)C(=O)NC1CC1)C(C)(C)C]

### Step 1: Methyl ((S)-1-((2SA(S or R))-4-cyclobutyl-2-(((S)-5,5-difluoro-1,1-dimethoxyhexan-2-yl)carbamoyl)pyrrolidin-1-yl)-3,3-dimethyl-1-oxobutan-2-yl)carbamate

To a stirred solution of (2S)-4(R or S)-cyclobutyl-1-((S)-2-((methoxycarbonyl)amino)-3,3-dimethylbutanoyl) pyrrolidine-2-carboxylic acid (100 mg, 0.29 mmol), (S)-5,5-difluoro-1,1-dimethoxyhexan-2-amine (63.7 mg, 0.323 mmol), N-methylmorpholine (129 μL, 1.18 mmol) and DMF (2.94 mL) was added HATU (140 mg, 0.37 mmol) and the reaction was stirred at ambient temperature overnight. The reaction was diluted with EtOAc and then washed with saturated aqueous NaHCO₃, and brine. The organic phase was dried (MgSO₄), filtered, and concentrated under reduced pressure. The residue was purified by column chromatography on silica gel eluting with 0-100% EtOAc in hexanes to give the title compound. LRMS m/z: (M+H)⁺ calculated 520.3; found 520.3.

### Step 2: Methyl ((S)-1-((2SA(S or R))-4-cyclobutyl-2-(((S)-5,5-difluoro-1-oxohexan-2-yl)carbamoyl)pyrrolidin-1-yl)-3,3-dimethyl-1-oxobutan-2-yl)carbamate

Dowex® 50WX8 Hydrogen Form 200-400 mesh (450 mg) was added to a solution of methyl ((S)-1-((2S,4(S or R))-4-cyclobutyl-2-(((S)-5,5-difluoro-1,1-dimethoxyhexan-2-yl)carbamoyl)pyrrolidin-1-yl)-3,3-dimethyl-1-oxobutan-2-yl)carbamate (126 mg, 0.242 mmol) in acetone (2.43 mL) and water (2.43 mL) and shaken at 70° C. for 48 h. The reaction mixture was filtered through a syringe filter, washing with acetone. The filtrate was concentrated under reduced pressure and the residue was azeotroped with THF to give the title compound. LRMS m/z: (M+H)⁺ calculated 474.3; found 474.3.

### Step 3: (3S)-3-((2S,4(S or R))-4-Cyclobutyl-1-((S)-2-((methoxycarbonyl)amino)-3,3-dimethylbutanoyl)pyrrolidine-2-carboxamido)-1-(cyclopropylamino)-6,6-difluoro-1-oxoheptan-2-yl acetate

To a stirred solution of methyl ((S)-1-((2S,4(S or R))-4-cyclobutyl-2-(((S)-5,5-difluoro-1-oxohexan-2-yl)carbamoyl)pyrrolidin-1-yl)-3,3-dimethyl-1-oxobutan-2-yl)carbamate (110 mg, 0.23 mmol) and CH₂Cl₂ (2.32 mL) at 0° C. was added cyclopropyl isocyanide (23.8 μL, 0.348 mmol) followed by acetic acid (20.0 μL, 0.348 mmol). The reaction was stirred at 0° C. for 5 mn followed by removal of the cooling bath. After another 1 hour at ambient temperature, the reaction was quenched with saturated aqueous NaHCO₃ and then extracted with EtOAc. The organic portion was separated, washed with brine, dried (MgSO₄), filtered, and concentrated under reduced pressure to give the title compound. LRMS m/z: (M+H)⁺ calculated 601.3; found 601.6.

### Step 4: Methyl ((2S)-1-((2S,4(S or R))-4-cyclobutyl-2-(((3S)-1-(cyclopropylamino)-6,6-difluoro-2-hydroxy-1-oxoheptan-3-yl)carbamoyl)pyrrolidin-1-yl)-3,3-dimethyl-1-oxobutan-2-yl)carbamate

To a stirred solution of (3S)-3-((2S,4(S or R))-4-cyclobutyl-1-((S)-2-((methoxycarbonyl)amino)-3,3-dimethylbutanoyl)pyrrolidine-2-carboxamido)-1-(cyclopropylamino)-6,6-difluoro-1-oxoheptan-2-yl acetate (135 mg, 0.225 mmol) in THF (899 μL) and ethanol (899 μL) was added 1 M lithium hydroxide (449 μL, 0.449 mmol). After 20 minutes, the reaction was quenched with saturated aqueous NaHCO₃ and then extracted with EtOAc. The organic portion was separated, washed with brine, dried (MgSO₄), filtered, and concentrated under reduced pressure to give the title compound. LRMS m/z: (M+H)⁺ calculated 559.3; found 559.4.

### Step 5: Methyl ((S)-1-((2S,4(S or R))-4-cyclobutyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate

A stirred solution of methyl ((2S)-1-((2S,4(S or R))-4-cyclobutyl-2-(((3S)-1-(cyclopropylamino)-6,6-difluoro-2-hydroxy-1-oxoheptan-3-yl)carbamoyl)pyrrolidin-1-yl)-3,3-dimethyl-1-oxobutan-2-yl)carbamate (120 mg, 0.22 mmol) in DCM (2.15 mL) was cooled to 0° C. and then treated with sodium bicarbonate (72.2 mg, 0.859 mmol) followed by Dess-Martin periodinane (137 mg, 0.322 mmol). The mixture was stirred for 5 minutes at 0° C. and then warmed to ambient temperature and stirred for additional 30 minutes. The reaction mixture was quenched with saturated aqueous sodium thiosulfate and saturated aqueous NaHCO₃ and stirred for 15 minutes. The mixture was extracted with two portions of DCM. The combined organic portions were dried over Na₂SO₄, filtered, and concentrated under reduced pressure. The residue was purified by column chromatography on silica gel eluting with 0-100% EtOAc in hexanes to give the title compound. LRMS m/z: (M+H)⁺ calculated 557.3; found 557.4. ¹H NMR (500 MHz, DMSO-d₆) δ 8.85-8.59 (m, 1H), 8.31 (d, J=7.3 Hz, 1H), 7.07 (d, J=8.8 Hz, 1H), 4.95 (t, J=6.4 Hz, 1H), 4.49-4.31 (m, 1H), 4.14 (d, J=8.8 Hz, 1H), 3.71-3.61 (m, 1H), 3.53 (s, 3H), 2.80-2.70 (m, 1H), 2.32 (dq, J=14.5, 8.8, 7.1 Hz, 1H), 2.16 (dq, J=16.2, 8.0 Hz, 1H), 2.06-1.54 (m, 16H), 0.94 (s, 9H), 0.71-0.54 (m, 4H).

The following compounds were prepared in a similar manner:

| Ex. | | | LRMS m/z |
|---|---|---|---|
| No. | Structure | Name | (M + H)⁺ |
| 2 | 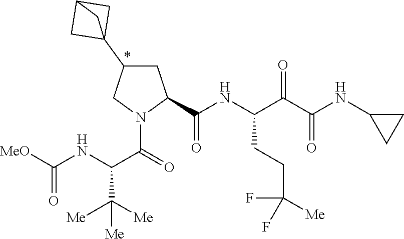 [SMILES: COC(=O)N[C@H](C(=O)N1CC(C23CC(C2)C3)C[C@H]1C(=O)N[C@@H](CCC(C)(F)F)C(=O)C(=O)NC1CC1)C(C)(C)C] | Methyl ((S)-1-((2S,4(R or S))-4-(bicyclo[1.1.1]pentan- 1-yl)-2-(((S)-1- (cyclopropylamino)-6,6- difluoro-1,2-dioxoheptan-3- yl)carbamoyl)pyrrolidin-1- yl)-3,3-dimethyl-1- oxobutan-2-yl)carbamate | Calculated: 569.3 Found: 569.4 |
| 3 | 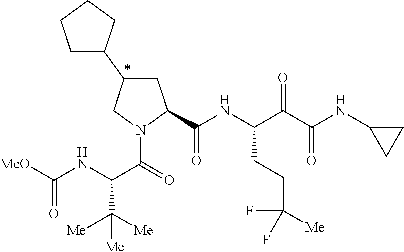 [SMILES: COC(=O)N[C@H](C(=O)N1CC(C2CCCC2)C[C@H]1C(=O)N[C@@H](CCC(C)(F)F)C(=O)C(=O)NC1CC1)C(C)(C)C] | Methyl ((S)-1-((2S,4(R or S))-4-cyclopentyl-2-(((S)-1- (cyclopropylamino)-6,6- difluoro-1,2-dioxoheptan-3- yl)carbamoyl)pyrrolidin-1- yl)-3,3-dimethyl-1- oxobutan-2-yl)carbamate | Calculated: 571.3 Found: 571.4 |
| 4 | 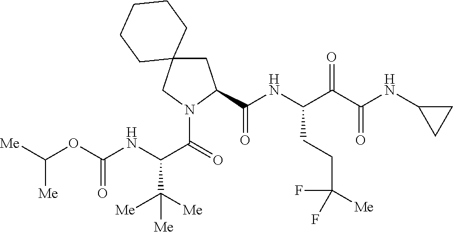 [SMILES: CC(C)OC(=O)N[C@H](C(=O)N1CC2(CCCCC2)C[C@H]1C(=O)N[C@@H](CCC(C)(F)F)C(=O)C(=O)NC1CC1)C(C)(C)C] | Isopropyl ((S)-1-((S)-3- (((S)-1-(cyclopropylamino)- 6,6-difluoro-1,2- dioxoheptan-3- yl)carbamoyl)-2- azaspiro[4.5]decan-2-yl)- 3,3-dimethyl-1-oxobutan-2- yl)carbamate | Calculated: 599.4 Found: 599.6 |
| 5 | 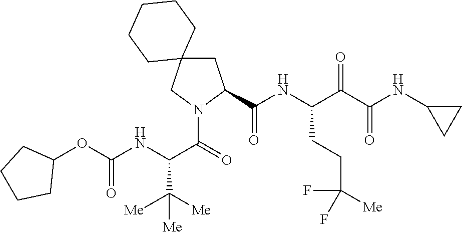 [SMILES: CC(F)(F)CC[C@H](NC(=O)[C@@H]1CC2(CCCCC2)CN1C(=O)[C@@H](NC(=O)OC1CCCC1)C(C)(C)C)C(=O)C(=O)NC1CC1] | Cyclopentyl ((S)-1-((S)-3- (((S)-1-(cyclopropylamino)- 6,6-difluoro-1,2- dioxoheptan-3- yl)carbamoyl)-2- azaspiro[4.5]decan-2-yl)- 3,3-dimethyl-1-oxobutan-2- yl)carbamate | Calculated: 625.4 Found: 625.6 |

### From Scheme D

### Example 6

### Methyl ((S)-1-((1R,2S,5S)-2-(((S)-1-(cyclopropylamino)-6,6-difluoro-1,2-dioxoheptan-3-yl)carbamoyl)-6,6-dimethyl-3-azabicyclo[3.1.0]hexan-3-yl)-3,3-dimethyl-1-oxobutan-2-yl)carbamate

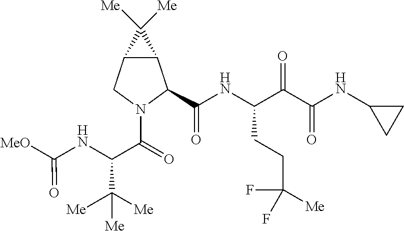 [SMILES: COC(=O)N[C@H](C(=O)N1C[C@H]2[C@@H]([C@H]1C(=O)N[C@@H](CCC(C)(F)F)C(=O)C(=O)NC1CC1)C2(C)C)C(C)(C)C]

### Step 1: Benzyl (1R,2S,5S)-2-(((S)-5,5-difluoro-1,1-dimethoxyhexan-2-yl)carbamoyl)-6,6-dimethyl-3-azabicyclo[3.1.0]hexane-3-carboxylate

To a suspension of PS-Carbodiimide (1.35 mmol/g) (693 mg, 0.963 mmol) and (1R,2S,5S)-3-((benzyloxy)carbonyl)-6,6-dimethyl-3-azabicyclo[3.1.0]hexane-2-carboxylic acid (209 mg, 0.723 mmol) in DCM (2 mL) was added a solution of (S)-5,5-difluoro-1,1-dimethoxyhexan-2-amine (95 mg, 0.48 mmol) in DCM (1 mL). The reaction was shaken at room temperature for 3 days. The reaction was filtered and the resin was washed with DCM. The filtrate was concentrated under reduced pressure to give the title compound. LRMS m/z: (M+H)⁺ calculated 469.3; found 469.3.

### Step 2: (1R,2S,5S)—N—((S)-5,5-Difluoro-1,1-dimethoxyhexan-2-yl)-6,6-dimethyl-3-azabicyclo[3.1.0]hexane-2-carboxamide

To a stirred solution of benzyl (1R,2S,5S)-2-(((S)-5,5-difluoro-1,1-dimethoxyhexan-2-yl)carbamoyl)-6,6-dimethyl-3-azabicyclo[3.1.0]hexane-3-carboxylate (226 mg, 0.482 mmol) in EtOH (3.2 ml) was added 20 wt % palladium hydroxide (34 mg, 0.048 mmol). The flask was purged, placed under an atmosphere of H₂ using a balloon and stirred at RT for 40 min. The mixture was filtered through a prepacked Celite filter and the catalyst was washed with EtOAc. The filtrate was concentrated to give the title compound. LRMS m/z: (M+H)⁺ calculated 335.2; found 335.3.

### Step 3: Methyl ((S)-1-((1R,2S,5S)-2-(((S)-5,5-difluoro-1,1-dimethoxyhexan-2-yl)carbamoyl)-6,6-dimethyl-3-azabicyclo[3.1.0]hexan-3-yl)-3,3-dimethyl-1-oxobutan-2-yl)carbamate

To a suspension of PS-Carbodiimide (1.35 mmol/g) (693 mg, 0.963 mmol) and (S)-2-((methoxycarbonyl)amino)-3,3-dimethylbutanoic acid (159 mg, 0.843 mmol) in DCM (5 mL) was added a solution of (1R,2S,5S)—N—((S)-5,5-difluoro-1,1-dimethoxyhexan-2-yl)-6,6-dimethyl-3-azabicyclo[3.1.0]hexane-2-carboxamide (161 mg, 0.482 mmol) in DCM (1 mL). The reaction was shaken at RT for 1 hour. The reaction was filtered and the resin was washed with DCM. The filtrate was concentrated under reduced pressure and the crude material was purified by column chromatography on silica gel eluting with 0-100% EtOAc/hexanes. Product-containing fractions were combined and concentrated to give the title compound. LRMS m/z: (M+H)⁺ calculated 506.3; found 506.4.

### Step 4: Methyl ((S)-1-((1R,2S,5S)-2-(((S)-5,5-difluoro-1-oxohexan-2-yl)carbamoyl)-6,6-dimethyl-3-azabicyclo[3.1.0]hexan-3-yl)-3,3-dimethyl-1-oxobutan-2-yl)carbamate

A solution of methyl ((S)-1-((1R,2S,5S)-2-(((S)-5,5-difluoro-1,1-dimethoxyhexan-2-yl)carbamoyl)-6,6-dimethyl-3-azabicyclo[3.1.0]hexan-3-yl)-3,3-dimethyl-1-oxobutan-2-yl)carbamate (193 mg, 0.382 mmol) in acetone (2.3 mL) and water (2.3 mL) was treated with Dowex® 50WX8 Hydrogen Form 200-400 mesh (400 mg) and then shaken and heated to 70° C. for 20 hours. The mixture was filtered and the solvent was removed under reduced pressure, azeotroping with MeCN to give the title compound. LRMS m/z: (M+H)⁺ calculated 460.3; found 460.4.

### Step 5: (3S)-1-(cyclopropylamino)-6,6-difluoro-3-((1R,2S,5S)-3-((S)-2-((methoxycarbonyl)amino)-3,3-dimethylbutanoyl)-6,6-dimethyl-3-azabicyclo[3.1.0]hexane-2-carboxamido)-1-oxoheptan-2-yl acetate

To a stirred solution of methyl ((S)-1-((1R,2S,5S)-2-(((S)-5,5-difluoro-1-oxohexan-2-yl)carbamoyl)-6,6-dimethyl-3-azabicyclo[3.1.0]hexan-3-yl)-3,3-dimethyl-1-oxobutan-2-yl)carbamate (175 mg, 0.381 mmol) in CH₂Cl₂ (3.8 mL) at 0° C. was added cyclopropyl isocyanide (32.5 μL, 0.476 mmol) followed by acetic acid (27.3 μL, 0.476 mmol). The reaction was stirred at 0° C. for 5 min followed by removal of the cooling bath. After 1 hour at ambient temperature, the reaction was quenched with saturated aq. NaHCO₃ and extracted with DCM. The organic portion was separated, washed with brine, dried (MgSO₄), filtered, and concentrated under reduced pressure to give the title compound. LRMS m/z: (M+H)⁺ calculated 587.3; found 587.5.

### Step 6: Methyl ((2S)-1-((1R,2S,5S)-2-(((3S)-1-(cyclopropylamino)-6,6-difluoro-2-hydroxy-1-oxoheptan-3-yl)carbamoyl)-6,6-dimethyl-3-azabicyclo[3.1.0]hexan-3-yl)-3,3-dimethyl-1-oxobutan-2-yl)carbamate

To a stirred solution of (3S)-1-(cyclopropylamino)-6,6-difluoro-3-((1R,2S,5S)-3-((S)-2-((methoxycarbonyl)amino)-3,3-dimethylbutanoyl)-6,6-dimethyl-3-azabicyclo[3.1.0]hexane-2-carboxamido)-1-oxoheptan-2-yl acetate (223 mg, 0.380 mmol) in THF (1.5 mL), ethanol (1.5 mL), and water (0.76 mL) was added lithium hydroxide (13.7 mg, 0.570 mmol). After 5 minutes, the reaction was filtered and washed with DMF. The crude filtrate was purified directly by reverse-phase HPLC (Waters XBridge Prep C18 OBD 5 μm 30×250 mm) eluting with 10-100% MeCN/water+0.1% ammonium bicarbonate over 25 min at 50 mL/min. Pure fractions were combined and partitioned between DCM and water. The aqueous phase was extracted with DCM. The organic phase was dried over Na₂SO₄, filtered and concentrated to give the title compound. LRMS m/z: (M+H)⁺ calculated 545.3; found 545.4.

### Step 7: Methyl ((S)-1-((1R,2S,5S)-2-(((S)-1-(cyclopropylamino)-6,6-difluoro-1,2-dioxoheptan-3-yl)carbamoyl)-6,6-dimethyl-3-azabicyclo[3.1.0]hexan-3-yl)-3,3-dimethyl-1-oxobutan-2-yl)carbamate

A solution of methyl ((2S)-1-((1R,2S,5S)-2-(((3S)-1-(cyclopropylamino)-6,6-difluoro-2-hydroxy-1-oxoheptan-3-yl)carbamoyl)-6,6-dimethyl-3-azabicyclo[3.1.0]hexan-3-yl)-3,3-dimethyl-1-oxobutan-2-yl)carbamate (139 mg, 0.254 mmol) in DCM (2.5 mL) was cooled to 0° C. and treated with sodium bicarbonate (85 mg, 1.0 mmol) followed by Dess-Martin periodinane (162 mg, 0.381 mmol). The mixture was stirred for 5 minutes at 0° C. and then warmed to ambient temperature and stirred for additional 30 minutes. The reaction mixture was quenched with saturated aq. sodium thiosulfate and saturated aq. NaHCO₃ and stirred for 15 minutes. The mixture was extracted twice with DCM. The combined organic portions were dried over Na₂SO₄, filtered, and concentrated under reduced pressure. The residue was purified by column chromatography on silica gel eluting with 0-100% EtOAc/hexanes. Product-containing fractions were combined and concentrated to give the title compound. LRMS m/z: (M+H)⁺ calculated 543.3; found 543.4. ¹H NMR (500 MHz, methanol-d₄) δ 4.49-4.08 (m, 2H), 4.08-3.84 (m, 2H), 3.63 (d, J=10.6 Hz, 3H), 2.71 (m, 1H), 2.31-1.86 (m, 3H), 1.80-1.31 (m, 7H), 1.11-0.91 (m, 15H), 0.87-0.36 (m, 4H).

The following compounds were prepared in a similar manner:

| | | | LRMS |
|---|---|---|---|
| Ex. | | | m/z |
| No. | Structure | Name | (M + H)⁺ |
| 7 | 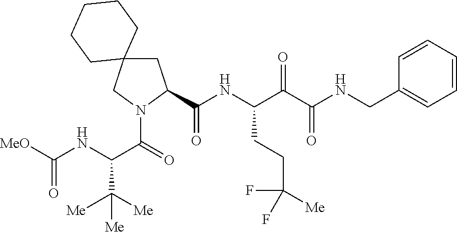 [SMILES: COC(=O)N[C@H](C(=O)N1CC2(CCCCC2)C[C@H]1C(=O)N[C@@H](CCC(C)(F)F)C(=O)C(=O)NCc1ccccc1)C(C)(C)C] | Methyl ((S)-1-((S)-3-(((S)- 1-(benzylamino)-6,6- difluoro-1,2-dioxoheptan- 3-yl)carbamoyl)-2- azaspiro[4.5]decan-2-yl)- 3,3-dimethyl-1-oxobutan- 2-yl)carbamate | Calculated: 621.4 Found: 621.3 |
| 8 | 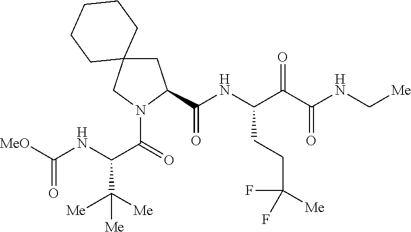 [SMILES: CCNC(=O)C(=O)[C@H](CCC(C)(F)F)NC(=O)[C@@H]1CC2(CCCCC2)CN1C(=O)[C@@H](NC(=O)OC)C(C)(C)C] | Methyl ((S)-1-((S)-3-(((S)- 1-(ethylamino)-6,6- difluoro-1,2-dioxoheptan- 3-yl)carbamoyl)-2- azaspiro[4.5]decan-2-yl)- 3,3-dimethyl-1-oxobutan- 2-yl)carbamate | Calculated: 559.3 Found: 559.4 |
| 9 | 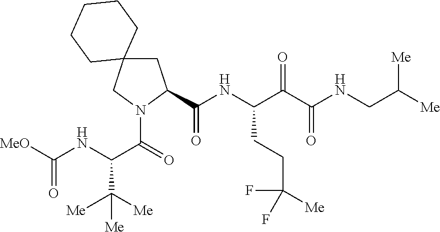 [SMILES: COC(=O)N[C@H](C(=O)N1CC2(CCCCC2)C[C@H]1C(=O)N[C@@H](CCC(C)(F)F)C(=O)C(=O)NCC(C)C)C(C)(C)C] | Methyl ((S)-1-((S)-3-(((S)- 6,6-difluoro-1- (isobutylamino)-1,2- dioxoheptan-3- yl)carbamoyl)-2- azaspiro[4.5]decan-2-yl)- 3,3-dimethyl-1-oxobutan- 2-yl)carbamate | Calculated: 587.4 Found: 587.4 |
| 10 | 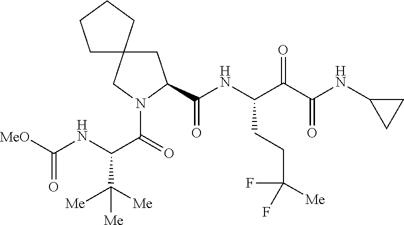 [SMILES: COC(=O)N[C@H](C(=O)N1CC2(CCCC2)C[C@H]1C(=O)N[C@@H](CCC(C)(F)F)C(=O)C(=O)NC1CC1)C(C)(C)C] | Methyl ((S)-1-((S)-3-(((S)- 1-(cyclopropylamino)-6,6- difluoro-1,2-dioxoheptan- 3-yl)carbamoyl)-2- azaspiro[4.4]nonan-2-yl)- 3,3-dimethyl-1-oxobutan- 2-yl)carbamate | Calculated: 557.3 Found: 557.3 |
| 11 | 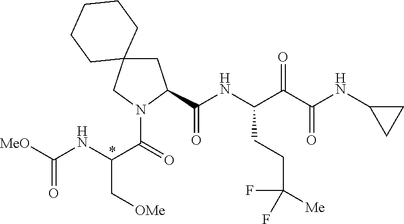 [SMILES: COCC(NC(=O)OC)C(=O)N1CC2(CCCCC2)C[C@H]1C(=O)N[C@@H](CCC(C)(F)F)C(=O)C(=O)NC1CC1] | Methyl ((R or S)-1-((S)-3- (((S)-1- (cyclopropylamino)-6,6- difluoro-1,2-dioxoheptan- 3-yl)carbamoyl)-2- azaspiro[4.5]decan-2-yl)- 3-methoxy-1-oxopropan- 2-yl)carbamate | Calculated: 559.3 Found: 559.3 |
| 12 | 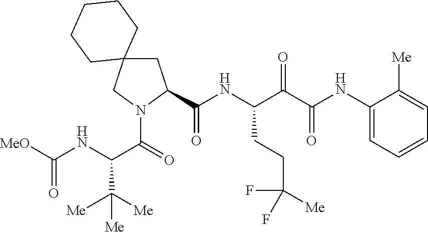 [SMILES: COC(=O)N[C@H](C(=O)N1CC2(CCCCC2)C[C@H]1C(=O)N[C@@H](CCC(C)(F)F)C(=O)C(=O)Nc1ccccc1C)C(C)(C)C] | Methyl ((S)-1-((S)-3-(((S)- 6,6-difluoro-1,2-dioxo-1- (o-tolylamino)heptan-3- yl)carbamoyl)-2- azaspiro[4.5]decan-2-yl)- 3,3-dimethyl-1-oxobutan- 2-yl)carbamate | Calculated: 621.4 Found: 621.3 |
| 13 | 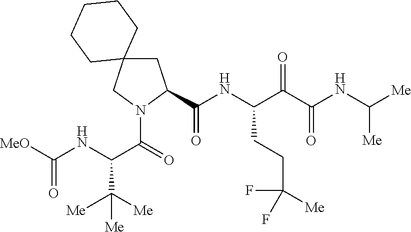 [SMILES: COC(=O)N[C@H](C(=O)N1CC2(CCCCC2)C[C@H]1C(=O)N[C@@H](CCC(C)(F)F)C(=O)C(=O)NC(C)C)C(C)(C)C] | Methyl ((S)-1-((S)-3-(((S)- 6,6-difluoro-1- (isopropylamino)-1,2- dioxoheptan-3- yl)carbamoyl)-2- azaspiro[4.5]decan-2-yl)- 3,3-dimethyl-1-oxobutan- 2-yl)carbamate | Calculated: 573.4 Found: 573.4 |
| 14 | 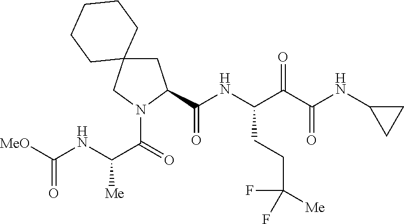 [SMILES: COC(=O)N[C@@H](C)C(=O)N1CC2(CCCCC2)C[C@H]1C(=O)N[C@@H](CCC(C)(F)F)C(=O)C(=O)NC1CC1] | Methyl ((S)-1-((S)-3-(((S)- 1-(cyclopropylamino)-6,6- difluoro-1,2-dioxoheptan- 3-yl)carbamoyl)-2- azaspiro[4.5]decan-2-yl)- 1-oxopropan-2- yl)carbamate | Calculated: 529.3 Found: 529.5 |
| 15 | 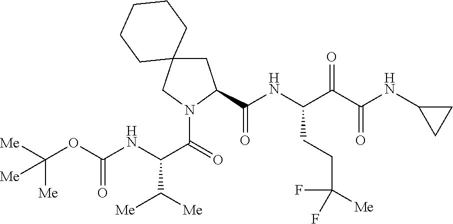 [SMILES: CC(C)[C@H](NC(=O)OC(C)(C)C)C(=O)N1CC2(CCCCC2)C[C@H]1C(=O)N[C@@H](CCC(C)(F)F)C(=O)C(=O)NC1CC1] | Tert-butyl ((S)-1-((S)-3- (((S)-1- (cyclopropylamino)-6,6- difluoro-1,2-dioxoheptan- 3-yl)carbamoyl)-2- azaspiro[4.5]decan-2-yl)- 3-methyl-1-oxobutan-2- yl)carbamate | Calculated: 599.4 Found: 599.4 |
| 16 | 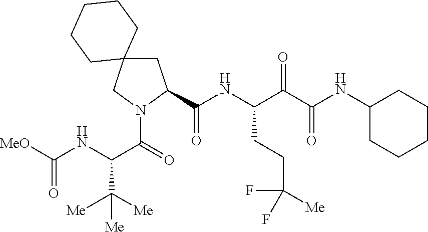 [SMILES: COC(=O)N[C@H](C(=O)N1CC2(CCCCC2)C[C@H]1C(=O)N[C@@H](CCC(C)(F)F)C(=O)C(=O)NC1CCCCC1)C(C)(C)C] | Methyl ((S)-1-((S)-3-(((S)- 1-(cyclohexylamino)-6,6- difluoro-1,2-dioxoheptan- 3-yl)carbamoyl)-2- azaspiro[4.5]decan-2-yl)- 3,3-dimethyl-1-oxobutan- 2-yl)carbamate | Calculated: 613.4 Found: 613.5 |
| 17 | 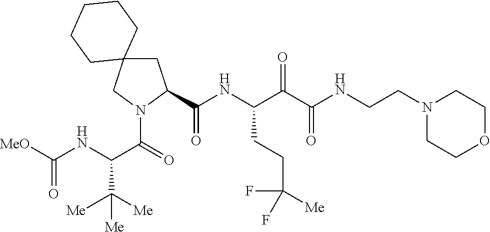 [SMILES: COC(=O)N[C@H](C(=O)N1CC2(CCCCC2)C[C@H]1C(=O)N[C@@H](CCC(C)(F)F)C(=O)C(=O)NCCN1CCOCC1)C(C)(C)C] | Methyl ((S)-1-((S)-3-(((S)- 6,6-difluoro-1-((2- morpholinoethyl)amino)- 1,2-dioxoheptan-3- yl)carbamoyl)-2- azaspiro[4.5]decan-2-yl)- 3,3-dimethyl-1-oxobutan- 2-yl)carbamate | Calculated: 644.4 Found: 644.4 |
| 18 | 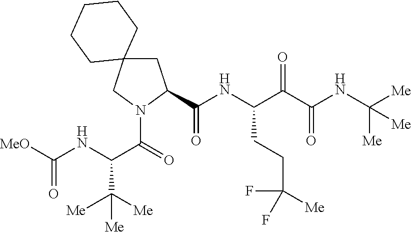 [SMILES: COC(=O)N[C@H](C(=O)N1CC2(CCCCC2)C[C@H]1C(=O)N[C@@H](CCC(C)(F)F)C(=O)C(=O)NC(C)(C)C)C(C)(C)C] | Methyl ((S)-1-((S)-3-(((S)- 1-(tert-butylamino)-6,6- difluoro-1,2-dioxoheptan- 3-yl)carbamoyl)-2- azaspiro[4.5]decan-2-yl)- 3,3-dimethyl-1-oxobutan- 2-yl)carbamate | Calculated: 587.4 Found: 587.5 |
| 19 | 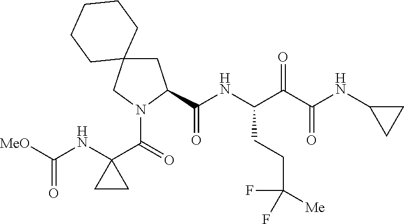 [SMILES: COC(=O)NC1(C(=O)N2CC3(CCCCC3)C[C@H]2C(=O)N[C@@H](CCC(C)(F)F)C(=O)C(=O)NC2CC2)CC1] | Methyl (1-((S)-3-(((S)-1- (cyclopropylamino)-6,6- difluoro-1,2-dioxoheptan- 3-yl)carbamoyl)-2- azaspiro[4.5]decane-2- carbonyl)cyclopropyl) carbamate | Calculated: 541.3 Found: 541.4 |

### From Scheme A

### Example 20

### Methyl ((S)-1-((S)-5-(((S)-1-amino-6,6-difluoro-1,2-dioxoheptan-3-yl)carbamoyl)-6-azaspiro[2.5]octan-6-yl)-3,3-dimethyl-1-oxobutan-2-yl)carbamate

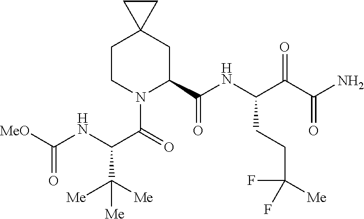 [SMILES: COC(=O)N[C@H](C(=O)N1CCC2(CC2)C[C@H]1C(=O)N[C@@H](CCC(C)(F)F)C(=O)C(N)=O)C(C)(C)C]

### Step 1: Methyl (S)-6-((S)-2-((methoxycarbonyl)amino)-3,3-dimethylbutanoyl)-6-azaspiro[2.5]octane-5-carboxylate

To a stirred solution of (S)-2-((methoxycarbonyl)amino)-3,3-dimethylbutanoic acid (506 mg, 2.67 mmol), methyl (S)-6-azaspiro[2.5]octane-5-carboxylate hydrochloride (500 mg, 2.43 mmol), N-methylmorpholine (1.07 mL, 9.72 mmol) and DMF (9.72 mL) at ambient temperature was added HATU (1.16 g, 3.04 mmol). The mixture was stirred for 18 hours. The reaction was diluted with H₂O and then extracted with EtOAc (2×10 mL). The combined organic portions were washed with saturated aqueous NaHCO₃ and brine, dried (MgSO₄), filtered, concentrated under reduced pressure. The residue was purified by column chromatography on silica gel eluting with 0-100% EtOAc in hexanes to give the title compound. LRMS m/z: (M+H)⁺ calculated 341.2; found 341.1.

### Step 2: (S)-6-((S)-2-((methoxycarbonyl)amino)-3,3-dimethylbutanoyl)-6-azaspiro[2.5]octane-5-carboxylic acid

To a stirred solution of methyl (S)-6-((S)-2-((methoxycarbonyl)amino)-3,3-dimethylbutanoyl)-6-azaspiro[2.5]octane-5-carboxylate (800 mg, 2.35 mmol) in ethanol (4.70 mL) and THF (4.70 mL) was added LiOH·H₂O (197 mg, 4.70 mmol) dissolved in water (2.35 mL). The mixture was heated to 40° C. for 1 hour, cooled to ambient temperature and then acidified with 1 N HCl. The mixture was extracted with EtOAc. The organic portion was separated, washed with brine, dried (MgSO₄), filtered, and concentrated under reduced pressure to give the title compound. LRMS m/z: (M+H)⁺ calculated 327.2; found 327.4.

### Step 3: Methyl ((2S)-1-((5S)-5-(((3S)-1-amino-6,6-difluoro-2-hydroxy-1-oxoheptan-3-yl)carbamoyl)-6-azaspiro[2.5]octan-6-yl)-3,3-dimethyl-1-oxobutan-2-yl)carbamate

To a stirred solution of (S)-6-((S)-2-((methoxycarbonyl)amino)-3,3-dimethylbutanoyl)-6-azaspiro[2.5]octane-5-carboxylic acid (80 mg, 0.25 mmol), (3S)-3-amino-6,6-difluoro-2-hydroxyheptanamide hydrochloride (57 mg, 0.25 mmol), and N-methylmorpholine (108 μL, 0.980 mmol) in DMF (1.63 mL) was added HATU (140 mg, 0.37 mmol) and the reaction was stirred at room temperature overnight. The reaction was diluted with DCM and then washed with saturated aqueous NaHCO₃ and brine, dried (MgSO₄), filtered, and concentrated. The residue was purified by column chromatography on silica gel eluting with 0-100% 3:1 EtOAc:EtOH in hexanes to give the title compound. LRMS m/z: (M+H)⁺ calculated 505.3; found 505.6.

### Step 4: Methyl ((S)-1-((S)-5-(((S)-1-amino-6,6-difluoro-1,2-dioxoheptan-3-yl)carbamoyl)-6-azaspiro[2.5]octan-6-yl)-3,3-dimethyl-1-oxobutan-2-yl)carbamate

A stirred solution of methyl ((2S)-1-((5S)-5-(((3S)-1-amino-6,6-difluoro-2-hydroxy-1-oxoheptan-3-yl)carbamoyl)-6-azaspiro[2.5]octan-6-yl)-3,3-dimethyl-1-oxobutan-2-yl)carbamate (120 mg, 0.238 mmol) in dichloromethane (2.38 mL) was cooled to 0° C. and then treated with sodium bicarbonate (80 mg, 0.95 mmol) followed by Dess-Martin periodinane (151 mg, 0.357 mmol). The mixture was stirred for 5 minutes at 0° C., warmed to room temperature, and then stirred for additional 30 minutes. The mixture was quenched with saturated aqueous sodium thiosulfate followed by saturated aqueous NaHCO₃ and stirred for 15 minutes and then the mixture was extracted with two portions of DCM. The combined organic phases were dried over Na₂SO₄, filtered, and concentrated under reduced pressure. The residue was purified by column chromatography on silica gel eluting with 0-100% EtOAc in hexanes to give the title compound. LRMS m/z: (M+H)⁺ calculated 503.3; found 503.4. ¹H NMR (500 MHz, DMSO-d₆) δ 8.17 (dd, J=66.8, 7.1 Hz, 1H), 8.06-7.92 (m, 1H), 7.86-7.65 (m, 1H), 6.97 (dd, J=81.3, 8.8 Hz, 1H), 5.17 (dd, J=27.9, 5.3 Hz, 1H), 5.07-4.88 (m, 1H), 4.62-4.27 (m, 2H), 4.01 (dd, J=14.9, 7.9 Hz, 1H), 3.52 (d, J=18.1 Hz, 3H), 2.10-1.81 (m, 4H), 1.81-1.70 (m, 1H), 1.69-1.46 (m, 6H), 0.94 (d, J=3.9 Hz, 9H), 0.29-0.17 (m, 4H).

### Example 21

### Methyl ((S)-1-((S)-5-(((S)-6,6-difluoro-1-(methylamino)-1,2-dioxoheptan-3-yl)carbamoyl)-6-azaspiro[2.5]octan-6-yl)-3,3-dimethyl-1-oxobutan-2-yl)carbamate

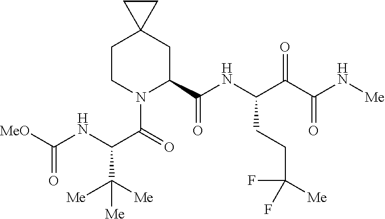 [SMILES: CNC(=O)C(=O)[C@H](CCC(C)(F)F)NC(=O)[C@@H]1CC2(CCN1C(=O)[C@@H](NC(=O)OC)C(C)(C)C)CC2]

### Step 1: Methyl (S)-6-((S)-2-((methoxycarbonyl)amino)-3,3-dimethylbutanoyl)-6-azaspiro[2.5]octane-5-carboxylate

To a stirred solution of (S)-2-((methoxycarbonyl)amino)-3,3-dimethylbutanoic acid (506 mg, 2.67 mmol), methyl (S)-6-azaspiro[2.5]octane-5-carboxylate hydrochloride (500 mg, 2.43 mmol), N-methylmorpholine (1.07 mL, 9.72 mmol) and DMF (9.7 mL) at ambient temperature was added HATU (1.16 g, 3.04 mmol). The mixture was stirred for 18 hours. The reaction was diluted with H₂O and then extracted with EtOAc (2×10 mL). The combined organic portions were washed with sat aq. NaHCO₃ and brine, dried (MgSO₄), filtered, and concentrated under reduced pressure. The residue was purified by column chromatography on silica gel eluting with 0-100% EtOAc/hexanes. Product-containing fractions were combined and concentrated to give the title compound. LRMS m/z: (M+H)⁺ calculated 341.2; found 341.1.

### Step 2: (S)-6-((S)-2-((Methoxycarbonyl)amino)-3,3-dimethylbutanoyl)-6-azaspiro[2.5]octane-5-carboxylic acid

To a stirred solution of methyl (S)-6-((S)-2-((methoxycarbonyl)amino)-3,3-dimethylbutanoyl)-6-azaspiro[2.5]octane-5-carboxylate (800 mg, 2.35 mmol) in ethanol (4.7 mL) and THF (4.7 mL) was added LiOH·H₂O (197 mg, 4.70 mmol) dissolved in water (2.35 mL). The mixture was heated to 40° C. for 1 hour, cooled to room temperature, and then acidified with 1 N HCl. The mixture was extracted with EtOAc. The organic portion was separated, washed with brine, dried (MgSO₄), filtered, and concentrated under reduced pressure to give the title compound. LRMS m/z: (M+H)⁺ calculated 327.2; found 327.4.

### Step 3: Methyl ((2S)-1-((5S)-5-(((3S)-6,6-difluoro-2-hydroxy-1-(methylamino)-1-oxoheptan-3-yl)carbamoyl)-6-azaspiro[2.5]octan-6-yl)-3,3-dimethyl-1-oxobutan-2-yl)carbamate

To a suspension of PS-Carbodiimide (1.39 mmol/g) (705 mg, 0.980 mmol) and (S)-6-((S)-2-((methoxycarbonyl)amino)-3,3-dimethylbutanoyl)-6-azaspiro[2.5]octane-5-carboxylic acid (160 mg, 0.49 mmol) in DCM (2.5 mL) was added a suspension of (2S,3S)-3-amino-6,6-difluoro-2-hydroxy-N-methylheptanamide hydrochloride and DIPEA (214 uL, 1.23 mmol) in DCM (1 mL). The reaction was shaken at room temperature for 1 hour. The reaction was filtered and the resin was washed with DCM. The filtrate was concentrated under reduced pressure and the material purified by reverse-phase HPLC (Waters Xbridge Prep C18 OBD 5 μm 50×250 mm) eluting with 10-60% MeCN/H₂O (5 mM NH₄HCO₃ modifier in H₂O) over 15 min. Pure product-containing fractions were concentrated to afford the title compound. LRMS m/z: (M+H)⁺ calculated 519.3; found 519.6.

### Step 4: Methyl ((S)-1-((S)-5-(((S)-6,6-difluoro-1-(methylamino)-1,2-dioxoheptan-3-yl)carbamoyl)-6-azaspiro[2.5]octan-6-yl)-3,3-dimethyl-1-oxobutan-2-yl)carbamate

A solution of methyl ((2S)-1-((5S)-5-(((3S)-6,6-difluoro-2-hydroxy-1-(methylamino)-1-oxoheptan-3-yl)carbamoyl)-6-azaspiro[2.5]octan-6-yl)-3,3-dimethyl-1-oxobutan-2-yl)carbamate (75.2 mg, 0.145 mmol) in DCM (1.45 mL), was cooled to 0° C. and then treated with sodium bicarbonate (48.7 mg, 0.580 mmol) followed by Dess-Martin periodinane (92 mg, 0.22 mmol). The mixture was stirred for 5 minutes at 0° C. and then warmed to ambient temperature and stirred for an additional 1.5 h. The reaction mixture was quenched with saturated aq. sodium thiosulfate and saturated aq. NaHCO₃ and stirred for 15 minutes. The mixture was extracted with two portions of DCM. The combined organic portions were dried over Na₂SO₄, filtered, and concentrated under reduced pressure. The residue was purified by column chromatography on silica gel eluting with 0-100% EtOAc/hexanes. Product-containing fractions were combined and concentrated to give the title compound. LRMS m/z: (M+H)⁺ calculated 517.3; found 517.8. ¹H NMR (500 MHz, DMSO-d₆) δ 8.62 (d, J=4.8 Hz, 1H), 8.17 (d, J=7.0 Hz, 1H), 7.06 (d, J=8.9 Hz, 1H), 5.28-5.05 (m, 1H), 5.03-4.81 (m, 1H), 4.66-3.86 (m, 3H), 3.63-3.41 (m, 4H), 2.80-2.56 (m, 3H), 2.19-1.69 (m, 5H), 1.68-1.40 (m, 4H), 1.13-0.80 (m, 10H), 0.61-0.14 (m, 4H).

### Example 22

### Methyl ((S)-1-((2S,4R)-2-(((S)-6,6-difluoro-1-(methylamino)-1,2-dioxoheptan-3-yl)carbamoyl)-4-(trifluoromethyl)piperidin-1-yl)-3,3-dimethyl-1-oxobutan-2-yl)carbamate

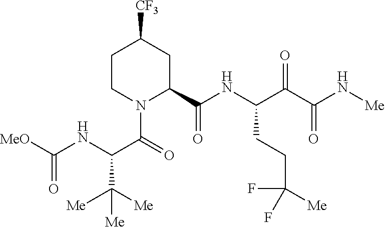 [SMILES: CNC(=O)C(=O)[C@H](CCC(C)(F)F)NC(=O)[C@@H]1C[C@H](C(F)(F)F)CCN1C(=O)[C@@H](NC(=O)OC)C(C)(C)C]

### Step 1: Methyl (2S,4R)-1-((S)-2-((methoxycarbonyl)amino)-3,3-dimethylbutanoyl)-4-(trifluoromethyl)piperidine-2-carboxylate

To a stirred solution of (S)-2-((methoxycarbonyl)amino)-3,3-dimethylbutanoic acid (112 mg, 0.591 mmol), methyl (2S,4R)-4-(trifluoromethyl)piperidine-2-carboxylate hydrochloride (133 mg, 0.537 mmol), DIPEA (281 μL, 1.61 mmol) and DMF (2.7 mL) at ambient temperature was added HATU (245 mg, 0.644 mmol). The mixture was stirred for 21 hours. The mixture was directly purified by reverse-phase HPLC (Waters SunFire Prep C18 OBD 10 μm 50×250 mm) eluting with 5-80% MeCN/H₂O (0.1% TFA modifier) over 30 min. Product-containing fractions were concentrated to give the title compound. LRMS m/z: (M+H)⁺ calculated 383.2; found 383.3.

### Step 2: (2S,4R)-1-((S)-2-((Methoxycarbonyl)amino)-3,3-dimethylbutanoyl)-4-(trifluoromethyl)piperidine-2-carboxylic acid

To a stirred solution of methyl (2S,4R)-1-((S)-2-((methoxycarbonyl)amino)-3,3-dimethylbutanoyl)-4-(trifluoromethyl)piperidine-2-carboxylate (218 mg, 0.570 mmol) in THF (3.8 mL), methanol (0.95 mL), and water (0.95 mL) was added lithium hydroxide (20.5 mg, 0.855 mmol). The mixture was heated to 60° C. for 30 min, cooled to ambient temperature and concentrated to remove THF and MeOH. The suspension was diluted with 1 mL water and then acidified with 1 N HCl. The mixture was extracted with DCM. The organic portion was separated, washed with brine, dried (MgSO₄), filtered, and concentrated under reduced pressure to give the title compound. LRMS m/z: (M+H)⁺ calculated 369.2; found 369.4.

### Step 3: Methyl ((2S)-1-((2S,4R)-2-(((3S)-6,6-difluoro-2-hydroxy-1-(methylamino)-1-oxoheptan-3-yl)carbamoyl)-4-(trifluoromethyl)piperidin-1-yl)-3,3-dimethyl-1-oxobutan-2-Yl)carbamate

A solution of (2S,4R)-1-((S)-2-((methoxycarbonyl)amino)-3,3-dimethylbutanoyl)-4-(trifluoromethyl)piperidine-2-carboxylic acid (70 mg, 0.19 mmol), (2S,3S)-3-amino-6,6-difluoro-2-hydroxy-N-methylheptanamide hydrochloride (47.9 mg, 0.228 mmol) and 7-azabenzotriazol-1-yl oxytris(dimethylamino)phosphonium hexafluorophosphonate (168 mg, 0.380 mmol) in DMF (0.95 mL) was treated with DIPEA (100 μL, 0.57 mmol). The reaction was stirred at room temperature for 1 hour and was purified directly by reverse-phase HPLC (Waters XBridge Prep C18 OBD 5 μm 50×250 mm) eluting with 10-70% MeCN/H₂O (5 mM NH₄HCO₃ modifier in H₂O) over 15 min. Pure product-containing fractions were concentrated to afford the title compound. LRMS m/z: (M+H)⁺ calculated 561.3; found 561.7.

### Step 4: Methyl ((S)-1-((2S,4R)-2-(((S)-6,6-difluoro-1-(methylamino)-1,2-dioxoheptan-3-yl)carbamoyl)-4-(trifluoromethyl)piperidin-1-yl)-3,3-dimethyl-1-oxobutan-2-yl)carbamate

A solution of methyl ((2S)-1-((2S,4R)-2-(((3S)-6,6-difluoro-2-hydroxy-1-(methylamino)-1-oxoheptan-3-yl)carbamoyl)-4-(trifluoromethyl)piperidin-1-yl)-3,3-dimethyl-1-oxobutan-2-yl)carbamate (79.7 mg, 0.142 mmol) in DCM (1.4 mL) was cooled to 0° C. and then treated with sodium bicarbonate (47.8 mg, 0.569 mmol) followed by Dess-Martin periodinane (90 mg, 0.21 mmol). The mixture was stirred for 5 minutes at 0° C. and then warmed to ambient temperature and stirred for 2 h. The reaction mixture was quenched with saturated aq. sodium thiosulfate and saturated aq. NaHCO₃ and stirred for 15 minutes. The mixture was extracted with two portions of DCM. The combined organic portions were dried over Na₂SO₄, filtered, and concentrated under reduced pressure. The residue was purified by column chromatography on silica gel eluting with 0-100% EtOAc/hexanes. Product-containing fractions were combined and concentrated to give the title compound. LRMS m/z: (M+H)⁺ calculated 559.3; found 559.6. ¹H NMR (500 MHz, CD₃OD) δ 4.50 (d, J=5.0 Hz, 1H), 4.41 (ddd, J=29.4, 12.6, 5.8 Hz, 1H), 4.18-3.95 (m, 2H), 3.64 (s, 3H), 3.61-3.52 (m, 1H), 2.76 (t, J=17.2 Hz, 3H), 2.52 (dt, J=8.9, 4.4 Hz, 1H), 2.26-2.04 (m, 3H), 2.00-1.66 (m, 4H), 1.64-1.48 (m, 4H), 1.03-0.96 (m, 9H).

### Example 23

### Methyl ((S)-1-((2(S or R),3(R or S))-3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate

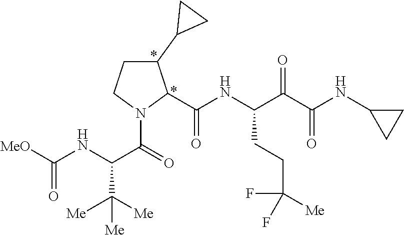 [SMILES: COC(=O)N[C@H](C(=O)N1CCC(C2CC2)C1C(=O)N[C@@H](CCC(C)(F)F)C(=O)C(=O)NC1CC1)C(C)(C)C]

### Step 1: Methyl (2(S or R),3(R or S))-3-cyclopropyl-1-((S)-2-((methoxycarbonyl)amino)-3,3-dimethylbutanoyl)pyrrolidine-2-carboxylate

To a solution of (S)-2-((methoxycarbonyl)amino)-3,3-dimethylbutanoic acid (180 mg, 0.95 mmol), methyl (2(S or R),3(R or S))-3-cyclopropylpyrrolidine-2-carboxylate hydrochloride (161 mg, 0.951 mmol) in DIEA (0.498 mL, 2.85 mmol) in DMF (0.5 mL) was added AOP (506 mg, 1.14 mmol) at 25° C. The mixture was stirred at 25° C. for 16 h. The reaction mixture was purified directly by RP-HPLC (Boston Green ODS 5 μm 150×30 mm) eluting with 40-60% acetonitrile/water+0.10% TFA over 10 minutes at 25 mL/min to give the title compound. LRMS m/z: (M+H)⁺ calculated 341.2; found 341.2.

### Step 2: (2(S or R),3(R or S))-3-Cyclopropyl-1-((S)-2-((methoxycarbonyl)amino)-3,3-dimethylbutanoyl)pyrrolidine-2-carboxylic acid

A solution of methyl (2(S or R),3(R or S))-3-cyclopropyl-1-((S)-2-((methoxycarbonyl)amino)-3,3-dimethylbutanoyl)pyrrolidine-2-carboxylate (170 mg, 0.50 mmol) in 3:1 MeOH:H₂O (3 mL) was stirred at 25° C. for 2 hours. The solvent was removed under reduced pressure. Water (5 mL) was added to the residue. The pH of the mixture was adjusted to 5 by addition of 1 N HCl. The mixture was extracted with DCM (3×10 mL). The combined organic fractions were washed with brine (10 mL), dried (Na₂SO₄), filtered, and concentrated under reduced pressure to give the title compound. LRMS m/z: (M+H)⁺ calculated 327.2; found 327.2.

### Step 3: Methyl ((2S)-1-((2(S or R),3(R or S))-3-cyclopropyl-2-(((3S)-1-(cyclopropylamino)-6,6-difluoro-2-hydroxy-1-oxoheptan-3-yl)carbamoyl)pyrrolidin-1-yl)-3,3-dimethyl-1-oxobutan-2-yl)carbamate

To a solution of (2(S or R),3(R or S))-3-cyclopropyl-1-((S)-2-((methoxycarbonyl)amino)-3,3-dimethylbutanoyl)pyrrolidine-2-carboxylic acid (60 mg, 0.18 mmol), (3S)-3-amino-N-cyclopropyl-6,6-difluoro-2-hydroxyheptanamide (43.4 mg, 0.184 mmol) and DIEA (0.032 mL, 0.18 mmol) in DMF (2 mL) was added AOP (81 mg, 0.18 mmol) at 25° C. The mixture was stirred at 25° C. for 16 hours. The reaction mixture was purified directly by RP-HPLC (Boston Green ODS 5 μm 150×30 mm) eluting with 37-57% acetonitrile/Water+0.1% TFA over 10 minutes at 25 mL/min to give title compound. LRMS m/z: (M+H)⁺ calculated 545.3; found 545.3.

### Step 4: Methyl ((S)-1-((2(S or R),3(R or S))-3-cyclopropyl-2-(((S)-1-(cyclopropylamino)-6,6-difluoro-1,2-dioxoheptan-3-yl)carbamoyl)pyrrolidin-1-yl)-3,3-dimethyl-1-oxobutan-2-yl)carbamate

To a solution of methyl ((2S)-1-((2(S or R),3(R or S))-3-cyclopropyl-2-(((3S)-1-(cyclopropylamino)-6,6-difluoro-2-hydroxy-1-oxoheptan-3-yl)carbamoyl)pyrrolidin-1-yl)-3,3-dimethyl-1-oxobutan-2-yl)carbamate (55 mg, 0.081 mmol) in DCM (5 mL) was added NaHCO₃ (20.4 mg, 0.242 mmol) and Dess-Martin periodinane (103 mg, 0.242 mmol). The mixture was stirred for 1 hour at 25° C. Na₂SO₃ (30 mg) was added to the reaction and the mixture was stirred at 20° C. for 15 min. The mixture was filtered and the filtrate was concentrated under reduced pressure. The residue was purified by RP-HPLC (Boston Green ODS 5 μm 150×30 mm) eluting with 38-68% acetonitrile/Water+10 mM NH₄HCO₃ over 10 minutes to give title compound. LRMS m/z: (M+H)⁺ calculated 543.3; found 543.2.

¹H NMR (400 MHz, CD₃OD) δ 4.37-3.87 (m, 4H), 3.77-3.55 (m, 4H), 2.83-2.61 (m, 1H), 2.28-1.89 (m, 3H), 1.86-1.44 (m, 7H), 1.01 (s, 9H), 0.93-0.66 (m, 3H), 0.65-0.33 (m, 5H), 0.31-0.10 (m, 1H).

### Example 24

### Methyl ((S)-1-((1S,3aR,6aS)-1-(((S)-6,6-difluoro-1-(methylamino)-1,2-dioxoheptan-3-yl)carbamoyl)hexahydrocyclopenta[c]pyrrol-2(1H)-yl)-3,3-dimethyl-1-oxobutan-2-yl)carbamate

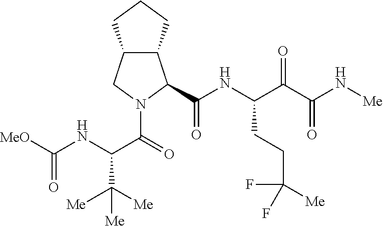 [SMILES: CNC(=O)C(=O)[C@H](CCC(C)(F)F)NC(=O)[C@@H]1[C@H]2CCC[C@H]2CN1C(=O)[C@@H](NC(=O)OC)C(C)(C)C]

### Step 1: Ethyl (1S,3aR,6aS)-2-((S)-2-((methoxycarbonyl)amino)-3,3-dimethylbutanoyl) octahydrocyclopenta[c]pyrrole-1-carboxylate

To a stirred solution of (S)-2-((methoxycarbonyl)amino)-3,3-dimethylbutanoic acid (71 mg, 0.38 mmol), ethyl (1S,3aR,6aS)-octahydrocyclopenta[c]pyrrole-1-carboxylate hydrochloride (75 mg, 0.34 mmol) and HATU (156 mg, 0.410 mmol) in DMF (1.7 mL) at ambient temperature was added DIPEA (178 μL, 1.02 mmol). The mixture was stirred for 1 hour and then diluted with EtOAc and washed with water and brine. The aqueous layer was extracted with two portions of EtOAc. The combined organic layers were dried over Na₂SO₄, filtered, and concentrated to give the title compound. LRMS m/z: (M+H)⁺ calculated 355.2; found 355.3.

### Step 2: (1S,3aR,6aS)-2-((S)-2-((Methoxycarbonyl)amino)-3,3-dimethylbutanoyl) octahydrocyclopenta[c]pyrrole-1-carboxylic acid

A solution of ethyl (1S,3aR,6aS)-2-((S)-2-((methoxycarbonyl)amino)-3,3-dimethylbutanoyl)octahydrocyclopenta[c]pyrrole-1-carboxylate (116 mg, 0.327 mmol) in THF (1.6 mL) was treated with lithium hydroxide (1 N in water) (556 μL, 0.556 mmol), and MeOH (545 μL) and the mixture was stirred at RT. After 40 min, additional lithium hydroxide (15.7 mg, 0.655 mmol) was added and stirring was continued. After an additional 30 min, the mixture was carefully treated with 6 M HCl (273 μL, 1.64 mmol), then diluted with DCM, water, and brine. The aqueous layer was extracted with three portions of DCM. The combined organic layers were dried over Na₂SO₄, filtered, and concentrated. The residue was purified by reverse-phase HPLC (Waters SunFire Prep C18 OBD 5 μm 30×150 mm) eluting with 5-85% MeCN/H₂O+0.1% TFA over 20 min. Product-containing fractions were concentrated to give the title compound. LRMS m/z: (M+H)⁺ calculated 327.2; found 327.5.

### Step 3: Methyl ((2S)-1-((2S,4R)-2-(((3S)-6,6-difluoro-2-hydroxy-1-(methylamino)-1-oxoheptan-3-yl)carbamoyl)-4-(trifluoromethyl)piperidin-1-yl)-3,3-dimethyl-1-oxobutan-2-yl)carbamate

A solution of (1S,3aR,6aS)-2-((S)-2-((methoxycarbonyl)amino)-3,3-dimethylbutanoyl) octahydrocyclopenta[c]pyrrole-1-carboxylic acid (52.5 mg, 0.161 mmol), (2S,3S)-3-amino-6,6-difluoro-2-hydroxy-N-methylheptanamide hydrochloride (40.6 mg, 0.193 mmol) and 7-azabenzotriazol-1-yl oxytris(dimethylamino)phosphonium hexafluorophosphonate (143 mg, 0.322 mmol) in DMF (0.8 mL) was treated with DIPEA (84 uL, 0.48 mmol). The reaction was stirred at RT for 1 hour, then purified directly by reverse-phase HPLC (Waters Xbridge C18 OBD 5 μm 50×250 mm) eluting with 10-60% MeCN/H₂O (5 mM NH₄HCO₃ modifier in H₂O) over 15 min. Pure product-containing fractions were concentrated to afford the title compound. LRMS m/z: (M+H)⁺ calculated 519.3; found 519.6.

### Step 4: Methyl ((S)-1-((1S,3aR,6aS)-1-(((S)-6,6-difluoro-1-(methylamino)-1,2-dioxoheptan-3-yl)carbamoyl)hexahydrocyclopenta[c]pyrrol-2(1H)-yl)-3,3-dimethyl-1-oxobutan-2-yl)carbamate

A solution of methyl ((2S)-1-((2S,4R)-2-(((3S)-6,6-difluoro-2-hydroxy-1-(methylamino)-1-oxoheptan-3-yl)carbamoyl)-4-(trifluoromethyl)piperidin-1-yl)-3,3-dimethyl-1-oxobutan-2-yl)carbamate (60.6 mg, 0.117 mmol) in DCM (1.17 mL) was cooled to 0° C. and then treated with sodium bicarbonate (39.3 mg, 0.467 mmol) followed by Dess-Martin Periodinane (74.3 mg, 0.175 mmol). The mixture was stirred for 5 minutes at 0° C. and then warmed to room temperature and stirred for 2 h. The reaction mixture was quenched with saturated aq. sodium thiosulfate and saturated aq. NaHCO₃ and stirred for 15 minutes. The mixture was extracted twice with DCM. The combined organic portions were dried over Na₂SO₄, filtered, and concentrated under reduced pressure. The residue was purified by column chromatography on silica gel eluting with 0-100% EtOAc/hexanes. Product-containing fractions were combined and concentrated to give the title compound. LRMS m/z: (M+H)⁺ calculated 517.3; found 517.6. ¹H NMR (500 MHz, DMSO-d₆) δ 8.61 (q, J=4.6 Hz, 1H), 8.37 (d, J=7.3 Hz, 1H), 7.07 (d, J=8.4 Hz, 1H), 4.98 (td, J=8.4, 7.4, 3.7 Hz, 1H), 4.28-3.98 (m, 2H), 3.70 (dt, J=46.8, 8.9 Hz, 2H), 3.52 (s, 3H), 2.68-2.61 (m, 3H), 2.46 (dq, J=7.8, 3.9 Hz, 1H), 2.10-1.49 (m, 13H), 1.40 (dt, J=11.7, 5.6 Hz, 1H), 0.92 (d, J=11.5 Hz, 9H).

### Example 25

### Methyl ((S)-1-((1R,2S,5R)-2-(((S)-1-(cyclopropylamino)-6,6-difluoro-1,2-dioxoheptan-3-yl)carbamoyl)-7,7-dimethyl-3-azabicyclo[3.2.0]heptan-3-yl)-3,3-dimethyl-1-oxobutan-2-yl)carbamate

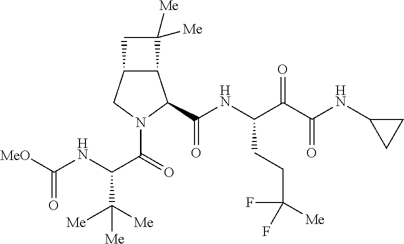 [SMILES: COC(=O)N[C@H](C(=O)N1C[C@@H]2CC(C)(C)[C@@H]2[C@H]1C(=O)N[C@@H](CCC(C)(F)F)C(=O)C(=O)NC1CC1)C(C)(C)C]

### Step 1: Benzyl (1R,2S,5R)-3-((S)-2-((methoxycarbonyl)amino)-3,3-dimethylbutanoyl)-7,7-dimethyl-3-azabicyclo[3.2.0]heptane-2-carboxylate

A solution of benzyl (1R,2S,5R)-7,7-dimethyl-3-azabicyclo[3.2.0]heptane-2-carboxylate (100 mg, 0.39 mmol), (S)-2-((methoxycarbonyl)amino)-3,3-dimethylbutanoic acid (146 mg, 0.771 mmol), AOP (205 mg, 0.463 mmol) and DIEA (0.202 mL, 1.16 mmol) in DMF (2 mL) was stirred at 25° C. for 16 hours. The reaction mixture was purified directly by RP-HPLC (Boston Green ODS 5 μm 150×30 mm) eluting with a gradient of 65-85% acetonitrile/water+0.1% TFA over 6 minutes at 25 mL/min to give the title compound. LRMS m/z: (M+H)⁺ calculated 431.3; found 431.5.

### Step 2: (1R,2S,5R)-3-((S)-2-((Methoxycarbonyl)amino)-3,3-dimethylbutanoyl)-7,7-dimethyl-3-azabicyclo[3.2.0]heptane-2-carboxylic acid

To a mixture of benzyl (1R,2S,5R)-3-((S)-2-((methoxycarbonyl)amino)-3,3-dimethylbutanoyl)-7,7-dimethyl-3-azabicyclo[3.2.0]heptane-2-carboxylate (120 mg, 0.28 mmol) in EtOAc (3 mL) was added 10 wt % Pd/C (30 mg, 0.028 mmol) at 25° C. The vessel was degassed and backfilled with H₂ three times. The reaction mixture was stirred at 25° C. for 1 hour under H₂ balloon. The mixture was filtered, and the solvent was removed under reduced pressure to give the title compound. LRMS m/z: (M+H)⁺ calculated 341.2; found 341.2.

### Step 3: Methyl ((2S)-1-((1R,2S,5R)-2-(((3S)-1-(cyclopropylamino)-6,6-difluoro-2-hydroxy-1-oxoheptan-3-yl)carbamoyl)-7,7-dimethyl-3-azabicyclo[3.2.0]heptan-3-yl)-3,3-dimethyl-1-oxobutan-2-yl)carbamate

A solution of (1R,2S,5R)-3-((S)-2-((methoxycarbonyl)amino)-3,3-dimethylbutanoyl)-7,7-dimethyl-3-azabicyclo[3.2.0]heptane-2-carboxylic acid (95 mg, 0.28 mmol), (3S)-3-amino-N-cyclopropyl-6,6-difluoro-2-hydroxyheptanamide (72.5 mg, 0.307 mmol), AOP (148 mg, 0.335 mmol) and DIEA (0.146 mL, 0.837 mmol) in DMF (2 mL) was stirred at 25° C. for 16 hours. The reaction mixture was purified directly by RP-HPLC (Boston Green ODS 5 μm 150×30 mm) eluting with 36-56% acetonitrile/water+0.10% TFA over 8 minutes to give title compound. LRMS m/z: (M+H)⁺ calculated 559.3; found 559.2.

### Step 4: Methyl ((S)-1-((1R,2S,5R)-2-(((S)-1-(cyclopropylamino)-6,6-difluoro-1,2-dioxoheptan-3-yl)carbamoyl)-7,7-dimethyl-3-azabicyclo[3.2.0]heptan-3-yl)-3,3-dimethyl-1-oxobutan-2-yl)carbamate

To a solution of methyl ((2S)-1-((1R,2S,5R)-2-(((3S)-1-(cyclopropylamino)-6,6-difluoro-2-hydroxy-1-oxoheptan-3-yl)carbamoyl)-7,7-dimethyl-3-azabicyclo[3.2.0]heptan-3-yl)-3,3-dimethyl-1-oxobutan-2-yl)carbamate (90 mg, 0.16 mmol) in DCM (3 mL) was added NaHCO₃ (40.6 mg, 0.483 mmol) and DMP (205 mg, 0.483 mmol) at 0° C. The reaction mixture was warmed to 25° C. and stirred for 3 hours. Na₂SO₃ (60 mg) was added to the reaction and stirred at 20° C. for 15 min. The mixture was filtered and the solvent was removed under reduced pressure. The residue was purified directly by RP-HPLC (Boston Green ODS 5 μm 150×30 mm) eluting with 36-66% acetonitrile/water+10 mM NH₄HCO₃ over 6 minutes to give the title compound. LRMS m/z: (M+H)⁺ calculated 557.3; found 557.3. ¹H NMR (400 MHz, CD₃OD) δ 4.63-4.73 (m, 1H), 4.28 (br d, J=4.16 Hz, 1H), 3.80-3.90 (m, 1H), 3.59-3.70 (m, 1H), 3.55 (s, 3H), 2.84-2.99 (m, 1H), 2.35-2.67 (m, 2H), 1.76-1.94 (m, 3H), 1.29-1.68 (m, 7H), 1.12 (s, 3H), 0.84 (S, 9H), 0.76-0.86 (m, 3H), 0.59-0.73 (m, 2H), 0.35-0.54 (m, 2H).

The following compounds were prepared according to Scheme A and in a similar manner to the procedures described in Examples 20-25:

| Ex. | | | LRMS m/z |
|---|---|---|---|
| No. | Structure | Name | (M + H)⁺ |
| 26 | 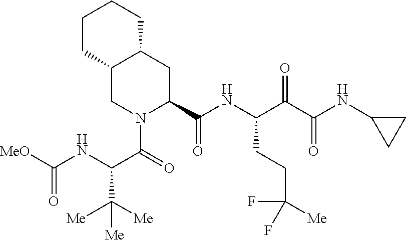 [SMILES: COC(=O)N[C@H](C(=O)N1C[C@@H]2CCCC[C@@H]2C[C@H]1C(=O)N[C@@H](CCC(C)(F)F)C(=O)C(=O)NC1CC1)C(C)(C)C] | Methyl ((S)-1-((3S,4aR,8aR)-3- (((S)-1-(cyclopropylamino)-6,6- difluoro-1,2-dioxoheptan-3- yl)carbamoyl)octahydroiso- quinolin-2(1H)-yl)-3,3-dimethyl-1- oxobutan-2-yl)carbamate | Calculated: 571.3 Found: 571.3 |
| 27 | 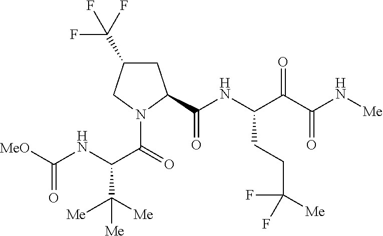 [SMILES: CNC(=O)C(=O)[C@H](CCC(C)(F)F)NC(=O)[C@@H]1C[C@@H](C(F)(F)F)CN1C(=O)[C@@H](NC(=O)OC)C(C)(C)C] | Methyl ((S)-1-((2S,4R)-2-(((S)- 6,6-difluoro-1-(methylamino)- 1,2-dioxoheptan-3- yl)carbamoyl)-4- (trifluoromethyl)pyrrolidin-1-yl)- 3,3-dimethyl-1-oxobutan-2- yl)carbamate | Calculated: 545.2 Found: 545.4 |
| 28 | 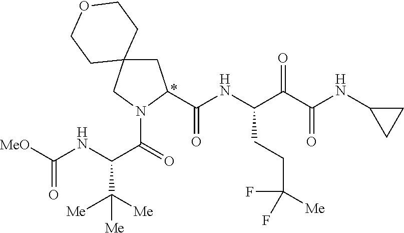 [SMILES: COC(=O)N[C@H](C(=O)N1CC2(CCOCC2)CC1C(=O)N[C@@H](CCC(C)(F)F)C(=O)C(=O)NC1CC1)C(C)(C)C] | Methyl ((S)-1-((R or S)-(3-(((S)- 1-(cyclopropylamino)-6,6- difluoro-1,2-dioxoheptan-3- yl)carbamoyl)-8-oxa-2- azaspiro[4.5]decan-2-yl)-3,3- dimethyl-1-oxobutan-2- yl)carbamate | Calculated: 573.3 Found: 573.4 |
| 29 | 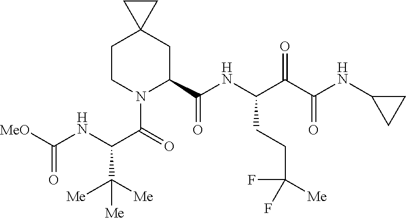 [SMILES: COC(=O)N[C@H](C(=O)N1CCC2(CC2)C[C@H]1C(=O)N[C@@H](CCC(C)(F)F)C(=O)C(=O)NC1CC1)C(C)(C)C] | Methyl ((S)-1-((S)-5-(((S)-1- (cyclopropylamino)-6,6-difluoro- 1,2-dioxoheptan-3- yl)carbamoyl)-6- azaspiro[2.5]octan-6-yl)-3,3- dimethyl-1-oxobutan-2- yl)carbamate | Calculated: 543.3 Found: 543.5 |
| 30 | 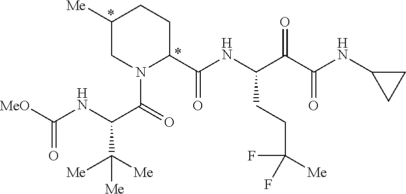 [SMILES: COC(=O)N[C@H](C(=O)N1CC(C)CCC1C(=O)N[C@@H](CCC(C)(F)F)C(=O)C(=O)NC1CC1)C(C)(C)C] | Methyl ((S)-1-(2(R or S), 5(R or S)-(((S)-1-(cyclopropylamino)- 6,6-difluoro-1,2-dioxoheptan-3- yl)carbamoyl)-5- methylpiperidin-1-yl)-3,3- dimethyl-1-oxobutan-2- yl)carbamate | Calculated: 531.3 Found: 531.6 |
| 31 | 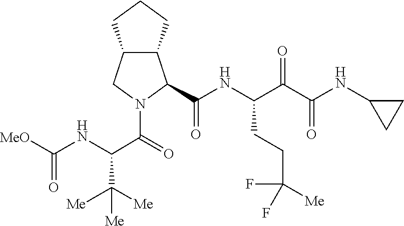 [SMILES: COC(=O)N[C@H](C(=O)N1C[C@@H]2CCC[C@@H]2[C@H]1C(=O)N[C@@H](CCC(C)(F)F)C(=O)C(=O)NC1CC1)C(C)(C)C] | Methyl ((S)-1-((1S,3aR,6aS)-1- (((S)-1-(cyclopropylamino)-6,6- difluoro-1,2-dioxoheptan-3- yl)carbamoyl)hexahydrocyclo- penta[c]pyrrol-2(1H)-yl)-3,3- dimethyl-1-oxobutan-2- yl)carbamate | Calculated: 543.3 Found: 543.5 |
| 32 | 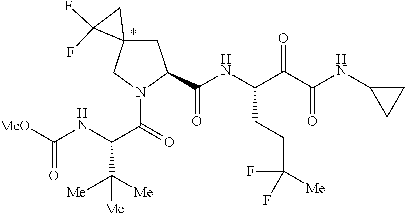 [SMILES: COC(=O)N[C@H](C(=O)N1CC2(C[C@H]1C(=O)N[C@@H](CCC(C)(F)F)C(=O)C(=O)NC1CC1)CC2(F)F)C(C)(C)C] | Methyl ((S)-1-((3(R or S),6S)-6- (((S)-1-(cyclopropylamino)-6,6- difluoro-1,2-dioxoheptan-3- yl)carbamoyl)-1,1-difluoro-5- azaspiro[2.4]heptan-5-yl)-3,3- dimethyl-1-oxobutan-2- yl)carbamate | Calculated: 565.3 Found: 565.5 |
| 33 | 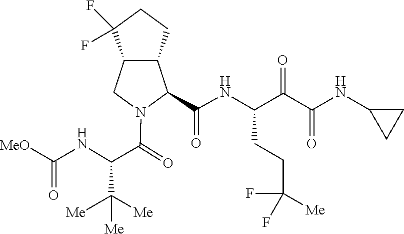 [SMILES: COC(=O)N[C@H](C(=O)N1C[C@H]2[C@H](CCC2(F)F)[C@H]1C(=O)N[C@@H](CCC(C)(F)F)C(=O)C(=O)NC1CC1)C(C)(C)C] | Methyl ((S)-1-((1S,3aR,6aS)-1- (((S)-1-(cyclopropylamino)-6,6- difluoro-1,2-dioxoheptan-3- yl)carbamoyl)-4,4- difluorohexahydrocyclopenta[c] pyrrol-2(1H)-yl)-3,3-dimethyl-1- oxobutan-2-yl)carbamate | Calculated: 579.3 Found: 579.5 |
| 34 | 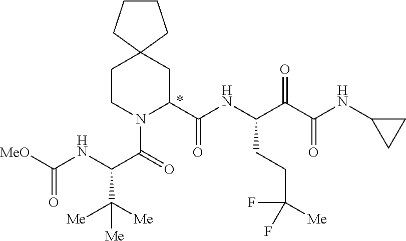 [SMILES: COC(=O)N[C@H](C(=O)N1CCC2(CCCC2)CC1C(=O)N[C@@H](CCC(C)(F)F)C(=O)C(=O)NC1CC1)C(C)(C)C] | Methyl ((S)-1-(R or S)-(7-(((S)- 1-(cyclopropylamino)-6,6- difluoro-1,2-dioxoheptan-3- yl)carbamoyl)-8- azaspiro[4.5]decan-8-yl)-3,3- dimethyl-1-oxobutan-2- yl)carbamateazaspiro[4.5]decan- 8-yl)-3,3-dimethyl-1-oxobutan- 2-yl)carbamate | Calculated: 571.3 Found: 571.5 |
| 35 | 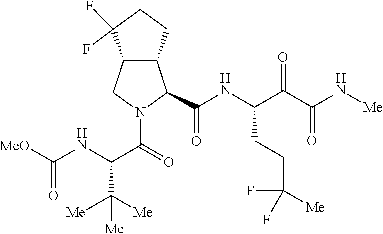 [SMILES: CNC(=O)C(=O)[C@H](CCC(C)(F)F)NC(=O)[C@@H]1[C@H]2CCC(F)(F)[C@H]2CN1C(=O)[C@@H](NC(=O)OC)C(C)(C)C] | Methyl ((S)-1-((1S,3aR,6aS)-1- (((S)-6,6-difluoro-1- (methylamino)-1,2-dioxoheptan- 3-yl)carbamoyl)-4,4- difluorohexahydrocyclopenta[c] pyrrol-2(1H)-yl)-3,3-dimethyl-1- oxobutan-2-yl)carbamate | Calculated: 553.3 Found: 553.5 |
| 36 | 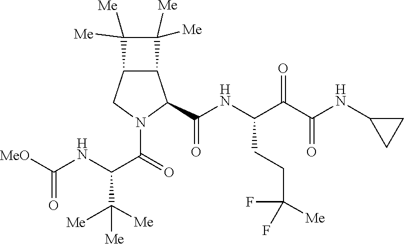 [SMILES: COC(=O)N[C@H](C(=O)N1C[C@H]2[C@@H]([C@H]1C(=O)N[C@@H](CCC(C)(F)F)C(=O)C(=O)NC1CC1)C(C)(C)C2(C)C)C(C)(C)C] | Methyl ((S)-1-((1R,2S,5S)-2- (((S)-1-(cyclopropylamino)-6,6- difluoro-1,2-dioxoheptan-3- yl)carbamoyl)-6,6,7,7- tetramethyl-3- azabicyclo[3.2.0]heptan-3-yl)- 3,3-dimethyl-1-oxobutan-2- yl)carbamate | Calculated: 585.4 Found: 585.4 |
| 37 | 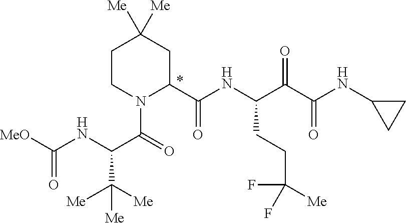 [SMILES: COC(=O)N[C@H](C(=O)N1CCC(C)(C)CC1C(=O)N[C@@H](CCC(C)(F)F)C(=O)C(=O)NC1CC1)C(C)(C)C] | Methyl ((S)-1-((R or S)-(2-(((S)- 1-(cyclopropylamino)-6,6- difluoro-1,2-dioxoheptan-3- yl)carbamoyl)-4,4- dimethylpiperidin-1-yl)-3,3- dimethyl-1-oxobutan-2- yl)carbamate | Calculated: 545.3 Found: 545.5 |
| 38 | 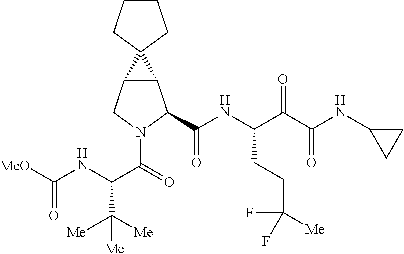 [SMILES: COC(=O)N[C@H](C(=O)N1C[C@H]2[C@@H]([C@H]1C(=O)N[C@@H](CCC(C)(F)F)C(=O)C(=O)NC1CC1)C21CCCC1)C(C)(C)C] | Methyl ((S)-1-((1R,2S,5S)-2- (((S)-1-(cyclopropylamino)-6,6- difluoro-1,2-dioxoheptan-3- yl)carbamoyl)-3- azaspiro[bicyclo[3.1.0]hexane- 6,1′-cyclopentan]-3-yl)-3,3- dimethyl-1-oxobutan-2- yl)carbamate | Calculated: 569.3 Found: 569.3 |
| 39 | 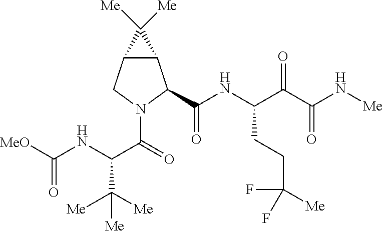 [SMILES: CNC(=O)C(=O)[C@H](CCC(C)(F)F)NC(=O)[C@@H]1[C@@H]2[C@H](CN1C(=O)[C@@H](NC(=O)OC)C(C)(C)C)C2(C)C] | Methyl ((S)-1-((1R,2S,5S)-2- (((S)-6,6-difluoro-1- (methylamino)-1,2-dioxoheptan- 3-yl)carbamoyl)-6,6-dimethyl-3- azabicyclo[3.1.0]hexan-3-yl)- 3,3-dimethyl-1-oxobutan-2- yl)carbamate | Calculated: 517.3 Found: 517.6 |
| 40 | 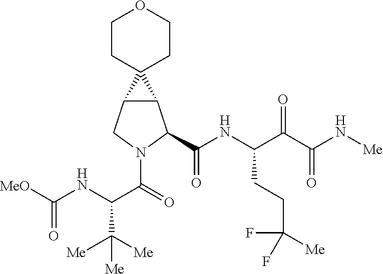 [SMILES: CNC(=O)C(=O)[C@H](CCC(C)(F)F)NC(=O)[C@@H]1[C@@H]2[C@H](CN1C(=O)[C@@H](NC(=O)OC)C(C)(C)C)C21CCOCC1] | Methyl ((S)-1-((1R,2S,5S)-2- (((S)-6,6-difluoro-1- (methylamino)-1,2-dioxoheptan- 3-yl)carbamoyl)tetrahydro-3- azaspiro[bicyclo[3.1.0]hexane- 6,4′-pyran]-3-yl)-3,3-dimethyl-1- oxobutan-2-yl)carbamate | Calculated: 559.3 Found: 559.5 |
| 41 | 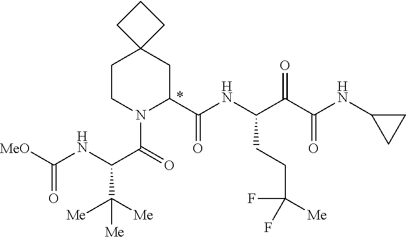 [SMILES: COC(=O)N[C@H](C(=O)N1CCC2(CCC2)CC1C(=O)N[C@@H](CCC(C)(F)F)C(=O)C(=O)NC1CC1)C(C)(C)C] | Methyl ((S)-1-((R or S)-(6-(((S)- 1-(cyclopropylamino)-6,6- difluoro-1,2-dioxoheptan-3- yl)carbamoyl)-7- azaspiro[3.5]nonan-7-yl)-3,3- dimethyl-1-oxobutan-2- yl)carbamate | Calculated: 557.3 Found: 557.5 |
| 42 | 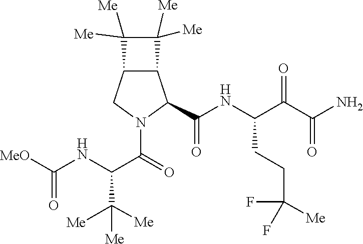 [SMILES: COC(=O)N[C@H](C(=O)N1C[C@H]2[C@@H]([C@H]1C(=O)N[C@@H](CCC(C)(F)F)C(=O)C(N)=O)C(C)(C)C2(C)C)C(C)(C)C] | Methyl ((S)-1-((1R,2S,5S)-2- (((S)-1-amino-6,6-difluoro-1,2- dioxoheptan-3-yl)carbamoyl)- 6,6,7,7-tetramethyl-3- azabicyclo[3.2.0]heptan-3-yl)- 3,3-dimethyl-1-oxobutan-2- yl)carbamate | Calculated: 545.3 Found: 545.2 |
| 43 | 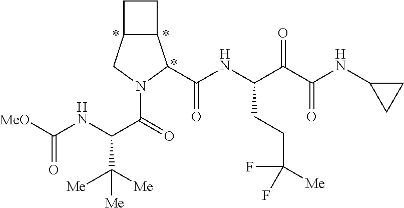 [SMILES: COC(=O)N[C@H](C(=O)N1CC2CCC2C1C(=O)N[C@@H](CCC(C)(F)F)C(=O)C(=O)NC1CC1)C(C)(C)C] | Methyl ((S)-1-((1(R or S), 2(R or S),5(R or S))-(2-(((S)-1- (cyclopropylamino)-6,6-difluoro- 1,2-dioxoheptan-3- yl)carbamoyl)-3- azabicyclo[3.2.0]heptan-3-yl)- 3,3-dimethyl-1-oxobutan-2- yl)carbamate | Calculated: 529.3 Found: 529.5 |
| 44 | 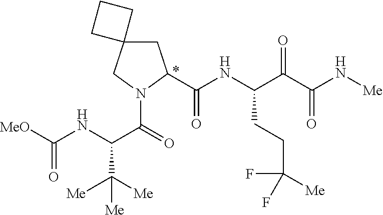 [SMILES: CNC(=O)C(=O)[C@H](CCC(C)(F)F)NC(=O)C1CC2(CCC2)CN1C(=O)[C@@H](NC(=O)OC)C(C)(C)C] | Methyl ((S)-1-((R or S)-7-(((S)- 6,6-difluoro-1-(methylamino)- 1,2-dioxoheptan-3- yl)carbamoyl)-6- azaspiro[3.4]octan-6-yl)-3,3- dimethyl-1-oxobutan-2- yl)carbamate | Calculated: 517.3 Found: 517.5 |
| 45 | 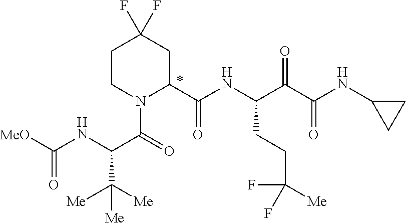 [SMILES: COC(=O)N[C@H](C(=O)N1CCC(F)(F)CC1C(=O)N[C@@H](CCC(C)(F)F)C(=O)C(=O)NC1CC1)C(C)(C)C] | Methyl ((S)-1-(R or S)-(2-(((S)- 1-(cyclopropylamino)-6,6- difluoro-1,2-dioxoheptan-3- yl)carbamoyl)-4,4- difluoropiperidin-1-yl)-3,3- dimethyl-1-oxobutan-2- yl)carbamate | Calculated: 553.3 Found: 553.6 |
| 46 | 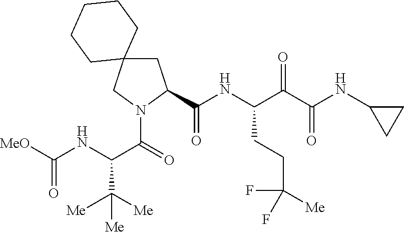 [SMILES: COC(=O)N[C@H](C(=O)N1CC2(CCCCC2)C[C@H]1C(=O)N[C@@H](CCC(C)(F)F)C(=O)C(=O)NC1CC1)C(C)(C)C] | Methyl ((R)-3-((S)-3-(((S)-1- (cyclopropylamino)-6,6-difluoro- 1,2-dioxoheptan-3- yl)carbamoyl)-2- azaspiro[4.5]decan-2-yl)-1,1,1- trifluoro-3-oxopropan-2- yl)carbamate | Calculated: 583.3 Found: 583.2 |
| 47 | 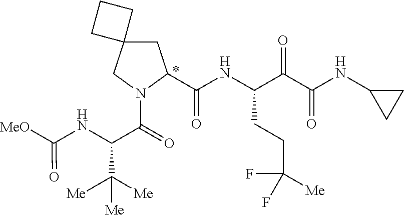 [SMILES: COC(=O)N[C@H](C(=O)N1CC2(CCC2)CC1C(=O)N[C@@H](CCC(C)(F)F)C(=O)C(=O)NC1CC1)C(C)(C)C] | Methyl ((S)-1-(R or S)-7-(((S)-1- (cyclopropylamino)-6,6-difluoro- 1,2-dioxoheptan-3- yl)carbamoyl)-6- azaspiro[3.4]octan-6-yl)-3,3- dimethyl-1-oxobutan-2- yl)carbamate | Calculated: 543.3 Found: 543.5 |
| 48 | 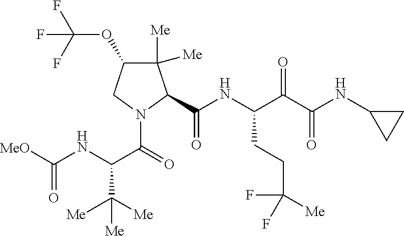 [SMILES: COC(=O)N[C@H](C(=O)N1C[C@H](OC(F)(F)F)C(C)(C)[C@H]1C(=O)N[C@@H](CCC(C)(F)F)C(=O)C(=O)NC1CC1)C(C)(C)C] | Methyl ((S)-1-((2S,4R)-2-(((S)- 1-(cyclopropylamino)-6,6- difluoro-1,2-dioxoheptan-3- yl)carbamoyl)-3,3-dimethyl-4- (trifluoromethoxy)pyrrolidin-1- yl)-3,3-dimethyl-1-oxobutan-2- yl)carbamate | Calculated: 615.3 Found: 615.5 |
| 49 | 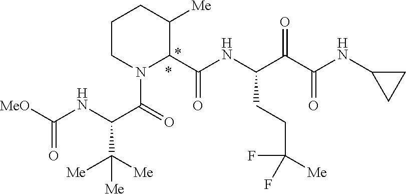 [SMILES: COC(=O)N[C@H](C(=O)N1CCCC(C)C1C(=O)N[C@@H](CCC(C)(F)F)C(=O)C(=O)NC1CC1)C(C)(C)C] | Methyl ((S)-1-((2(R or S),3(R or S))-2-(((S)-1- (cyclopropylamino)-6,6-difluoro- 1,2-dioxoheptan-3- yl)carbamoyl)-3- methylpiperidin-1-yl)-3,3- dimethyl-1-oxobutan-2- yl)carbamate | Calculated: 531.3 Found: 531.5 |
| 50 | 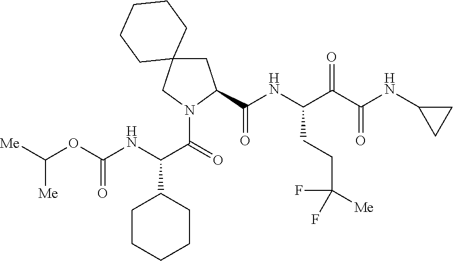 [SMILES: CC(C)OC(=O)N[C@H](C(=O)N1CC2(CCCCC2)C[C@H]1C(=O)N[C@@H](CCC(C)(F)F)C(=O)C(=O)NC1CC1)C1CCCCC1] | Isopropyl ((S)-1-cyclohexyl-2- ((S)-3-(((S)-1- (cyclopropylamino)-6,6-difluoro- 1,2-dioxoheptan-3- yl)carbamoyl)-2- azaspiro[4.5]decan-2-yl)-2- oxoethyl)carbamate | Calculated: 625.4 Found: 625.3 |
| 51 | 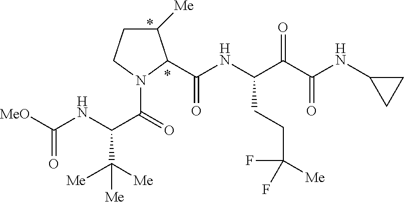 [SMILES: COC(=O)N[C@H](C(=O)N1CCC(C)C1C(=O)N[C@@H](CCC(C)(F)F)C(=O)C(=O)NC1CC1)C(C)(C)C] | Methyl ((S)-1-((2(R or S),3(R or S))-2-(((S)-1- (cyclopropylamino)-6,6-difluoro- 1,2-dioxoheptan-3- yl)carbamoyl)-3- methylpyrrolidin-1-yl)-3,3- dimethyl-1-oxobutan-2- yl)carbamate | Calculated: 517.3 Found: 517.5 |
| 52 | 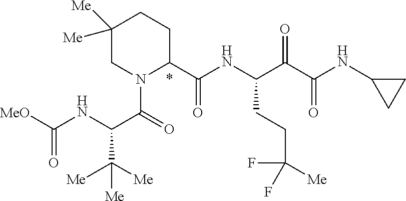 [SMILES: COC(=O)N[C@H](C(=O)N1CC(C)(C)CCC1C(=O)N[C@@H](CCC(C)(F)F)C(=O)C(=O)NC1CC1)C(C)(C)C] | Methyl ((S)-1-(R or S)-2-(((S)-1- (cyclopropylamino)-6,6-difluoro- 1,2-dioxoheptan-3- yl)carbamoyl)-5,5- dimethylpiperidin-1-yl)-3,3- dimethyl-1-oxobutan-2- yl)carbamate | Calculated: 545.3 Found: 545.7 |
| 53 | 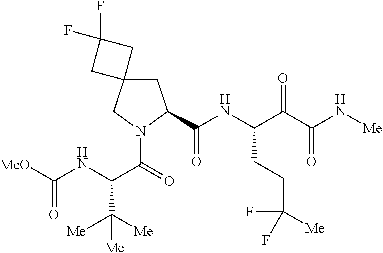 [SMILES: CNC(=O)C(=O)[C@H](CCC(C)(F)F)NC(=O)[C@@H]1CC2(CN1C(=O)[C@@H](NC(=O)OC)C(C)(C)C)CC(F)(F)C2] | Methyl ((S)-1-((S)-7-(((S)-6,6- difluoro-1-(methylamino)-1,2- dioxoheptan-3-yl)carbamoyl)- 2,2-difluoro-6- azaspiro[3.4]octan-6-yl)-3,3- dimethyl-1-oxobutan-2- yl)carbamate | Calculated: 553.3 Found: 553.5 |
| 54 | 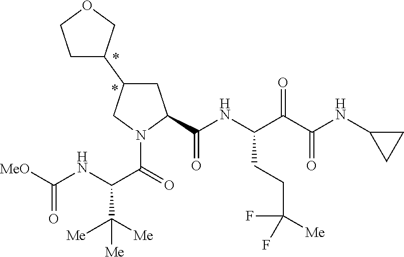 [SMILES: COC(=O)N[C@H](C(=O)N1CC(C2CCOC2)C[C@H]1C(=O)N[C@@H](CCC(C)(F)F)C(=O)C(=O)NC1CC1)C(C)(C)C] | Methyl ((S)-1-((2S,4(R or S))-2- (((S)-1-(cyclopropylamino)-6,6- difluoro-1,2-dioxoheptan-3- yl)carbamoyl)-4-((R or S)- tetrahydrofuran-3-yl)pyrrolidin- 1-yl)-3,3-dimethyl-1-oxobutan- 2-yl)carbamate | Calculated: 573.3 Found: 573.5 |
| 55 | 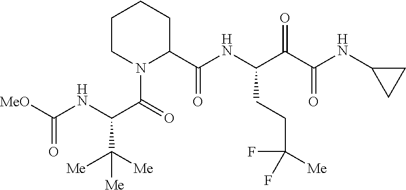 [SMILES: COC(=O)N[C@H](C(=O)N1CCCCC1C(=O)N[C@@H](CCC(C)(F)F)C(=O)C(=O)NC1CC1)C(C)(C)C] | Methyl ((S)-1-((S or R)-2-(((S)- 1-(cyclopropylamino)-6,6- difluoro-1,2-dioxoheptan-3- yl)carbamoyl)piperidin-1-yl)- 3,3-dimethyl-1-oxobutan-2- yl)carbamate: methyl ((S)-1-((R or S)-2-(((S)-1- (cyclopropylamino)-6,6-difluoro- 1,2-dioxoheptan-3- yl)carbamoyl)piperidin-1-yl)- 3,3-dimethyl-1-oxobutan-2- yl)carbamate | Calculated: 517.3 Found: 517.4 |
| | | 3:2 mix of diastereomers | |
| 56 | 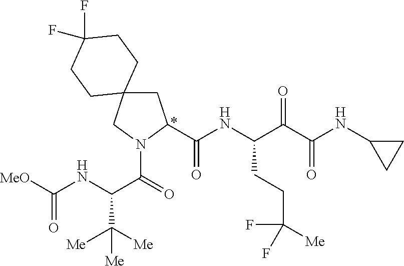 [SMILES: COC(=O)N[C@H](C(=O)N1CC2(CCC(F)(F)CC2)CC1C(=O)N[C@@H](CCC(C)(F)F)C(=O)C(=O)NC1CC1)C(C)(C)C] | Methyl ((S)-1-((R or S)-(3-(((S)- 1-(cyclopropylamino)-6,6- difluoro-1,2-dioxoheptan-3- yl)carbamoyl)-8,8-difluoro-2- azaspiro[4.5]decan-2-yl)-3,3- dimethyl-1-oxobutan-2- yl)carbamate | Calculated: 607.3 Found: 607.2 |
| 57 | 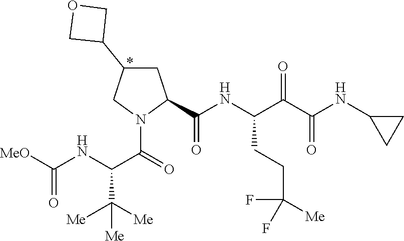 [SMILES: COC(=O)N[C@H](C(=O)N1CC(C2COC2)C[C@H]1C(=O)N[C@@H](CCC(C)(F)F)C(=O)C(=O)NC1CC1)C(C)(C)C] | Methyl ((S)-1-((2S, 4(R or S))-2- (((S)-1-(cyclopropylamino)-6,6- difluoro-1,2-dioxoheptan-3- yl)carbamoyl)-4-(oxetan-3- yl)pyrrolidin-1-yl)-3,3-dimethyl- 1-oxobutan-2-yl)carbamate | Calculated: 559.3 Found: 559.4 |
| 58 | 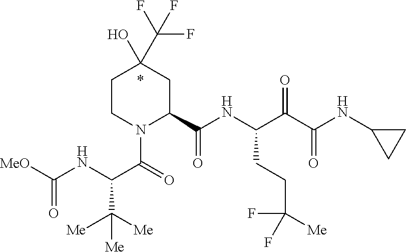 [SMILES: COC(=O)N[C@H](C(=O)N1CCC(O)(C(F)(F)F)C[C@H]1C(=O)N[C@@H](CCC(C)(F)F)C(=O)C(=O)NC1CC1)C(C)(C)C] | Methyl ((S)-1-((2S, 4(R or S))-2- (((S)-1-(cyclopropylamino)-6,6- difluoro-1,2-dioxoheptan-3- yl)carbamoyl)-4-hydroxy-4- (trifluoromethyl)piperidin-1-yl)- 3,3-dimethyl-1-oxobutan-2- yl)carbamate | Calculated: 601.3 Found: 601.4 |
| 59 | 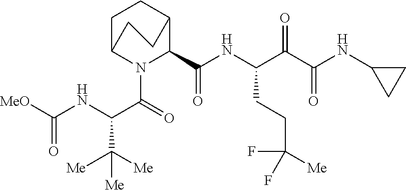 [SMILES: COC(=O)N[C@H](C(=O)N1C2CCC(CC2)[C@H]1C(=O)N[C@@H](CCC(C)(F)F)C(=O)C(=O)NC1CC1)C(C)(C)C] | Methyl ((S)-1-((S)-3-(((S)-1- (cyclopropylamino)-6,6-difluoro- 1,2-dioxoheptan-3- yl)carbamoyl)-2- azabicyclo[2.2.2]octan-2-yl)-3,3- dimethyl-1-oxobutan-2- yl)carbamate | Calculated: 543.3 Found: 543.3 |
| 60 | 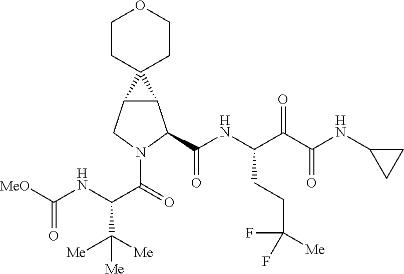 [SMILES: COC(=O)N[C@H](C(=O)N1C[C@H]2[C@@H]([C@H]1C(=O)N[C@@H](CCC(C)(F)F)C(=O)C(=O)NC1CC1)C21CCOCC1)C(C)(C)C] | Methyl ((S)-1-((1R,2S,5S)-2- (((S)-1-(cyclopropylamino)-6,6- difluoro-1,2-dioxoheptan-3- yl)carbamoyl)tetrahydro-3- azaspiro[bicyclo[3.1.0]hexane- 6,4′-pyran]-3-yl)-3,3-dimethyl-1- oxobutan-2-yl)carbamate | Calculated: 585.3 Found: 585.5 |

### From Scheme B

### Example 61

### Methyl ((S)-1-((2S,4R)-2-(((S)-1-(cyclopropylamino)-6,6-difluoro-1,2-dioxoheptan-3-yl)carbamoyl)-4-(trifluoromethyl)piperidin-1-yl)-3,3-dimethyl-1-oxobutan-2-yl)carbamate

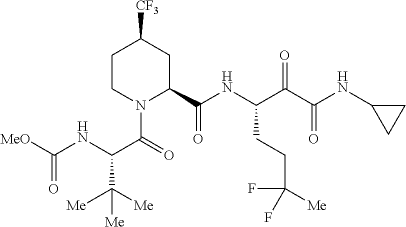 [SMILES: COC(=O)N[C@H](C(=O)N1CC[C@@H](C(F)(F)F)C[C@H]1C(=O)N[C@@H](CCC(C)(F)F)C(=O)C(=O)NC1CC1)C(C)(C)C]

### Step 1: tert-Butyl (2S,4R)-2-(((2S,3S)-1-(cyclopropylamino)-6,6-difluoro-2-hydroxy-1-oxoh eptan-3-yl)carbamoyl)-4-(trifluoromethyl)piperidine-1-carboxylate

A solution of (2S,4R)-1-(tert-butoxycarbonyl)-4-(trifluoromethyl)piperidine-2-carboxylic acid (300 mg, 1.0 mmol), (2S,3S)-3-amino-N-cyclopropyl-6,6-difluoro-2-hydroxyheptanamide hydrochloride (330 mg, 1.2 mmol), HATU (499 mg, 1.31 mmol), and DIPEA (529 μL, 3.03 mmol) in CH₂Cl₂ (10.1 mL) was stirred at 25° C. for 18 hours. The reaction mixture was diluted with DCM, washed three times with saturated aq. NaHCO₃ and once with brine, dried over MgSO₄, filtered, and concentrated under reduced pressure to give the title compound. LRMS m/z: (M+H)⁺ calculated 516.2; found 516.4.

### Step 2: (2S,4R)—N-((2S,3S)-1-(Cyclopropylamino)-6,6-difluoro-2-hydroxy-1-oxoheptan-3-yl)-4-(trifluoromethyl)piperidine-2-carboxamide hydrochloride

To tert-butyl (2S,4R)-2-(((2S,3S)-1-(cyclopropylamino)-6,6-difluoro-2-hydroxy-1-oxoheptan-3-yl)carbamoyl)-4-(trifluoromethyl)piperidine-1-carboxylate (550 mg, 1.1 mmol) was added 4 M HCl in dioxane (5.33 mL, 21.3 mmol) at 25° C. and the mixture was stirred for 2 hours. The solvent was removed under reduced pressure to give the title compound. LRMS m/z: (M+H)⁺ calculated 416.2; found 416.3.

### Step 3: Methyl ((S)-1-((2S,4R)-2-(((2S,3S)-1-(cyclopropylamino)-6,6-difluoro-2-hydroxy-1-oxoheptan-3-yl)carbamoyl)-4-(trifluoromethyl)piperidin-1-yl)-3,3-dimethyl-1-oxobutan-2-yl)carbamate

A solution of (2S,4R)—N-((2S,3S)-1-(cyclopropylamino)-6,6-difluoro-2-hydroxy-1-oxoheptan-3-yl)-4-(trifluoromethyl)piperidine-2-carboxamide hydrochloride (500 mg, 1.1 mmol), (S)-2-((methoxycarbonyl)amino)-3,3-dimethylbutanoic acid (314 mg, 1.66 mmol), 7-azabenzotriazol-1-yloxytris(dimethylamino)phosphonium hexafluorophosphate (736 mg, 1.66 mmol), and DIPEA (966 μL, 5.53 mmol) was added to DMF (5.53 mL) at 25° C. and the reaction was sonicated for 30 seconds and then stirred for 18 hours. The reaction was quenched with saturated aq. NaHCO₃ and diluted with EtOAc. The organic phase was separated, washed three times with water, dried over MgSO₄, filtered, and concentrated under reduced pressure. The residue was purified by column chromatography on silica gel eluting with 0-100% 3:1 EtOAc:EtOH in hexanes to give the title compound. LRMS m/z: (M+H)⁺ calculated 587.3; found 587.5.

### Step 4: Methyl ((S)-1-((2S,4R)-2-(((S)-1-(cyclopropylamino)-6,6-difluoro-1,2-dioxoheptan-3-yl)carbamoyl)-4-(trifluoromethyl)piperidin-1-yl)-3,3-dimethyl-1-oxobutan-2-yl)carbamate

To a stirred mixture of methyl ((S)-1-((2S,4R)-2-(((2S,3S)-1-(cyclopropylamino)-6,6-difluoro-2-hydroxy-1-oxoheptan-3-yl)carbamoyl)-4-(trifluoromethyl)piperidin-1-yl)-3,3-dimethyl-1-oxobutan-2-yl)carbamate (480 mg, 0.82 mmol) and sodium bicarbonate (137 mg, 1.64 mmol) in CH₂Cl₂ (8.18 mL) at 25° C. was added Dess-Martin periodinane (521 mg, 1.23 mmol) and the reaction was stirred at 25° C. for 2 hours. The reaction was quenched with saturated aq. Na₂SO₃ and saturated NaHCO₃. The organic phase was separated, washed with saturated aq. NaHCO₃ and brine, dried over MgSO₄, filtered, and concentrated under reduced pressure. The residue was purified by column chromatography on silica gel eluting with 0-100% EtOAc in hexanes to give the title compound. LRMS m/z: (M+H)⁺ calculated 585.3; found 585.5. ¹H NMR (500 MHz, DMSO-d₆) δ 8.74 (d, J=5.1 Hz, 1H), 8.37 (d, J=7.3 Hz, 1H), 7.06 (d, J=8.4 Hz, 1H), 4.93 (t, J=6.8 Hz, 1H), 4.53-4.36 (m, 2H), 4.00-3.88 (m, 1H), 3.53 (s, 3H), 3.48-3.35 (m, 1H), 2.73 (tq, J=9.0, 4.1 Hz, 1H), 2.67-2.53 (m, 2H), 2.13 (dd, J=8.0, 4.5 Hz, 1H), 2.07-1.84 (m, 4H), 1.73-1.51 (m, 6H), 0.93 (s, 9H), 0.69-0.62 (m, 2H), 0.56 (dt, J=8.1, 4.2 Hz, 2H).

### Example 62

### Methyl ((S)-1-((S)-3-(((S)-6,6-difluoro-1-(methylamino)-1,2-dioxoheptan-3-yl)carbamoyl)-2-azaspiro[4.5]decan-2-yl)-3,3-dimethyl-1-oxobutan-2-yl)carbamate

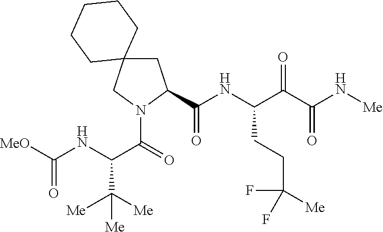 [SMILES: CNC(=O)C(=O)[C@H](CCC(C)(F)F)NC(=O)[C@@H]1CC2(CCCCC2)CN1C(=O)[C@@H](NC(=O)OC)C(C)(C)C]

### Step 1: tert-Butyl (3S)-3-(((3S)-6,6-difluoro-2-hydroxy-1-(methylamino)-1-oxoheptan-3-yl)carbamoyl)-2-azaspiro[4.5]decane-2-carboxylate

To a solution of (S)-2-(tert-butoxycarbonyl)-2-azaspiro[4.5]decane-3-carboxylic acid (300 mg, 1.1 mmol) and (3S)-3-amino-6,6-difluoro-2-hydroxy-N-methylheptanamide (223 mg, 1.06 mmol) in DMF (5.3 mL) was added N-ethyl-N-isopropylpropan-2-amine (553 μL, 3.18 mmol) and HATU (604 mg, 1.59 mmol). The mixture was stirred at RT. After 2 h, the mixture was directly purified by reverse-phase HPLC (Waters SunFire Prep C18 OBD 10 μm 50×250 mm) eluting with 5-100% MeCN/H₂O (0.1% TFA modifier) over 35 min. Product-containing fractions were concentrated to afford the title compound. LRMS m/z: (M+H)⁺ calculated 476.3; found 476.5.

### Step 2: (3S)—N-((3S)-6,6-Difluoro-2-hydroxy-1-(methylamino)-1-oxoheptan-3-yl)-2-azaspiro[4.5]decane-3-carboxamide hydrochloride

A solution of tert-butyl (3S)-3-(((3S)-6,6-difluoro-2-hydroxy-1-(methylamino)-1-oxoheptan-3-yl)carbamoyl)-2-azaspiro[4.5]decane-2-carboxylate (339 mg, 0.713 mmol) in HCl, 4 M in dioxane (5.35 mL, 21.4 mmol) was stirred at RT. After 1 h, the mixture was concentrated to afford the title compound. LRMS m/z: (M+H)⁺ calculated 376.2; found 376.4.

### Step 3: Methyl ((2S)-1-((3S)-3-(((3S)-6,6-difluoro-2-hydroxy-1-(methylamino)-1-oxoheptan-3-yl)carbamoyl)-2-azaspiro[4.5]decan-2-yl)-3,3-dimethyl-1-oxobutan-2-yl)carbamate

A suspension of (3S)—N-((3S)-6,6-difluoro-2-hydroxy-1-(methylamino)-1-oxoheptan-3-yl)-2-azaspiro[4.5]decane-3-carboxamide hydrochloride (294 mg, 0.714 mmol) and methyl (S)-(1-amino-3,3-dimethyl-1-oxobutan-2-yl)carbamate (148 mg, 0.785 mmol) in DMF (2.38 mL) was treated with DIPEA (373 μL, 2.14 mmol) and HATU (326 mg, 0.856 mmol). The mixture was stirred at RT. After 3 h, the mixture was filtered and directly purified by reverse-phase HPLC (Waters SunFire Prep C18 OBD 10 μm 50×250 mm eluting with 5-85% MeCN/H₂O (0.1% TFA modifier) over 30 min. Product-containing fractions were concentrated to afford title compound. LRMS m/z: (M+H)⁺ calculated 547.3; found 547.5.

### Step 4: Methyl ((S)-1-((S)-3-(((S)-6,6-difluoro-1-(methylamino)-1,2-dioxoheptan-3-yl)carbamoyl)-2-azaspiro[4.5]decan-2-yl)-3,3-dimethyl-1-oxobutan-2-yl)carbamate

A solution of methyl ((2S)-1-((3S)-3-(((3S)-6,6-difluoro-2-hydroxy-1-(methylamino)-1-oxoheptan-3-yl)carbamoyl)-2-azaspiro[4.5]decan-2-yl)-3,3-dimethyl-1-oxobutan-2-yl)carbamate (400 mg, 0.25 mmol) in DCM (7.3 mL), was cooled to 0° C. and then treated with sodium bicarbonate (246 mg, 2.93 mmol) followed by Dess-Martin periodinane (621 mg, 1.46 mmol). The mixture was stirred for 5 minutes at 0° C. and then warmed to ambient temperature and stirred for additional 2 hours, after which the mixture was treated with additional Dess-Martin periodinane (310 mg, 0.732 mmol). After stirring for an additional 1 hour, the reaction mixture was quenched with saturated aq. sodium thiosulfate and saturated aq. NaHCO₃ and stirred for 15 minutes. The mixture was extracted with two portions of DCM. The combined organic layers were dried over Na₂SO₄, filtered, and concentrated under reduced pressure. The residue was purified by column chromatography on silica gel eluting with 0-100% EtOAc/hexanes. Product-containing fractions were concentrated under reduced pressure. The residue was repurified by reverse-phase HPLC (Waters XBridge Prep C18 OBD 5 μm 50×250 mm) eluting with 10-100% MeCN/H₂O (5 mM NH₄HCO₃ modifier in H₂O) over 15 min. Pure product-containing fractions were concentrated to afford the title compound. LRMS m/z: (M+H)⁺ calculated 545.3; found 545.5. ¹H NMR (500 MHz, DMSO-d₆) δ 8.62 (q, J=4.5 Hz, 1H), 8.31 (d, J=7.3 Hz, 1H), 7.04 (d, J=8.4 Hz, 1H), 5.11-4.87 (m, 1H), 4.35 (t, J=8.5 Hz, 1H), 4.14 (d, J=8.5 Hz, 1H), 3.83 (d, J=9.8 Hz, 1H), 3.33 (s, 3H), 3.15 (d, J=9.8 Hz, 1H), 2.66 (d, J=4.8 Hz, 3H), 2.15-1.82 (m, 4H), 1.67-1.52 (m, 3H), 1.52-1.20 (m, 12H), 0.94 (s, 9H).

The following compounds were prepared in a similar manner to the methods described in Examples 61 and 62:

| | | | LRMS |
|---|---|---|---|
| Ex. | | | m/z |
| No. | Structure | Name | (M + H)⁺ |
| 63 | 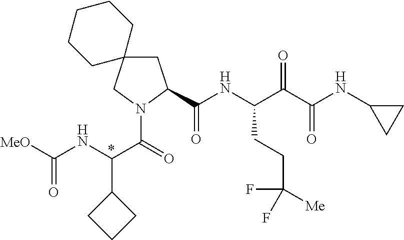 [SMILES: COC(=O)NC(C(=O)N1CC2(CCCCC2)C[C@H]1C(=O)N[C@@H](CCC(C)(F)F)C(=O)C(=O)NC1CC1)C1CCC1] | Methyl ((R or S)-1- cyclobutyl-2-((S)-3-(((S)-1- (cyclopropylamino)-6,6- difluoro-1,2-dioxoheptan-3- yl)carbamoyl)-2- azaspiro[4.5]decan-2-yl)-2- oxoethyl)carbamate | Calculated: 569.3 Found: 569.3 |
| 64 | 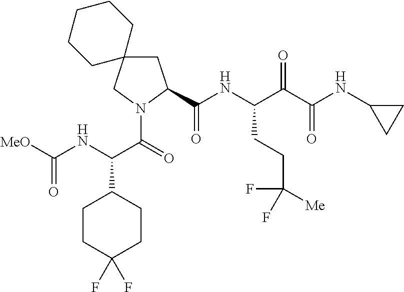 [SMILES: COC(=O)N[C@H](C(=O)N1CC2(CCCCC2)C[C@H]1C(=O)N[C@@H](CCC(C)(F)F)C(=O)C(=O)NC1CC1)C1CCC(F)(F)CC1] | Methyl ((S)-2-((2S,4R)-2- (((S)-1-(cyclopropylamino)- 6,6-difluoro-1,2-dioxoheptan- 3-yl)carbamoyl)-4- (trifluoromethyl)pyrrolidin-1- yl)-1-(4,4- difluorocyclohexyl)-2- oxoethyl)carbamate | Calculated: 633.3 Found: 633.3 |
| 65 | 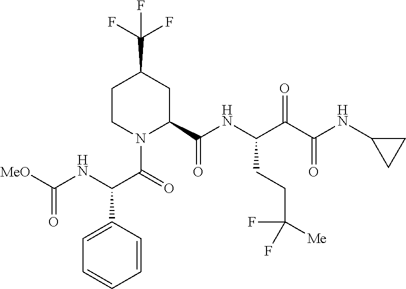 [SMILES: COC(=O)N[C@H](C(=O)N1CC[C@@H](C(F)(F)F)C[C@H]1C(=O)N[C@@H](CCC(C)(F)F)C(=O)C(=O)NC1CC1)c1ccccc1] | Methyl ((S)-2-((2S,4R)-2- (((S)-1-(cyclopropylamino)- 6,6-difluoro-1,2-dioxoheptan- 3-yl)carbamoyl)-4- (trifluoromethyl)piperidin-1- yl)-2-oxo-1- phenylethyl)carbamate | Calculated: 605.2 Found: 605.3 |
| 66 | 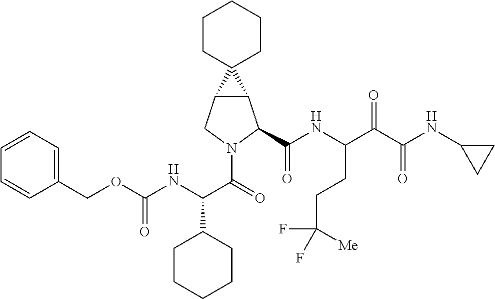 [SMILES: CC(F)(F)CCC(NC(=O)[C@@H]1[C@@H]2[C@H](CN1C(=O)[C@@H](NC(=O)OCc1ccccc1)C1CCCCC1)C21CCCCC1)C(=O)C(=O)NC1CC1] | Benzyl ((S)-1-cyclohexyl-2- ((1R,2S,5S)-2-(((S)-1- (cyclopropylamino)-6,6- difluoro-1,2-dioxoheptan-3- yl)carbamoyl)-3- azaspiro[bicyclo[3.1.0]hexan e-6,1'-cyclohexan]-3-yl)-2- oxoethyl)carbamate | Calculated: 685.4 Found: 685.3 |
| 67 | 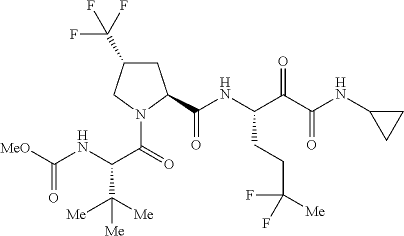 [SMILES: COC(=O)N[C@H](C(=O)N1C[C@H](C(F)(F)F)C[C@H]1C(=O)N[C@@H](CCC(C)(F)F)C(=O)C(=O)NC1CC1)C(C)(C)C] | Methyl ((S)-1-((2S,4R)-2- (((S)-1-(cyclopropylamino)- 6,6-difluoro-1,2-dioxoheptan- 3-yl)carbamoyl)-4- (trifluoromethyl)pyrrolidin-1- yl)-3,3-dimethyl-1-oxobutan- 2-yl)carbamate | Calculated: 571.3 Found: 571.4 |
| 68 | 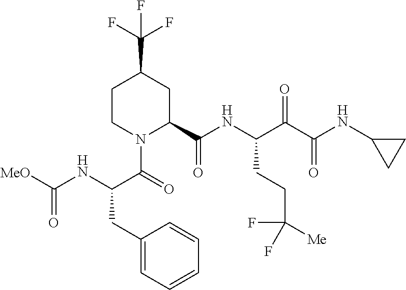 [SMILES: COC(=O)N[C@@H](Cc1ccccc1)C(=O)N1CC[C@@H](C(F)(F)F)C[C@H]1C(=O)N[C@@H](CCC(C)(F)F)C(=O)C(=O)NC1CC1] | Methyl ((S)-1-((2S,4R)-2- (((S)-1-(cyclopropylamino)- 6,6-difluoro-1,2-dioxoheptan- 3-yl)carbamoyl)-4- (trifluoromethyl)piperidin-1- yl)-1-oxo-3-phenylpropan-2- yl)carbamate | Calculated: 619.3 Found: 619.4 |
| 69 | 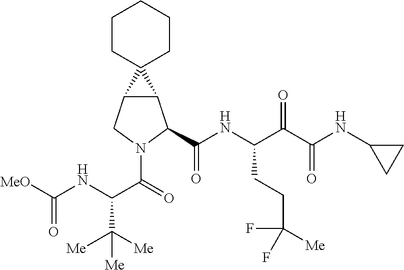 [SMILES: COC(=O)N[C@H](C(=O)N1C[C@H]2[C@@H]([C@H]1C(=O)N[C@@H](CCC(C)(F)F)C(=O)C(=O)NC1CC1)C21CCCCC1)C(C)(C)C] | Methyl ((S)-1-((1R,2S,5S)-2- (((S)-1-(cyclopropylamino)- 6,6-difluoro-1,2-dioxoheptan- 3-yl)carbamoyl)-3- azaspiro[bicyclo[3.1.0]hexan e-6,1'-cyclohexan]-3-yl)-3,3- dimethyl-1-oxobutan-2- yl)carbamate | Calculated: 583.3 Found: 583.3 |
| 70 | 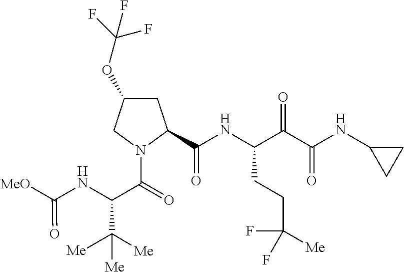 [SMILES: COC(=O)N[C@H](C(=O)N1C[C@H](OC(F)(F)F)C[C@H]1C(=O)N[C@@H](CCC(C)(F)F)C(=O)C(=O)NC1CC1)C(C)(C)C] | Methyl ((S)-1-((2S,4R)-2- (((S)-1-(cyclopropylamino)- 6,6-difluoro-1,2-dioxoheptan- 3-yl)carbamoyl)-4- (trifluoromethoxy)pyrrolidin- 1-yl)-3,3-dimethyl-1- oxobutan-2-yl)carbamate | Calculated: 587.3 Found: 587.4 |
| 71 | 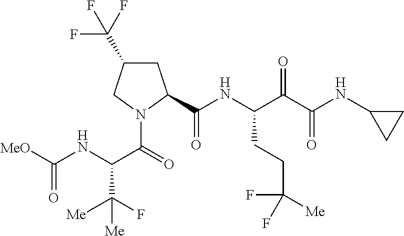 [SMILES: COC(=O)N[C@H](C(=O)N1C[C@H](C(F)(F)F)C[C@H]1C(=O)N[C@@H](CCC(C)(F)F)C(=O)C(=O)NC1CC1)C(C)(C)F] | Methyl ((R)-1-((2S,4R)-2- (((S)-1-(cyclopropylamino)- 6,6-difluoro-1,2-dioxoheptan- 3-yl)carbamoyl)-4- (trifluoromethyl)pyrrolidin-1- yl)-3-fluoro-3-methyl-1- oxobutan-2-yl)carbamate | Calculated: 575.2 Found: 575.3 |
| 72 | 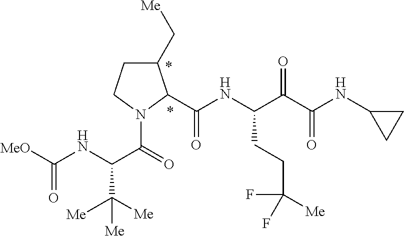 [SMILES: CCC1CCN(C(=O)[C@@H](NC(=O)OC)C(C)(C)C)C1C(=O)N[C@@H](CCC(C)(F)F)C(=O)C(=O)NC1CC1] | Methyl ((S)-1-((2(R or S),3(R or S))-2-(((S)-1- (cyclopropylamino)-6,6- difluoro-1,2-dioxoheptan-3- yl)carbamoyl)-3- ethylpyrrolidin-1-yl)-3,3- dimethyl-1-oxobutan-2- yl)carbamate | Calculated: 531.3 Found: 531.5 |
| 73 | 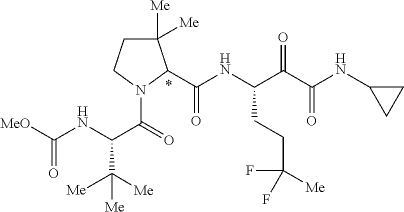 [SMILES: COC(=O)N[C@H](C(=O)N1CCC(C)(C)C1C(=O)N[C@@H](CCC(C)(F)F)C(=O)C(=O)NC1CC1)C(C)(C)C] | Methyl ((S)-1-((R or S)-2- (((S)-1-(cyclopropylamino)- 6,6-difluoro-1,2-dioxoheptan- 3-yl)carbamoyl)-3,3- dimethylpyrrolidin-1-yl)-3,3- dimethyl-1-oxobutan-2- yl)carbamate | Calculated: 531.3 Found: 531.4 |
| 74 | 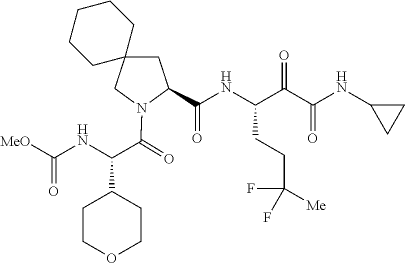 [SMILES: COC(=O)N[C@H](C(=O)N1CC2(CCCCC2)C[C@H]1C(=O)N[C@@H](CCC(C)(F)F)C(=O)C(=O)NC1CC1)C1CCOCC1] | Methyl ((S)-2-((S)-3-(((S)-1- (cyclopropylamino)-6,6- difluoro-1,2-dioxoheptan-3- yl)carbamoyl)-2- azaspiro[4.5]decan-2-yl)-2- ox0-1-(tetrahydro-2H-pyran- 4-yl)ethyl)carbamate | Calculated: 599.3 Found: 599.3 |
| 75 | 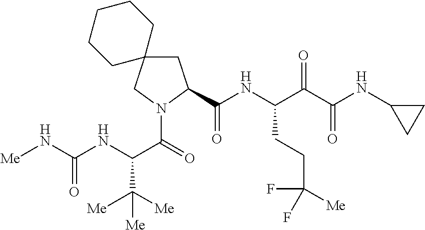 [SMILES: CNC(=O)N[C@H](C(=O)N1CC2(CCCCC2)C[C@H]1C(=O)N[C@@H](CCC(C)(F)F)C(=O)C(=O)NC1CC1)C(C)(C)C] | (S)-N-((S)-1- (cyclopropylamino)-6,6- difluoro-1,2-dioxoheptan-3- yl)-2-((S)-3,3-dimethyl-2-(3- methylureido)butanoyl)-2- azaspiro[4.5]decane-3- carboxamide | Calculated: 570.4 Found: 570.3 |
| 76 | 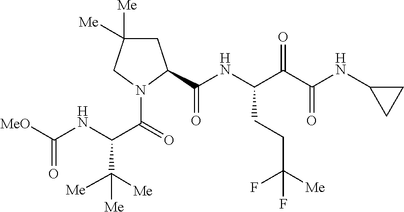 [SMILES: COC(=O)N[C@H](C(=O)N1CC(C)(C)C[C@H]1C(=O)N[C@@H](CCC(C)(F)F)C(=O)C(=O)NC1CC1)C(C)(C)C] | Methyl ((S)-1-((S)-2-(((S)-1- (cyclopropylamino)-6,6- difluoro-1,2-dioxoheptan-3- yl)carbamoyl)-4,4- dimethylpyrrolidin-1-yl)-3,3- dimethyl-1-oxobutan-2- yl)carbamate | Calculated: 531.3 Found: 531.5 |
| 77 | 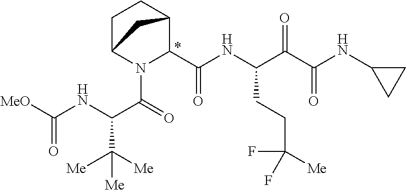 [SMILES: COC(=O)N[C@H](C(=O)N1C(C(=O)N[C@@H](CCC(C)(F)F)C(=O)C(=O)NC2CC2)[C@H]2CC[C@@H]1C2)C(C)(C)C] | Methyl ((S)-1-((1R, 3(R or S),4S)-3-(((S)-1- (cyclopropylamino)-6,6- difluoro-1,2-dioxoheptan-3- yl)carbamoyl)-2- azabicyclo[2.2.1]heptan-2- yl)-3,3-dimethyl-1-oxobutan- 2-yl)carbamate | Calculated: 529.3 Found: 529.6 |

### From Scheme F

### Example 78

### Methyl ((S)-1-((S)-3-(((S)-1-amino-6,6-difluoro-1,2-dioxoheptan-3-yl)carbamoyl)-2-azaspiro[4.5]decan-2-yl)-3,3-dimethyl-1-oxobutan-2-yl)carbamate

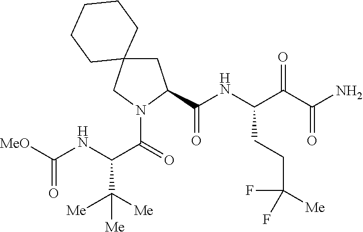 [SMILES: COC(=O)N[C@H](C(=O)N1CC2(CCCCC2)C[C@H]1C(=O)N[C@@H](CCC(C)(F)F)C(=O)C(N)=O)C(C)(C)C]

### Step 1: Methyl (S)-2-((S)-2-((methoxycarbonyl)amino)-3,3-dimethylbutanoyl)-2-azaspiro[4.5]decane-3-carboxylate

To a solution of (S)-2-((methoxycarbonyl)amino)-3,3-dimethylbutanoic acid (300 mg, 1.6 mmol) and methyl (S)-2-azaspiro[4.5]decane-3-carboxylate (313 mg, 1.59 mmol) in DMF (5 mL) were added EDCl (456 mg, 2.38 mmol), HOBt (300 mg, 2.2 mmol) and DIEA (0.692 mL, 3.96 mmol) at 25° C. The mixture was stirred at 25° C. for 16 hours. Water (1 mL) was added to the mixture and the mixture was purified by RP-HPLC (Boston Green ODS 5 μm 150×30 mm) eluting with a gradient of 55-75% acetonitrile/water+0.1% TFA over 10 minutes at 25 mL/min to give title compound. LRMS m/z: (M+H)⁺ calculated 369.3; found 369.5.

### Step 2: (S)-2-((S)-2-((Methoxycarbonyl)amino)-3,3-dimethylbutanoyl)-2-azaspiro[4.5]decane-3-carboxylic acid

To a solution of methyl (S)-2-((S)-2-((methoxycarbonyl)amino)-3,3-dimethylbutanoyl)-2-azaspiro[4.5]decane-3-carboxylate (340 mg, 0.92 mmol) in THF (0.6 mL) and H₂O (0.2 mL) was added lithium hydroxide (110 mg, 4.6 mmol) at 25° C. The mixture was stirred at 25° C. for 4 hours. The solvent was removed under reduced pressure. HCl (0.5 M) was added to the mixture to adjust the pH to 5. Water (20 mL) was added and the mixture was extracted with DCM (3×30 mL). The combined organic fractions were washed with brine (20 mL), dried (Na₂SO₄), filtered and concentrated under reduced pressure to give the title compound. LRMS m/z: (M+H)⁺ calculated 355.2; found 355.1.

### Step 3: Methyl ((S)-1-((S)-3-(((S)-5,5-difluoro-1,1-dimethoxyhexan-2-yl)carbamoyl)-2-azaspiro[4.5]decan-2-yl)-3,3-dimethyl-1-oxobutan-2-yl)carbamate

To a solution of (S)-2-((S)-2-((methoxycarbonyl)amino)-3,3-dimethylbutanoyl)-2-azaspiro[4.5]decane-3-carboxylic acid (110 mg, 0.31 mmol), (S)-5,5-difluoro-1,1-dimethoxyhexan-2-amine (122 mg, 0.621 mmol) and DIEA (0.163 mL, 0.931 mmol) in DMF (0.5 mL) was added AOP (165 mg, 0.372 mmol) at 25° C. The mixture was stirred at 25° C. for 16 hours. Water (1 mL) was added to the mixture and the mixture was purified by RP-HPLC (Boston Green ODS 5 μm 150×25 mm) eluting with a gradient of 45-75% acetonitrile/Water+10 mM NH₄HCO₃ over 10 minutes to give the title compound. LRMS m/z: (M+H-32)⁺ calculated 502.3; found 502.4.

### Step 4: Methyl ((S)-1-((S)-3-(((S)-5,5-difluoro-1-oxohexan-2-yl)carbamoyl)-2-azaspiro[4.5]decan-2-yl)-3,3-dimethyl-1-oxobutan-2-yl)carbamate

To a solution of methyl ((S)-1-((S)-3-(((S)-5,5-difluoro-1,1-dimethoxyhexan-2-yl)carbamoyl)-2-azaspiro[4.5]decan-2-yl)-3,3-dimethyl-1-oxobutan-2-yl)carbamate (100 mg, 0.19 mmol) in acetone (5 mL) and water (5 mL) was added Dowex® 50WX8 (1 g). The resulting mixture was stirred at 40° C. for 16 hours. The resin was filtered off and the filter cake was washed with 1:1 acetone:water (4×4 mL). Acetone was removed with a stream of N₂ and the aqueous portion was lyophilized to give the title compound. LRMS m/z: (M+H)⁺ calculated 488.3; found 488.4.

### Step 5: Methyl ((2S)-1-((3S)-3-(((2S)-1-cyano-5,5-difluoro-1-hydroxyhexan-2-yl)carbamoyl)-2-azaspiro[4.5]decan-2-yl)-3,3-dimethyl-1-oxobutan-2-yl)carbamate

To a solution of methyl ((S)-1-((S)-3-(((S)-5,5-difluoro-1-oxohexan-2-yl)carbamoyl)-2-azaspiro[4.5]decan-2-yl)-3,3-dimethyl-1-oxobutan-2-yl)carbamate (60 mg, 0.12 mmol) and CsF (18.7 mg, 0.123 mmol) in MeOH (1 mL) was added dropwise trimethylsilanecarbonitrile (0.040 mL, 0.30 mmol) at 0° C. under an atmosphere of nitrogen. The mixture was stirred at 25° C. for 16 hours. The solvent was removed under reduced pressure to give the title compound. LRMS m/z: (M+H)⁺ calculated 515.3; found 515.2.

### Step 6: Methyl ((2S)-1-((3S)-3-(((3S)-1-amino-6,6-difluoro-2-hydroxy-1-oxoheptan-3-yl)carbamoyl)-2-azaspiro[4.5]decan-2-yl)-3,3-dimethyl-1-oxobutan-2-yl)carbamate

To a solution of methyl ((2S)-1-((3S)-3-(((2S)-1-cyano-5,5-difluoro-1-hydroxyhexan-2-yl)carbamoyl)-2-azaspiro[4.5]decan-2-yl)-3,3-dimethyl-1-oxobutan-2-yl)carbamate (60 mg, 0.12 mmol) in DMSO (3 mL) were added K₂CO₃ (27.4 mg, 0.198 mmol) and H₂O₂ (0.119 mL, 1.17 mmol) at 0° C. under an atmosphere of nitrogen. The mixture was stirred for 16 hours at 25° C. Na₂SO₃ (60 mg) was added and the mixture was stirred at 25° C. for 15 minutes. The mixture was filtered and the filtrate was purified by RP-HPLC (Waters Xbridge BEH C18 5 μm 100×25 mm) eluting with a gradient of 25-45% acetonitrile/water+0.10% TFA over 12 minutes at 25 mL/min to give the title compound as the second-eluting peak. LRMS m/z: (M+H)⁺ calculated 533.3; found 533.3.

### Step 7: Methyl ((S)-1-((S)-3-(((S)-1-amino-6,6-difluoro-1,2-dioxoheptan-3-yl)carbamoyl)-2-azaspiro[4.5]decan-2-yl)-3,3-dimethyl-1-oxobutan-2-yl)carbamate

To a solution of methyl ((2S)-1-((3S)-3-(((3S)-1-amino-6,6-difluoro-2-hydroxy-1-oxoheptan-3-yl)carbamoyl)-2-azaspiro[4.5]decan-2-yl)-3,3-dimethyl-1-oxobutan-2-yl)carbamate (30 mg, 0.056 mmol) in DCM (5 mL) was added NaHCO₃ (14.2 mg, 0.169 mmol) and DMP (71.7 mg, 0.169 mmol). The mixture was stirred for 1 hour at 25° C. The mixture was filtered and the filtrate was purified by RP-HPLC (Welch Xtimate C18 5 μm 150×25 mm) eluting with a 35-65% acetonitrile/water+10 mM NH₄HCO₃ over 11 minutes to give the title compound. LRMS m/z: (M+H)⁺ calculated 531.3; found 531.2. ¹H NMR (500 MHz, CD₃OD) δ 4.58 (s, 2H), 4.48-3.90 (m, 3H), 3.64 (s, 3H), 2.25-2.06 (m, 2H), 2.05-1.63 (m, 3H), 1.62-1.23 (m, 14H), 1.02 (d, J=4.0 Hz, 9H).

### From Scheme E

### Example 79

### Methyl ((S)-1-((S)-3-(((S)-6,6-difluoro-1,2-dioxo-1-((pyridin-4-ylmethyl)amino)heptan-3-yl)carbamoyl)-2-azaspiro[4.5]decan-2-yl)-3,3-dimethyl-1-oxobutan-2-yl)carbamate

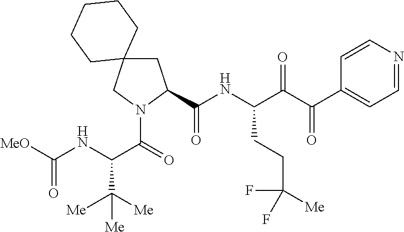 [SMILES: COC(=O)N[C@H](C(=O)N1CC2(CCCCC2)C[C@H]1C(=O)N[C@@H](CCC(C)(F)F)C(=O)C(=O)c1ccncc1)C(C)(C)C]

### Step 1: Methyl (3S)-6,6-difluoro-2-hydroxy-3-((S)-2-((S)-2-((methoxycarbonyl)amino)-3,3-dimethylbutanoyl)-2-azaspiro[4.5]decane-3-carboxamido)heptanoate

To a solution of methyl ((2S)-1-((3S)-3-(((2S)-1-cyano-5,5-difluoro-1-hydroxyhexan-2-yl)carbamoyl)-2-azaspiro[4.5]decan-2-yl)-3,3-dimethyl-1-oxobutan-2-yl)carbamate (300 mg, 0.58 mmol) in DCM (2 mL) was added 4 M HCl in MeOH (2 mL) at 25° C. The resulting mixture was stirred at 60° C. for 15 hours and then the solvent was removed under reduced pressure. To the residue was added THF (2 mL) followed by water (2 mL). The mixture was stirred at 25° C. for 1 hour. The THF was removed under reduced pressure. The mixture was filtered and the filtrate was purified by RP-HPLC (Boston Green ODS 5 μm 150×30 mm) eluting with 47-67% acetonitrile/water+0.10% TFA over 10 minutes to give the title compound. LRMS m/z: (M+H)⁺ calculated 548.3; found 548.2.

### Step 2: (3S)-6,6-Difluoro-2-hydroxy-3-((S)-2-((S)-2-((methoxycarbonyl)amino)-3,3-dimethylbutanoyl)-2-azaspiro[4.5]decane-3-carboxamido)heptanoic acid

To a solution of methyl (3S)-6,6-difluoro-2-hydroxy-3-((S)-2-((S)-2-((methoxycarbonyl)amino)-3,3-dimethylbutanoyl)-2-azaspiro[4.5]decane-3-carboxamido)heptanoate (55 mg, 0.10 mmol) in THF (0.3 mL) and H₂O (0.1 mL) was added lithium hydroxide (12.0 mg, 0.502 mmol) at 25° C. The mixture was stirred at 25° C. for about 16 h. The mixture was concentrated under reduced pressure. HCl (0.5 M) was added to adjust the pH of the mixture to 5. Water (10 mL) was added and the mixture was extracted with DCM (3×10 mL). The combined organic fractions were washed with brine (10 mL), dried (Na₂SO₄), filtered, and concentrated under reduced pressure to give the title compound. LRMS m/z: (M+H)⁺ calculated 534.3; found 534.3.

### Step 3: Methyl ((2S)-1-((3S)-3-(((3S)-6,6-difluoro-2-hydroxy-1-oxo-1-((pyridin-4-ylmethyl)amino)heptan-3-yl)carbamoyl)-2-azaspiro[4.5]decan-2-yl)-3,3-dimethyl-1-oxobutan-2-yl)carbamate

To a solution of (3S)-6,6-difluoro-2-hydroxy-3-((S)-2-((S)-2-((methoxycarbonyl)amino)-3,3-dimethylbutanoyl)-2-azaspiro[4.5]decane-3-carboxamido)heptanoic acid (50 mg, 0.094 mmol), pyridin-4-ylmethanamine (10 mg, 0.094 mmol) and DIEA (0.049 mL, 0.28 mmol) in DMF (0.5 mL) was added AOP (49.8 mg, 0.112 mmol) at 25° C. The mixture was stirred at 25° C. for 16 hours. The mixture was purified directly by RP-HPLC (Boston Green ODS 5 μm 150×30 mm) eluting with a gradient of 35-55% acetonitrile/water+0.10% TFA over 10 minutes at 25 mL/min to give the title compound. LRMS m/z: (M+H)⁺ calculated 624.4; found 624.3.

### Step 4: Methyl ((S)-1-((S)-3-(((S)-6,6-difluoro-1,2-dioxo-1-((pyridin-4-ylmethyl)amino)heptan-3-yl)carbamoyl)-2-azaspiro[4.5]decan-2-yl)-3,3-dimethyl-1-oxobutan-2-yl)carbamate

To a solution of methyl ((2S)-1-((3S)-3-(((3S)-6,6-difluoro-2-hydroxy-1-oxo-1-((pyridin-4-ylmethyl)amino)heptan-3-yl)carbamoyl)-2-azaspiro[4.5]decan-2-yl)-3,3-dimethyl-1-oxobutan-2-yl)carbamate (35 mg, 0.056 mmol) in DCM (3 mL) was added NaHCO₃ (14.1 mg, 0.168 mmol) and DMP (71.4 mg, 0.168 mmol). Na₂SO₃ (60 mg) was added and the mixture was stirred at 25° C. for 15 min. The mixture was filtered and the filtrate was concentrated under reduced pressure. The residue was purified by RP-HPLC (Welch Xtimate C18 5 μm 150×25 mm) eluting with a gradient of 33-63% acetonitrile/water+10 mM NH₄HCO₃ over 11 minutes at 25 mL/min to give the title compound. LRMS m/z: (M+H)⁺ calculated 622.3; found 622.4. ¹H NMR (400 MHz, CD₃OD) δ 8.54-8.40 (m, 2H), 7.45-7.27 (m, 2H), 4.54-4.26 (m, 4H), 4.25-3.90 (m, 2H), 3.63 (s, 3H), 3.29-3.21 (m, 1H), 2.22-1.71 (m, 4H), 1.62-1.28 (m, 15H), 1.09-0.97 (m, 9H).

### ADDITIONAL EXAMPLES

Additional examples were prepared in accordance to the schemes described above.

| Ex. | | | | |
|---|---|---|---|---|
| No. | Scheme | Structure | Name | LRMS m/z |
| 80 | B | 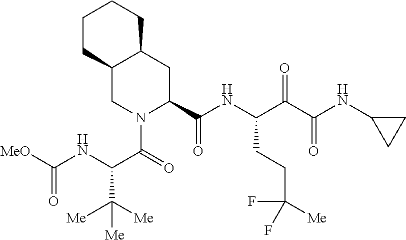 [SMILES: COC(=O)N[C@H](C(=O)N1C[C@H]2CCCC[C@H]2C[C@H]1C(=O)N[C@@H](CCC(C)(F)F)C(=O)C(=O)NC1CC1)C(C)(C)C] | methyl ((S)-1- ((3S,4aS,8aS)-3-(((S)-1- (cyclopropylamino)-6,6- difluoro-1,2-dioxoheptan-3- yl)carbamoyl)octahydroisoq uinolin-2(1H)-yl)-3,3- dimethyl-1-oxobutan-2- yl)carbamate | Calculated: 570.3 Found: 571.3 (M + H)⁺ |
| 81 | B | 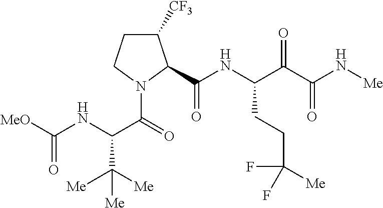 [SMILES: CNC(=O)C(=O)[C@H](CCC(C)(F)F)NC(=O)[C@@H]1[C@@H](C(F)(F)F)CCN1C(=O)[C@@H](NC(=O)OC)C(C)(C)C] | methyl ((S)-1-((2S,3S)-2- (((S)-6,6-difluoro-1- (methylamino)-1,2- dioxoheptan-3- yl)carbamoyl)-3- (trifluoromethyl)pyrrolidin- 1-yl)-3,3-dimethyl-1- oxobutan-2-yl)carbamate | Calculated: 544.2 Found: 545.3 (M + H)⁺ |
| 82 | B | 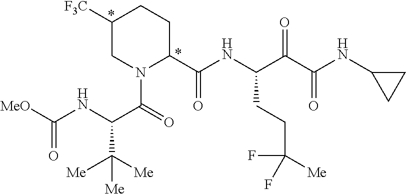 [SMILES: COC(=O)N[C@H](C(=O)N1CC(C(F)(F)F)CCC1C(=O)N[C@@H](CCC(C)(F)F)C(=O)C(=O)NC1CC1)C(C)(C)C] | methyl ((S)-1-((2S,5S or 2R,5R)-2-(((S)-1- (cyclopropylamino)-6,6- difluoro-1,2-dioxoheptan-3- yl)carbamoyl)-5- (trifluoromethyl)piperidin- 1-yl)-3,3-dimethyl-1- oxobutan-2-yl)carbamate | Calculated: 584.3 Found: 585.5 (M + H)⁺ |
| 83 | B | 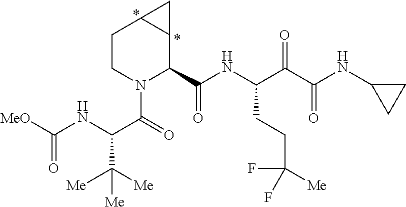 [SMILES: COC(=O)N[C@H](C(=O)N1CCC2CC2[C@H]1C(=O)N[C@@H](CCC(C)(F)F)C(=O)C(=O)NC1CC1)C(C)(C)C] | methyl ((S)-1-((1R,2S,6R or 1R,2S,6S)-2-(((S)-1- (cyclopropylamino)-6,6- difluoro-1,2-dioxoheptan-3- yl)carbamoyl)-3- azabicyclo[4.1.0]heptan-3- yl)-3,3-dimethyl-1- oxobutan-2-yl)carbamate | Calculated: 528.3 Found: 529.4 (M + H)⁺ |
| 84 | B | 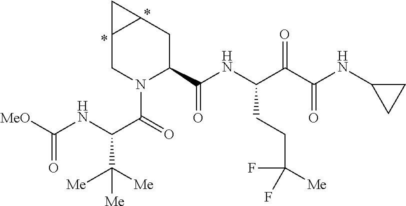 [SMILES: COC(=O)N[C@H](C(=O)N1CC2CC2C[C@H]1C(=O)N[C@@H](CCC(C)(F)F)C(=O)C(=O)NC1CC1)C(C)(C)C] | methyl ((S)-1-((1S,4S,6S or 1R,4S,6R)-4-(((S)-1- (cyclopropylamino)-6,6- difluoro-1,2-dioxoheptan-3- yl)carbamoyl)-3- azabicyclo[4.1.0]heptan-3- yl)-3,3-dimethyl-1- oxobutan-2-yl)carbamate | Calculated: 528.3 Found: 529.4 (M + H)⁺ |
| 85 | B | 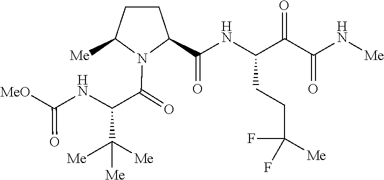 [SMILES: CNC(=O)C(=O)[C@H](CCC(C)(F)F)NC(=O)[C@@H]1CC[C@H](C)N1C(=O)[C@@H](NC(=O)OC)C(C)(C)C] | methyl ((S)-1-((2S,5S)-2- (((S)-6,6-difluoro-1- (methylamino)-1,2- dioxoheptan-3- yl)carbamoyl)-5- methylpyrrolidin-1-yl)-3,3- dimethyl-1-oxobutan-2- yl)carbamate | Calculated: 490.3 Found: 491.3 (M + H)⁺ |
| 86 | A | 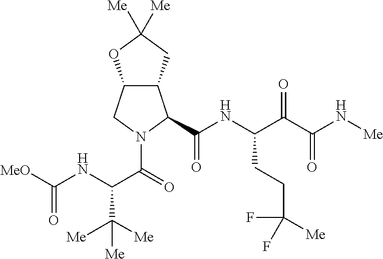 [SMILES: CNC(=O)C(=O)[C@H](CCC(C)(F)F)NC(=O)[C@@H]1[C@H]2CC(C)(C)O[C@H]2CN1C(=O)[C@@H](NC(=O)OC)C(C)(C)C] | methyl ((S)-1- ((3aR,4S,6aR)-4-(((S)-6,6- difluoro-1-(methylamino)- 1,2-dioxoheptan-3- yl)carbamoyl)-2,2- dimethylhexahydro-5H- furo[2,3-c]pyrrol-5-yl)-3,3- dimethyl-1-oxobutan-2- yl)carbamate | Calculated: 546.3 Found: 547.5 (M + H)⁺ |
| 87 | A | 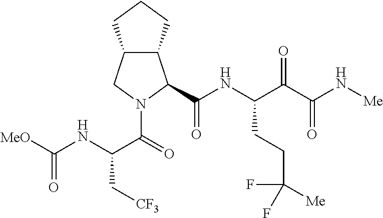 [SMILES: CNC(=O)C(=O)[C@H](CCC(C)(F)F)NC(=O)[C@@H]1[C@H]2CCC[C@H]2CN1C(=O)[C@H](CC(F)(F)F)NC(=O)OC] | methyl ((S)-1- ((1S,3aR,6aS)-1-(((S)-6,6- difluoro-1-(methylamino)- 1,2-dioxoheptan-3- yl)carbamoyl)hexahydrocyc lopenta[c]pyrrol-2(1H)-yl)- 4,4,4-trifluoro-1-oxobutan- 2-yl)carbamate | Calculated: 542.2 Found: 543.4 (M + H)⁺ |
| 88 | A | 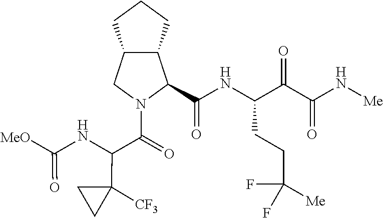 [SMILES: CNC(=O)C(=O)[C@H](CCC(C)(F)F)NC(=O)[C@@H]1[C@H]2CCC[C@H]2CN1C(=O)C(NC(=O)OC)C1(C(F)(F)F)CC1] | methyl (2-((1S,3aR,6aS)-1- (((S)-6,6-difluoro-1- (methylamino)-1,2- dioxoheptan-3- yl)carbamoyl)hexahydrocyc lopenta[c]pyrrol-2(1H)-yl)- 2-oxo-1-(1- (trifluoromethyl)cyclopropy 1)ethyl)carbamate | Calculated: 568.2 Found: 569.5 (M + H)⁺ |
| 89 | A | 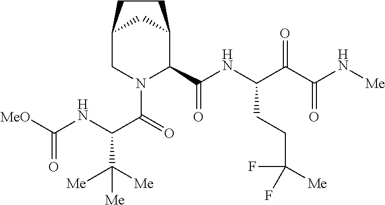 [SMILES: CNC(=O)C(=O)[C@H](CCC(C)(F)F)NC(=O)[C@@H]1[C@@H]2CC[C@@H](C2)CN1C(=O)[C@@H](NC(=O)OC)C(C)(C)C] | methyl ((S)-1-((1R,2S,5S)- 2-(((S)-6,6-difluoro-1- (methylamino)-1,2- dioxoheptan-3- yl)carbamoyl)-3- azabicyclo[3.2.1]octan-3- yl)-3,3-dimethyl-1- oxobutan-2-yl)carbamate | Calculated: 516.3 Found: 517.4 (M + H)⁺ |
| 90 | A | 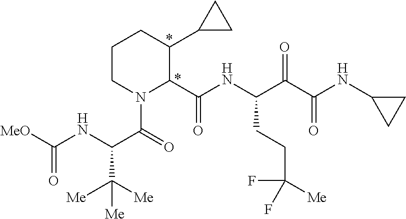 [SMILES: COC(=O)N[C@H](C(=O)N1CCCC(C2CC2)C1C(=O)N[C@@H](CCC(C)(F)F)C(=O)C(=O)NC1CC1)C(C)(C)C] | methyl ((S)-1-((2S,3R or 2R,3S)-3-cyclopropyl-2- (((S)-1-(cyclopropylamino)- 6,6-difluoro-1,2- dioxoheptan-3- yl)carbamoyl)piperidin-1- yl)-3,3-dimethyl-1- oxobutan-2-yl)carbamate | Calculated: 556.3 Found: 557.3 (M + H)⁺ |
| 91 | B | 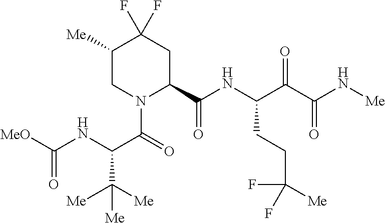 [SMILES: CNC(=O)C(=O)[C@H](CCC(C)(F)F)NC(=O)[C@@H]1CC(F)(F)[C@@H](C)CN1C(=O)[C@@H](NC(=O)OC)C(C)(C)C] | methyl ((S)-1-((2S,5S)-2- (((S)-6,6-difluoro-1- (methylamino)-1,2- dioxoheptan-3- yl)carbamoyl)-4,4-difluoro- 5-methylpiperidin-1-yl)-3,3- dimethyl-1-oxobutan-2- yl)carbamate | Calculated: 540.3 Found: 541.2 (M + H)⁺ |
| 92 | B | 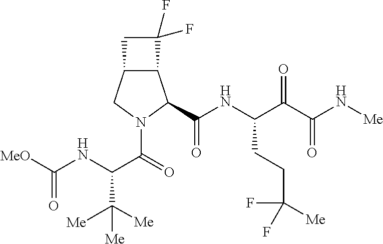 [SMILES: CNC(=O)C(=O)[C@H](CCC(C)(F)F)NC(=O)[C@@H]1[C@@H]2[C@H](CN1C(=O)[C@@H](NC(=O)OC)C(C)(C)C)CC2(F)F] | methyl ((S)-1-((1S,2S,5R)- 2-(((S)-6,6-difluoro-1- (methylamino)-1,2- dioxoheptan-3- yl)carbamoyl)-7,7-difluoro- 3-azabicyclo[3.2.0]heptan- 3-yl)-3,3-dimethyl-1- oxobutan-2-yl)carbamate | Calculated: 538.2 Found: 539.2 (M + H)⁺ |
| 93 | A | 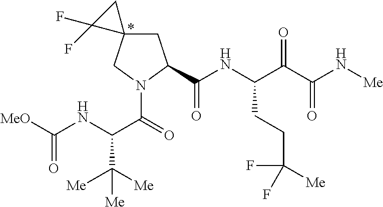 [SMILES: CNC(=O)C(=O)[C@H](CCC(C)(F)F)NC(=O)[C@@H]1CC2(CN1C(=O)[C@@H](NC(=O)OC)C(C)(C)C)CC2(F)F] | methyl ((S)-1-((3(S or R),6S)-6-(((S)-6,6-difluoro- 1-(methylamino)-1,2- dioxoheptan-3- yl)carbamoyl)-1,1-difluoro- 5-azaspiro[2.4]heptan-5-yl)- 3,3-dimethyl-1-oxobutan-2- yl)carbamate | Calculated: 538.2 Found: 539.4 (M + H)⁺ |
| 94 | B | 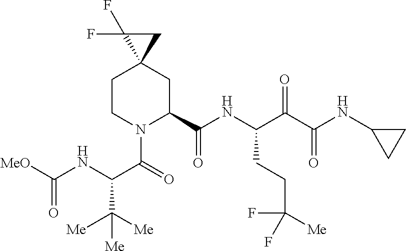 [SMILES: COC(=O)N[C@H](C(=O)N1CC[C@]2(C[C@H]1C(=O)N[C@@H](CCC(C)(F)F)C(=O)C(=O)NC1CC1)CC2(F)F)C(C)(C)C] | methyl ((S)-1-((3S,5S)-5- (((S)-1-(cyclopropylamino)- 6,6-difluoro-1,2- dioxoheptan-3- yl)carbamoyl)-1,1-difluoro- 6-azaspiro[2.5]octan-6-yl)- 3,3-dimethyl-1-oxobutan-2- yl)carbamate | Calculated: 578.3 Found: 579.3 (M + H)⁺ |
| 95 | A | 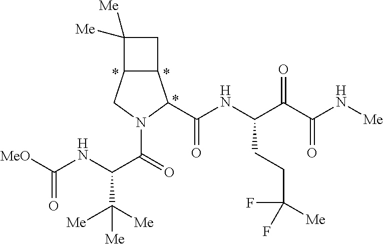 [SMILES: CNC(=O)C(=O)[C@H](CCC(C)(F)F)NC(=O)C1C2CC(C)(C)C2CN1C(=O)[C@@H](NC(=O)OC)C(C)(C)C] | methyl ((S)-1-((1S,2S,5S or 1R,2R,5R)-2-(((S)-6,6- difluoro-1-(methylamino)- 1,2-dioxoheptan-3- yl)carbamoyl)-6,6-dimethyl- 3-azabicyclo[3.2.0]heptan- 3-yl)-3,3-dimethyl-1- oxobutan-2-yl)carbamate | Calculated: 530.3 Found: 531.5 (M + H)⁺ |
| 96 | A | 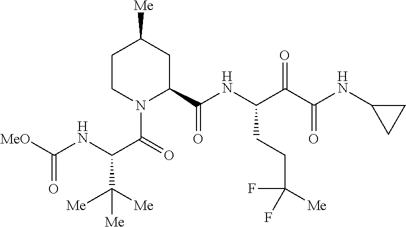 [SMILES: COC(=O)N[C@H](C(=O)N1CC[C@@H](C)C[C@H]1C(=O)N[C@@H](CCC(C)(F)F)C(=O)C(=O)NC1CC1)C(C)(C)C] | methyl ((S)-1-((2S,4R)-2- (((S)-1-(cyclopropylamino)- 6,6-difluoro-1,2- dioxoheptan-3- yl)carbamoyl)-4- methylpiperidin-1-yl)-3,3- dimethyl-1-oxobutan-2- yl)carbamate | Calculated: 530.3 Found: 531.5 (M + H)⁺ |
| 97 | B | 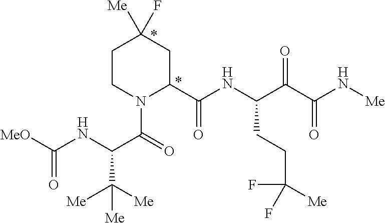 [SMILES: CNC(=O)C(=O)[C@H](CCC(C)(F)F)NC(=O)C1CC(C)(F)CCN1C(=O)[C@@H](NC(=O)OC)C(C)(C)C] | methyl ((S)-1-((2S,4S or 2R,4R)-2-(((S)-6,6-difluoro- 1 -(methylamino)-1,2- dioxoheptan-3- yl)carbamoyl)-4-fluoro-4- methylpiperidin-1-yl)-3,3- dimethyl-1-oxobutan-2- yl)carbamate | Calculated: 522.3 Found: 523.5 (M + H)⁺ |
| 98 | A | 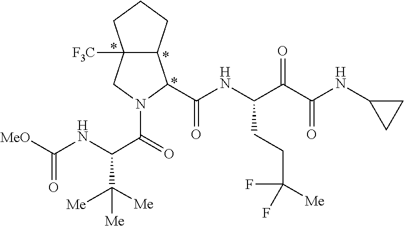 [SMILES: COC(=O)N[C@H](C(=O)N1CC2(C(F)(F)F)CCCC2C1C(=O)N[C@@H](CCC(C)(F)F)C(=O)C(=O)NC1CC1)C(C)(C)C] | methyl ((S)-1-((1S,3aS,6aS or 1R,3aR,6aR)-1-(((S)-1- (cyclopropylamino)-6,6- difluoro-1,2-dioxoheptan-3- yl)carbamoyl)-3a- (trifluoromethyl)hexahydroc yclopenta[c]pyrrol-2(1H)- yl)-3,3-dimethyl-1- oxobutan-2-yl)carbamate | Calculated: 610.3 Found: 611.5 (M + H)⁺ |
| 99 | B | 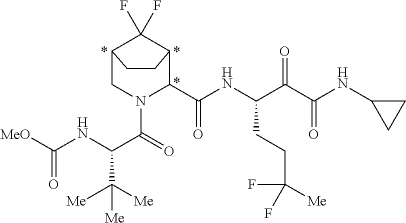 [SMILES: COC(=O)N[C@H](C(=O)N1CC2CCC(C1C(=O)N[C@@H](CCC(C)(F)F)C(=O)C(=O)NC1CC1)C2(F)F)C(C)(C)C] | methyl ((S)-1-((1(S or R),2(S or R),5(R or S))-2- (((S)-1-(cyclopropylamino)- 6,6-difluoro-1,2- dioxoheptan-3- yl)carbamoyl)-8,8-difluoro- 3-azabicyclo[3.2.1]octan-3- yl)-3,3-dimethyl-1- oxobutan-2-yl)carbamate | Calculated: 578.3 Found: 579.3 (M + H)⁺ |
| 100 | A | 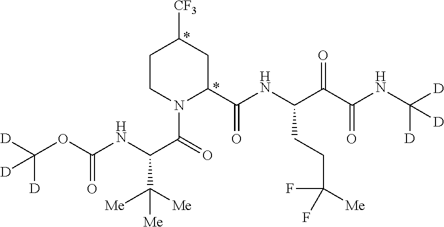 [SMILES: [2H]C([2H])([2H])NC(=O)C(=O)[C@H](CCC(C)(F)F)NC(=O)C1CC(C(F)(F)F)CCN1C(=O)[C@@H](NC(=O)OC([2H])([2H])[2H])C(C)(C)C] | methyl-d3 ((S)-1-((2(S or R),4(S or R))-2-(((S)-6,6- difluoro-1-((methyl- d3)amino)-1,2-dioxoheptan- 3-yl)carbamoyl)-4- (trifluoromethyl)piperidin- 1-yl)-3,3-dimethyl-1- oxobutan-2-yl)carbamate | Calculated: 564.3 Found: 587.3 (M + Na)⁺ |
| 101 | A | 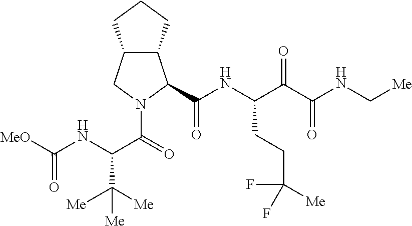 [SMILES: CCNC(=O)C(=O)[C@H](CCC(C)(F)F)NC(=O)[C@@H]1[C@H]2CCC[C@H]2CN1C(=O)[C@@H](NC(=O)OC)C(C)(C)C] | methyl ((S)-1- ((1S,3aR,6aS)-1-(((S)-1- (ethylamino)-6,6-difluoro- 1,2-dioxoheptan-3- yl)carbamoyl)hexahydrocyc lopenta[c]pyrrol-2(1H)-yl)- 3,3-dimethyl-1-oxobutan-2- yl)carbamate | Calculated: 530.3 Found: 531.4 (M + H)⁺ |
| 102 | A | 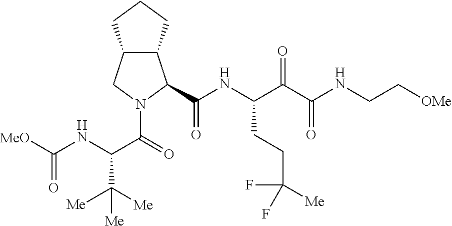 [SMILES: COCCNC(=O)C(=O)[C@H](CCC(C)(F)F)NC(=O)[C@@H]1[C@H]2CCC[C@H]2CN1C(=O)[C@@H](NC(=O)OC)C(C)(C)C] | methyl ((S)-1- ((1S,3aR,6aS)-1-(((S)-6,6- difluoro-1-((2- methoxyethyl)amino)-1,2- dioxoheptan-3- yl)carbamoyl)hexahydrocyc lopenta[c]pyrrol-2(1H)-yl)- 3,3-dimethyl-1-oxobutan-2- yl)carbamate | Calculated: 560.3 Found: 561.4 (M + H)⁺ |
| 103 | A | 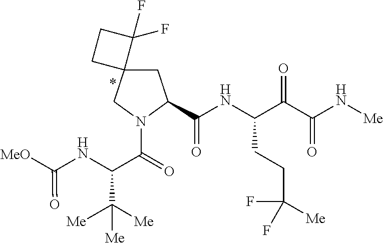 [SMILES: CNC(=O)C(=O)[C@H](CCC(C)(F)F)NC(=O)[C@@H]1CC2(CCC2(F)F)CN1C(=O)[C@@H](NC(=O)OC)C(C)(C)C] | methyl ((S)-1-((4(S or R),7S)-7-(((S)-6,6-difluoro- 1-(methylamino)-1,2- dioxoheptan-3- yl)carbamoyl)-1,1-difluoro- 6-azaspiro[3.4]octan-6-yl)- 3,3-dimethyl-1-oxobutan-2- yl)carbamat | Calculated: 552.3 Found: 553.1 (M + H)⁺ |
| 104 | A | 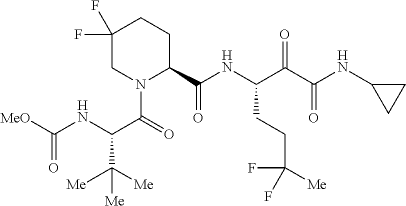 [SMILES: COC(=O)N[C@H](C(=O)N1CC(F)(F)CC[C@H]1C(=O)N[C@@H](CCC(C)(F)F)C(=O)C(=O)NC1CC1)C(C)(C)C] | methyl ((S)-1-((S)-2-(((S)- 1-(cyclopropylamino)-6,6- difluoro-1,2-dioxoheptan-3- yl)carbamoyl)-5,5- difluoropiperidin-1-yl)-3,3- dimethyl-1-oxobutan-2- yl)carbamate | Calculated: 552.3 Found: 553.3 (M + H)⁺ |
| 105 | A | 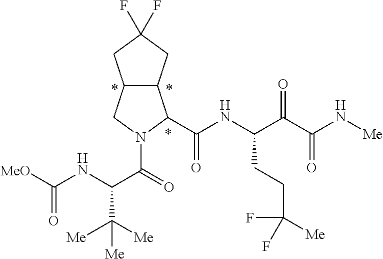 [SMILES: CNC(=O)C(=O)[C@H](CCC(C)(F)F)NC(=O)C1C2CC(F)(F)CC2CN1C(=O)[C@@H](NC(=O)OC)C(C)(C)C] | methyl ((S)-1-((1S,3aR,6aS or 1R,3aS,6aR or 1S,3aS,6aR or 1R,3aR,6aS)- 1-(((S)-6,6-difluoro-1- (methylamino)-1,2- dioxoheptan-3- yl)carbamoyl)-5,5- difluorohexahydrocyclopent a[c]pyrrol-2(1H)-yl)-3,3- dimethyl-1-oxobutan-2- yl)carbamate | Calculated: 552.3 Found: 553.1 (M + H)⁺ |
| 106 | A | 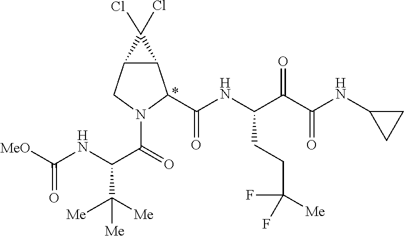 [SMILES: COC(=O)N[C@H](C(=O)N1C[C@H]2[C@@H](C1C(=O)N[C@@H](CCC(C)(F)F)C(=O)C(=O)NC1CC1)C2(Cl)Cl)C(C)(C)C] | methyl ((S)-1-((1S,2(S or R),5R)-6,6-dichloro-2-(((S)- 1-(cyclopropylamino)-6,6- difluoro-1,2-dioxoheptan-3- yl)carbamoyl)-3- azabicyclo[3.1.0]hexan-3- yl)-3,3-dimethyl-1- oxobutan-2-yl)carbamate | Calculated: 582.2 Found: 583.2 (M + H)⁺ |
| 107 | A | 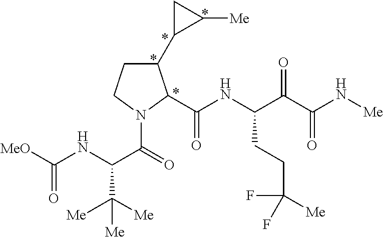 [SMILES: CNC(=O)C(=O)[C@H](CCC(C)(F)F)NC(=O)C1C(C2CC2C)CCN1C(=O)[C@@H](NC(=O)OC)C(C)(C)C] | methyl ((S)-1-((2(S or R),3(S or R))-2-(((S)-6,6- difluoro-1-(methylamino)- 1,2-dioxoheptan-3- yl)carbamoyl)-3-((1(S or R),2(R or S))-2- methylcyclopropyl)pyrrolidi n-1-yl)-3,3-dimethyl-1- oxobutan-2-yl)carbamate | Calculated: 530.3 Found: 531.3 (M + H)⁺ |
| 108 | A | 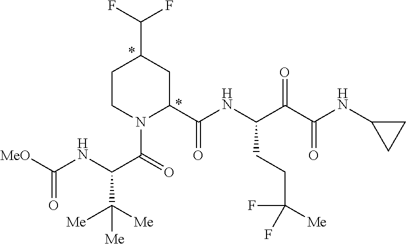 [SMILES: COC(=O)N[C@H](C(=O)N1CCC(C(F)F)CC1C(=O)N[C@@H](CCC(C)(F)F)C(=O)C(=O)NC1CC1)C(C)(C)C] | methyl ((S)-1-((2(S or R),4(R or S)-2-(((S)-1- (cyclopropylamino)-6,6- difluoro-1,2-dioxoheptan-3- yl)carbamoyl)-4- (difluoromethyl)piperidin-1- yl)-3,3-dimethyl-1- oxobutan-2-yl)carbamate | Calculated: 566.3 Found: 567.2 (M + H)⁺ |
| 109 | A | 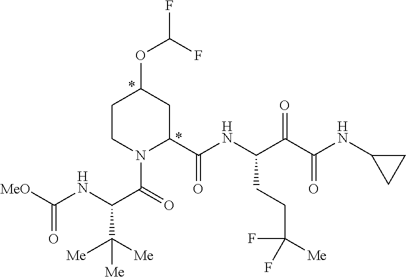 [SMILES: COC(=O)N[C@H](C(=O)N1CCC(OC(F)F)CC1C(=O)N[C@@H](CCC(C)(F)F)C(=O)C(=O)NC1CC1)C(C)(C)C] | methyl ((S)-1-((2R,4R or 2S,4S)-2-(((S)-1- (cyclopropylamino)-6,6- difluoro-1,2-dioxoheptan-3- yl)carbamoyl)-4- (difluoromethoxy)piperidin- 1-yl)-3,3-dimethyl-1- oxobutan-2-yl)carbamate | Calculated: 582.3 Found: 349.2 (M- C₁₀H₁₅F₂N₂ O₂)⁺ |
| 110 | A | 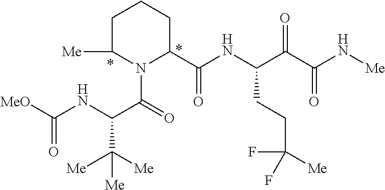 [SMILES: CNC(=O)C(=O)[C@H](CCC(C)(F)F)NC(=O)C1CCCC(C)N1C(=O)[C@@H](NC(=O)OC)C(C)(C)C] | methyl ((S)-1-((2S,6S or 2R,6R)-2-(((S)-6,6-difluoro- 1-(methylamino)-1,2- dioxoheptan-3- yl)carbamoyl)-6- methylpiperidin-1-yl)-3,3- dimethyl-1-oxobutan-2- yl)carbamate | Calculated: 504.3 Found: 505.2 M + H |
| 111 | A | 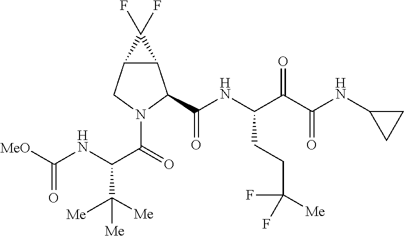 [SMILES: COC(=O)N[C@H](C(=O)N1C[C@H]2[C@@H]([C@H]1C(=O)N[C@@H](CCC(C)(F)F)C(=O)C(=O)NC1CC1)C2(F)F)C(C)(C)C] | methyl ((S)-1-((1S,2S,5R)- 2-(((S)-1- (cyclopropylamino)-6,6- difluoro-1,2-dioxoheptan-3- yl)carbamoyl)-6,6-difluoro- 3-azabicyclo[3.1.0]hexan-3- yl)-3,3-dimethyl-1- oxobutan-2-yl)carbamate | Calculated: 550.2 Found: 551.3 M + H |
| 112 | A | 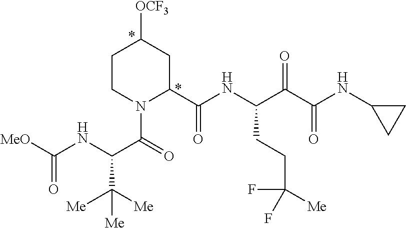 [SMILES: COC(=O)N[C@H](C(=O)N1CCC(OC(F)(F)F)CC1C(=O)N[C@@H](CCC(C)(F)F)C(=O)C(=O)NC1CC1)C(C)(C)C] | methyl ((S)-1-((2S,4S or 2R,4R)-2-(((S)-1- (cyclopropylamino)-6,6- difluoro-1,2-dioxoheptan-3- yl)carbamoyl)-4- (trifluoromethoxy)piperidin- 1-yl)-3,3-dimethyl-1- oxobutan-2-yl)carbamate | Calculated: 600.3 Found: 601.2 M + H |
| 113 | A | 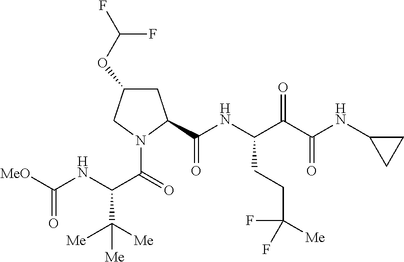 [SMILES: COC(=O)N[C@H](C(=O)N1C[C@H](OC(F)F)C[C@H]1C(=O)N[C@@H](CCC(C)(F)F)C(=O)C(=O)NC1CC1)C(C)(C)C] | methyl ((S)-1-((2S,4R)-2- (((S)-1-(cyclopropylamino)- 6,6-difluoro-1,2- dioxoheptan-3- yl)carbamoyl)-4- (difluoromethoxy)pyrrolidin -1-yl)-3,3-dimethyl-1- oxobutan-2-yl)carbamate | Calculated: 568.3 Found: 569.2 M + H |
| 114 | A | 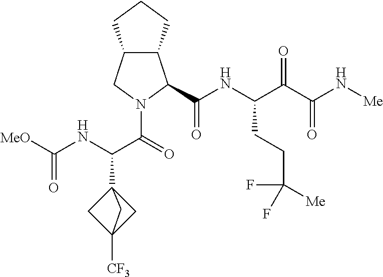 [SMILES: CNC(=O)C(=O)[C@H](CCC(C)(F)F)NC(=O)[C@@H]1[C@H]2CCC[C@H]2CN1C(=O)[C@@H](NC(=O)OC)C12CC(C(F)(F)F)(C1)C2] | methyl ((S)-2- ((1S,3aR,6aS)-1-(((S)-6,6- difluoro-1-(methylamino)- 1,2-dioxoheptan-3- yl)carbamoyl)hexahydrocyc lopenta[c]pyrrol-2(1H)-yl)- 2-oxo-1-(3- (trifluoromethyl)bicyclo[1.1 1]pentan-1- yl)ethyl)carbamate | Calculated: 594.3 Found: 595.3 M + H |
| 115 | A | 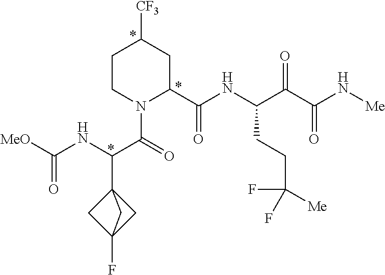 [SMILES: CNC(=O)C(=O)[C@H](CCC(C)(F)F)NC(=O)C1CC(C(F)(F)F)CCN1C(=O)C(NC(=O)OC)C12CC(F)(C1)C2] | methyl ((R or S)-2-((2(S or R),4(R or S))-2-(((S)-6,6- difluoro-1-(methylamino)- 1,2-dioxoheptan-3- yl)carbamoyl)-4- (trifluoromethyl)piperidin- 1-yl)-1 -(3- fluorobicyclo[1.1.1]pentan- 1-yl)-2-oxoethyl)carbamate | Calculated: 586.2 Found: 587.2 M + H |
| 116 | B | 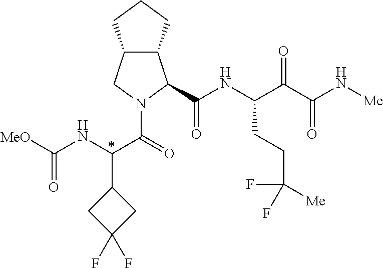 [SMILES: CNC(=O)C(=O)[C@H](CCC(C)(F)F)NC(=O)[C@@H]1[C@H]2CCC[C@H]2CN1C(=O)C(NC(=O)OC)C1CC(F)(F)C1] | methyl ((S or R)-2- ((1S,3aR,6aS)-1-(((S)-6,6- difluoro-1-(methylamino)- 1,2-dioxoheptan-3- yl)carbamoyl)hexahydrocyc lopenta[c]pyrrol-2(1H)-yl)- 1-(3,3-difluorocyclobutyl)- 2-oxoethyl)carbamate | Calculated: 550.2 Found: 551.2 M + H |
| 117 | B | 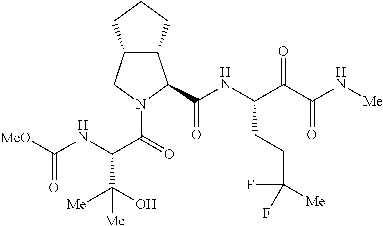 [SMILES: CNC(=O)C(=O)[C@H](CCC(C)(F)F)NC(=O)[C@@H]1[C@H]2CCC[C@H]2CN1C(=O)[C@@H](NC(=O)OC)C(C)(C)O] | methyl ((S)-1- ((1S,3aR,6aS)-1-(((S)-6,6- difluoro-1-(methylamino)- 1,2-dioxoheptan-3- yl)carbamoyl)hexahydrocyc lopenta[c]pyrrol-2(1H)-yl)- 3-hydroxy-3-methyl-1- oxobutan-2-yl)carbamate | Calculated: 518.3 Found: 519.2 M + H |
| 118 | A | 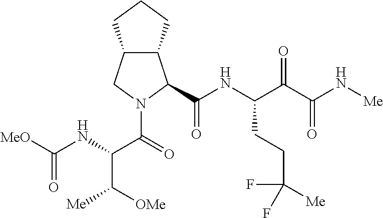 [SMILES: CNC(=O)C(=O)[C@H](CCC(C)(F)F)NC(=O)[C@@H]1[C@H]2CCC[C@H]2CN1C(=O)[C@@H](NC(=O)OC)[C@@H](C)OC] | methyl ((2S,3R)-1- ((1S,3aR,6aS)-1-(((S)-1- (cyclopropylamino)-6,6- difluoro-1,2-dioxoheptan-3- yl)carbamoyl)hexahydrocyc lopenta[c]pyrrol-2(1H)-yl)- 3-methoxy-1-oxobutan-2- yl)carbamate | Calculated: 544.3 Found: 545.2 M + H |
| 119 | A | 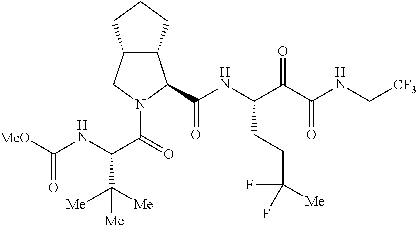 [SMILES: COC(=O)N[C@H](C(=O)N1C[C@@H]2CCC[C@@H]2[C@H]1C(=O)N[C@@H](CCC(C)(F)F)C(=O)C(=O)NCC(F)(F)F)C(C)(C)C] | methyl ((S)-1- ((1S,3aR,6aS)-1-(((S)-6,6- difluoro-1,2-dioxo-1- ((2,2,2- trifluoroethyl)amino)heptan -3- yl)carbamoyl)hexahydrocyc lopenta[c]pyrrol-2(1H)-yl)- 3,3-dimethyl-1-oxobutan-2- yl)carbamate | Calculated: 584.2 Found: 585.3 M + H |
| 120 | A | 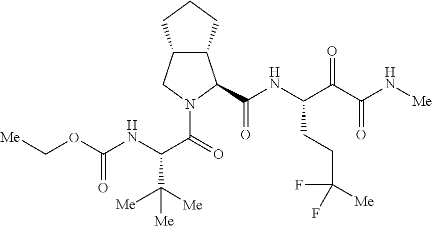 [SMILES: CCOC(=O)N[C@H](C(=O)N1C[C@@H]2CCC[C@@H]2[C@H]1C(=O)N[C@@H](CCC(C)(F)F)C(=O)C(=O)NC)C(C)(C)C] | ethyl ((S)-1-((1S,3aR,6aS)- 1-(((S)-6,6-difluoro-1- (methylamino)-1,2- dioxoheptan-3- yl)carbamoyl)hexahydrocyc lopenta[c]pyrrol-2(1H)-yl)- 3,3-dimethyl-1-oxobutan-2- yl)carbamate | Calculated: 530.3 Found: 531.3 M + H |
| 121 | A | 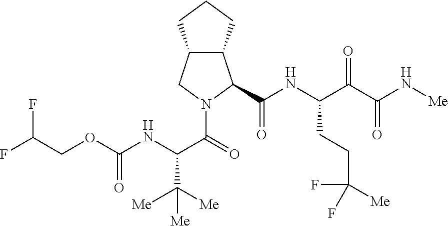 [SMILES: CNC(=O)C(=O)[C@H](CCC(C)(F)F)NC(=O)[C@@H]1[C@H]2CCC[C@H]2CN1C(=O)[C@@H](NC(=O)OCC(F)F)C(C)(C)C] | 2,2-difluoroethyl ((S)-1- ((1S,3aR,6aS)-1-(((S)-6,6- difluoro-1-(methylamino)- 1,2-dioxoheptan-3- yl)carbamoyl)hexahydrocyc lopenta[c]pyrrol-2(1H)-yl)- 3,3-dimethyl-1-oxobutan-2- yl)carbamate | Calculated: 566.3 Found: 567.2 M + H |
| 122 | B | 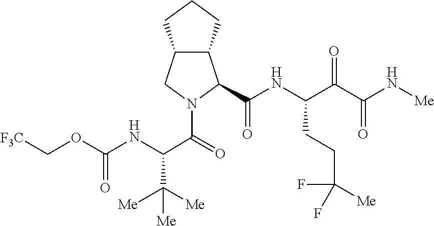 [SMILES: CNC(=O)C(=O)[C@H](CCC(C)(F)F)NC(=O)[C@@H]1[C@H]2CCC[C@H]2CN1C(=O)[C@@H](NC(=O)OCC(F)(F)F)C(C)(C)C] | 2,2,2-trifluoroethyl ((S)-1- ((1S,3aR,6aS)-1-(((S)-6,6- difluoro-1 -(methylamino)- 1,2-dioxoheptan-3- yl)carbamoyl)hexahydrocyc lopenta[c]pyrrol-2(1H)-yl)- 3,3-dimethyl-1-oxobutan-2- yl)carbamate | Calculated: 584.3 Found: 585.3 M + H |
| 123 | B | 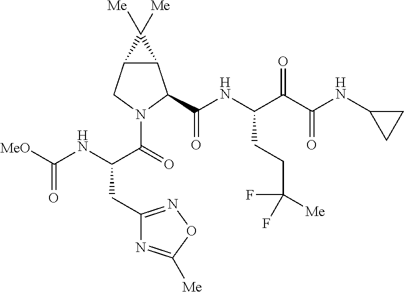 [SMILES: COC(=O)N[C@@H](Cc1noc(C)n1)C(=O)N1C[C@H]2[C@@H]([C@H]1C(=O)N[C@@H](CCC(C)(F)F)C(=O)C(=O)NC1CC1)C2(C)C] | methyl ((S)-1-((1R,2S,5S)- 2-(((S)-1- (cyclopropylamino)-6,6- difluoro-1,2-dioxoheptan-3- yl)carbamoyl)-6,6-dimethyl- 3-azabicyclo[3.1.0]hexan-3- yl)-3-(5-methyl-1,2,4- oxadiazol-3-yl)-1- oxopropan-2-yl)carbamate | Calculated: 582.3 Found: 583.4 M + H |

### SARS2 Coronavirus 3CL Protease Assay

The enzymatic activity of SARS2 coronavirus 3CL protease was determined in a FRET (fluorescence resonance energy transfer)-based assay measuring the cleavage of a peptide substrate by recombinantly expressed and purified enzyme. Cleavage of the peptide SEQ ID NO:1 (CPC Scientific) by SARS2 3CL protease was measured in reaction buffer (50 mM Hepes pH 7.5, 0.01% Triton X-100, 0.01% BSA, 2 mM DTT). SARS2 3CL protease (5 nM final concentration) was pre-incubated with compound for 30 minutes before reaction initiation with peptide substrate (15 uM final concentration). Room temperature reactions (4 h) were quenched by addition of a high dose of inhibitor and read on an appropriate plate reader (excitation wavelength=495 nm, emission wavelength=520 nm). Data were analyzed by a standard 4 parameter fit to determine IC₅₀ values.

The compounds of the instant invention were tested in the assay described above and the results appear in the table below.

TABLE I
IC₅₀ values (nM) for Examples in the SARS2 3CLPro FRET assay

| Example | |
|---|---|
| No. | IC₅₀ (nM) |
| 1 | 28 |
| 2 | 29 |
| 3 | 58 |
| 4 | 60 |
| 5 | 81 |
| 6 | 17 |
| 7 | 11 |
| 8 | 18 |
| 9 | 21 |
| 10 | 26 |
| 11 | 40 |
| 12 | 41 |
| 13 | 49 |
| 14 | 69 |
| 15 | 113 |
| 16 | 136 |
| 17 | 137 |
| 18 | 42 |
| 19 | 497 |
| 20 | 7.4 |
| 21 | 6.5 |
| 22 | 10 |
| 23 | 10 |
| 24 | 8.2 |
| 25 | 8.7 |
| 26 | 22 |
| 27 | 14 |
| 28 | 297 |
| 29 | 7.7 |
| 30 | 40 |
| 31 | 10 |
| 32 | 15 |
| 33 | 9.7 |
| 34 | 7.4 |
| 35 | 8.2 |
| 36 | 8.2 |
| 37 | 9.2 |
| 38 | 9.7 |
| 39 | 12 |
| 40 | 12 |
| 41 | 13 |
| 42 | 22 |
| 43 | 22 |
| 44 | 27 |
| 45 | 34 |
| 46 | 34 |
| 47 | 38 |
| 48 | 45 |
| 49 | 60 |
| 50 | 62 |
| 51 | 97 |
| 52 | 144 |
| 53 | 196 |
| 54 | 201 |
| 55 | 239 |
| 56 | 291 |
| 57 | 305 |
| 58 | 347 |
| 59 | 37 |
| 60 | 19 |
| 61 | 16 |
| 62 | 8.8 |
| 63 | 32 |
| 64 | 30 |
| 65 | 24 |
| 66 | 9.9 |
| 67 | 20 |
| 68 | 28 |
| 69 | 31 |
| 70 | 36 |
| 71 | 43 |
| 72 | 48 |
| 73 | 60 |
| 74 | 69 |
| 75 | 82 |
| 76 | 87 |
| 77 | 133 |
| 78 | 12 |
| 79 | 30 |
| 80 | 12 |
| 81 | 44 |
| 82 | 51 |
| 83 | 83 |
| 84 | 96 |
| 85 | 257 |
| 86 | 20 |
| 87 | 7 |
| 88 | 21 |
| 89 | 13 |
| 90 | 16 |
| 91 | 19 |
| 92 | 29 |
| 93 | 28 |
| 94 | 9 |
| 95 | 5 |
| 96 | 15 |
| 97 | 19 |
| 98 | 77 |
| 99 | 100 |
| 100 | 14 |
| 101 | 22 |
| 102 | 61 |
| 103 | 32 |
| 104 | 740 |
| 105 | 11 |
| 106 | 8 |
| 107 | 14 |
| 108 | 22 |
| 109 | 50 |
| 110 | 61 |
| 111 | 88 |
| 112 | 146 |
| 113 | 46 |
| 114 | 27 |
| 115 | 35 |
| 116 | 16 |
| 117 | 14 |
| 118 | 14 |
| 119 | 77 |
| 120 | 21 |
| 121 | 15 |
| 122 | 34 |
| 123 | 29 |

### Sequences

ESATLQSGLRKAKNH2 having a CPQ2 chromophore on the terminal lysine residue (SEQ ID NO:1)

1. A compound of Formula I 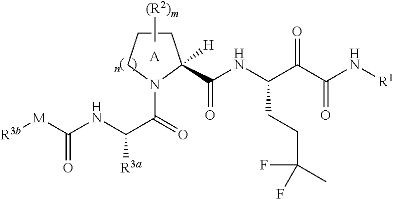
or a pharmaceutically acceptable salt thereof, wherein:
R¹ is
(a) C₁-C₆ alkyl,
(b) C₁-C₆ alkoxy,
(c) C₁-C₆ fluoroalkyl,
(d) —(CH₂)ₚ—R^{1c}, wherein R^{1c} is:
(i) C₃-C₆ cycloalkyl;
(ii) a 5- to 6-membered saturated heterocycloalkyl containing 1 to 2 heteroatoms independently selected from N, O, or S;
(iii) a 5- to 6-membered heteroaryl containing 1 to 3 heteroatoms independently selected from N, O, or S; or
(iv) phenyl;
wherein R^{1c} is unsubstituted or substituted by halo, C₁-C₃ alkyl, C₁-C₃ fluoroalkyl, —O—C₁-C₃ alkyl, —O—C₁-C₃ fluoroalkyl, or —C₃-C₄ cycloalkyl; or
(e) H;
each R² is independently fluoro, hydroxy, C₁-C₃ alkyl, C₁-C₃ fluoroalkyl, —O—C₁-C₃ alkyl, —O—C₁-C₃ fluoroalkyl, or ring R^{2cy},
wherein ring R^{2cy} is cyclopropyl, cyclobutyl, cyclopentyl, oxetanyl, tetrahydrofuryl, or bicyclo[1.1.1]pentyl;
wherein ring R^{2cy} is unsubstituted or substituted by 1 to 2 R^{2ca} substituents independently selected from fluoro, chloro, hydroxy, C₁-C₃ alkyl, C₁-C₃ fluoroalkyl, —O—C₁-C₃ alkyl, or —O—C₁-C₃ fluoroalkyl;
or alternatively, any two R², together with the carbon atom(s) to which they are attached form ring A′ to form a bicyclic ring system with illustrated ring A;
wherein ring A′ is a 3- to 6-membered cycloalkyl, tetrahydrofuran or tetrahydropyranyl ring;
wherein ring A′ is unsubstituted or substituted by 1 to 4 R^{a1} substituents independently selected from the group consisting of fluoro, hydroxy, C₁-C₃ alkyl, C₁-C₃ fluoroalkyl, —O—C₁-C₃ alkyl, or —O—C₁-C₃ fluoroalkyl;
or alternatively, two R^{a1} substituents, when substituted on a common carbon atom, together with the carbon atom to which they are attached, form ring A″;
wherein ring A″ is a 3- to 6-membered cycloalkyl, tetrahydrofuran or tetrahydropyranyl ring;
R^{3a} is
(a) C₁-C₆ alkyl,
(b) C₁-C₆ methoxy,
(c) C₁-C₃ fluoroalkyl,
(d) —CH₂O—(C₁-C₆ alkyl),
(e) a group of the formula 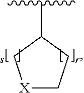
wherein X is —CH₂—, —CF₂— or —O—;
(f) a group of the formula —(CH₂)ₜ—Y^{3c}
wherein Y^{3c} is phenyl or a 5- to 6-membered heteroaryl containing 1 to 3 heteroatoms independently selected from N, O, or S,
wherein Y^{3c} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl;
M is —O— or —N(H)—;
R^{3b} is
(a) C₁-C₆ alkyl, or
(b) a group of the formula —(CH₂)ᵤ—Y^{3b}
wherein Y^{3b} is:
(i) phenyl;
(ii) C₃-C₆ cycloalkyl;
(iii) a 5- to 6-membered saturated heterocycloalkyl containing 1 to 2 heteroatoms independently selected from N, O, or S; or
(iv) a 5- to 6-membered heteroaryl containing 1 to 3 heteroatoms independently selected from N, O, or S;
wherein Y^{3b} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl;
subscript m is 0, 1, 2 or 3;
subscript n is 1 or 2;
subscript p is 0, 1, or 2;
subscript r is 0, 1, or 2;
subscript s is 0, 1, or 2;
subscript t is 0 or 1; and
subscript u is 0 or 1.

2. The compound of claim 1 or a pharmaceutically acceptable salt thereof, wherein the group 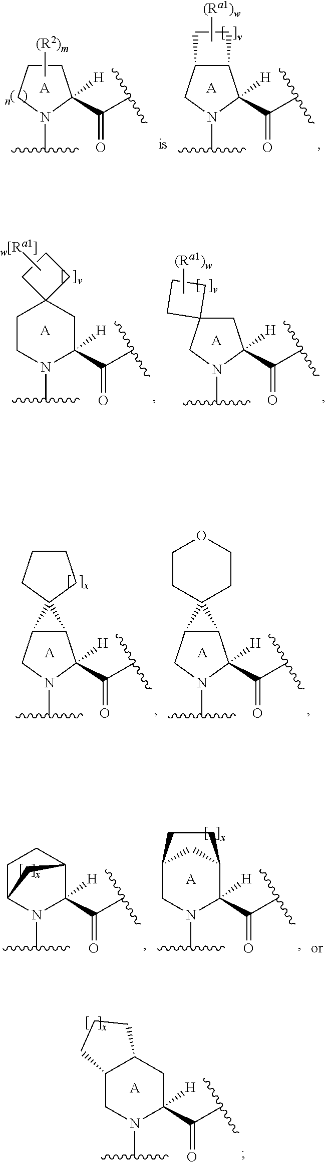
subscript v is 0, 1, 2, or 3;
subscript w is 0, 1, 2, 3, or 4; and
subscript x is 1 or 2.

3. The compound of claim 1 or a pharmaceutically acceptable salt thereof, wherein the group

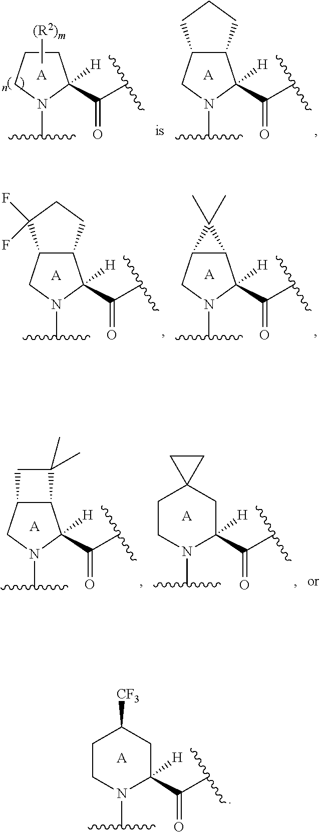

4. The compound of claim 1 or a pharmaceutically acceptable salt thereof, wherein R¹ is C₁-C₄ alkyl or C₃-C₆ cycloalkyl.

5. The compound of claim 1 or a pharmaceutically acceptable salt thereof, wherein R¹ is methyl or cyclopropyl.

6. The compound of claim 1 or a pharmaceutically acceptable salt thereof, wherein R^{3a} is t-butyl.

7. The compound of claim 1 or a pharmaceutically acceptable salt thereof, wherein M is
—O—.

8. The compound of claim 1 or a pharmaceutically acceptable salt thereof, wherein R^{3b} is
(a) C₁-C₆ alkyl, or
(b) a group of the formula —(CH₂)ᵤ—Y^{3b}
wherein Y^{3b} is phenyl or C₃-C₆ cycloalkyl,
wherein Y^{3b} is unsubstituted or substituted by 1 to 3 substituents independently selected from fluoro, hydroxy, C₁-C₃ alkyl, C₁-C₃ fluoroalkyl, —O—C₁-C₃ alkyl, or —O—C₁-C₃ fluoroalkyl.

9. The compound of claim 8 or a pharmaceutically acceptable salt thereof, wherein R^{3b} is C₁-C₆ alkyl.

10. The compound of claim 9 or a pharmaceutically acceptable salt thereof, wherein R^{3b} is methyl.

11. The compound of claim 1 or a pharmaceutically acceptable salt thereof, wherein:
the group 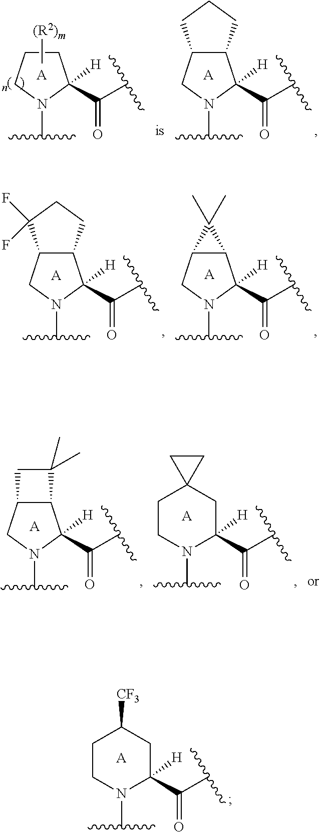
R¹ is methyl or cyclopropyl;
R^{3a} is t-butyl;
M is —O—; and
R^{3b} is methyl.

12. The compound of claim 1 or a pharmaceutically acceptable salt thereof, wherein the compound is selected from any one of Examples 1-123.

13. A pharmaceutical composition comprising the compound of claim 1 or a pharmaceutically acceptable salt thereof and a pharmaceutically acceptable carrier.

14. The pharmaceutical composition of claim 13 in the form of an orally administered tablet or capsule.

15. The pharmaceutical composition of claim 13, further comprising a second therapeutic agent.

16. A method for prophylaxis or treatment of a coronavirus infection, comprising administering an effective amount of the compound of claim 1 or a pharmaceutically acceptable salt thereof to a patient in need thereof.

17. The method of claim 16, wherein the coronavirus infection is a SARS-CoV, SARS-CoV-2 or MERS-CoV infection.

18. The method of claim 17, wherein the coronavirus infection is a SARS-CoV-2 infection.

19. The method of claim 16, further comprising administering a second therapeutic agent to the patient.

20. (canceled)

21. (canceled)

22. (canceled)


SEQUENCE LISTING (1 sequence)

SEQ ID NO: 1 - protein, 13 aa, synthetic construct
  SITE 13: note = lysine-CPQ2 chromophore-amide
ESATLQSGLRKAK